(19) United States
(12) Patent Application Publication
(10) Pub. No.: US 2026/0021161 A1
(43) Pub. Date: Jan. 22, 2026

(54) SILK PEPTIDES TOPICAL FORMULATIONS AND METHODS OF THEIR USE

(71) Applicant: SILKLYFE INC., Palm Beach, FL (US)

(72) Inventors: Mike SANTOS, Fairhaven, MA (US); Charles KOPPEL, London (GB)

(21) Appl. No.: 19/339,974

(22) Filed: Sep. 25, 2025

Related U.S. Application Data
(63) Continuation of application No. 19/094,004, filed on Mar. 28, 2025, now abandoned.
(60) Provisional application No. 63/571,619, filed on Mar. 29, 2024.

(51) Int. Cl. A61K 38/17; A61K 8/11; A61P 17/00; A61P 17/02; A61P 17/06; A61P 17/10; A61P 31/14; A61Q 19/02; A61Q 19/08

(52) U.S. Cl. CPC A61K 38/1767; A61K 8/11; A61P 17/00; A61P 17/02; A61P 17/06; A61P 17/10; A61P 31/14; A61Q 19/02; A61Q 19/08

(57) ABSTRACT

A composition comprising fragments of silk fibroin peptides for use in the treatment of a disorder or condition associated with a skin disorder. The composition may be also used prophylactically. Several embodiments are proposed in which the composition may be a topical composition in varying forms such as a hydrogel, powder, suspension, emulsion, foam, film, solution, or the like. The composition can be modified to include additional agents for use in the treatment of various skin disorders.

### CROSS-REFERENCE TO RELATED APPLICATIONS

This application is a Continuation of U.S. application Ser. No. 19/094,004, filed on Mar. 28, 2025, which claims benefit of provisional Application No. 63/571,619, filed on Mar. 29, 2024. The entire contents of the above-identified applications are hereby fully incorporated herein by reference.

### BACKGROUND OF THE DISCLOSURE

Silk is a naturally occurring polymer. Most silk fibers are derived from silkworm moth (Bombyx mori) cocoons and include silk fibroin and sericin proteins. Silk fibroin is a fibrous material that forms a polymeric matrix bonded together with sericin. In nature, silk is formed from a concentrated solution of these proteins that are extruded through silkworm spinnerets to produce a highly insoluble fiber. These fibers have been used for centuries to form threads used in garments and other textiles. Humans have long appreciated silk for its lustrous appeal and remarkable physical properties, yet as modern uses of silk are explored, it becomes apparent that this outstanding biopolymer is more than textile fiber, but additionally a biopolymer having medicinal properties.

Many properties of silk make it an attractive candidate for products serving a variety of industries. Polymer strength and flexibility has supported classical uses of silk in textiles and materials, while silk biocompatibility has gained attention more recently for applications in the fields of medicine and agriculture.

Although a variety of products and uses related to silk are being developed, there remains a need for methods of producing and processing silk and silk-based products that can meet the demands of modern medicine. Additionally, there remains a need for silk-based products that can leverage silk polymer strength, flexibility, and biocompatibility to meet needs in the fields of medicine, agriculture, and material sciences. The present disclosure addresses these needs by providing methods for producing and processing silk as well as formulations of silk-based products useful in a variety of therapeutic applications. Silk in its solubilized aqueous form has been investigated for a range of therapeutic applications, including treatment of diabetes, See. Enzyme Hydrolysis of Silk Fibroin and the Anti-diabetic Activity of the Hydrolysates (C. L. Hu, J. Y Cui, F. Z. Ren, C. Peng, Int. J. Food Eng. 2008); chronic wounds, See. Fibroin and Sericin from Bombyx mori Silk Stimulate Cell Migration through Upregulation and Phosphorylation of c-Jun (C. Martinez-Mora, A. Mrowiec, E. M. Garcia-Vizcaino, A. Alcaraz, J. L. Cenis, F. J. Nicolas, PLoS One 2012, 7, e42271) and inflammation See. Enhancement of Anti-Inflammatory Activity of PEP-1-FK506 Binding Protein by Silk Fibroin Peptide (D. W. Kim, H. S. Hwang, D. S. Kim, S. H. Sheen, D. H. Heo, G. Hwang, S. H. Kang, H. Kweon, Y Y Jo, S. W. Kang, K. G. Lee, J. Park, W. S. Eum, Y J. Cho, S. Y Choi, J. Microb. Biotechnol. 2012, 22, 494). Additional studies have investigated the utility of regenerated silk fibroin solution in preclinical animal models for the treatment of ocular conditions, including dry eye, See. Effects of silk fibroin in murine dry eye (C. E. Kim, J. H. Lee, Y K. Yeon, C. H. Park, J. Yang, Sci. Rep. 2017, 7, 44364). While silk has shown potential in a variety of therapeutics indications, its anti-inflammatory properties indicate a potential for a variety of topical conditions that are inflammatory in nature. Additionally, its regenerative effect on skin and hair follicles provides potential for skin rejuvenation and hair regrowth.

### SUMMARY OF THE DISCLOSURE

In some embodiments, the present disclosure provides silk-based product (SBP) formulations that comprise processed silk fibroin peptides with optionally one or more excipients, wherein the processed silk fibroin peptides comprise or are derived from natural or synthetic sources and combinations thereof. The SBP formulation may comprises or may be combined with one or more members selected from the group consisting of: (a) a therapeutic agent; (b) a cargo; (c) a microorganism; and (d) a biological system. The processed silk fibroin peptides and/or other SBP component (excipient, therapeutic agent, microbe, cargo, and/or biological system) may be present in SBP formulations at a concentration (by weight, volume, or concentration) of from about 0.0001% to about to about 99.9%, or greater than 99.9%.

The processed silk fibroin peptides and/or other SBP components include formulations of various silk-based product formulations and related methods of preparation and processing for uses in fields of human therapeutics and veterinary medicine as set forth in WO 2020/247594 entitled: Silk-Based Products, Formulations, and Methods of Use and US 2024/0300998 entitled: Methods for reducing impurities in silk fibroin preparations, the contents of which are incorporated in their entirety. Additional formulations and methods or their preparation of SBP formulations are set forth in U.S. Pat. No. 11,633,455, the contents of which are also incorporated in their entirety.

The SBP formulation may have processed silk fibroin peptides and/or other SBP components (excipient, therapeutic agent, microbe, cargo, and/or biological system) present at a concentration of from about 0.01 pg/mL to about 200 mg/mL.

The SBP formulation may have processed silk fibroin peptides alone and/or other SBP components (excipient, therapeutic agent, microbe, cargo, and/or biological system) present in SBPs at a concentration of from about 0.01 pg/kg to about 10 mg/kg per kg of patient.

The processed silk fibroin peptides of the SBP formulation may comprise silk fibroin at a concentration between 0.1% and 100%. In one illustrative embodiment, the silk fibroin is present at a concentration of 0.5%. In one aspect, the silk fibroin is present at a concentration of 1%. In one aspect, the silk fibroin is present at a concentration of 2.5%. In one aspect, the silk fibroin is present at a concentration of 3%. In one aspect, the silk fibroin is present at a concentration of 5%.

The SBP formulation may be in powder form or in a solution which may be, but is not limited to, phosphate buffer, borate buffer, and phosphate buffered saline. The solution may further comprise propylene glycol, sucrose and/or trehalose. Propylene glycol may be present in a concentration of about 1%. Sucrose may be present in a concentration such as, but not limited to, 10 mM, 50 mM, 100 mM and 150 mM. Trehalose may be present in a concentration such as, but not limited to, 10 mM, 50 mM, 100 mM and 150 mM.

The SBP may be formulated, and the formulation may be as hydrogels, powders, suspensions, emulsions, and solutions. The silk fibroin concentration in the solution may be below 1% (w/v). The SBP may be a solution, and the SBP may be stressed. The SBP may be a hydrogel, and the SBP may be stressed. The SBP may be a solution, and the solution may shear thin. The solutions may have the viscosity of a gel at a lower shear rate. The solutions may have the viscosity of a fluid at higher shear rates. The SBP may be formulated for topical administration.

In some embodiments, the present disclosure provides a method of preparing the SBP formulations comprising: (a) preparing the processed silk fibroin peptides, wherein the processed silk fibroin peptides comprise or are derived from natural or synthetic sources; and (b) preparing the SBP formulation using the processed silk fibroin peptides. In some embodiments, the present disclosure provides a method of treating inflammation. In other embodiments, the present disclosure provides a method and composition for hair regrowth and maintenance. In further embodiments, the present disclosure provides a method and composition for rejuvenation and maintenance of skin and hair health.

The SBP may be administered topically in the form of creams, ointments, pastes, plasters, oil in water suspensions, solutions, hydrogels, transdermal patches and buffered solutions.

Additionally other active compounds or active pharmaceutical ingredients could be added to the formulation. It is further completed within the scope of the disclosure that GRAS excipients or nanoparticulate formulations could be used to help the silk peptides penetrate and target the dermal layers and hair follicles. It's also contemplated within the scope of the disclosure that active ingredients, ayurvedics can be co-administered producing a combinatorial or synergistic effect.

In a further aspect of the disclosure, the silk fibroin peptides may be delivered via a shampoo bar and/or a face and body bar according to the formulations described herein.

In a further aspect of the disclosure, surfactants, penetration enhancers, polymeric and solid, semi-solid, or gel nanoparticle formulations to prevent silk peptide fragment degradation and/or the degraded silk fibroin peptides/amino acids to have a similar anti-inflammatory effect.

The SBP formulation may have processed silk fibroin peptides and/or other SBP components (excipient, therapeutic agent, microbe, cargo, and/or biological system) present at a concentration of from about 0.01 pg/mL to about 1 μg/mL, from about 0.05 pg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 pg/mL, from about 16 μg/mL to about 36 μg/mL, from about 18 μg/mL to about 38 μg/mL, from about 20 μg/mL to about 40 μg/mL, from about 22 pg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 ng/mL to about 1 ng/mL, from about 0.05 ng/mL to about 2 ng/mL, from about 1 ng/mL to about 5 ng/mL, from about 2 ng/mL to about 10 ng/mL, from about 4 ng/mL to about 16 ng/mL, from about 5 ng/mL to about 20 ng/mL, from about 8 ng/mL to about 24 ng/mL, from about 10 ng/mL to about 30 ng/mL, from about 12 ng/mL to about 32 ng/mL, from about 14 ng/mL to about 34 ng/mL, from about 16 ng/mL to about 36 ng/mL, from about 18 ng/mL to about 38 ng/mL, from about 20 ng/mL to about 40 ng/mL, from about 22 ng/mL to about 42 ng/mL, from about 24 ng/mL to about 44 ng/mL, from about 26 ng/mL to about 46 ng/mL, from about 28 ng/mL to about 48 ng/mL, from about 30 ng/mL to about 50 ng/mL, from about 35 ng/mL to about 55 ng/mL, from about 40 ng/mL to about 60 ng/mL, from about 45 ng/mL to about 65 ng/mL, from about 50 ng/mL to about 75 ng/mL, from about 60 ng/mL to about 240 ng/mL, from about 70 ng/mL to about 350 ng/mL, from about 80 ng/mL to about 400 ng/mL, from about 90 ng/mL to about 450 ng/mL, from about 100 ng/mL to about 500 ng/mL, from about 0.01 g/mL to about 1 μg/mL, from about 0.05 pg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 μg/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 g/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 65 mg/mL, from about 50 mg/mL to about 75 mg/mL, from about 60 mg/mL to about 240 mg/mL, from about 70 mg/mL to about 350 mg/mL, from about 80 mg/mL to about 400 mg/mL, from about 90 mg/mL to about 450 mg/mL, from about 100 mg/mL to about 500 mg/mL, from about 0.01 g/mL to about 1 g/mL, from about 0.05 g/mL to about 2 g/mL, from about 1 g/mL to about 5 g/mL, from about 2 g/mL to about 10 g/mL, from about 4 g/mL to about 16 g/mL, or from about 5 g/mL to about 20 g/mL.

The SBP formulation may have processed silk fibroin peptides and/or other SBP components (excipient, therapeutic agent, microbe, cargo, and/or biological system) present in SBPs at a concentration of from about 0.01 pg/kg to about 1 μg/kg, from about 0.05 pg/kg to about 2 μg/kg, from about 1 μg/kg to about 5 μg/kg, from about 2 μg/kg to about 10 μg/kg, from about 4 μg/kg to about 16 μg/kg, from about 5 μg/kg to about 20 μg/kg, from about 8 μg/kg to about 24 μg/kg, from about 10 μg/kg to about 30 μg/kg, from about 12 μg/kg to about 32 μg/kg, from about 14 μg/kg to about 34 μg/kg, from about 16 μg/kg to about 36 μg/kg, from about 18 μg/kg to about 38 μg/kg, from about 20 μg/kg to about 40 μg/kg, from about 22 μg/kg to about 42 μg/kg, from about 24 μg/kg to about 44 μg/kg, from about 26 μg/kg to about 46 μg/kg, from about 28 μg/kg to about 48 μg/kg, from about 30 μg/kg to about 50 μg/kg, from about 35 μg/kg to about 55 μg/kg, from about 40 μg/kg to about 60 μg/kg, from about 45 μg/kg to about 65 μg/kg, from about 50 μg/kg to about 75 μg/kg, from about 60 μg/kg to about 240 μg/kg, from about 70 μg/kg to about 350 μg/kg, from about 80 μg/kg to about 400 μg/kg, from about 90 μg/kg to about 450 μg/kg, from about 100 μg/kg to about 500 μg/kg, from about 0.01 ng/kg to about 1 ng/kg, from about 0.05 ng/kg to about 2 ng/kg, from about 1 ng/kg to about 5 ng/kg, from about 2 ng/kg to about 10 ng/kg, from

The processed silk fibroin peptides of the SBP formulation may comprise silk fibroin peptides at a concentration between 0.001% and 25%. In one aspect, the silk fibroin peptides are present at a concentration of 0.5%. In one aspect, the silk fibroin peptides are present at a concentration of 1%. In one aspect, the silk fibroin peptides are present at a concentration of 2.5%. In one aspect, the silk fibroin peptides are present at a concentration of 3%. In one aspect, the silk fibroin peptides are present at a concentration of 5%.

The SBP formulation is in a solution which may be, but is not limited to, phosphate buffer, borate buffer, and phosphate buffered saline. The solution may further comprise propylene glycol, sucrose and/or trehalose. Propylene glycol may be present in a concentration of about 1%. Sucrose may be present in a concentration such as, but not limited to, 10 mM, 50 mM, 100 mM and 150 mM. Trehalose may be present in a concentration such as, but not limited to, 10 mM, 50 mM, 100 mM and 150 mM.

In some embodiments, the present disclosure provides a processed silk-based product (SBP). The SBP may include from about 0.0001% to about 35% (w/v) of silk fibroin. The SBP may include from about 0.0001% to about 100% (w/v) of silk fibroin. In an aspect, fibroin is produced by providing raw silk (e.g., unpurified silk such as silk yarn), the raw silk comprising fibers containing silk fibroin and sericin. First, the raw silk is degummed in a salt solution, specifically a sodium carbonate solution with a sodium carbonate concentration of 0.02 to 0.5 M sodium carbonate at a temperature of about 60 to about 100° C., and for a time of greater than 60 minutes to about 480 minutes. In a preferred aspect, degumming is performed in 0.5 M sodium carbonate at 85° C. for either 240 or 360 minutes. In an aspect, degumming provides degummed silk fibers having a sericin concentration of 0-0.5 wt %. After the degumming, the silk fibroin fibers are further processed by dissolving, preferably in aqueous solution. Dissolving preferably includes using 5M to 13M lithium bromide for 1 hour to overnight at 50° C. to 100° C. to provide dissolved silk fibers, or dissolving the degummed silk fibers using a mixture of calcium chloride, ethanol, and water in a molar ratio of 1:2:8 for 1 hour to overnight at 50° C. to 100° C. to provide dissolved silk fibers. In a specific aspect, 10 wt % to 20 wt % silk fibroin is dissolved in 9.3M lithium bromide at 60° C. for 16 hours (overnight). In another specific aspect, 10 wt % to 20 wt % silk fibroin is dissolved in a mixture of calcium chloride, ethanol, and water in a molar ratio of 1:2:8 at 80° C. for 2 hours. Solvents used to dissolve processed silk may include a buffer.

In some embodiments, solvent used is an organic solvent. Organic solvents include, but are not limited to hexafluoroisopropanol (HFIP), methanol, isopropanol, ethanol, or combinations thereof.

In some embodiments, solvents include a mixture of an organic solvent and water or an aqueous solution. Solvents may include water or aqueous solutions. Aqueous solutions may include aqueous salt solutions that include one or more salts. Such salts may include but are not limited to lithium bromide (LiBr), lithium thiocyanate, Ajisawa's reagent, a chaotropic agent, calcium nitrate, or other salts capable of solubilizing silk, including any of those disclosed in U.S. Pat. No. 9,623,147 (the content of which is herein incorporated by reference in its entirety).

In some embodiments, solvents used in processed silk fibroin peptide solutions include high salt solutions. Ajisawa's reagent comprises a mixture of calcium chloride, ethanol, and water in a molar ratio of 1:2:8 respectively.

In one illustrative embodiment, the degummed silk fibroin is dissolved in 5 to 13 M LiBr. The concentration of LiBr may be 9.3 M. Dissolving in LiBr can be done at 60° C. for 16 hours (overnight).

In some embodiments, solvents used in processing silk solutions may include Ajisawa's reagent, as described in Zheng et al. (2016) Journal of Biomaterials Applications 31:450-463, the content of which is herein incorporated by reference in its entirety. After the silk fibroin fibers are dissolved, they can be diluted prior to further purification. In an aspect, the dissolved silk fibers are diluted in water to provide a concentration of 5 to 20% w/v silk fibroin fibers. Optionally the diluted fibroin solution is filtered through a polypropylene, polyethersulfone, nylon, or cellulose, diatomaceous earth, perlite depth prefilter to remove particulates and provide a clarified silk fiber solution. The diluted silk fibroin fibers can be purified using dialysis, diafiltration, or tangential flow filtration (TFF) using a regenerated cellulose or polyethersulfone filter and concentrating and recovering processed silk fibroin from the TFF. For example, the silk solution is concentrated 2×, and then diafiltered to remove the chaotropic agents, i.e., lithium bromide/calcium chloride and ethanol. Diafiltration can be performed against water at pH 3.0-11.0, salt solution, i.e., sodium chloride, potassium chloride, (10 mM-500 mM) at pH 3.0-11.0, buffer, i.e., sodium phosphate, potassium phosphate, tromethamine, at 10 mM-250 mM at pH 3.0-11.0, or buffer containing 10 mM-500 mM salt pH 3.0-11.0. Diafiltration is performed for 5-15 diavolumes, or until a sufficient amount of the chaotropic agent is removed as was performed in Cocoon (U.S. Patent Application No. 2024/0300998).

The SBP may include one or more excipients. The one or more excipients may include one or more of sucrose, lactose, phosphate salts, sodium chloride, potassium phosphate monobasic, potassium phosphate dibasic, sodium phosphate dibasic, sodium phosphate monobasic, polysor-bate 80, phosphate buffer, phosphate buffered saline, sodium hydroxide, sorbitol, mannitol, lactose USP, Starch 1500, microcrystalline cellulose, potassium chloride, sodium borate, boric acid, sodium borate decahydrate, magnesium chloride hexahydrate, calcium chloride dihydrate, sodium hydroxide, Avicel, dibasic calcium phosphate dehydrate, tartaric acid, citric acid, fumaric acid, succinic acid, malic acid, hydrochloric acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, trehalose, and sodium carboxymethylcellulose. One or more of the excipients may include phosphate buffer. One or more of the excipients may include phosphate buffered saline. One or more of the excipients may include sucrose. The excipients may include boric acid, sodium borate decahydrate, sodium chloride, potassium chloride, magnesium chloride hexahydrate, calcium chloride dihydrate, sodium hydroxide, and hydrochloric acid. The SBP may include at least one excipient selected from one or more members of the group consisting of sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremophor EL), cremophor RH 40, cremophor RH 60, dalpha-tocopherol, PEG 1000 succinate, polysorbate 20, polysorbate 80, solutol HS 15, sorbitan monooleate, poloxamer-407, polox-amer-188, Labrafil M-1944CS, Labrafil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cotton-seed oil, olive oil, peanut oil, peppermint oil, safflower oil, sesame oil, soybean oil, hydrogenated vegetable oils, hydro-genated soybean oil, medium chain triglycerides of coconut oil, medium chain triglycerides of palm seed oil, beeswax, d-alpha-tocopherol, oleic acid, medium-chain mono-glycer-ides, medium-chain di-glycerides, alpha-cyclodextrin, beta-cyclodextrin, hydroxypropyl-beta-cyclodextrin, sulfo-buty-lether-beta-cyclodextrin, hydrogenated soy phosphatidylcholine, distearoylphosphatidylglycerol, L-alpha dimyristoylphosphatidylcholine, L-alpha-dimyristoylphosphatidyl glycerol, PEG 300, PEG 300 caprylic/ca-pric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutylether-beta-cyclodextrin, transcutol, triacetin, 1-dodecylazacyclo-heptan-2-one, caprolactam, castor oil cottonseed oil, ethyl acetate, medium chain triglycerides, methyl acetate, oleic acid, safflower oil, sesame oil, soybean oil, tetrahydrofuran, glycerin, and PEG 4 kDa. The SBP may be formulated, and the formulation may be as hydrogels, suspensions, and solutions. The silk fibroin concentration in the solution may be below 1% (w/v). The SBP may be a solution, and the SBP may be stressed. The SBP may be a hydrogel, and the SBP may be stressed. The SBP may be a solution, and the solution may shear thin. The solutions may have the viscosity of a gel at a lower shear rate. The solutions may have the viscosity of a fluid at higher shear rates. The SBP may be a suspension and the SBP may be stressed.

The SBP may include any of the samples listed in any the Table below. In some embodiments, the present disclosure provides a method of preparing the SBP formulations comprising: (a) preparing the processed silk fibroin peptides, wherein the processed silk fibroin peptides comprise or are derived from natural or synthetic sources; and (b) preparing the SBP formulation using the processed silk fibroin peptides. In some embodiments, the present disclosure provides a method of treating topical conditions. The SBP may be administered via topical administration. The topical administration of SBP may be in the form of hydrogels, solutions, drops, creams, oil and water emulsions, sprays, and oils.

In an illustrative embodiment the silk fibroin peptides according to the disclosure are presented in a salt form that may include but are not limited to the following salt forms: hydrochloride, sodium, sulfate, acetate, phosphate or diphosphate, chloride, potassium, maleate, calcium, citrate, mesylate, nitrate, tartrate, aluminum, and gluconate.

In another illustrative embodiment, it is contemplated that the silk fibroin peptides according to the disclosure may have beneficial therapeutic effect on various dermatological indications. These dermatological indications include but are not limited to acne, psoriasis, atopic dermatitis, inflammatory rashes and other skin disorders caused by inflammation. In another illustrative embodiment it is thought that the silk fibroin peptides provide regenerative hair growth or the maintenance of existing hair due to its anti-inflammatory properties.

In a further illustrative embodiment, the silk fibroin peptides are combined with known active pharmaceutical ingredients having anti-inflammatory properties. In yet another illustrative embodiment, the silk fibroin peptides are combined with known active pharmaceutical ingredients having hair growth properties. In a further illustrative embodiment, the silk fibroin peptides are combined with natural ingredients having anti-inflammatory properties.

In another illustrative embodiment silk fibroin peptides according to the disclosure stimulate dermal papilla cell (DPC) migration, stimulate DPC VEGF levels (angiogenesis), and protect DPCs against oxidative damage and inhibit inflammatory cytokines. The stimulation of dermal papilla cells by a therapeutically effective amount of silk fibroin peptides can be used to treat hair loss (also as a side effect of infection/autoimmune disease), alopecia, and telogen effluvium.

In one illustrative embodiment silk fibroin peptides according to the disclosure inhibit keratinocyte proliferation, induce keratinocyte apoptosis, increase oxidative stress in keratinocytes that are in a hyperproliferative state. The inhibition of keratinocyte proliferation, induction of keratinocyte apoptosis and increase in oxidative stress by a therapeutically effective amount of silk fibroin peptides can be used to treat psoriasis, hyperkeratosis, eczema, warts formed by viruses such as HPV, calluses, corns, and allergic contact dermatitis.

In another illustrative embodiment silk fibroin peptides according to the disclosure inhibit inflammatory cytokine production in keratinocytes. The inhibition of inflammatory cytokine production in keratinocytes by a therapeutically effective amount of silk fibroin peptides can be used to treat psoriasis, hyperkeratosis, eczema, warts formed by viruses such as HPV, calluses, corns, and allergic contact dermatitis.

In a further illustrative embodiment, the silk fibroin peptides according to the disclosure improve skin barrier formation via increased cornified envelope formation in keratinocytes, improving skin barrier and inhibiting inflammatory cytokines. The improved skin barrier formation by a therapeutically effective amount of silk fibroin peptides can be used to treat atopic dermatitis and ichthyoses.

In another illustrative embodiment, the silk fibroin peptides according to the disclosure exhibit antibacterial activity against P. acnes. The antibacterial activity against P. acnes by a therapeutically effective amount of silk fibroin peptides can be used to treat acne vulgaris and sarcoidosis.

In a further illustrative embodiment, the silk fibroin peptides according to the disclosure inhibit sebum synthesis. The inhibition of sebum synthesis by a therapeutically effective amount of silk fibroin peptides can be used to treat acne vulgaris, sebaceous hyperplasia, and seborrheic dermatitis.

In another illustrative embodiment, the silk fibroin peptides according to the disclosure exhibit anti-inflammatory activity by inhibiting cytokines in sebocytes. The anti-inflammatory activity by inhibiting cytokines in sebocytes by a therapeutically effective amount of silk fibroin peptides can be used to treat acne vulgaris.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1a-1c illustrate the results of a study analyzing the increase in proliferation of Dermal Papilla Cells (DPCs) by BrdU assay.

FIGS. 2a-2c illustrate the results of a study analyzing increase in secretion of growth factors by Dermal Papilla Cells (DPCs).

FIGS. 3a-3g. illustrate the results of a study analyzing increase in migration potential of Dermal Papilla Cells (DPCs).

FIGS. 4a-4c. illustrate the results of a study analyzing cytoprotection in Human Dermal Papilla Cells (DPCs) against oxidative damage.

FIGS. 5a-5f. illustrate the results of a study analyzing anti-inflammatory activity by inhibition of cytokines in Human Dermal Papilla cells (DPCs)-IL-6 and IL-8.

FIGS. 6a-6c. illustrate the results of a study analyzing the inhibition of keratinocytes (HaCaT) proliferation for the treatment of psoriasis.

FIGS. 7a-7c. illustrate the results of a study analyzing the inhibition of mitochondrial membrane potential (MMP) in keratinocytes for the treatment of psoriasis.

FIGS. 8a-8c. illustrate the results of a study analyzing the increase in reactive oxygen species (ROS) generation in keratinocytes for the treatment of psoriasis.

FIGS. 9a-9c. illustrate the results of a study analyzing the increase in sub G0/G1 cells by cell cycle assay for the treatment of psoriasis.

FIGS. 10a-101. illustrate the results of a study analyzing the anti-inflammatory effect (IF-6, RANTES, IL-8, TNF-α) in keratinocytes for the treatment of psoriasis.

FIGS. 11a-11c. illustrates the results of a study analyzing the anti-angiogenic effect (VEGF inhibition) in keratinocytes for the treatment of psoriasis.

FIG. 12. illustrates the morphological representation of the effect of test items on formation of Cornified Envelope (CE) in keratinocytes.

FIGS. 13a-13c. illustrate the improvement of skin barrier formation by increased formulation of Cornified Envelope (CE).

FIGS. 14a-14i. illustrate the results of a study analyzing the improvement of skin barrier formation by increased in expression of surface proteins (FILAGGRIN, AQP3, INVOLUCRIN, LORICRIN).

FIGS. 15a-15i. illustrate the results of a study analyzing the anti-inflammatory activity in keratinocytes of cytokines (TSLP, RANTES, TNF-α).

FIG. 16a-16c. illustrate the results of a study analyzing the inhibition of IgE secretion in myeloma cell lines.

FIGS. 17a-17c. illustrate the results of a study analyzing the anti-bacterial activity against P. acnes.

FIGS. 18a-18c. illustrate the results of a study analyzing the sebostatic potential by inhibition of sebum synthesis (lipid accumulation) in sebocytes.

FIGS. 19a-19c. illustrate the results of a study analyzing the anti-inflammatory activity in Sebocytes by inhibition of cytokine (RANTES).

FIG. 20 illustrates the results of a study analyzing the virucidal activity of silk fibroin proteins against SARS-CoV-2.

FIGS. 21a-21c illustrate the results of a study analyzing the increase in proliferation of Endothelial Cells using silk fibroin proteins.

FIGS. 22a-22g illustrate the results of a study analyzing the increase in Cell Migration of Endothelial Cells for wound healing.

FIGS. 23a-23c illustrate the results of a study analyzing the increase in Secretion of Growth Factors in Endothelial Cells for wound healing.

FIGS. 24a-24c illustrate the results of a study analyzing the increase in fibroblast proliferation for skin health.

FIGS. 25a-25c illustrate the results of a study analyzing the increase in ECM (collagen) using for skin health.

FIGS. 26a-26f illustrate the results of a study analyzing the increase in ECM (HA, Elastin) using silk fibroin proteins.

FIGS. 27a-27g illustrate the results of a study analyzing the increase in the migration of fibroblasts for skin health.

FIGS. 28a-28i illustrate the results of a study analyzing the inhibition of MMPs (MMP-1, MMP-3, MMP-9) for skin health.

FIGS. 29a-29c illustrate the results of a study analyzing the increase in keratinocytes proliferation for skin health.

FIGS. 30a-30g illustrate the results of a study analyzing increase in migration of keratinocytes for skin health.

FIGS. 31a-31d illustrate the results of a study analyzing the increase in lipid content in keratinocytes (HaCaT) using silk fibroin proteins.

FIGS. 32a-32d illustrate the results of a study analyzing improvement of skin barrier formation by increased formation of cornified envelope (CE) for skin health.

FIGS. 33a-331 illustrate the results of a study analyzing increase in expression of surface markers for improvement of skin barrier (FLAGGRIN, APQ3, INVOLUCRIN, LORICRIN) for skin health.

FIGS. 34a-34c illustrate the results of a study analyzing the inhibition of ECM degradation enzyme (Elastase) for skin health.

FIG. 35 illustrates the results of a study analyzing DNA protection against oxidative+UV-B damage for anti-aging.

FIGS. 36a-36c illustrate the results of a study analyzing cytoprotection against UV-B induced damage for anti-aging.

FIGS. 37a-37c illustrate the results of a study analyzing the increase in mitochondrial membrane potential (MMP) for anti-aging.

FIGS. 38a-38c illustrate the results of a study analyzing inhibition in G0/G1 cells by cell cycle assay for anti-aging.

FIGS. 39a-39f illustrate the results of a study analyzing anti-inflammatory activity by inhibition of cytokines (IL-6, IL-8) for anti-aging.

FIGS. 40a-40c illustrate the results of a study analyzing cytoprotection in keratinocytes against UV-B induced damage for anti-aging.

FIGS. 41a-41c illustrate the results of a study analyzing increase in mitochondrial membrane potential (MMP) for anti-aging.

FIGS. 42a-42c illustrate the results of a study analyzing inhibition in sub G0/ G1 cells by cell cycle assay for anti-aging.

FIGS. 43a-431 illustrate the results of a study analyzing anti-inflammatory activity by inhibition of cytokines (IL-6, IL-8, IL-1-α, TNF-α) for anti-aging.

FIGS. 44a-44c illustrates the results of a study analyzing inhibition of melanin content for skin whitening.

FIGS. 45a-45g illustrate the results of a study analyzing inhibition of melanocytes migration for skin whitening.

FIGS. 46a-46c illustrates the results of a study analyzing inhibition of tyrosine kinase activity for skin whitening.

FIG. 47 illustrates the preparation of dilution test items.

FIGS. 48a-48c illustrate the results of a study analyzing skin health and the increase in cell proliferation in fibroblasts.

FIGS. 49a-49c illustrate the results of a study analyzing skin health and the increase in ECM (Collagen) using silk fibroin proteins.

FIGS. 50a-50c illustrate the results of a study analyzing skin health and the increase in ECM (HA, Elastin) using silk fibroin proteins.

FIGS. 51a-51i illustrate the results of a study analyzing skin health and the increase in cell migration using silk fibroin proteins.

FIGS. 52a-52i illustrate the results of a study analyzing skin health and the inhibition of MMPs (MMP-1, MMP-3, MMP-9) using silk fibroin proteins.

FIGS. 53a-53i illustrate the results of a study analyzing skin health and the increase of cell migration of keratinocytes using silk fibroin proteins.

FIGS. 54a-54c illustrate the results of a study analyzing skin health and the increase in lipid content in keratinocytes (HaCaT) using silk fibroin proteins.

FIGS. 55a-55d illustrates the results of a study analyzing skin health and the improvement of skin barrier formation by increased formulation of the cornified envelope (CE) using silk fibroin proteins.

FIGS. 56a-561 illustrates the results of a study analyzing skin health and the increase in expression of surface markers for improvement of skin barrier using silk fibroin proteins.

FIGS. 57a-57b illustrate the results of a study analyzing DNA protection against oxidative+UV-B damage for anti-aging potential using silk fibroin proteins.

FIGS. 58a-58c illustrate the results of a study analyzing anti-aging cytoprotection against UV-B induced damage using silk fibroin proteins.

FIGS. 59a-59c illustrate the results of a study analyzing anti-aging and the cytoprotection against t-BHP induced damage using silk fibroin proteins.

FIGS. 60a-60c illustrate the results of a study analyzing anti-aging and the restoration of mitochondrial membrane potential (MIMP) using silk fibroin proteins.

FIGS. 61a-61c illustrate the results of a study analyzing anti-aging and the increase in sub G0/G1 cells by cell cycle assay using silk fibroin proteins.

FIGS. 62a-62c illustrate the results of a study analyzing anti-aging and the decrease in ROS generation using silk fibroin proteins.

FIGS. 63a-63f illustrate the results of a study analyzing anti-aging anti-inflammatory activity by inhibition of cytokines using silk fibroin proteins.

FIGS. 64a-641 illustrate the results of a study analyzing anti-aging inhibition of cytokines using silk fibroin proteins.

FIGS. 65a-65c illustrate the results of a study analyzing anti-aging inhibition of histamine release using silk fibroin proteins.

FIGS. 66a-66g illustrate the results of evolution across time for overall dry skin when testing effectiveness of the Silk Soothing Lotion.

FIGS. 67a-67f illustrate the instrumental analysis for the study analyzing overall dry skin when testing effectiveness of the Silk Soothing Lotion.

FIGS. 68a-68c illustrate trial results for the Silk Soothing Lotion.

FIGS. 69a-69c illustrate trial results for the Silk face serum.

FIGS. 70a-70b Human Subject Repeat trial results for an Insult Patch Test Skin Irritation/Sensitization Evaluation testing a semi-occlusive patch.

### DETAILED DESCRIPTION

Embodiments of the present disclosure relate to silk-based products (SBPs), formulations and their methods of use. The term “silk” generally refers to a fibrous material formed by insects and some other species that includes tightly bonded protein filaments. Herein, the term “silk” is used in the broadest sense and may embrace any forms, variants, or derivatives of silk discussed.

Silk fibers from silkworm moth (Bombyx mori) cocoons include two main components, sericin (usually present in a range of 20-30%) and silk fibroin (usually present in a range of 70-80%). Structurally, silk fibroin forms the center of the silk fibers and sericin acts as the gum coating the fibers. Sericin is a gelatinous protein that holds silk fibers together with many of the characteristic properties of silk (see Qi et al. (2017) Int J Mo! Sci 18:237 and Deptuch et al. (2017) Materials 10:1417, the contents of each of which are herein incorporated by reference in their entireties). Silk fibroin is an insoluble fibrous protein consisting of layers of antiparallel beta sheets. Its primary structure mainly consists of recurrent serine, alanine, and glycine repeating units. The isoelectric point of silk fibroin has been determined to be around 4.2. Silk fibroin monomers include a complex of heavy chain (around 350 kDa) and light chain (around 25 kDa) protein components. Typically, the chains are joined by a disulfide bond. With some forms, heavy chain and light chain segments are non-covalently bound to a glycoprotein, p25. Polymers of silk fibroin monomers may form through hydrogen bonding between monomers, typically increasing mechanical strength (see Qi et 7. (2017) Int J Mo! Sci 18:237). During silk processing, fragments of silk fibroin monomers may be produced, including, but not limited to, fragments of heavy and/or light chains. These fragments may retain the ability to form hydrogen bonds with silk fibroin monomers and fragments thereof. Herein, the term “silk fibroin” is used in its broadest sense and embraces silk fibroin polymers, silk fibroin monomers, silk fibroin heavy and light chains, silk fibroin fragments, and variants, derivatives, or mixtures thereof from any of the wild type, genetically modified, or synthetic sources of silk described herein. The present disclosure includes methods and formulations utilizing processed silk fibroin peptides and SBPs, different forms of SBP formulations, and a variety of applications for utilizing processed silk fibroin peptides, SBPs, and SBP formulations alone or in combination with various compounds and compositions.

The oil-in-water emulsion of the present invention is suitable for application to skin or hair. The oil-in-water emulsion comprises an oil phase containing at least one lipophilic solvent; an aqueous phase; and an emulsifying system. Optionally, a polymeric additive may be incorporated into the aqueous phase of the emulsion to create an emulsion gel of varying viscosities depending on the amount and type of polymer used.

The oil-in-water emulsions of the present invention are exceptionally mild. The water-in-oil emulsions of the present invention surprisingly have also been found to have skin moisturizing properties combined with the anti-inflammatory properties of the silk fibroin peptides according to the disclosure.

The oil phase of the present invention comprises at least one lipophilic solvent and preferably other lipophilic cosmetic or pharmaceutically useful ingredients known to those of ordinary skill in the art. Preferably the lipophilic solvent is selected from the group consisting of liquid fatty alcohols from 16 to 22 carbon atoms per molecule, volatile hydrocarbon fluids, and vegetable oils. Preferred volatile silicone fluids which can be used in the oil phase of the present invention include cyclomethicone (cyclopentasiloxane, cyclohexasiloxane, cyclotetrasiloxane) and dimethicone (0.65 centistokes). A preferred volatile hydrocarbon fluid usable in the present invention is isododecane, sold under the tradename Permethyl 99A, by Presperse Inc., Piscataway, N.J. The oil phase may further comprise a lipophilic co-solvent selected from the group consisting of fatty acid esters, liquid branched chain fatty alcohols from 16 to 20 carbon atoms in length, and triglycerides. A preferred triglyceride is caprylic/capric triglyceride. To improve feel, dimethicone/vinyl dimethicone cross polymer may be added to the oil phase. The oil phase comprises from about 30% to about 70% by weight of the emulsion, preferably from about 40% to about 50%. In order to aid in matching refractive indexes of the aqueous and oil phases, the oil phase may include index adjusting agents known to those of ordinary skill in the art such as halogenated solvents.

The aqueous phase of the present invention comprises water and preferably other water-soluble cosmetically or pharmaceutically useful ingredients known to those of ordinary skill in the art. In particular, the aqueous phase of the present invention comprises a therapeutic effective amount of silk fibroin peptides according to the disclosure. The aqueous phase preferably includes one or more polyols selected from the group consisting of glycerin, polyethylene glycol, propylene glycol, polypropylene glycol, 1,3 butylene glycol, methylpropanediol, hexylene glycol and sorbitol. Preferably the polyol has a molecular weight from about 75 to about 10,000 daltons, more preferably from about 200 to about 5000 daltons, and most preferably from about 300 to about 1000 daltons. A preferred polyol for use in the present invention is polyethylene glycol 400, sold under the tradename Carbowax PEG 400 by Union Carbide, Houston, Tex., alone or in combination with glycerin. Preferably, the polyol is present in an amount of from about 10% to about 35% of the total composition, more preferably from about 15% to about 30% of the total composition, and most preferably it makes up from about 18% to about 25% of the total composition. Alternatively, to the polyol, the aqueous phase of the emulsion may contain polysaccharides or other agents known to those of ordinary skill in the art to be useful in adjusting the refractive index of the aqueous phase.

The emulsifying system includes at least one non-ethoxylated fatty acid ester emulsifier having an HLB from about 11 to about 16, preferably from about 13 to about 16. Preferably the emulsifier is a sucrose ester. More preferably the sucrose ester emulsifier is selected from the group consisting of sucrose laurate, sucrose stearate, sucrose palmitate, sucrose oleate, sucrose myristate, sucrose cocoate, and sucrose isostearate, or a combination thereof. Most preferably the non-ethoxylated emulsifier is a sucrose laurate or a sucrose palmitate. Preferably the percentage of the non-ethoxylated fatty acid ester is less than 5% by weight of the total composition, more preferably less than 3% by weight of the total composition, and most preferably less than 1.5%. When the non-ethoxylated fatty acid ester is a sucrose ester, the preferred range is from about 0.5 to 5% by weight of the total composition, and more preferably from about 1 to about 2.5% by weight of the total composition, and most preferably less than 1.5% of the total composition.

Optionally, a polymeric additive may be incorporated into the aqueous phase of the emulsion in order to achieve a desired viscosity or gel consistency. The polymeric additive is a water-soluble polymer selected from the group consisting of sclerotium gum, xanthan gum, sodium alginate, carbomer, cellulose ethers and acrylate polymers. Acrylate polymers usable in the present invention include: steareth-20 methylacrylate copolymer, sold under the tradename Aculyn 22 by Rohm & Haas Company, Philadelphia, Pa.; Pemulen TR-1 and TR-2 (C10-C30 alkyl acrylate crosspolymer), both sold by Goodrich Specialty Chemicals, Cleveland, Ohio; and Hypan QT1000 and SA100H, both acrylonitrogen copolymers, sold by Lipo Chemicals, Inc., Patterson, N.J. Preferred polymers for use in the present invention are Clearogel CS11D and Aculyn 22. Depending upon the viscosity to be achieved, the amount of polymeric additive can range from about 0.1% to about 2.5% by weight of the total composition, preferably 0.5% to 0.75% by weight of the total composition. In general, the greater the amount of the polymeric additive, the greater the viscosity.

The present oil and water suspension can be utilized in a wide range of cosmetic and pharmaceutical products, including, but not limited to, transparent deodorant gels, transparent skin and eye moisturizing gels, transparent hair conditioner and glosser gels, transparent auto bronzer gels, transparent sunscreen gels, transparent skin tightening gels and other transparent dermatologic vehicles for delivering silk fibroin peptides according to the disclosure and optionally combined with pharmaceutically active ingredients (e.g., ascorbic acid and retinol).

In order to produce the desired products, the basic components of the invention as described above may be combined with other cosmetic and pharmaceutical ingredients which are well known to cosmetic and pharmaceutical chemists. Examples of such additional components in addition to the SBPs include, but are not limited to, anti-seborrheic agents, anti-acne agents, antioxidants, skin lightening agents, depigmenting agents, anti-wrinkle agents, vitamins, sunscreen agents, self-tanning agents, topical analgesics, anti-inflammatory agents, antipruritic agents, deodorants, as well as purely cosmetic ingredients, such as pigments, water soluble emollients, humectants, stabilizers, and fragrances.

Sunscreen agents which are most suitable for use in the present invention include octyl methoxycinnamate, octyl salicylate, and avobenzone.

The oil-in-water emulsion of the present invention is prepared according to principles and techniques generally known to those skilled in the cosmetic and pharmaceutical arts.

### Processing and Purification of Silk Fibroin Peptides

The processed silk fibroin peptides and/or other SBP components include methods of processing and purification as set forth in WO 2023/251264 A1 entitled: Methods for reducing impurities in silk fibroin preparations, the contents of which are incorporated in their entirety.

In an aspect, a method of purifying a silk fibroin peptide comprises preparing an aqueous silk fibroin solution having a concentration of greater than or equal to 5% w/v silk fibroin from the silk fibroin preparation, wherein the silk fibroin preparation comprises a chaotropic salt; and exchanging the chaotropic salt from the silk fibroin solution at a pH below the isoelectric point of the silk fibroin, wherein the pH is between 2 and 5, replacing the chaotropic salt with a buffer comprising 10 to 300 mM of a second salt, or a combination thereof, to prepare the purified silk fibroin.

Described herein are methods of reducing impurities, particularly elemental impurities introduced during purification, of silk fibroin peptides. During purification of silk fibroin which has been prepared by a process including dissolution in lithium bromide or another chaotropic salt as is standard in the art, the inventors have found that standard methods using tangential flow filtration (TFF) with water only in the retentate/replacement feed or dialysis provide a final material that has much higher lithium than bromide, typically 500-3000 ppm Li and 20-200 ppm Br, normalized to the amount of silk fibroin. This is unexpected, as it would be expected that both Li and Br would be completely or almost completely removed during exhaustive TFF or dialysis. In addition, as the mass of Br is 11.6 times greater than that of lithium one would expect that the Br residuals would be 11.6 times greater than Li, not 100-fold less.

For example, U.S. Pat. No. 9,517,191 and related patents claim that silk fibroin preparations have 0 ppm to 500 ppm of “inorganic residuals”, such as lithium bromide residuals of 10 ppm to 300 ppm, “measurable using a high performance liquid chromatography lithium bromide assay”. However, while Example 5 describes a TFF process to remove lithium bromide, the specification does not provide any data demonstrating removal of the lithium and bromide to the levels of 10 ppm to 300 ppm. Further, the '191 patent does not disclose if the level of inorganic residuals is normalized to the amount of silk fibroin in solution, which would be necessary. For example, any solution could be diluted with DI water to lower the level of residuals to below 300 ppm in solution but would not demonstrate any improved removal or residuals as compared to the amount of silk. Further, as shown in the examples herein, repeating the examples of the '191 patent provides a silk fibroin material with 1000-2000 ppm Li and 40-100 ppm Br, normalized to the amount of silk fibroin. The '191 patent does not provide a pH or salt composition for the TFF solution purported to remove Li and Br residuals.

The inventors have unexpectedly found that using a retentate having a pH of 3 to 4.5, such as pH 4, and/or using a TFF replacement feed solution of a salt concentration of 10 to 300 mM NaCl, for example, resulted in a dramatic decrease in Li levels, specifically 20-500 ppm normalized to the amount of silk fibroin. The Br levels in these same samples can be 200-1500 ppm normalized to the amount silk fibroin. The reduction in Li levels is particularly important for product safety in pharmaceuticals and consumer products, for example. If one wanted to maintain the safe level of Li in a pharmaceutical product comprised of dried or concentrated silk fibroin in order to utilize the benefits of larger amounts of silk, these methods could be employed to ensure more complete removal of elemental impurities.

In an aspect, a method of purifying a silk fibroin peptides comprises preparing an aqueous silk fibroin solution having a concentration of greater than or equal to 5% w/v silk fibroin from the silk fibroin preparation, wherein the silk fibroin preparation comprises a chaotropic salt; and exchanging the chaotropic salt from the silk fibroin solution at a pH below the isoelectric point of the silk fibroin, wherein the pH is between 2 and 5, replacing the chaotropic salt with a buffer comprising 10 to 300 mM of a second salt, or a combination thereof, to prepare the purified silk fibroin.

Raw silk starting material can be obtained from the silkworm species Bombyx mori. Other examples of silk producer species include, but are not limited to, Bombyx mandarina, Bombyx sinesis, Anaphe moloneyi, Anaphe panda, Anaphe reticulate, Anaphe ambrizia, Anaphe carteri, Anaphe venata, Anapha infracta, Antheraea assamensis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Copaxa multifene strata, Coscinocera hercules, Callosamia promethea, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Sarnia Cynthia, and Sarnia ricini.

In an aspect, the silk fibroin preparation comprises a chaotropic salt. As used herein, a chaotropic salt is a salt that disrupts the structure of macromolecules, such as silk fibroin.

In an aspect, the method comprises preparing an aqueous silk fibroin solution having a concentration of greater than or equal to 5% w/v silk fibroin from the silk fibroin preparation, wherein the silk fibroin preparation was prepared by a process comprising dissolving silk fibroin fibers in 5M to 13 M LiBr.

In an aspect, fibroin is produced by providing raw silk (e.g., unpurified silk such as silk yam, cocoons), the raw silk comprising fibers containing silk fibroin and sericin. First, the raw silk is degummed in a salt solution, specifically a sodium carbonate solution with a sodium carbonate concentration of 0.05 to 1 M, specifically 0.1 to 1 M, more specifically 0.2 to 0.5 M sodium carbonate at a temperature of about 60 to about 90° C., and for a time of greater than 60 minutes to about 480 minutes. In a preferred aspect, degumming is performed in 0.5 M sodium carbonate at 85° C. for either 240 or 360 minutes. In an aspect, degumming provides degummed silk fibers having a sericin concentration of 0-0.5 wt %.

Of particular relevance to the present application, most prior art processes for purifying silk fibroin use 0.02 M sodium carbonate with boiling for 30 or 60 minutes to provide degummed silk fibroin. The inventors have found that this prior art process produces a material that is not favorable for subsequent processing steps, specifically TFF performed with a concentration of 5 to 20% w/v silk fibroin fibers. The prior art degumming process provides silk fibroin that is so viscous in solution it cannot be run at concentrations higher than about 1% w/v by TFF. Without being held to theory, it is believed that the molecular weight, polydispersity, and/or distribution of molecular weights of silk fibroin produced by prior art degumming processes is unfavorable for subsequent processing steps.

After the degumming, the silk fibroin fibers are further processed by dissolving, preferably in an aqueous solution comprising a chaotropic agent. Exemplary chaotropic agents include lithium bromide, lithium chloride, calcium chloride, ethanol, guanidinium chloride, and urea. Dissolving preferably includes using 5M to 13M lithium bromide for 1 hour to overnight at 50° C. to 100° C. to provide dissolved silk fibers, or dissolving the degummed silk fibers using a mixture of calcium chloride, ethanol, and water in a molar ratio of 1:2:8, respectively, for 1 hour to overnight at 50° C. to 100° C. to provide dissolved silk fibers. In a specific aspect, 10 wt % to 20 wt % silk fibroin is dissolved in 9.3M lithium bromide at about 60° C. for 16 hours (overnight). In an aspect, using the TFF/Dialysis methods described herein the purified silk fibroin peptide comprises 10 to 600 ppm lithium per mg silk. In another aspect, the purified silk fibroin peptide comprises 10 to 600 ppm bromine per mass of silk fibroin.

In another aspect, the silk fibroin peptide is prepared by a process comprising dissolving the degummed silk fibers using a mixture of calcium chloride, ethanol, and water in a molar ratio of 1:2:8 for 1 hour to overnight at 50° C. to 100° C.

After the silk fibroin fibers are dissolved, they can be diluted prior to further purification. In an aspect, the dissolved silk fibers are diluted in water to provide a concentration of 5 to 20% w/v silk fibroin fibers. Optionally the diluted fibroin solution is filtered through a polypropylene, polyethersulfone, nylon, or cellulose, diatomaceous earth, perlite depth prefilter to remove particulates and provide a clarified silk fiber solution.

The aqueous silk fibroin solution having a concentration of greater than or equal to 5% w/v silk fibroin is then purified by and exchanging the chaotropic salt from the silk fibroin solution at a pH below the isoelectric point of the silk fibroin, wherein the pH is between 2 and 5, replacing the chaotropic salt with a buffer comprising 10 to 300 mM of a second salt, or a combination thereof, to prepare the purified silk fibroin.

In an aspect, the diluted silk fibroin fibers are then purified by tangential flow filtration (TFF) using, for example, continuous diafiltration by tangential flow filtration (TFF). Diafiltration is the fractionation process that washes smaller molecules through a membrane and leaves larger molecules in the retentate without significantly changing concentration. It can be used to remove salts or exchange buffers. It can remove ethanol or other small solvents or additives.

In continuous diafiltration, the diafiltration solution (water, buffer, or a salt solution) is added to the sample feed reservoir at the same rate as filtrate is generated. In this way the volume in the sample reservoir remains constant, but the small molecules (e.g., salts) that can freely permeate through the membrane are washed away in the filtrate (also called the permeate). Using salt removal as an example, each additional diafiltration volume (DV; also referred to herein as a diavolume) reduces the salt concentration further as the salt ions are removed in the filtrate. (A diafiltration volume is the volume of sample before the diafiltration solution is added.) Anything that isn't filtered out is the “retentate”. In the present case, the retentate includes the majority of the silk fibroin.

In the process described herein, in one method, the “sample”, also called the retentate, which includes the silk fibroin, is pH adjusted down to pH 2 to 5, for example, from its original pH of 8.5-9.

In an aspect, exchanging salt ions from the aqueous silk fibroin solution is by continuous diafiltration by tangential flow filtration (TFF) with a 5 kDa to 10 kDa molecular weight cut-off membrane by a process comprising providing a reduced pH retentate and filtering with at least three diafiltration volumes with a replacement feed of water, wherein the reduced pH retentate is a retentate comprising the silk fibroin and having a pH of 2 to 5. In an aspect, prior to providing the reduced pH retentate, the method comprises filtering least 3 diafiltration volumes, preferably at least 5 diafiltration volumes, with a water replacement feed.

The reduced pH retentate is a retentate comprising the silk fibroin and having a pH of 2 to 5, preferably 3 to 4.5, more preferably 3 to 4, and most preferably 4.

In another aspect, exchanging salt ions from the aqueous silk fibroin solution is by dialysis against the buffer having a pH of 2-5, wherein a pH of 2-5 is maintained through at least a portion of the dialysis procedure, preferably through the entire dialysis procedure. [0024] In yet a further aspect, exchanging salt ions from the aqueous silk fibroin solution is by continuous diafiltration by tangential flow filtration (TFF) with a 5 kDa to 10 kDa molecular weight cut-off membrane by a process comprising filtering with at least three diafiltration volumes of a salt solution replacement feed, wherein the salt solution replacement feed comprises 10 to 300 mM of the salt. In an aspect, prior providing the salt solution replacement feed, the method comprises filtering least 3 diafiltration volumes, preferably at least 5 diafiltration volumes, with a water replacement feed.

In an aspect, the salt solution replacement feed, e.g., the second salt, comprises 10 to 300 mM of a Mg, Ca, K, or Na salt, specifically NaCl or CaCl₂, more specifically 150 mM NaCl. The pH of the salt solution replacement is not critical, but is preferably unbuffered, such as between pH 6 and 8.

In another aspect, exchanging salt ions from the aqueous silk fibroin solution is by dialysis in the buffer comprising 10 to 300 mM of the monovalent or divalent salt.

In another aspect, the method further comprises adjusting the pH of the purified silk fibroin preparation to a pH of 7-9, preferably 8.5-9.

In the following aspects, the silk fibroin preparation is prepared by a process comprising dissolving the degummed silk fibers using a mixture of calcium chloride, ethanol, and water in a molar ratio of 1:2:8 for 1 hour to overnight at 50° C. to 100° C.

In an aspect, exchanging salt ions comprises continuous diafiltration by tangential flow filtration (TFF), dialysis, or a combination thereof. In an aspect, the calcium ions are reduced to 10 to 500 ppm.

In this case, exchanging salt ions from the aqueous silk fibroin solution is by tangential flow filtration (TFF) with a 5 kDa to 10 kDa molecular weight cut-off membrane by a process comprising providing a reduced pH retentate and filtering with at least three diafiltration volumes with a replacement feed of water, wherein the reduced pH retentate is a retentate comprising the silk fibroin and having a pH of 2 to 5.

Alternatively, purifying the silk fibroin solution is by dialysis in the buffer having a pH of 2-5, wherein a pH of 2-5 is maintained through at least a portion of the dialysis procedure, preferably through the entire dialysis procedure.

In another alternative, exchanging salt ions from the aqueous silk fibroin solution is by continuous diafiltration by tangential flow filtration (TFF) with a 5 kDa to 10 kDa molecular weight cut-off membrane by a process comprising filtering with at least three diafiltration volumes of a salt solution replacement feed, wherein the salt solution replacement feed comprises 10 to 300 mM of the monovalent or divalent salt. [0033] In yet another alternative, exchanging salt ions from the aqueous silk fibroin solution is by dialysis in the buffer comprising 10 to 300 mM of the salt.

In any of the foregoing aspects, the silk fibroin preparation is prepared by degumming silk yarn in 0.05 to 1 M sodium carbonate at a temperature of about 60° C. to about 90° C., and for a time of greater than 60 minutes to about 480 minutes, to provide degummed silk fibers having a sericin concentration of 0-0.5 wt %.

Following TFF, the solution may be filtered through a ˜0.8-2 μm polypropylene, polyethersulfone, nylon, or cellulose, diatomaceous earth, perlite depth filter and stored at either frozen to −80° C. or stored at 4° C., preferably at a silk fibroin concentration of 5% to 20% (w/v).

The silk fibroin peptides prepared by the foregoing method preferably has a weight average molecular weight of less than 90 kDa or less than as measured by size exclusion chromatography depending upon the method used, or less than 20 kDa as determined by dynamic light scattering. It is important to note that the determined molecular weight of silk fibroin preparations is highly dependent upon the method used to determine molecular weight. The silk fibroin prepared by the foregoing method also preferably has polydispersity of less than 1.4 as determined by dynamic light scattering.

### EXAMPLES

The invention is further illustrated by the following examples, which are intended to illustrate and not limit the invention. The characterization for the silk fibroin used in the examples are identified as TF-133, TF-125 and TF-134. The TF-134 was prepared using the CaCl/EtOH/Water dissolution method, while the TF-125 and TF-133 were prepared using LiBr in water. The average molecular weight of the silk fibroin was measured by ultra-performance liquid chromatography size exclusion chromatography (UPLC-SEC). A Waters Acquity H-Class UPLC equipped with a Waters Acquity UPLC Protein BEH SEC Column, 200 Å, 1.7 μm, 4.6 mm×150 mm and Waters Acquity tuneable ultraviolet (TUV) detector was used. Sample temperature was maintained at 4° C. throughout the analysis. An isocratic flow rate of 0.3 mL/min was run using a mobile phase consisting of 100 mM Tris-HCl with 400 mM sodium perchlorate at pH 8.0. Ultraviolet detection was monitored at 280 nm. Molecular weights were calculated using Waters BEH 200 Å Protein SEC Standard Mix.

These examples utilized silk solutions having varying concentrations and parameters as set forth in the table below:

| S. No. | Parameter | TF-125 | TF-134 | TF-133 |
|---|---|---|---|---|
| 1 | Name | Silk | Silk | Silk |
| | | Fibroin | Fibroin | Fibroin |
| | | Solution | Solution | Solution |
| 2 | Strength | Ingredient | Ingredient | Ingredient |
| | | Strength | Strength | Strength |
| 3 | Concentration/ | 10.2% | 12.4% | 15.4% |
| | Content | (w/v) in | (w/v) in | (w/v) in |
| | | water | water | water |
| 4 | Batch No. | TF-125 | TF-134 | TF-133 |
| 6 | Physical | Clear, | Clear, | Clear, |
| | Description | yellow, | yellow, | yellow, |
| | | slightly | slightly | slightly |
| | | viscous | viscous | viscous |
| | | solution | solution | solution |
| 7 | Formulation | Aqueous | Aqueous | Aqueous |
| | Type | solution | solution | solution |
| 11 | Mol wt | 42.6 kDa | 40.4 kDa | 36.3 kDa |
| 12 | pH | 8.8 | 9.0 | 8.8 |
| 13 | Solubility | Freely | Freely | Freely |
| | | soluble in | soluble in | soluble in |
| | | water | water | water |

### A. Evaluation of Hair Growth Promoting Potential of Fibroin Proteins (Tf-125, Tf-133, Tf-134) Using In Vitro Human Follicular Dermal Papilla Cells Based Assays by Multi-Parametric Analysis

Hair is a protein filament that grows from follicles found in the dermis. Hair is one of the defining characteristics of mammals. The human body, apart from areas of glabrous skin, is covered in follicles which produce thick terminal and fine vellus hair. Each hair has a hair shaft and a hair root. The shaft is the visible part of the hair that sticks out of the skin. The hair root is in the skin and extends down to the deeper layers of the skin. It is surrounded by the hair follicle (a sheath of skin and connective tissue), which is also connected to a sebaceous gland.

At the base of the hair, the hair root widens to a round hair bulb. The hair papilla, which supplies the hair root with blood, is found inside the bottom of the hair bulb. New hair cells are constantly being made in the hair bulb, close to the papilla. The growth of the hair follicle is cyclical. Stages of rapid growth and elongation of the hair shaft alternate with periods of quiescence and regression driven by apoptotic signals. This cycle can be divided into three phases: anagen (growth), catagen (transition), and telogen (rest).

The bulk of the hair follicle is composed of keratinocytes, the epithelial cells that comprise the hair shaft itself as well as the encircling inner and outer root sheaths. However, a specialized mesenchymal population, the dermal papilla (DP), plays a critical role in directing the activities of these keratinocytes to form the follicle and generate the hair shaft. Active communication between the DP or its precursors and the epithelial compartment regulate many aspects of follicle biology.

The DP remains intimately associated with the epithelial progenitor populations of the follicle despite the dynamic changes in follicle structure as it goes through cycles of active growth (anagen), degeneration of the lower follicle (catagen), quiescence (telogen), and regeneration. The dermal papilla (DP) of the hair follicle is both a chemical and physical niche for epithelial progenitor cells that regenerate the cycling portion of the hair follicle and generate the hair shaft.

Hair dermal papilla cells are specialized mesenchymal cells that exist in the dermal papilla located at the bottom of hair follicles. These cells play pivotal roles in hair formation, growth, and cycling. Hair follicle formation is usually directed by an aggregation of dermal mesenchymal cells, the origin of dermal papilla cells, in the embryonic skin.

Vascular endothelial growth factor (VEGF, commonly referred to VEGF-A) is a crucial regulator of normal and pathological blood vessel growth. VEGF mRNA is strongly expressed in dermal papilla cells (DPC) in the anagen phase, but during the catagen and telogen phases, VEGF mRNA is less strongly expressed.
VEGF is a crucial regulator of physiological angiogenesis during skin development, and its overexpression in hair follicle epithelial cells is responsible for perifollicular vascularization and acceleration of hair regrowth in mice. Dysregulation of these growth factors together with the alteration of the hair cell cycle is the trigger factor of one of the most common hair disorders, alopecia. An increase in VEGF secretion in DPCs indicate hair growth promotion.
Minoxidil is reported to stimulate hair growth by inducing mitogenic effect on DPCs and by activating the cytoprotective prostaglandin synthase-1 enzyme and enhancing the expression of the vascular endothelial growth factor (VEGF) mRNA present in the dermal papillae.
Intrinsic or environmental stress results in oxidative damage to DPCs and results in hair loss. Dermal papilla cells (DPCs) taken from male androgenetic alopecia (AGA) patients undergo premature senescence in vitro in association with the expression of p16(INK4a), suggesting that DPCs from balding scalp are more sensitive to environmental stress than nonbalding cells.
There is a large body of research relating to stress and inflammation in animals and humans, that extensively examines cytokines and other pro-inflammatory effects of stress. Inflammation plays a key role as a damaging factor leading to hair loss.

There is a well-established link between inflammation and hair loss.

Recently, DPCs have emerged as the focus of intense interest for researchers in hair biology. DPCs serve as excellent model to investigate the mechanistic action of compounds on functioning of human follicles. Owing to their important role in hair growth, DPCs have been classically used in numerous studies as in vitro screening model to evaluate the effect of hair growth modulating agents at cellular and molecular level. Numerous studies have been reported where DPCs have been employed as a screening tool to understand the effect on hair growth promotion, using key end points such as increase in proliferation, migration, VEGF secretion, cytoprotection against oxidative damage and anti-inflammatory activity in DPCs.

Increase in DPCs proliferation indicated mitogenic activity and supports the hair growth promotion. Increase in VEGF secretion by DPCs suggests increased angiogenesis, enhanced vasculature and blood supply to enhance hair growth. Cytoprotective effect against oxidative stress is another key marker for hair growth promotion. Increase in cell migration supports hair growth promotion as well. Anti-inflammatory activity in DPCs by downregulation of secreted inflammatory cytokines also strengthens the hair growth promotion claim.

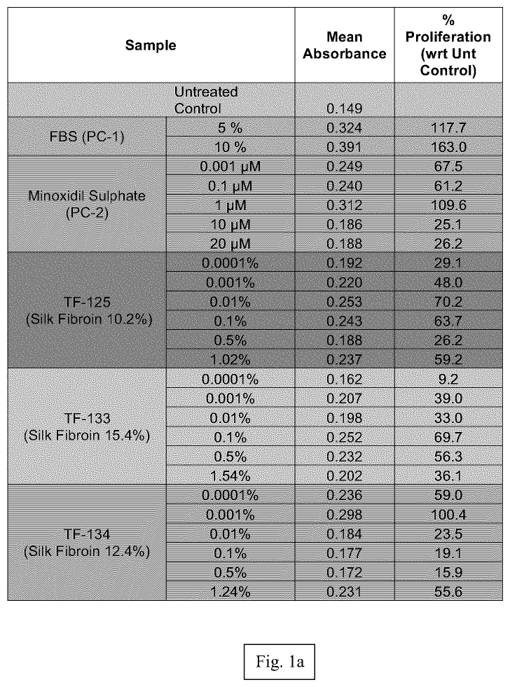

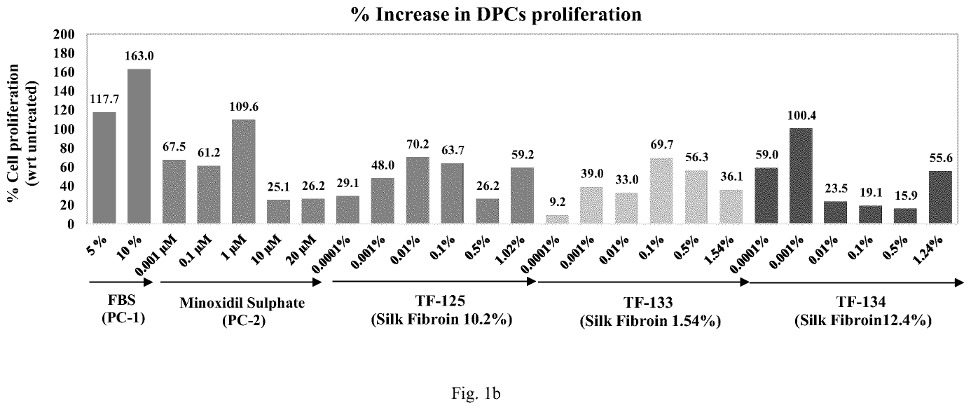

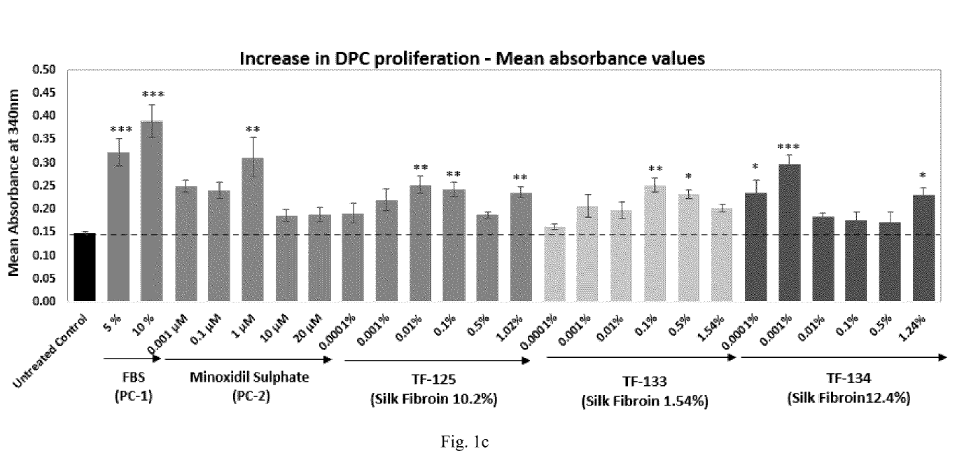

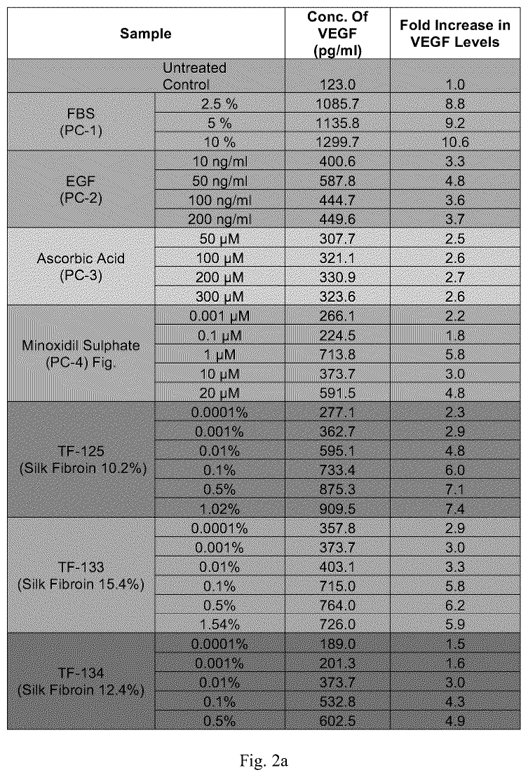

I. Increase in Proliferation of Dermal Papilla Cells (DPCs) by BrdU Assay (as Seen in FIGS. 1a-1c)

### Study Design

Test System—Human Dermal Papilla Cells (DPCs)
Number of cells plated—800 cells/well in 96 Well plate
FBS concentrations—0%
Test items—TF-125 (10.2%), TF-133 (15.4%), TF-134 (12.4%)
Time points—48h
Estimation method—BrdU assay
Positive Controls—FBS, EGF, Ascorbic acid, Minoxidil Sulphate

### Procedure

Cells were plated in 10% FBS and incubated for 24 h. Cells were then serum starved in 0% FBS for 24 h. Cells were treated with 3 Test items at various concentrations. After 48 h, the effect on proliferation was determined by BrdU assay. 10 μl of BrdU solution was added/well and cells were incubated for 90 minutes at 37° C. After removing medium, cells were fixed, and the DNA was denatured in one step by adding 200 μl of FixDenat. Cells were incubated for 30 min at RT (15-25° C.). The FixDenat solution was removed and 100 μl of Anti-BrdU-POD solution was added to each well. Cells were incubated for 90 min at RT (15-25° C.). Wells were washed 3 times using 200 μl of washing solution. 100 μl/well of substrate solution was added. Cells were incubated for 30 min at RT (15-25° C.). After incubation, a blue colored complex was formed. Absorbance of each well was measured at 370 nm.

### Calculations

Increase in proliferation of DPCs was calculated wrt control (Untreated cells). [(A−B)/B]*100; Where A=Test Item treated cells, B=Control (Untreated) cells

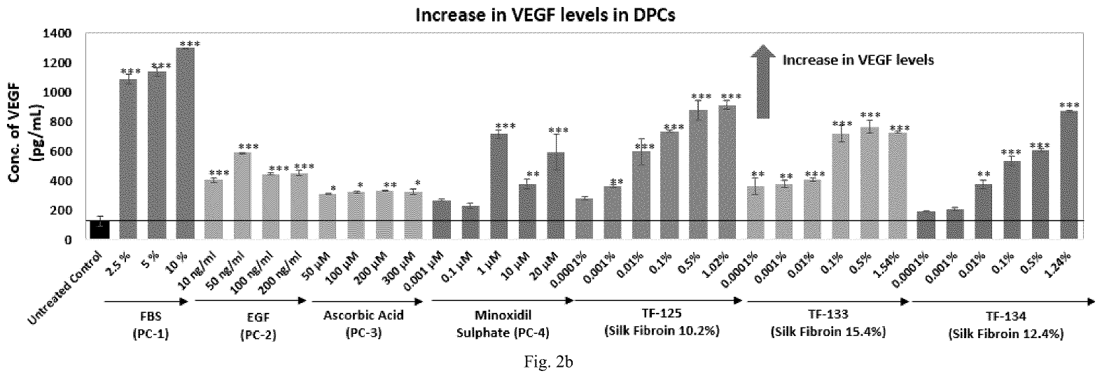

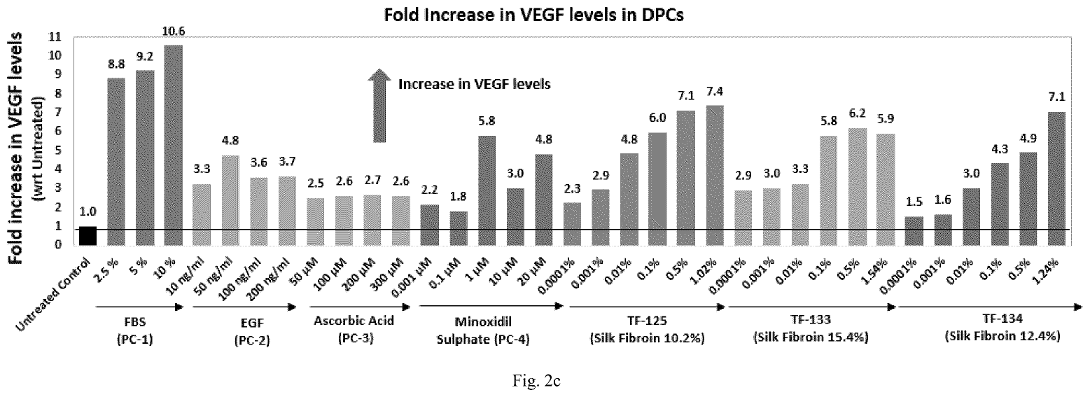

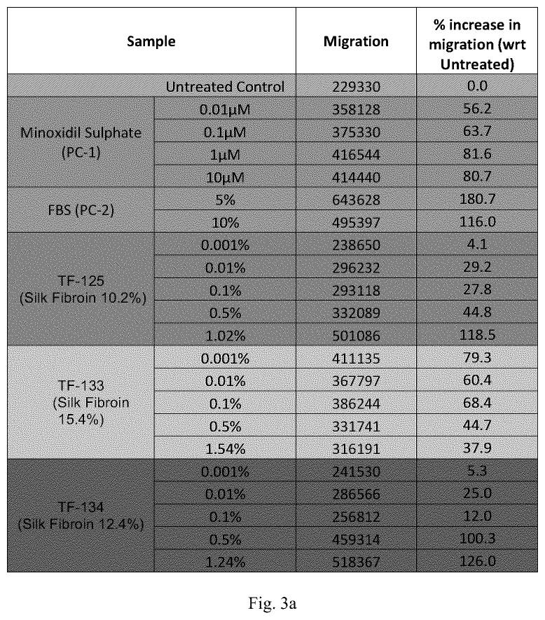

II. Assay 2-Increase in secretion of growth factors by Dermal Papilla Cells (DPCs) (as seen in FIGS. 2a-2c)

### Study Design

Test System—Human Dermal Papilla Cells (DPCs)
Number of cells plated—0.1×106 cells/well in 24 Well plate
FBS concentrations—0% FBS
Test items—TF-125 (10.2%), TF-133 (15.4%), TF-134 (12.4%)
Time points—24h
Estimation method—ELISA in culture supernatants/Lysates
Positive Control—FBS, EGF, Ascorbic acid, Minoxidil Sulphate

### Procedure

Cells were plated in 10% FBS and incubated for 24 h. Cells were then serum starved in 0% FBS for 24 hours. Cells were treated with 3 Test items at various concentrations for 24 hours. After 24 hours, culture supernatants were collected, and lysates were prepared. Levels of VEGF, KGF, IGF were estimated by ELISA. Assay diluent was added to each well. Respective kit standards and samples (supernatants of cells) were directly pipetted into the wells and incubated for 2 hr. at RT. After washing away any unbound substances for a total of 3 times, respective conjugate was added to each well and incubated for 1-2 hr. at RT. Following a wash (3 times) to remove any unbound conjugate, substrate solution was added to the wells and incubated for 30 min at RT in dark. The reaction was stopped by adding Stop solution to each well. The optical density of the color was measured at 450 nm

### Calculations

Increase in the expression of growth factors levels was calculated wrt control (untreated cells).

$$[ ( A - B ) / B ] * 100 ; Where A = Test Item treated cellls ,$$

$$B = Control ( Untreated ) cells$$

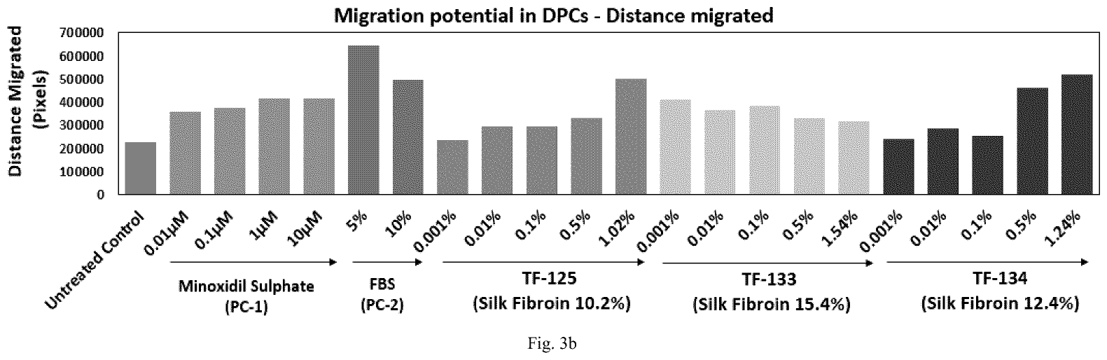

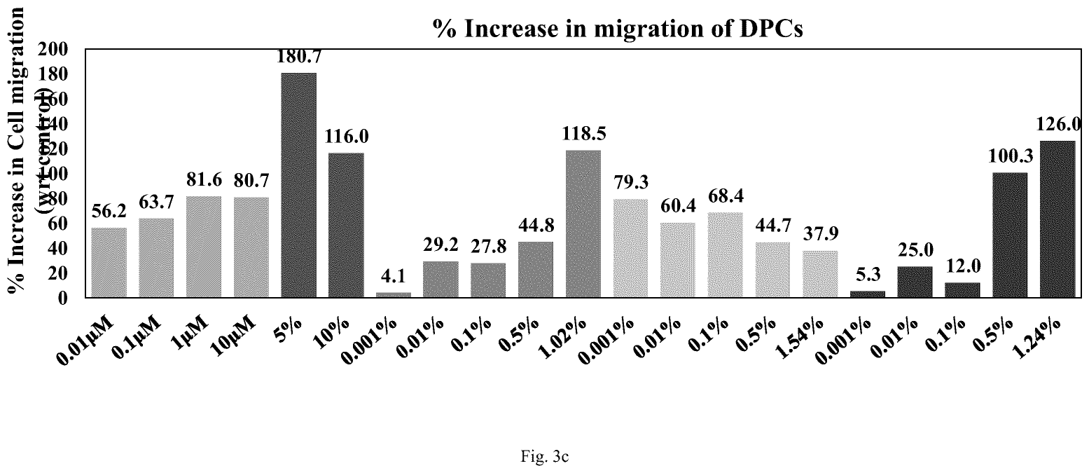

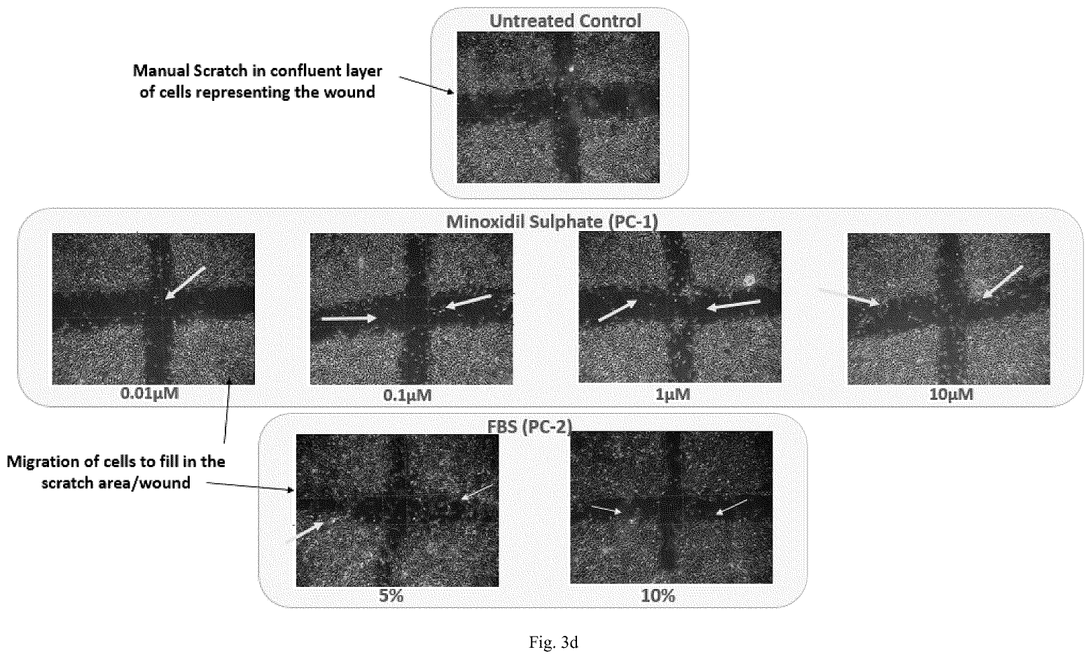

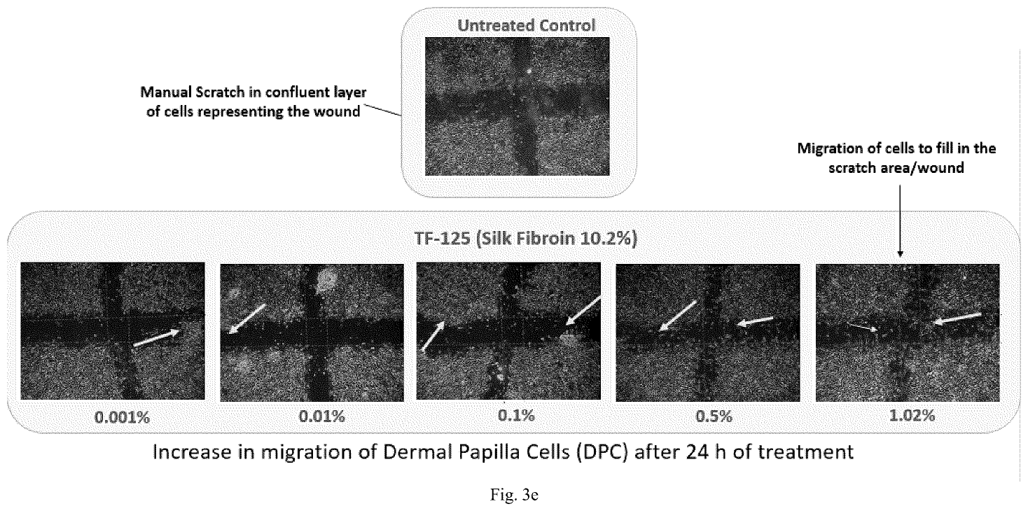

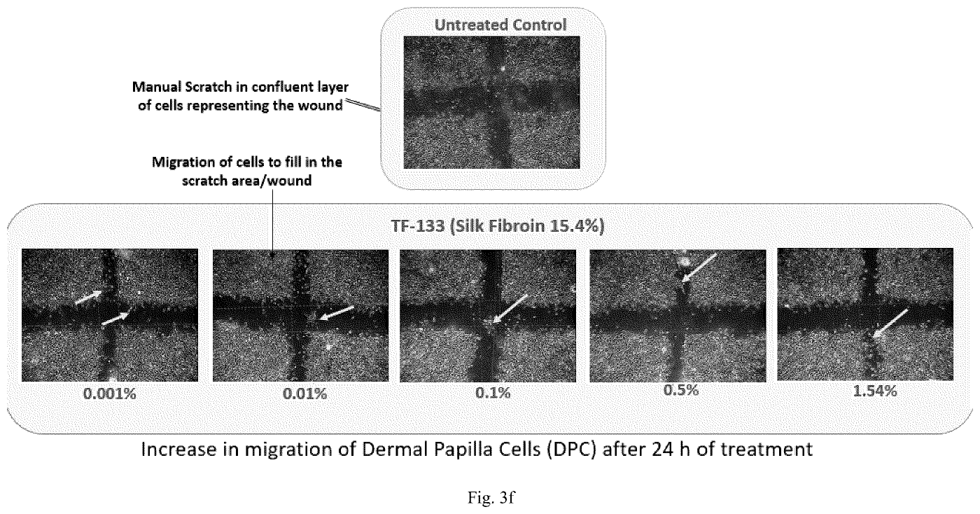

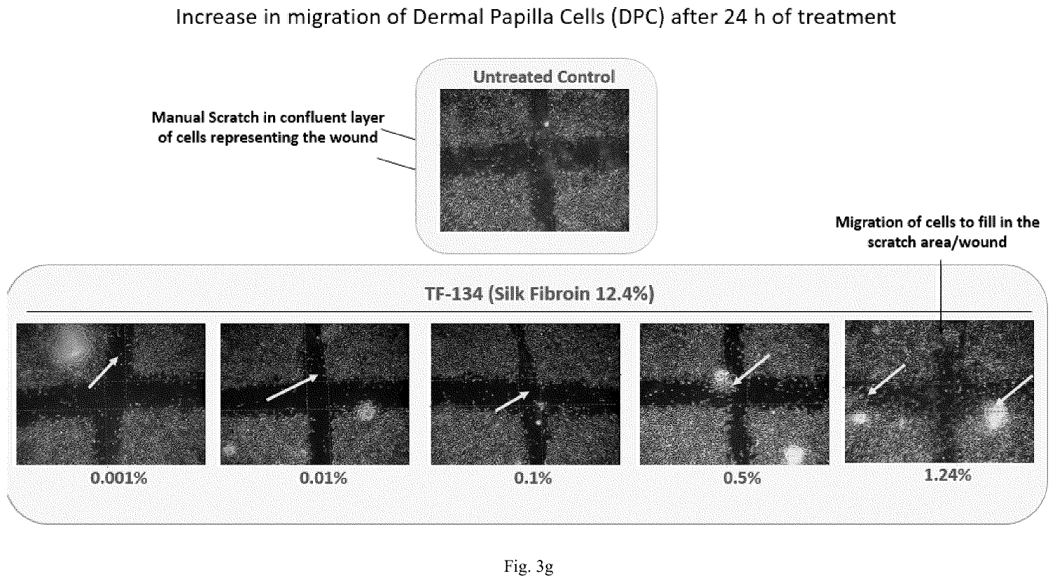

III. Assay 3—Increase in Migration Potential of Dermal Papilla Cells (DPCs) (as Seen in FIGS. 3a-3g)

### Study Design

Test System—Human Dermal Papilla Cells (DPCs)
Number of cells plated—0.2 million cells/well in 12 Well plate
FBS concentration—0%
Test Items—TF-125 (10.2%), TF-133 (15.4%), TF-134 (12.4%)
Time point—24h
Estimation method—Scratch assay

### Procedure

Cells were plated in 10% FBS in 12-well plate and incubated for 24 hours. Cells were then serum starved in 0% FBS for 24 hours. Scratch was created in each well using a sterile tip followed by treatment of cells with 3 Test items at various concentrations. Images were captured (0h). After 24 hours, the images were captured to determine the effect of Test Items on migration. Increase in migration was calculated using ImageJ software.

### Calculations

Increase in migration was calculated wrt control (Untreated cells).

$$[ ( A - B ) / B ] * 100 ; Where A = \% Migration in Test Item treated cells ,$$

$$B = \% Migration in Control ( Untreated ) cells$$

The extent of Migration/Distance migrated was calculated as:
Empty space (pixels) at 0 h—Empty space (pixels) at 24 hours.

For each sample, respective zero h control was taken into consideration.

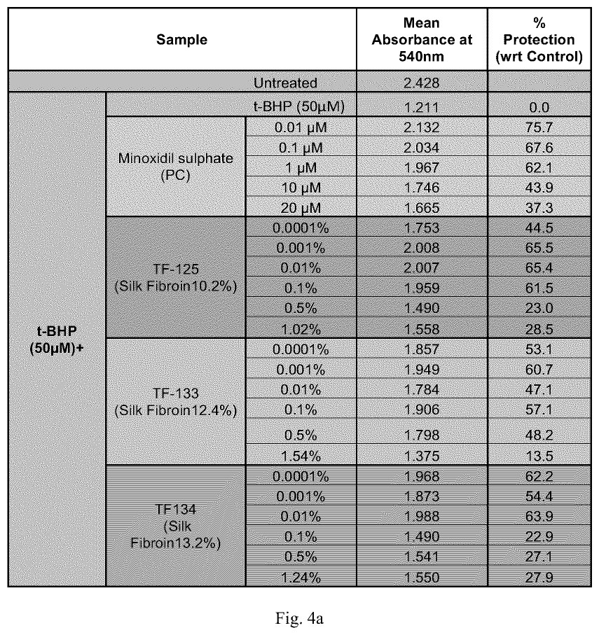

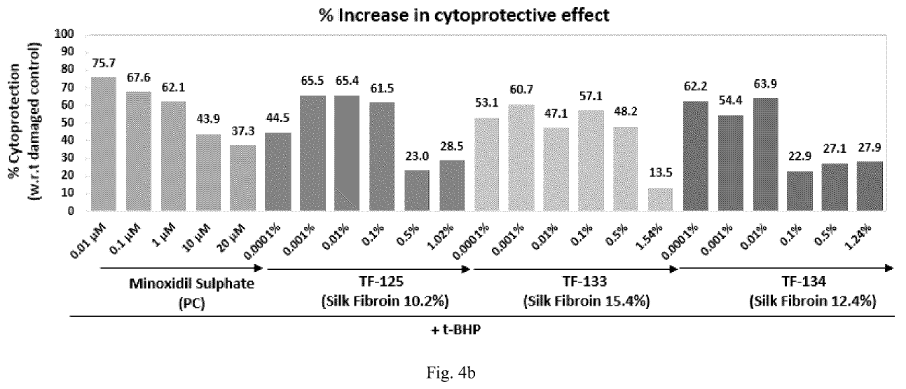

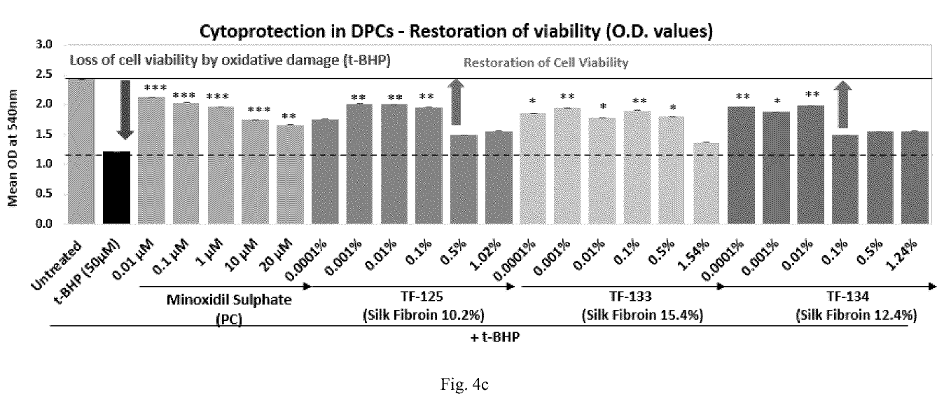

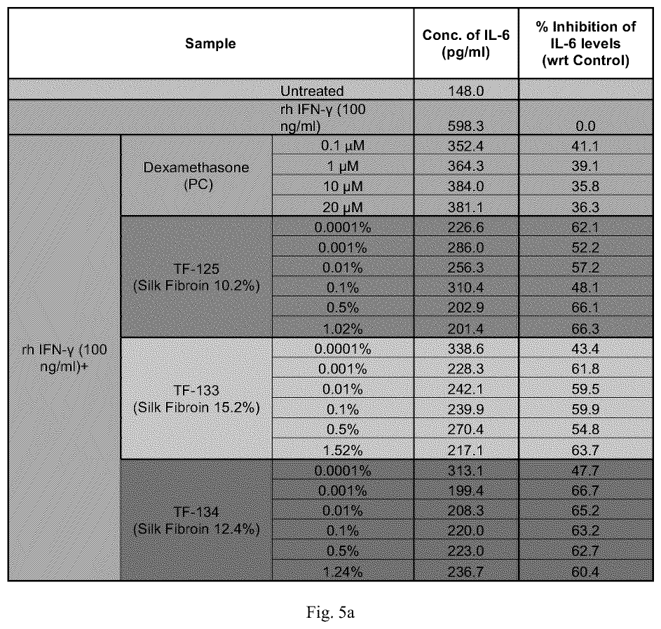

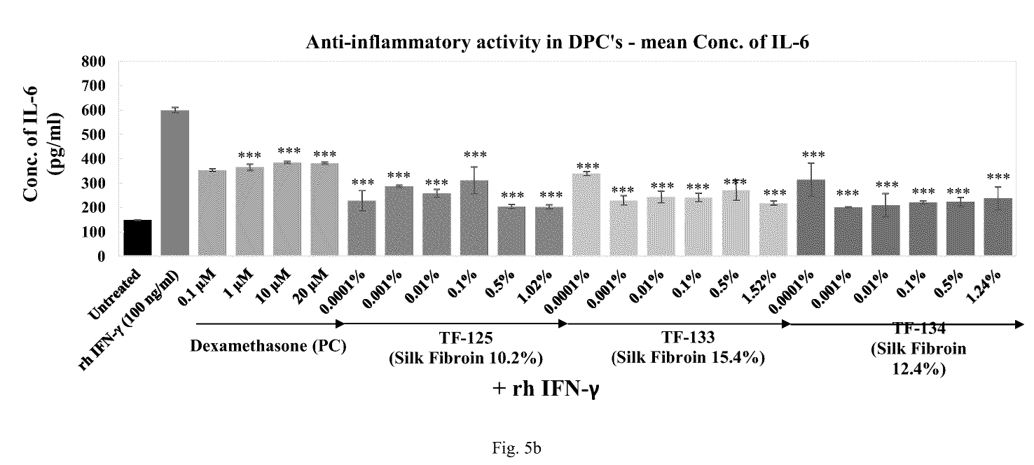

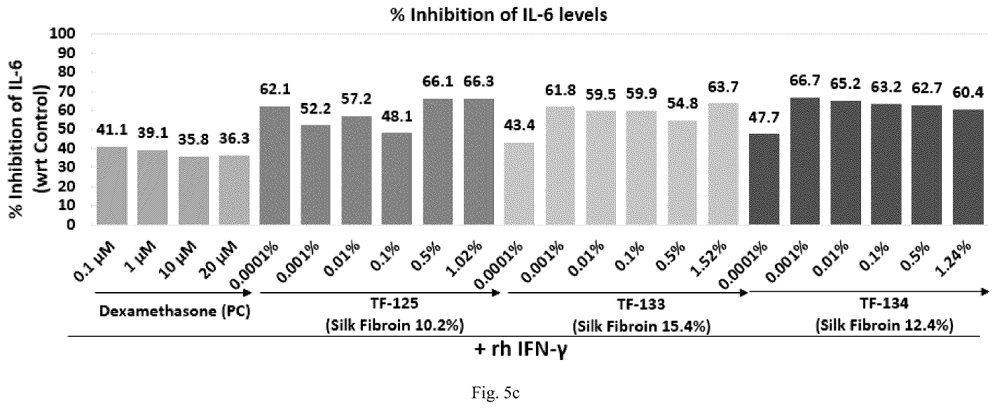

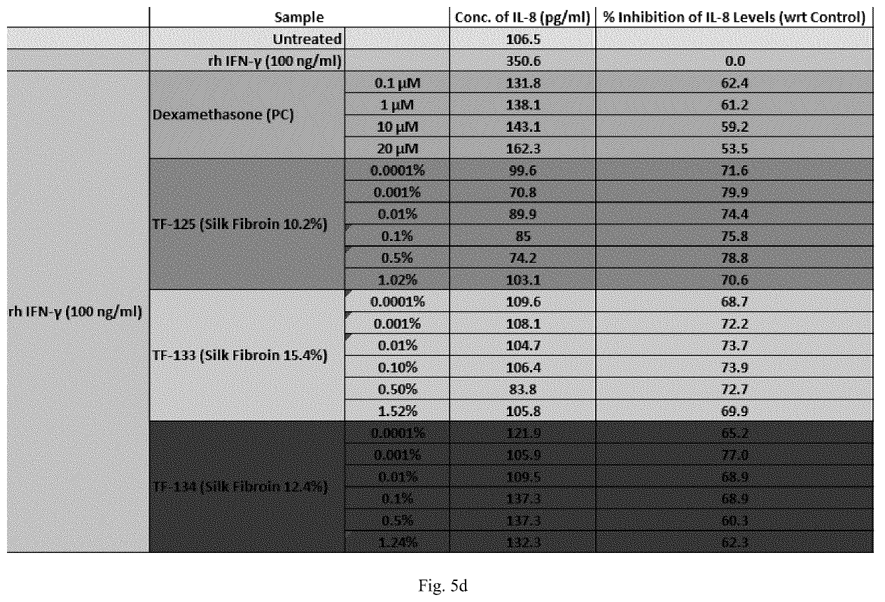

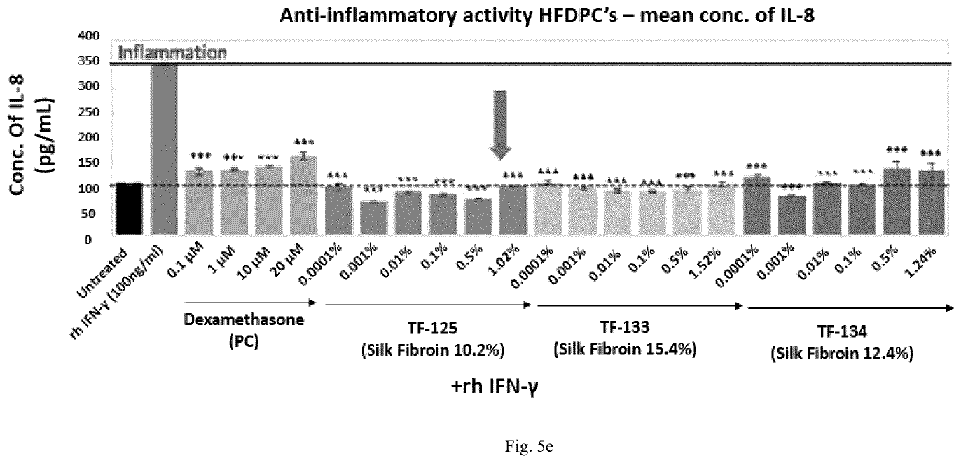

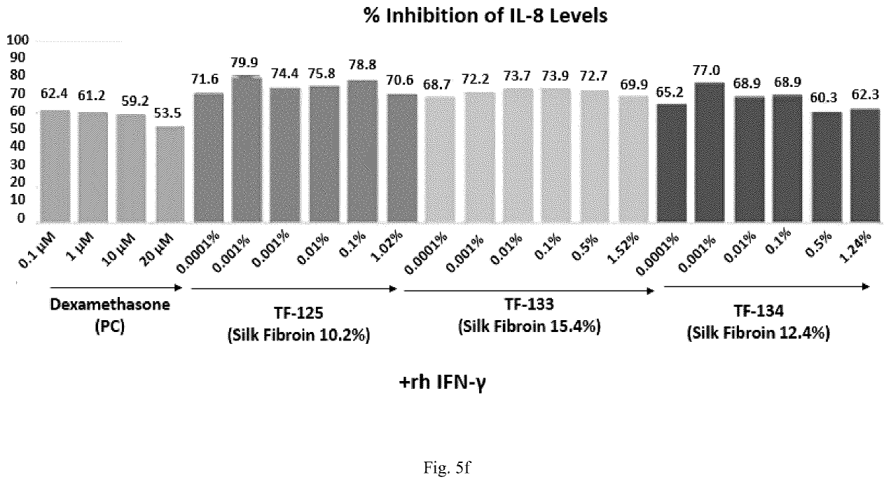

Assay 4—Cytoprotection in Human Dermal Papilla Cells (DPCs) against oxidative damage (as seen in FIGS. 4a-4c)

### Study Design

Test System
Human Dermal Papilla Cells (DPCs)
Number of cells plated
10000 cells/well in 96 Well plate
FBS concentrations
0.5%
Test items
TF 125 (10.2%), TF 133 (15.4%), TF 134 (12.4%)
Time points
24 h pretreatment, 24 h t BHP (50 μM) damage
Estimation method
MTT assay
Positive Control
Minoxidil Sulphate Procedure
Cells were plated in 10% FBS and incubated for 24 h.
Cells were pre-treated with 3 Test items in 0.5% FBS at various concentrations for 24 h.
Cells were treated with tBHP (50 μM) for 24 h for oxidative damage.
Cell viability was determined by MTT assay.
   20 μl of MTT solution was added to each well and cells were incubated for 3 h at 37° C.
   After incubation, supernatants were removed and 150 μl DMSO added to all wells to extract formazan crystals.
   A purple coloured formazan complex was formed.
   Absorbance of each well was measured at 540 nm.

### Calculations

Protective effect on cell viability was calculated wrt controls (untreated and tBHP damaged cells).
{(Absorbance of TI+tBHP))−(Absorbance of tBHP alone)/(Absorbance of Untreated)−(Absorbance of tBHP alone)}*100

### Study Design

Test System—Human Dermal Papilla Cells (DPCs)
Number of cells plated 0.1 million cells/well in 24 Well plate
FBS concentrations 0.1%,
Test items
TF 125 (10.2%), TF 133 (15.4%), TF 134 (12.4%)
Time points
48 h pre treatment, 24 h stimulation (hu IFNγ 10 0 ng/ml)
Estimation method
Human TNF alpha, IL 6, IL 8
Positive Control Dexamethasone

### Procedure

Cells were plated in 10% FBS in 24
well plates and incubated for 24 h.
Cells were serum starved in 0.10% FBS for 24 h.
Cells were treated with 3 Test items in 0.1% FBS at various concentrations for 48h.
After incubation, cells were stimulated with inflammatory stimulus (hu
IFN γ 10 0 ng/ml).
After 24h of stimulation, culture supernatants were collected.
Levels of cytokines (IL6, MIP 1 α, IL 8, TNF alpha) were determined using ELISA as follows
   Assay diluent was added to each well.
   Respective kit standards and samples (supernatants of cells) were directly pipetted into the wells and incubated for 2 h at RT.
   After washing away any unbound substances for a total of 3 times, respective conjugate was added to each well and incubated for 12 h at RT.
   Following a wash (3 times) to remove any unbound conjugate, substrate solution was added to the wells and incubated for 30 min a t RT in dark.
   The reaction was stopped by adding Stop solution to each well.
   The optical density of the color was measured at 450 nm.

### Calculations

Percent Inhibition in each sample was calculated wrt control (huIFN γ treated cells).

$$[ ( BA ) / B ] * 100 ; Where A = Test Item treated cellls ,$$

$$B = Control ( hu IFN γ treated ) cells .$$

### B. Evaluation of Beneficial Effect of Fibroin Proteins (Tf-125, TF-133, TF-134) on Dermatological Indications (Psoriasis, Atopic Dermatitis & Acne) Using In Vitro Skin Cell Based Assays by Multi-Parametric Analysis

Psoriasis vulgaris is a genetic autoimmune disorder that manifests in the skin. Clinically, red plaques with silver or white multi-layered scales characterize psoriasis, with a thickened acanthotic epidermis in patients.

Psoriasis is a multi-factorial skin disease with a complex pathogenesis. Various factors which have been suggested to play a key role in the pathogenesis are T cells, antigen presenting cells (APC's), keratinocytes, Langerhans' cells, macrophages, natural killer cells, an array of Th1 type cytokines, certain growth factors like vascular endothelial growth factor (VEGF), keratinocyte growth factor (KGF), and others.

It has been hypothesized that the disease starts with the activation of T cell by an unknown antigen, which leads to secretion of an array of cytokines by activated T cells, inflammatory cells, and keratinocytes. The characteristic lesion of psoriasis is due to the hyper-proliferation of the keratinocyte.

The histopathological observation of psoriatic lesions reveals epidermal acanthosis, rete ridges, immune-cell infiltration in the dermis, and increased angiogenesis. Although psoriasis has long been considered to be an immune-cell-dependent disease, keratinocytes' critical role in inducing the early pathogenic events and sustaining the prolonged phase of the disorder cannot be ignored.

The hyperproliferation and abnormal differentiation as a secondary phenomenon elicited by the immune response is the pathogenic function of keratinocytes in psoriasis. Keratinocytes respond to the psoriatic lesions with an overactive wound-healing procedure. Abnormal and hyper-proliferation, disturbed apoptosis and hyper-inflammation in keratinocytes is a hallmark feature of psoriasis.

Anti-psoriatic agents function by inhibition of proliferation, enhanced apoptosis and anti-inflammatory activity.

Psoriasis is an autoimmune disorder, characterized by the hyper proliferation and abnormal differentiation of keratinocytes, which leads to inflammation in dermis, epidermis and leukocyte infiltration.

Dysfunctional apoptosis has an important role in the development of several skin diseases. Psoriasis is a common chronic inflammatory skin disease characterized by hyperproliferation with incomplete differentiation of epidermal keratinocytes and decreased keratinocyte apoptosis. Inflammatory response occurs in both keratinocyte and immune cells and is key hallmark event in psoriasis. HaCaT cells are immortalized human epidermal keratinocytes, which are a commonly used cell model in psoriasis research. These cells are widely employed as a model system to assess anti psoriatic effect of test compounds. Anti-psoriatic activity of a compound is indicated by inhibition of cellular proliferation of keratinocytes.

Some natural compounds such as curcumin is widely reported to demonstrate antiproliferative potential in keratinocytes. Induction of apoptosis in abnormally hyperproliferating keratinocytes reflects anti-psoriatic potential. Downregulation of excessively secreted cytokines in keratinocytes also supports the anti-psoriatic claim. Inhibition of VEGF in keratinocytes demonstrates the anti-angiogenic potential that is helpful in psoriasis by targeting the increased vasculature leading to redness and inflammation. Anti-proliferative, pro-apoptotic, anti-inflammatory activity in abnormally hyperproliferating keratinocytes supports the anti-psoriatic potential.

### Psoriasis

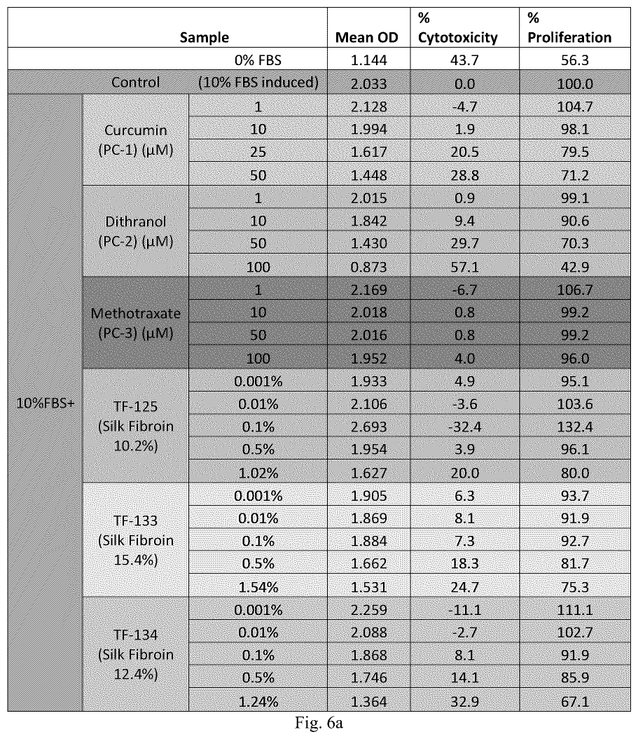

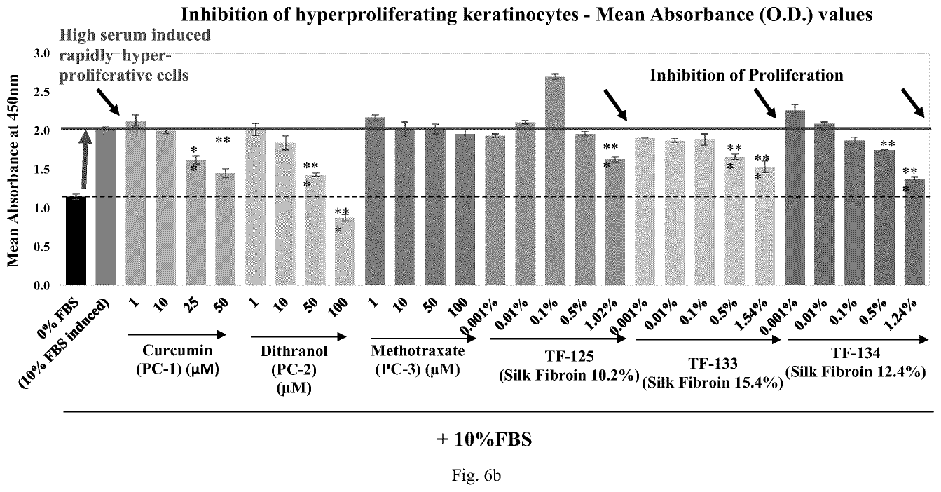

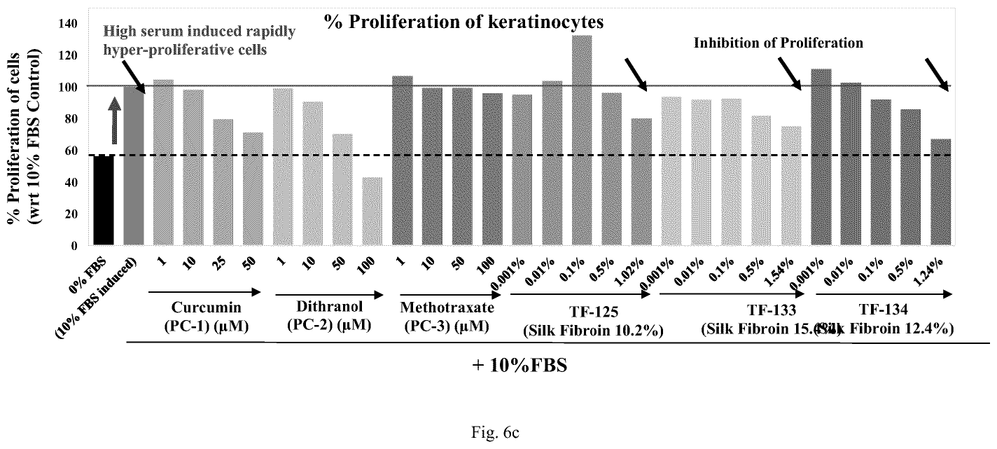

### 1a. Inhibition of Keratinocytes (HaCaT) Proliferation (as Seen in FIGS. 6a-6c)

### Study Design

Test System—Human immortalized Keratinocyte cell line (HaCaT) (cultured in high serum conditions for 2-3 passages to achieve hyperproliferative state)
Number of cells plated—10,000 cells/96-well plates
FBS concentrations—10% (to maintain hyperproliferative state)
Test items—TF-125 (10.2%), TF-133 (15.4%), TF-134 (12.4%)
Time points—3 days
Estimation method—MTT assay
Positive Control/s—Curcumin, Dithranol, Methotrexate

### Procedure

Cells were plated in 10% FBS in 96-well plates and incubated for 24 hours. Cells were then serum starved in 0% FBS for 24 hours. Cells were treated with 3 Test items in 10% FBS at various concentrations. After 72 hours the effect on inhibition of proliferation was determined by MTT assay. 20 μl of MTT solution was then added to each well and cells were incubated for 3 hours at 37° C. After incubation, supernatants were removed and 150 μl DMSO added to all wells to extract formazan crystals. A purple-colored formazan complex was formed. Absorbance of each well was measured at 540 nm.

### Calculations

Inhibition of proliferation was calculated wrt control (Untreated 10% FBS stimulated cells).

[(B−A)/B]*100; Where A=Absorbance in Test Item treated cells, B=Absorbance in Control (Untreated 10% FBS stimulated cells)

### Psoriasis

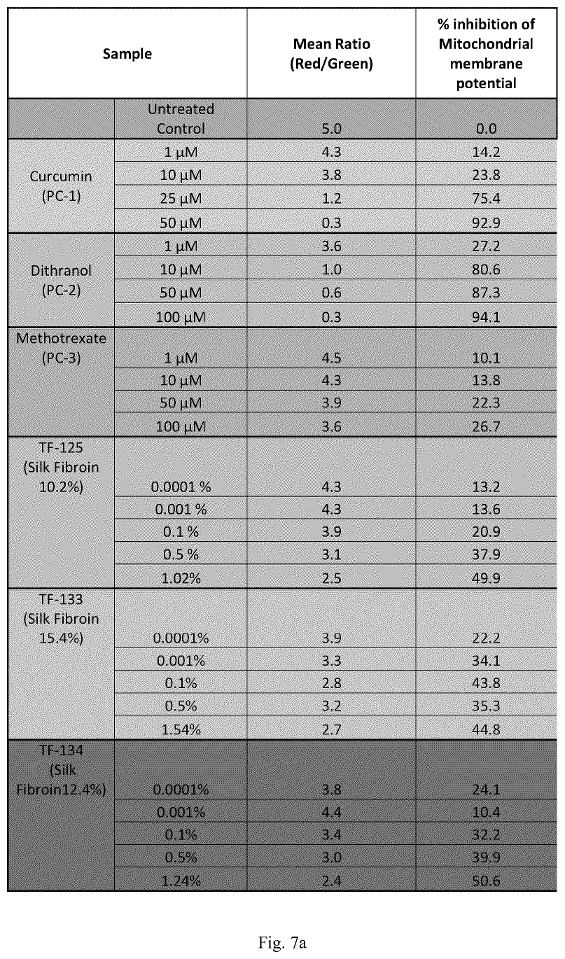

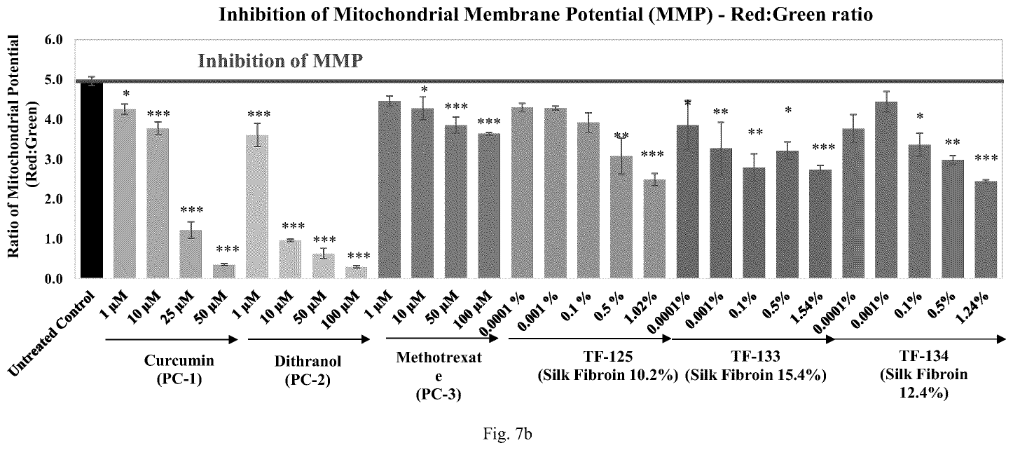

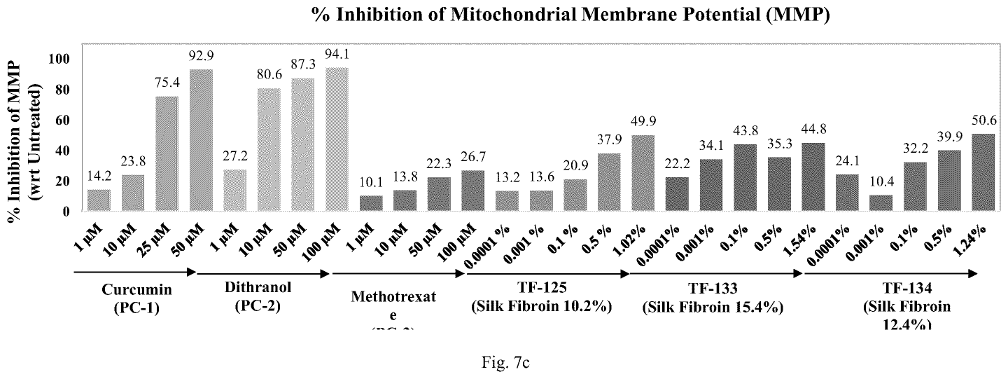

### 1b)—Pro-Apoptotic Effect—Inhibition of Mitochondrial Membrane Potential (MMP) in Keratinocytes (as Seen in FIGS. 7a-7c)

### Study Design

Test System—Human immortalized Keratinocyte cell line (HaCaT)
Number of cells plated—10,000 cells/96-well plates
FBS concentrations—10%,
Test items—TF-125 (10.2%), TF-133 (15.4%), TF-134 (12.4%)
Time points—48 hours
Estimation method—Mitochondrial Membrane Potential using JC-1 dye.
Positive Control/s—Curcumin, Dithranol, Methotrexate

### Procedure

Cells were plated in 10% FBS in 96-well plates and incubated for 24 hours. Cells were then treated with 3 Test items in 0.1% FBS at various concentrations. After 48 hours, the effect on Mitochondrial membrane Potential was determined by JC-1 assay. Active mitochondria in live cells exhibit brighter red fluorescence signal compared to mitochondria with lower membrane potential in apoptotic cells which fluoresce green with JC-1 dye. Ratio of Red: Green indicates degree of MMP. Inhibition of MMP shows depolarization of MMP. After incubation, medium from each well was removed. 100 μl of 10 μM JC-1 dye in PBS was then added to each well and cells were incubated for 3±1 h at 37° C. for 25 mins. After incubation, cells were rinsed with PBS to remove dye and Fluorescence was measured at 485/590 for red and 485/528 for green. Ratio of Red: green (health cells) was calculated.

### Calculations

Inhibition of Mitochondrial membrane potential was calculated wrt control (Untreated cells).

$$[ ( B - A ) / B ] * 100 ;$$

$$Where A = Ratio of Red : Green in Test Item treated cells ,$$

$$B = Ratio of Red : Green in Control ( Untreated ) cells$$

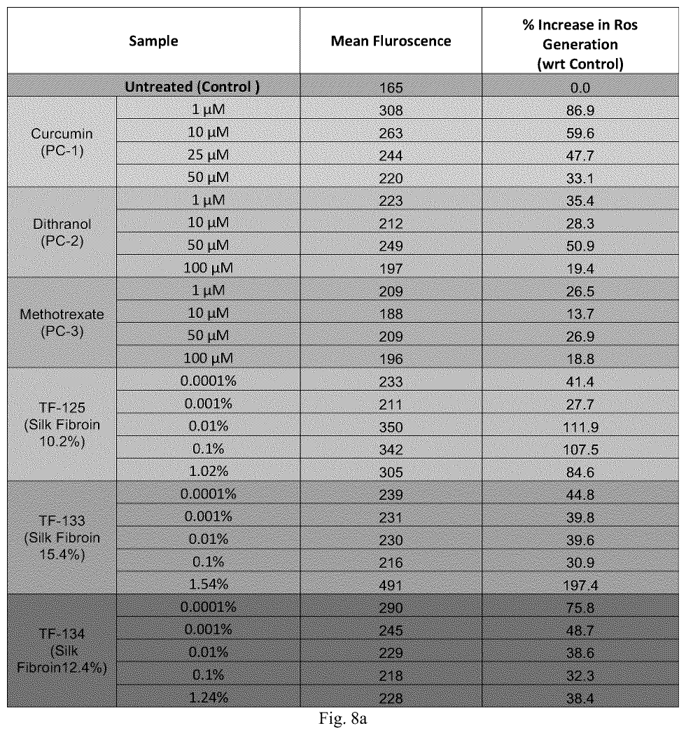

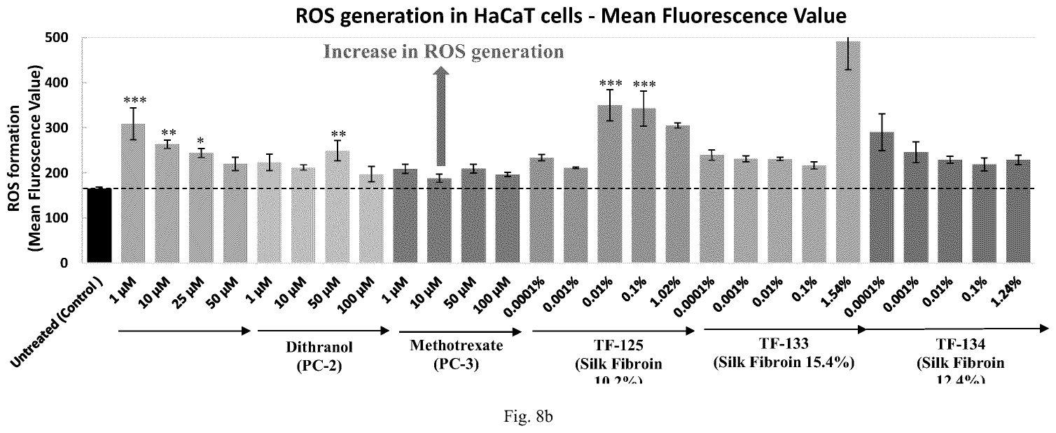

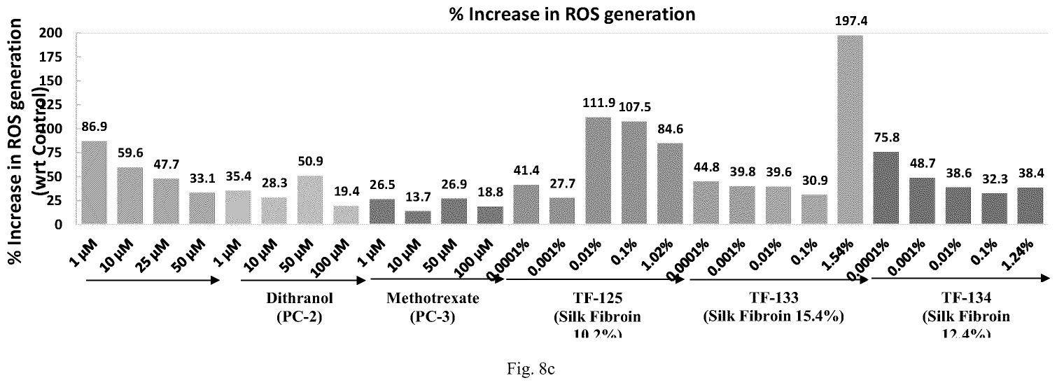

Assay 1b)—Pro-apoptotic effect—Increase in Reactive Oxygen Species (ROS) Generation in keratinocytes (as seen in FIGS. 8a-8c)

### Study Design

Test System—Human immortalized Keratinocyte cell line (HaCaT)
Number of cells plated—10,000 cells/96-well plates
FBS concentrations—10%,
Test items—TF-125 (10.2%), TF-133 (15.4%), TF-134 (12.4%)
Time points—48 hours
Estimation method—Reactive Oxygen Species (ROS) using H2DCFDA dye.
Positive Control/s—Curcumin, Dithranol, Methotrexate

### Procedure

Cells were plated in 10% FBS in 96-well plates and incubated for 24 hours. Cells were then treated with 3 Test items in 0.1% FBS at various concentrations. After 48 hours, the effect of Reactive Oxygen Species (ROS) was determined by using H2DCFDA dye. The cell-permeant 2′,7′-dichlorodihydrofluorescein diacetate (H2DCFDA) is used as an indicator for reactive oxygen species (ROS) in cells. After incubation, medium from each well was removed. 100 μl of 10 μM H2DCFDA dye in DMEM medium was added to each well and cells were incubated for 3±1 hr at 37° C. for 45 mins. After incubation, cells were rinsed with PBS to remove dye and Fluorescence was measured at 485/528 to determine the ROS generation.

### Calculations

Increase in ROS generation was calculated wrt control (Untreated cells).

$$[ ( A - B ) / B ] * 100 ;$$

$$Where A = Fluorescence in Test Item treated cells , B = Fluorescence in Control ( Untreated ) cells$$

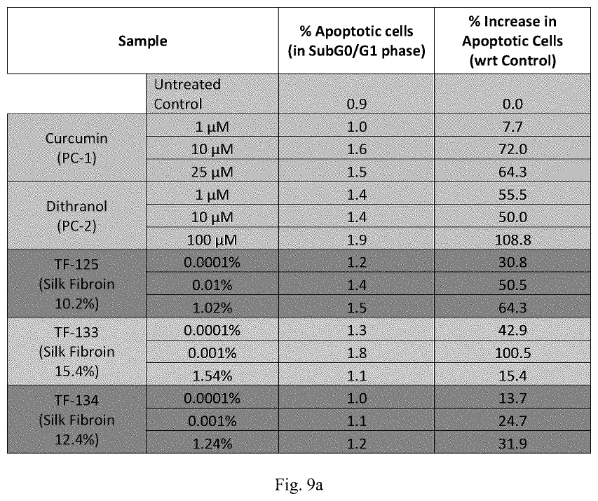

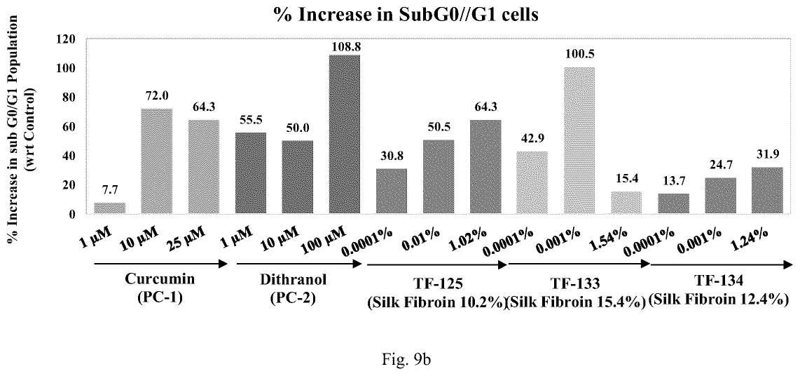

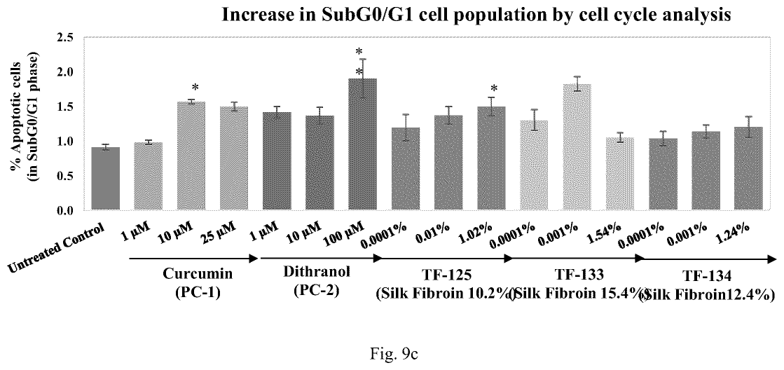

Assay 1b)—Pro-Apoptotic Effect—Increase in subG0/G1 Cells by Cell Cycle Assay (as Seen in FIGS. 9a-9c)

### Study Design

Test System—Human immortalized Keratinocyte cell line (HaCaT)
Number of cells plated—0.25 million cells/12-well plates
FBS concentrations—10%,
Test items—TF-125 (10.2%), TF-133 (15.4%), TF-134 (12.4%)
Time points—48 hours
Estimation method—SubG0/G1 apoptotic cells by Propidium iodide solution
Positive Control/s—Curcumin, Dithranol, Methotrexate

### Procedure

Cells were plated in 10% FBS in 96-well plates and incubated for 24 hours. Cells were then treated with 3 Test items in 0% FBS at various concentrations. After 48 hours, the pro-apoptotic effect was measured by Cell-cycle analysis. After incubation, medium from each well was removed and cells were harvested by trypsinization and centrifuged at 450 g for 5 min (low brake). Supernatant was discarded and cell pellet washed with 1×PBS at 450 g for 5 min (low brake). Cells were fixed with Ice-cold 70% ethanol (500 μl) and stored at 4° C. for 24 h prior to staining. For staining, Ethanol fixed cells were centrifuged at low brake and washed with PBS. 200 μl of cell cycle reagent was added to each sample at kept at RT for staining for 30 min in dark. After incubation, cells were acquired using flow cytometer.

### Calculations

Increase in apoptotic cell population (sub G0/G1) was calculated wrt control (Untreated cells).

$$[ ( A - B ) / B ] * 100 ; Where A = \% subG 0 / G 1 cells in Test Item treated ,$$

$$B = \% subG 0 / G 1 cells in Control ( Untreated )$$

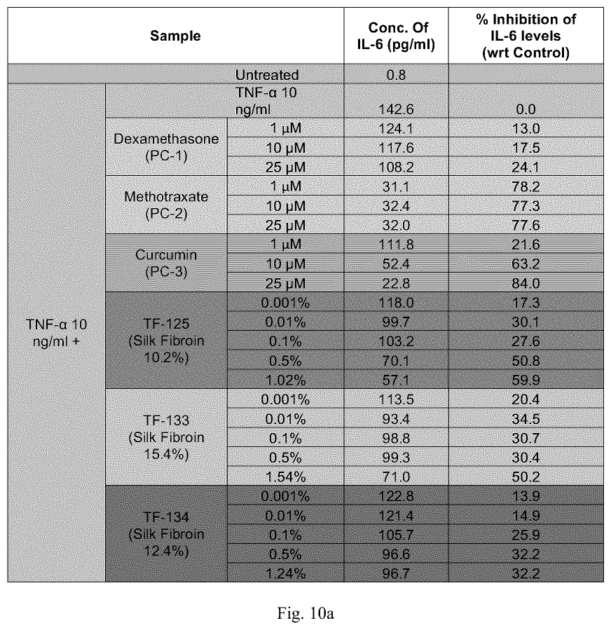

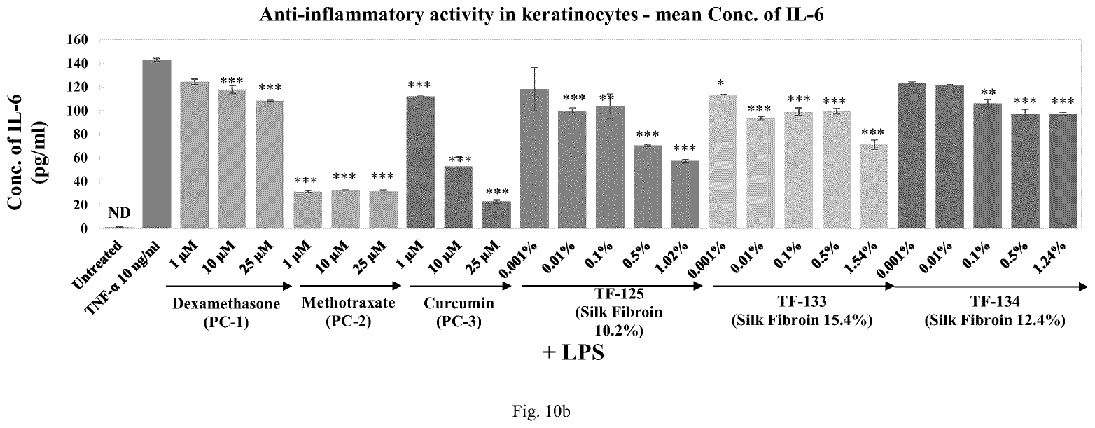

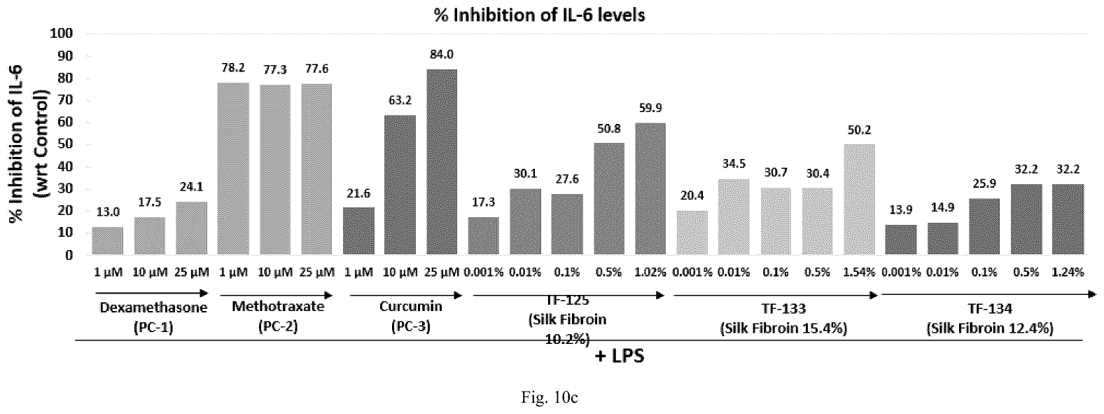

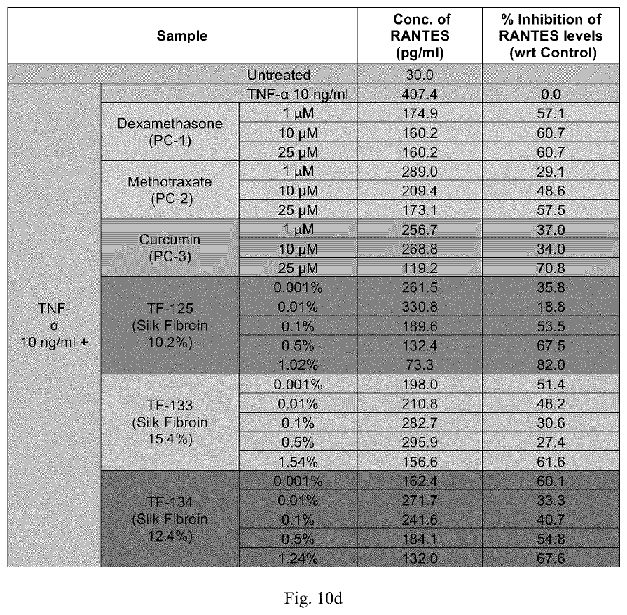

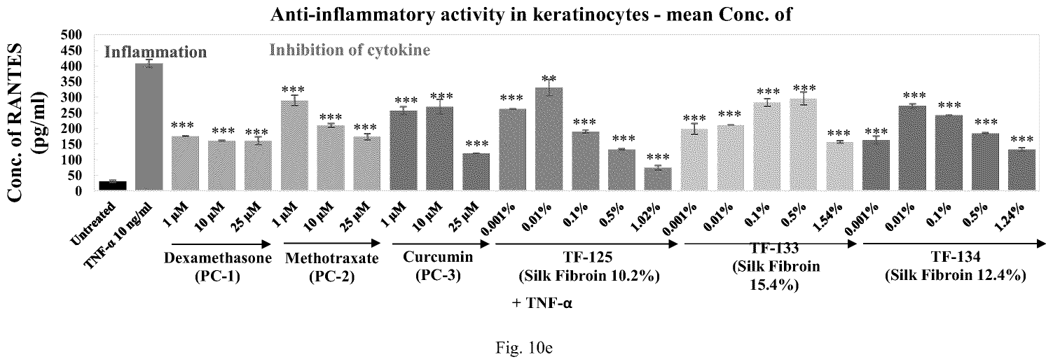

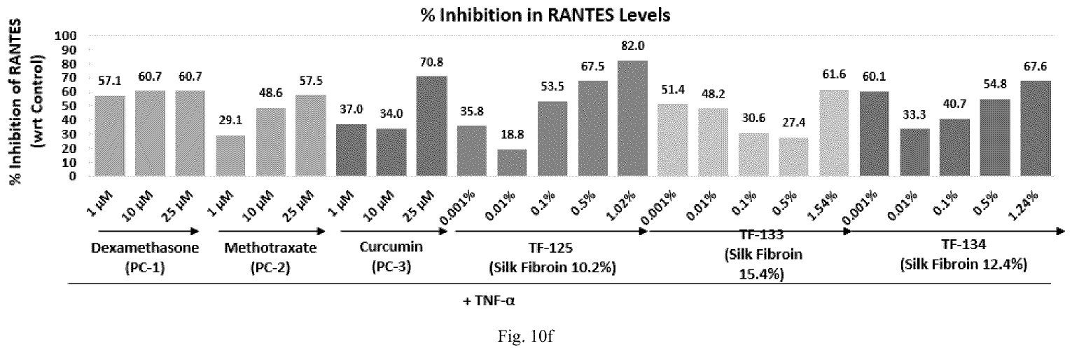

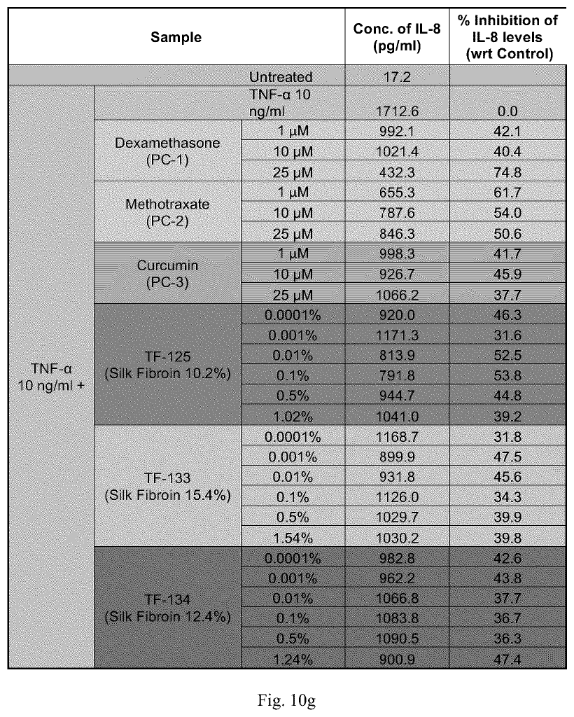

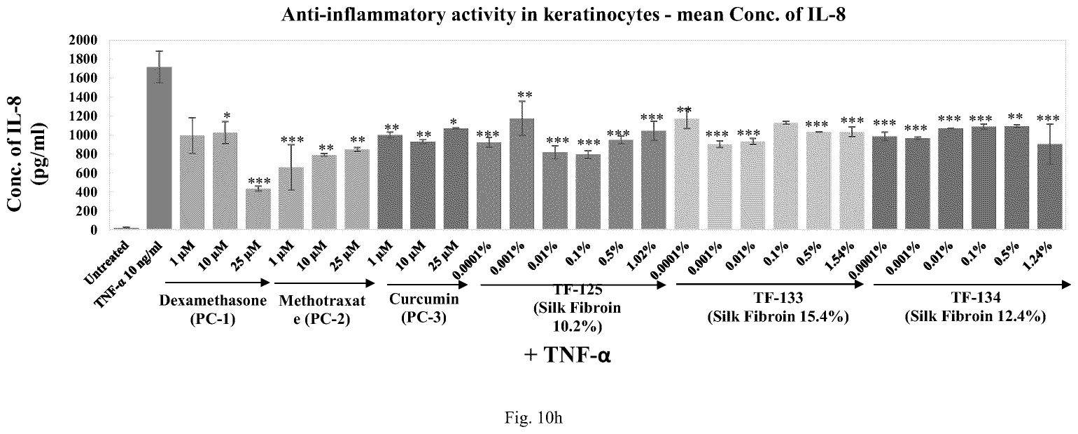

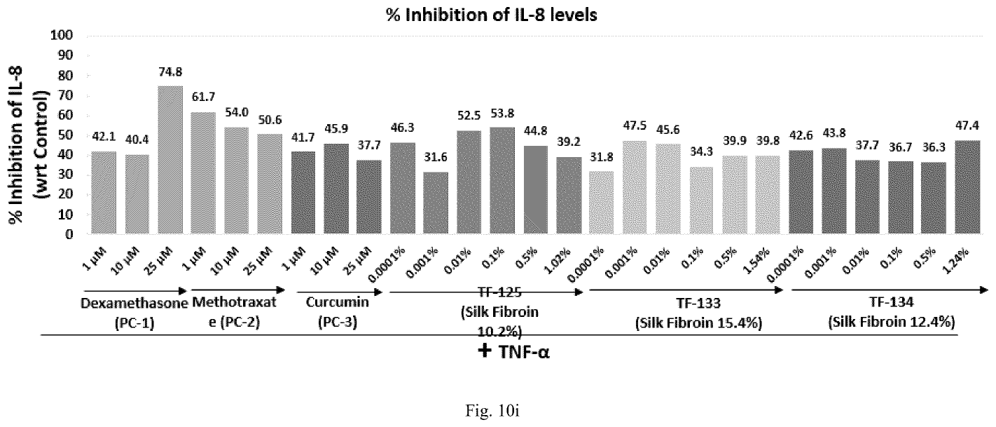

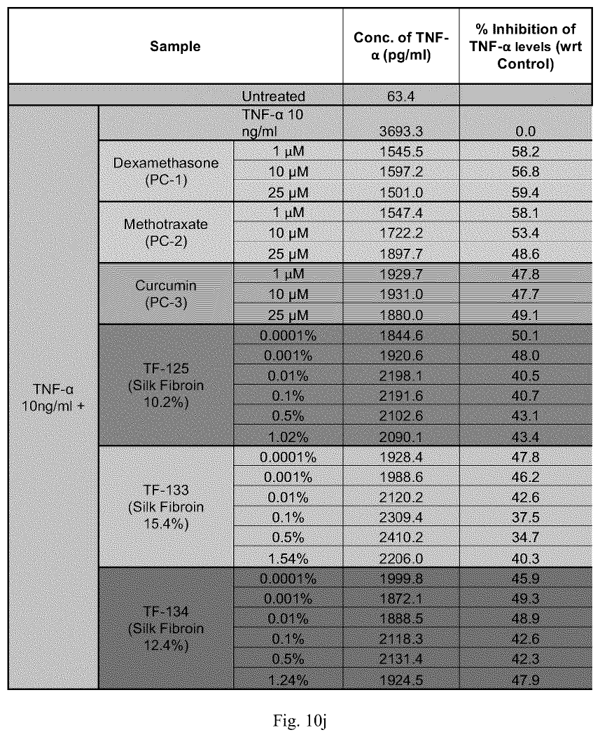

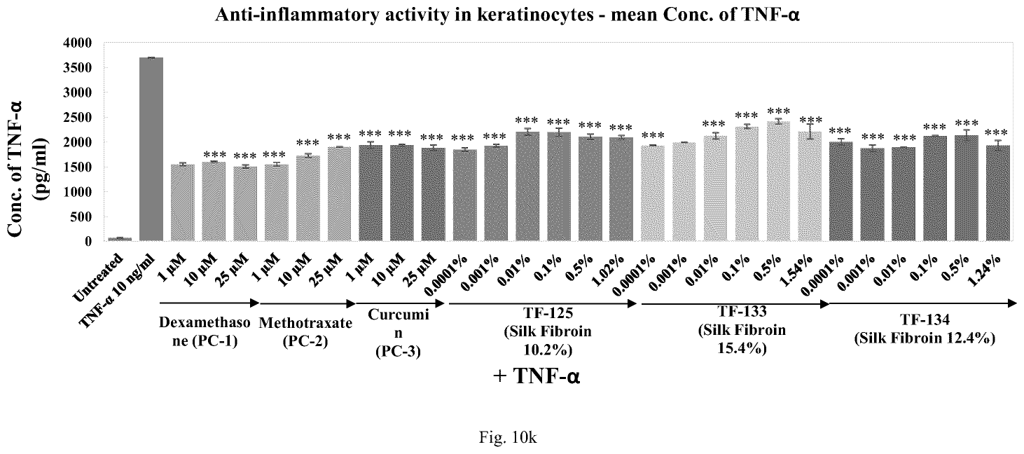

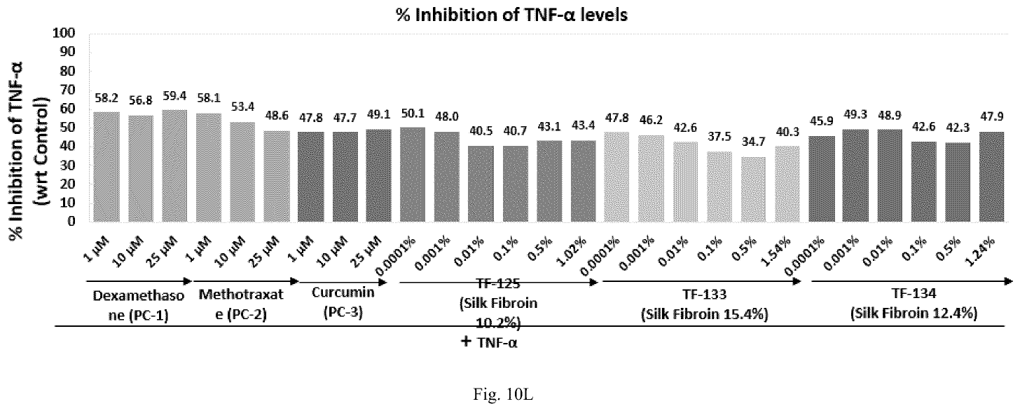

### 1c)—Anti-Inflammatory Effect (IL-6, RANTES, IL-8, TNF-α) in Keratinocytes (as Seen in FIGS. 10a-101)

### Study Design

Test System—Human immortalized Keratinocyte cell line (HaCaT)
Number of cells plated—0.1 million cells/24-well plates
FBS concentrations—0.1%,
Test items—TF-125 (10.2%), TF-133 (15.4%), TF-134 (12.4%)
Time points—IL-6, RANTES—24 h pre-treatment, 24 h stimulation (TNF-α10 ng/ml), IL-8, TNF-α-48 h pre-treatment, 24 h stimulation (TNF-α10 ng/ml)
Estimation method—ELISA
Positive Control/s—Curcumin, Dexamethasone, Methotrexate

### Procedure

Cells were plated in 10% FBS in 24-well plates and incubated for 24 hours. Cells were then serum starved in 0.1% FBS for 24 h hours. Cells were treated with 3 Test items in 0.1% FBS at non-cytotoxic concentrations for 24h for IL-6 and RNATES and 48h for IL-8 & TNF-α. After incubation, cells were stimulated with inflammatory stimulus (hu-TNF-α 10 ng/ml). After 24 hours of stimulation, culture supernatants were collected, and lysates were also prepared. Levels of IL-6, RANTES (supernatants) and IL-8, TNF-alpha (lysates) were determined using ELISA as follows-Assay diluent was added to each well. Respective kit standards and samples (supernatants/lysates) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm. Conc of cytokines were calculated from standard curve.

### Calculations

Inhibition in levels of cytokines was calculated wrt control (TNF-αstimulated cells).

$$[ ( B - A ) / B ] * 100 ;$$

$$Where A = Levels of cytokines in Test Item treated cells ,$$

$$B = Levels of cytokines in Control ( TNF - α stimulated cells ) .$$

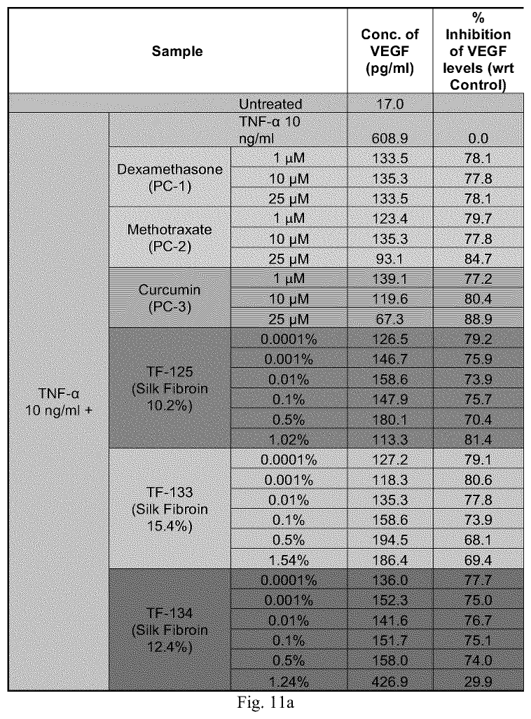

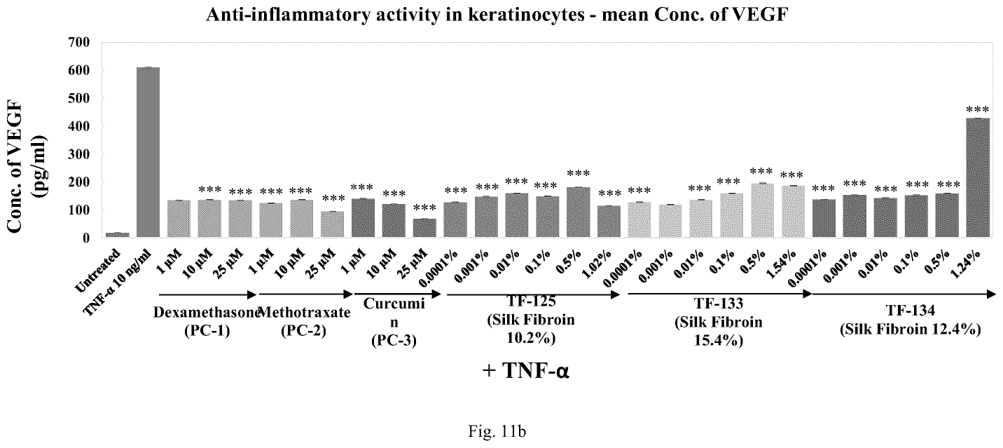

### 1d)—Anti-Angiogenic Effect (VEGF Inhibition) in Keratinocytes (as Seen in FIGS. 11a-11c)

### Study Design

Test System—Human immortalized Keratinocyte cell line (HaCaT)
Number of cells plated—0.1 million cells/24-well plates
FBS concentrations—0.1%,
Test items—TF-125 (10.2%), TF-133 (15.4%), TF-134 (12.4%)
Time points—48 h pre-treatment, 24 h stimulation (TNF-α10 ng/ml)
Estimation method—ELISA
Positive Control/s—Curcumin, Dexamethasone, Methotrexate

### Procedure

Cells were plated in 10% FBS in 24-well plates and incubated for 24 hours. Cells were then serum starved in 0.1% FBS for 24 hours. Cells were treated with 3 Test items in 0.1% FBS at non-cytotoxic concentrations for 48 hours. After incubation, cells were stimulated with inflammatory stimulus (hu-TNF-α 10 ng/ml). After 24 hours of stimulation, cell lysates were prepared. Levels of VEGF were determined using ELISA as follows: Assay diluent was added to each well. Respective kit standards and samples (lysates) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm. Conc of cytokines were calculated from standard curve.

### Calculations

Inhibition in levels of VEGF was calculated wrt control (TNF-α stimulated cells).

$$[ ( B - A ) / B ] * 100 ; Where A = Levels of VEGF in Test Item treated cells ,$$

$$B = Levels of VEGF in Control ( TNF - α stimulated cells )$$

### Atopic Dermatitis (AD)

Atopic dermatitis (AD) is a chronic inflammatory skin disease with specific genetic and immunological mechanisms. AD is a chronic multifactorial inflammatory skin disease. The pathogenesis of AD remains unclear, but the disease results from dysfunctions of skin barrier and immune response, where both genetic and environmental factors play a key role. Th2 cells circulating in the peripheral blood of AD patients result in elevated serum IgE and eosinophils. Skin injury by environmental allergens, scratching, or microbial toxins activates keratinocytes to release proinflammatory cytokines and chemokines that induce the expression of adhesion molecules on vascular endothelium and facilitate the extravasation of inflammatory cells into the skin. Keratinocyte-derived thymic stromal lymphopoietin (TSLP) and DC-derived IL-10 also enhance Th2 cell differentiation.

Skin is the interface between an organism and the external environment, and hence the stratum corneum (SC) is the first to withstand mechanical insults that, in certain conditions, may lead to integrity loss and the development of pressure ulcers. The SC is usually described by a “bricks and mortar” structural model, where the corneocytes are completely flattened and their keratin filled interior is surrounded by protein and lipid envelopes cornified envelopes and cornified lipid envelopes, CEs and CLEs, respectively. The cornified cell envelope structure is formed beneath the plasma membrane in terminally differentiating stratified squamous epithelia. It provides a vital physical barrier to these tissues in mammals and consists of a 10 nm thick layer of highly crosslinked insoluble proteins In cultured keratinocytes, CEs are developed in 5-7 days of culture in low calcium medium. CE can be prepared by exhaustive boiling cultured keratinocytes in a solution containing a surfactant such as SDS and recovering the insoluble fraction by removing the soluble components by such means as centrifugation.

Atopic dermatitis (AD) is an eczematous, pruritic skin disorder with extensive barrier dysfunction. The barrier dysfunction correlates with the downregulation of barrier-related molecules such as filaggrin (FLG), loricrin (LOR), and involucrin (IVL). IVL is expressed in the upper spinous layer, but mainly in the granular layers, and is involved in the initial step of cornified envelope formation. LOR is the most abundant component of the cornified envelope. It is very hydrophobic, insoluble, and is easily polymerized via disulfide crosslinking in ambient air, making it suitable as a protein that reinforces the cornified envelope. FLG is involved in aggregating the K1 and K10 filaments into higher-molecular-weight parallel structures that facilitate the incorporation of K1 and K10 into the cornified envelope and contribute to the thin granular keratinocyte shape. Aquaporins (AQPs), a family of membrane channel proteins that allow the osmotic movement of water and small neutral solutes. They play key role in keratinocyte early differentiation, keratinocyte proliferation and migration during wound healing; skin hydration (circadian rhythm); maintenance of epidermal water permeability barrier. Skin barrier is disrupted in AD, with poor expression of surface markers.

Atopic Dermatitis (AD) is one of the most common allergic mediated inflammatory skin diseases, and the most common form of childhood eczema. It is thought that a range of genetic and environmental factors contribute to the severity of pruritus from AD.

Acute AD is triggered by increase of CD4+ and T-Helper2 (Th2) cells in the epithelium. Th2 cells secrete a number of cytokines, including IL-4, IL-33, and IL-13, aiding in the promotion of Immunoglobulin-E (IgE) which is linked to causing hypersensitivity to allergens and impaired barrier function. Overexpression of pro-inflammatory cytokines in a hallmark feature in AD. Increased Thymic Stromal Lymphopoietin (TSLP) has also been found to be overexpressed in AD lesions. Skin barrier dysfunction is the initial step in the development of AD. Multiple factors, including immune dysregulation, filaggrin mutations, deficiency of antimicrobial peptides, and skin dysbiosis contribute to skin barrier defects. Increase in filaggrin, involucrin, loricrin and aqaporin-3 is reported for anti-AD activity. HaCaT are reported widely as model system for screening anti-AD properties of test compounds.

Improvement of skin barrier by formation of cornified envelope, increase in expression of surface proteins, anti-inflammatory activity in keratinocytes and inhibition of IgE secretion in immune cells reflects anti-AD potential.

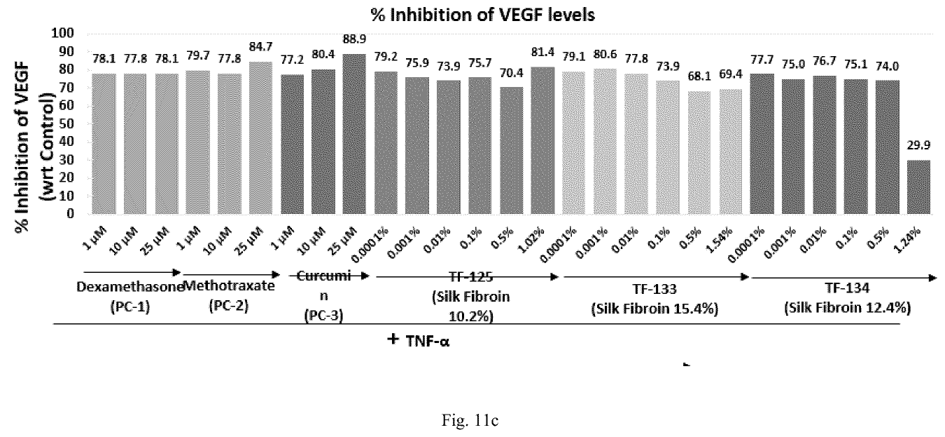

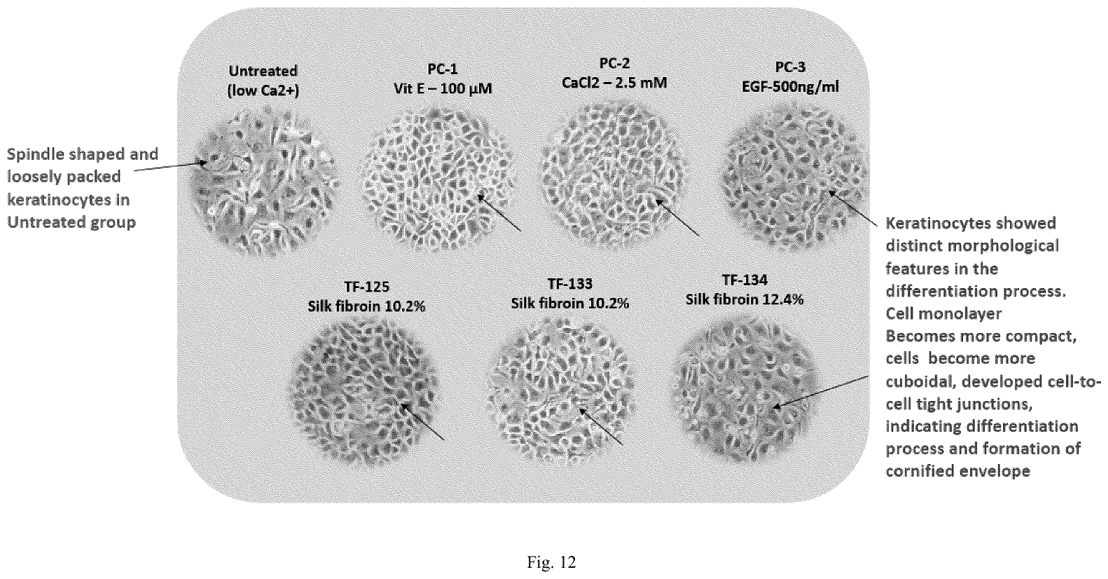

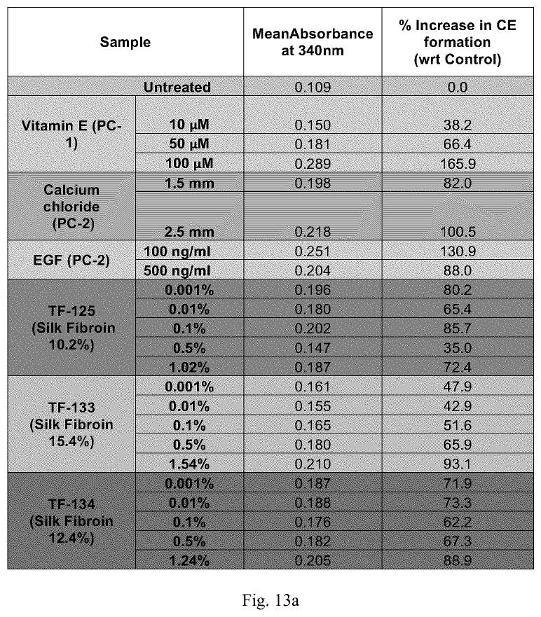

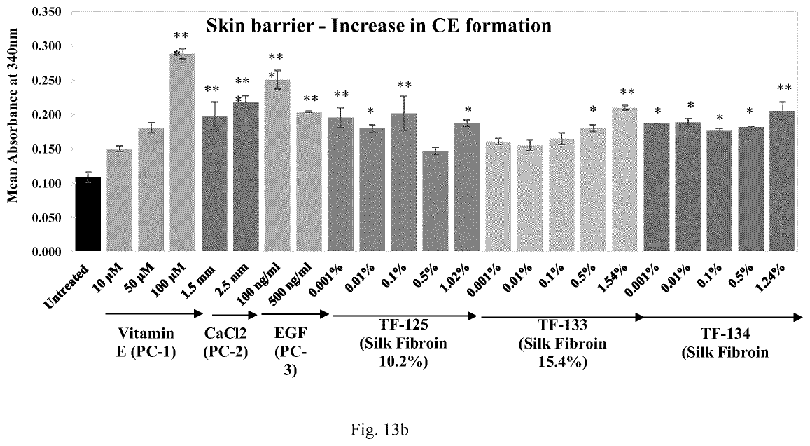

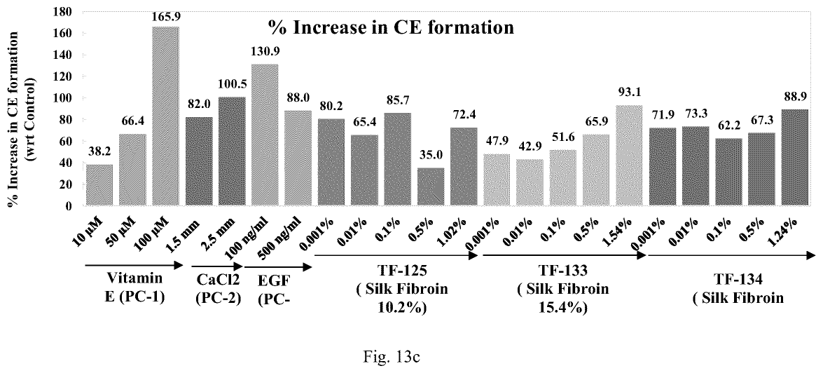

### 2a)—Improvement of Skin Barrier Formation by Increased in Cornified Envelope (as Seen in FIGS. 12 and 13a-13c)

### Study Design

Test System—Human immortalized Keratinocyte cell line (HaCaT)
Number of cells plated—1 million cells/6-well plates
FBS concentrations—0%, Low calcium
Test items—TF-125 (10.2%), TF-133 (15.4%), TF-134 (12.4%)
Time points—7 days, followed by re-addition of Test Item on every 3rd day.
Estimation method—Turbidity of membrane/Absorbance at 340 nm.
Positive Control/s—Vitamin E, Calcium chloride, EGF.

### Procedure

Cells were plated in 10% FBS in 6-well plates and incubated for 24 hours. Cells were then treated with 3 Test items at non-cytotoxic concentrations in calcium free medium for 7 days, replacing media every 3rd day. After incubation, cells processed for skin barrier strengthening potential measured as follows: Cells were harvested by trypsinization in pre-labelled Eppendorf tubes and centrifuged at 300 g for 5 mins. Supernatant was discarded and the cell pellet washed with 1×PBS to remove media residues. Cells were washed at 300 g for 5 mins. Supernatant was discarded and cell pellet was resuspended in 200 μl CE buffer (2% SDS, 20 mM DTT and 0.1M tris Buffer). The mixture was boiled at 95° C. for 5 mins to dissolve crosslinked envelopes. 90 μl of mixture transferred to 96 well plates in duplicate and absorbance was measured at 340 nm.

### Calculations

Increase in formation of Cornified envelopes in each sample was calculated wrt control (Untreated cells).

$$[ ( A - B ) / B ] * 100 ; Where A = Absorbance in Test Item treated cells ,$$

$$B = Absorbance Control ( Untreated ) cells .$$

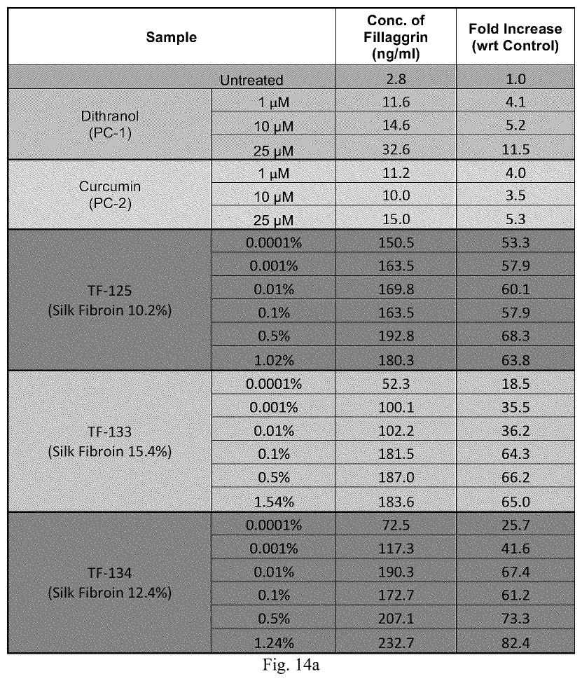

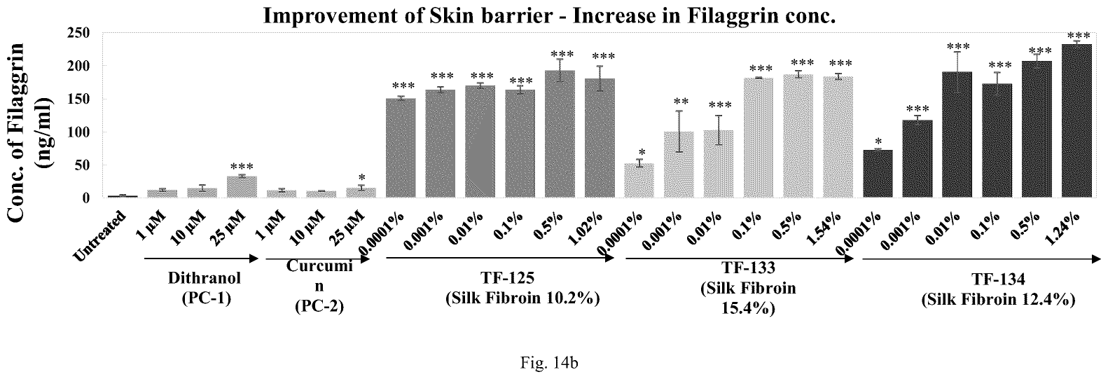

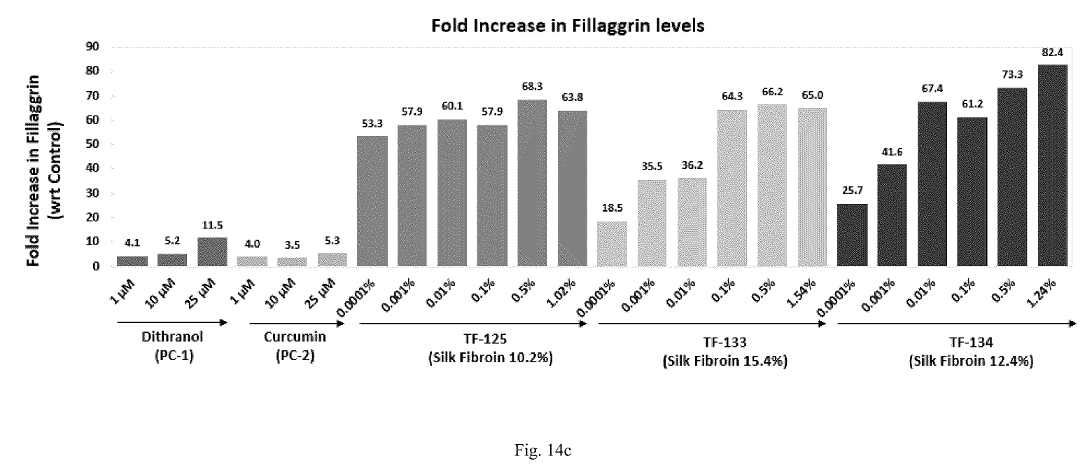

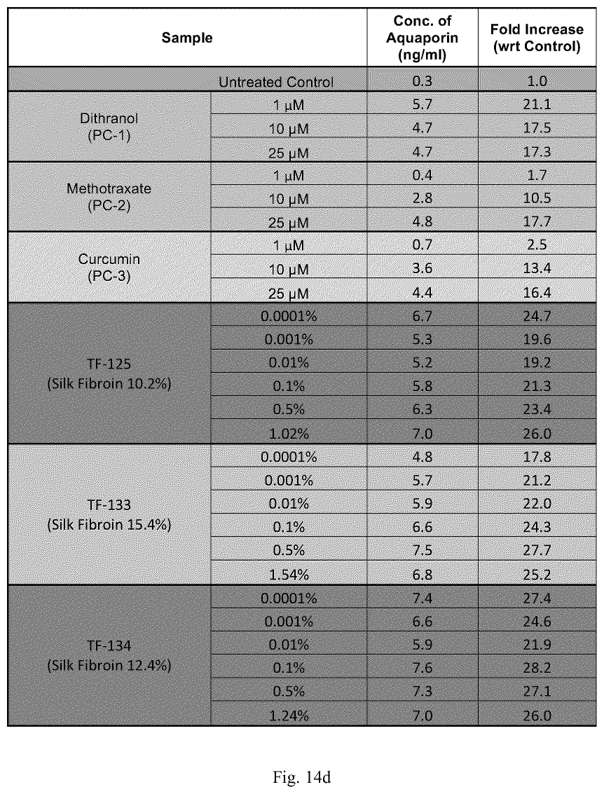

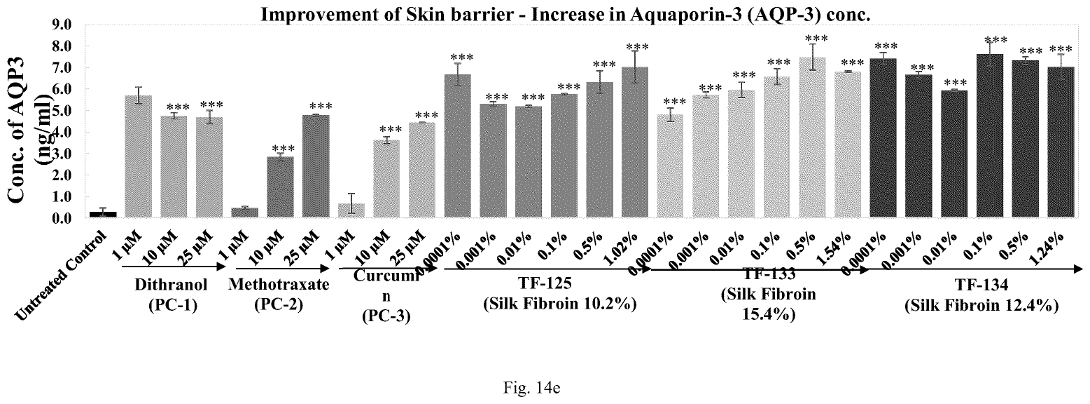

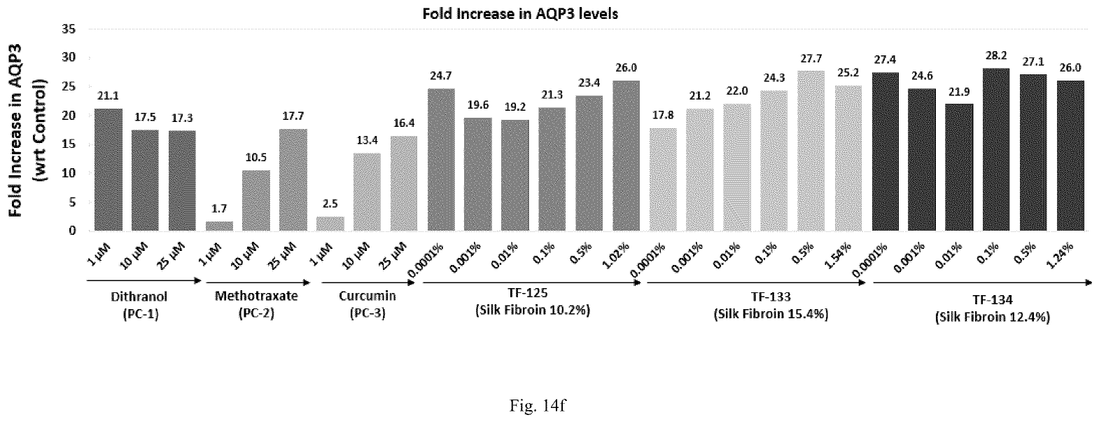

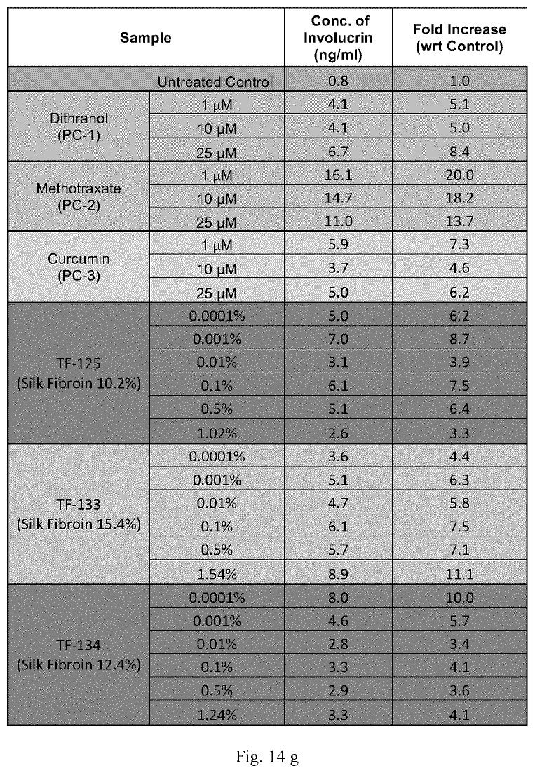

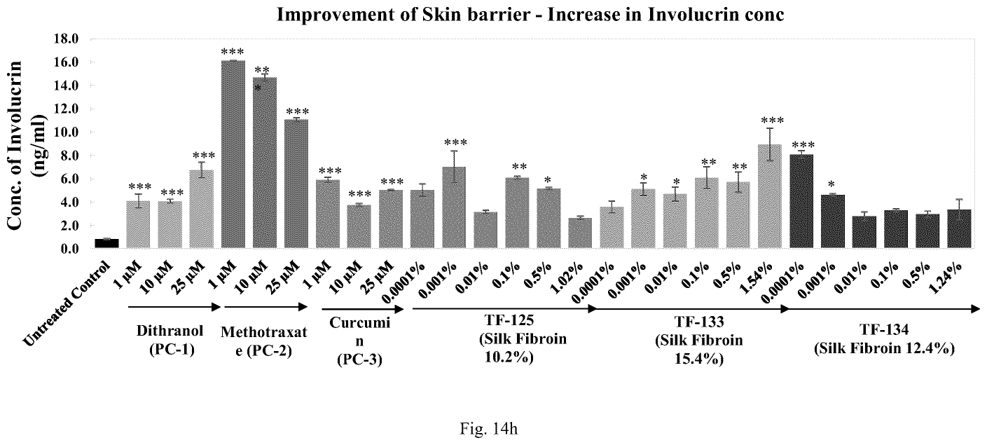

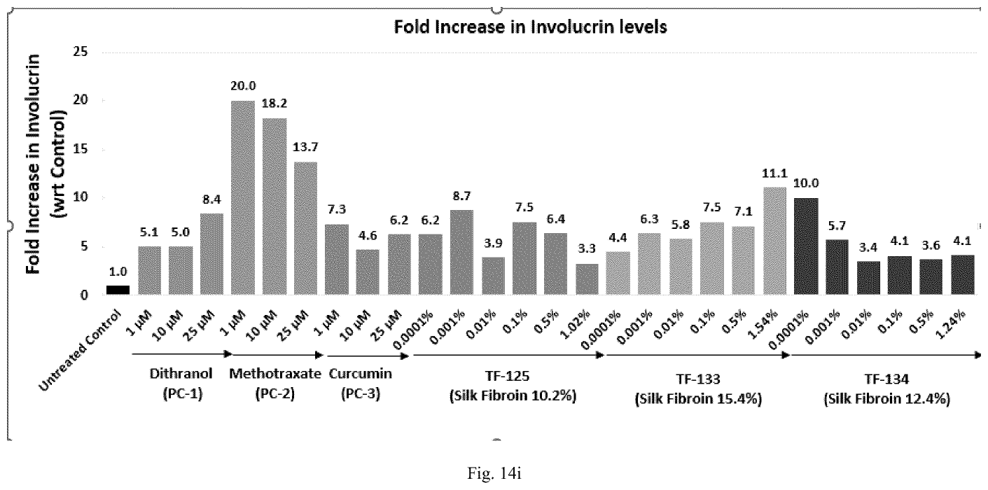

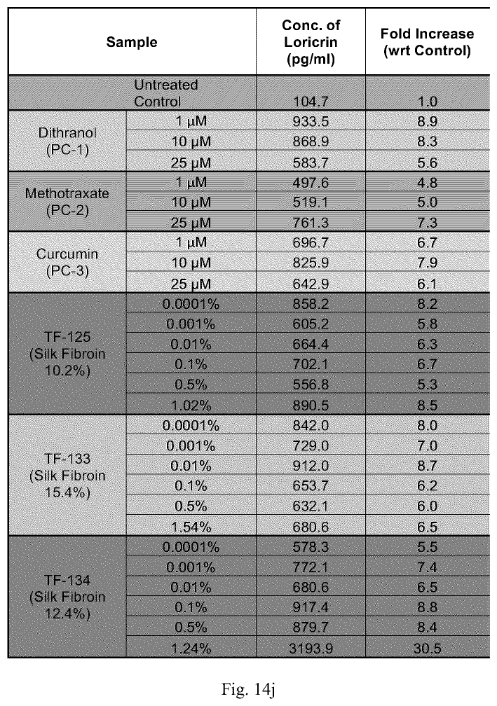

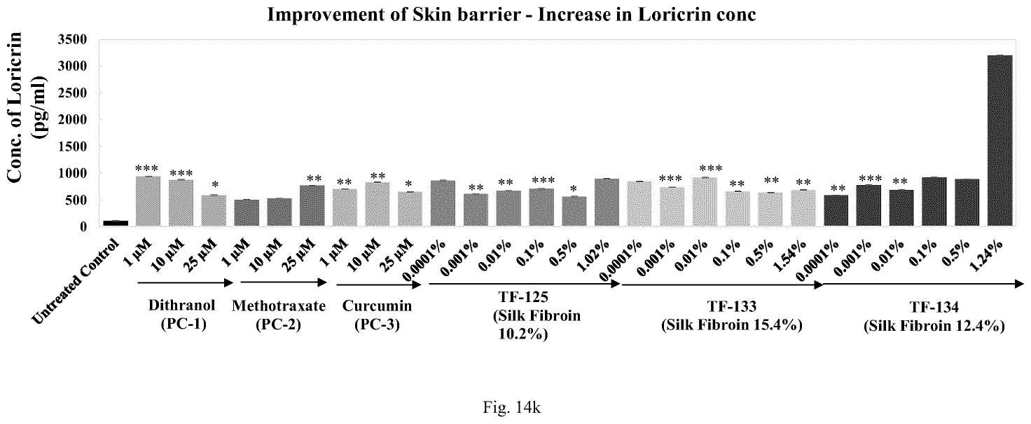

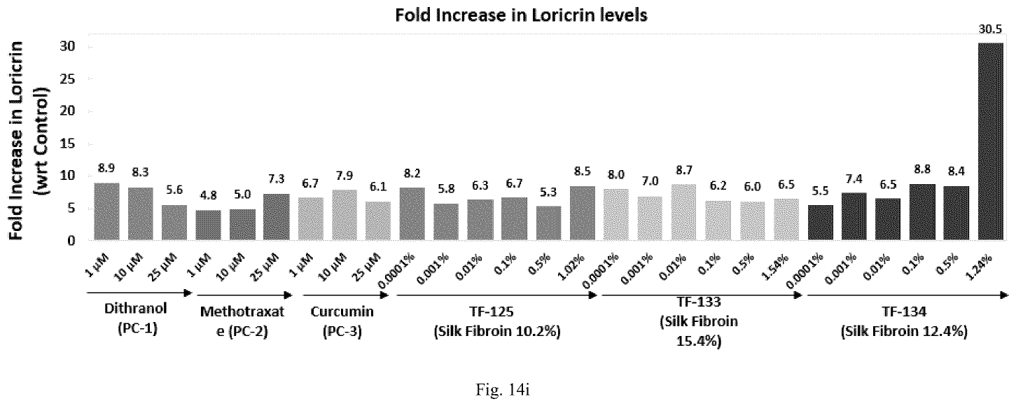

### 2b)—Improvement of Skin Barrier Formation by Increased in Expression of Surface Proteins (as Seen in FIGS. 14a-14i)

### Study Design

Test System—Human immortalized Keratinocyte cell line (HaCaT)
Number of cells plated—0.1 million cells/24-well plates
FBS concentrations—0%,
Test items—TF-125 (10.2%), TF-133 (15.4%), TF-134 (12.4%)
Time points—72h
Estimation method—ELISA
Positive Control/s—Methotrexate, Dithranol, Curcumin.

### Procedure

Cells were plated in 10% FBS in 24-well plates and incubated for 24 hours. Cells were then serum starved with 0% FBS for 24 hours. Cells were treated with 3 Test items in 0% FBS at non-cytotoxic concentrations for 72 hours. After incubation, culture supernatants were collected, and lysates were also prepared. Levels of Filaggrin, AQP3 (supernatants) and Involucrin, Loricrin (lysates) were determined using ELISA as follows: Respective kit standards and samples (supernatants of cells) were directly pipetted into the wells and incubated for 1-2 hours at 37° C. Liquid was removed from each well and plate was incubated at 37° C. for 1 hour after addition of Biotin-antibody. Following a wash (3 times) to remove any unbound conjugate, HRP-avidin solution was added to the wells and incubated for 30 min at 37° C. Plate was again washed for 3 times. TMB substrate was added to each well for 15-30 mins and plate followed by incubation at 37° C. The reaction was stopped by adding Stop solution to each well. The optical density of the color was measured at 450 nm.

### Calculations

Increase in Filaggrin levels in each sample was calculated wrt control (Untreated cells).

$$[ ( A - B ) / B ] * 100 ; Where A = Test Item treated cells ,$$

$$B = Control ( Untreated ) cells .$$

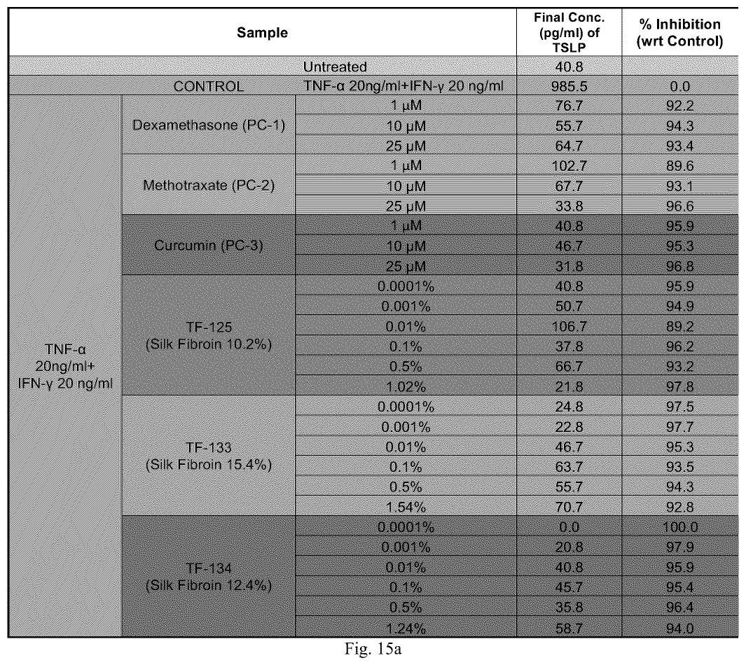

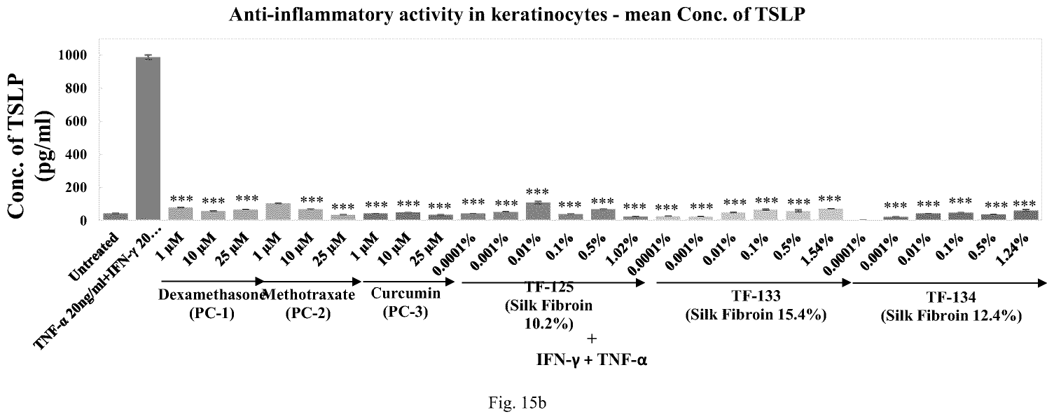

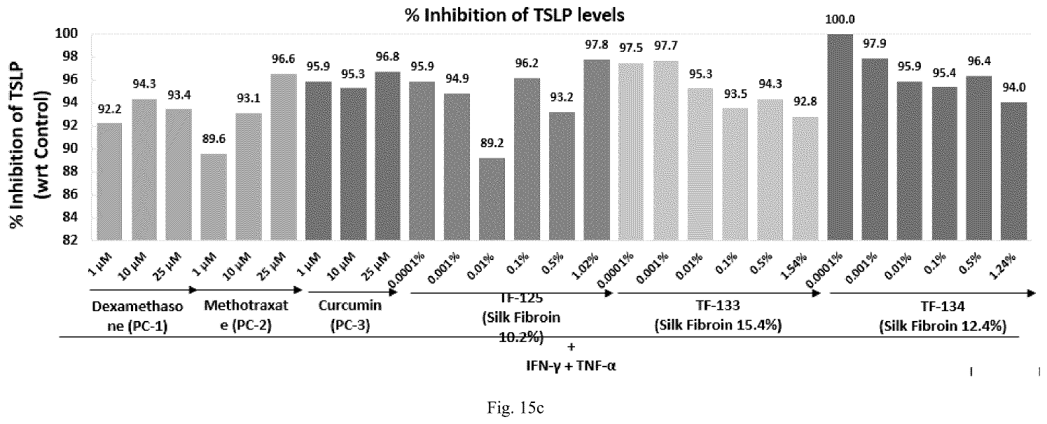

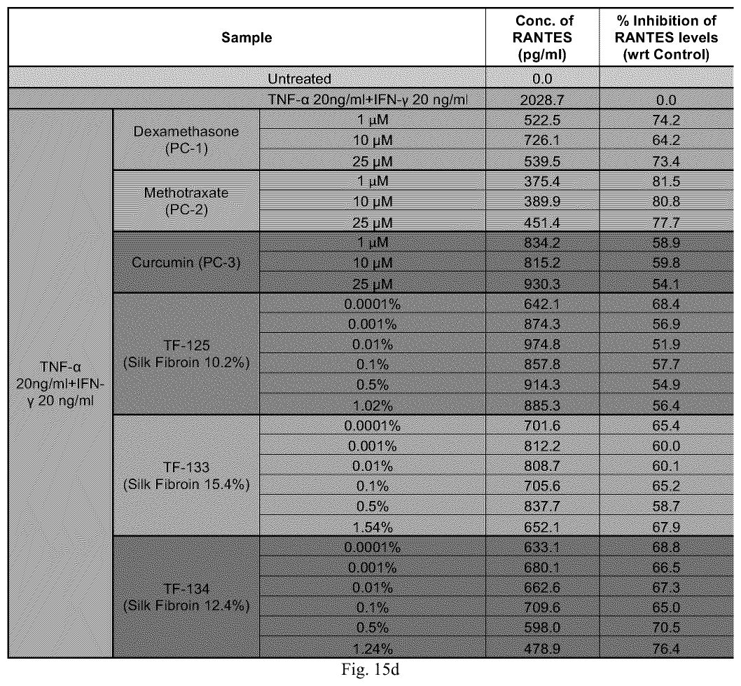

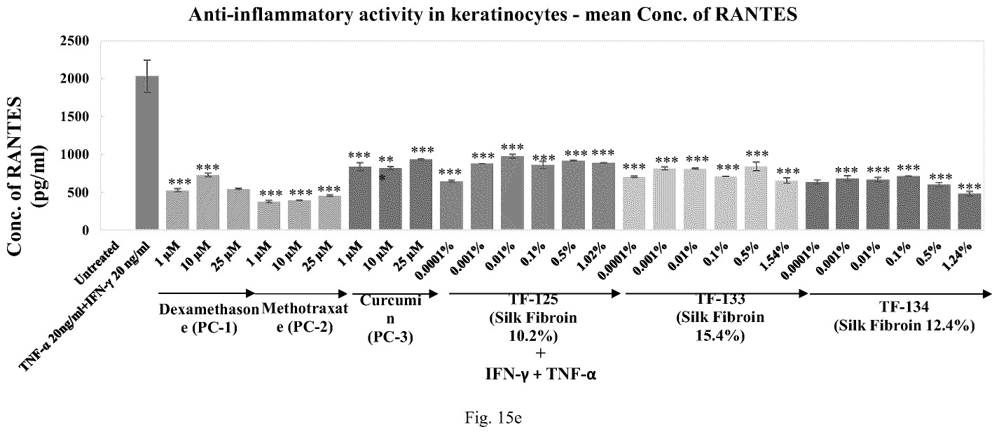

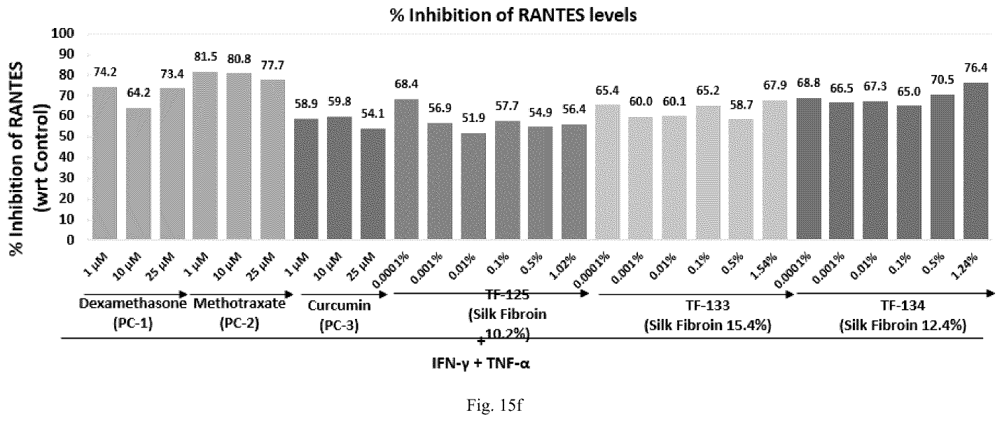

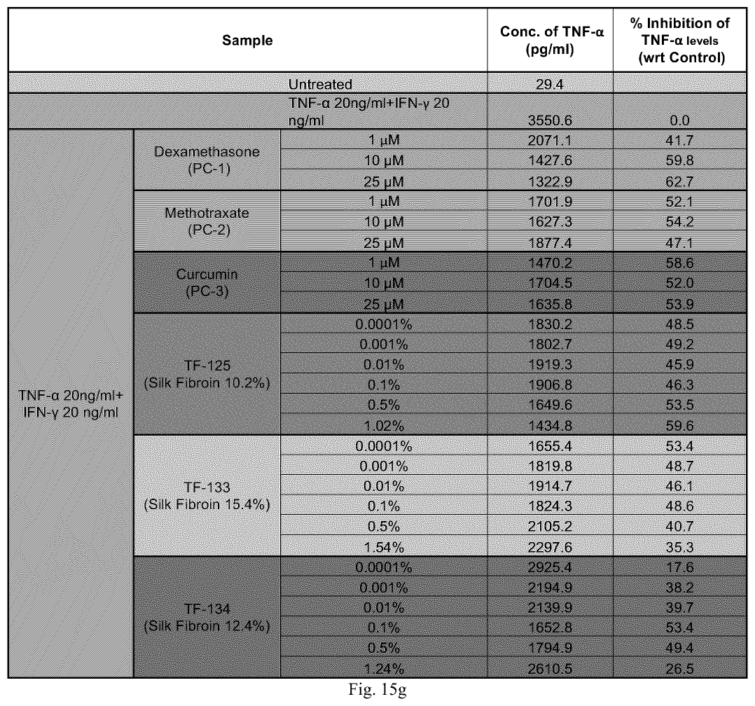

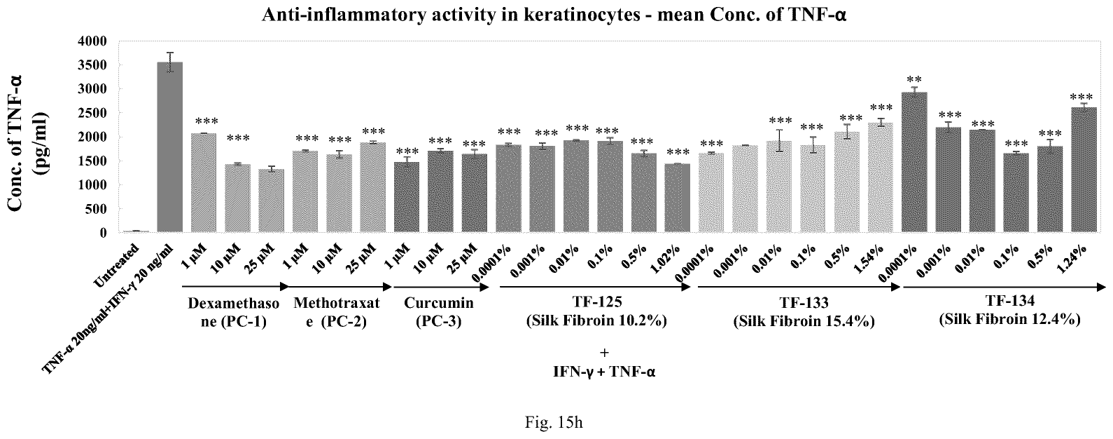

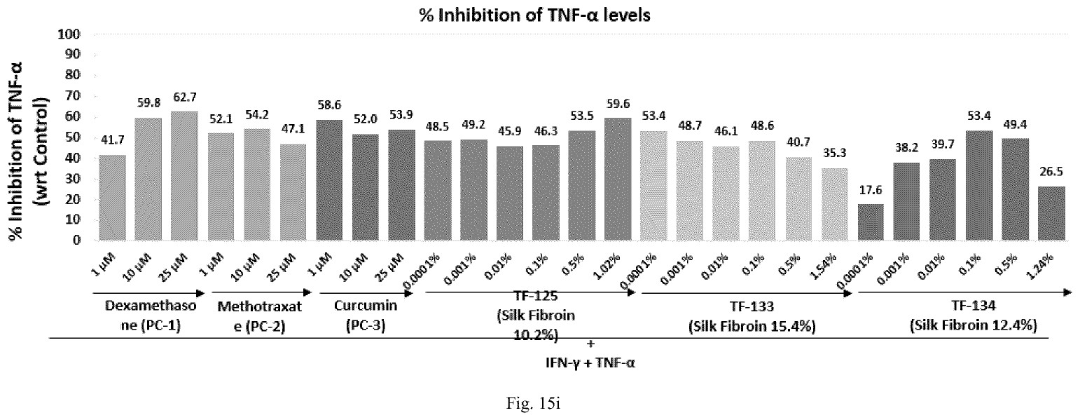

### 2c)—Anti-Inflammatory Effect in Keratinocytes (as Seen in FIGS. 15a-15i)

### Study Design

Test System—Human immortalized Keratinocyte cell line (HaCaT)
Number of cells plated—0.1 million cells/24-well plates
FBS concentrations—0.1%,
Test items—TF-125 (10.2%), TF-133 (15.4%), TF-134 (12.4%)
Time points—24h Pre-treatment, 24h stimulation
Estimation method—Human TNF-alpha, TSLP, TARC, RANTES ELISA.
Positive Control/s—Curcumin, Dexamethasone, Methotrexate

### Procedure

Cells were plated in 10% FBS in 24-well plates and incubated for 24 hours. Cells were then serum starved in 0.1% FBS for 24 hours. Cells were treated with 3 Test items in 0.1% FBS at non-cytotoxic concentrations for 24 hours. After 24 hours, cells were stimulated with inflammatory stimulus (hu-TNF-α 20 ng/ml+hu-IFN-□20 ng/ml). After 24 hours of stimulation, cell lysates were prepared. Levels of cytokines (TSLP, TARC, IL-8, TNF-α) were determined using ELISA as follows: Assay diluent was added to each well. Respective kit standards and samples (supernatants of cells) were directly pipetted into the wells and incubated for 2 hours at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm

### Calculations

Percent Inhibition in each sample was calculated wrt control (hu-TNF-α+hu-IFN-□treated cells).

$$[ ( B - A ) / B ] * 100 ; Where A = Test Item treated cells ,$$

$$B = Control ( hu - TNF - α hu - IFN - □treated ) cells .$$

Levels of TARCH remained unaffected

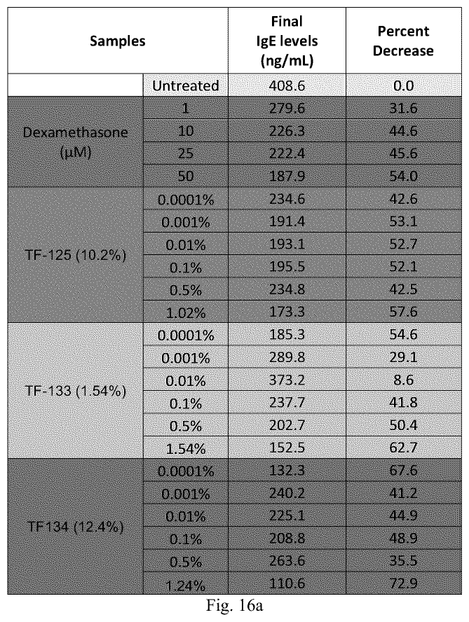

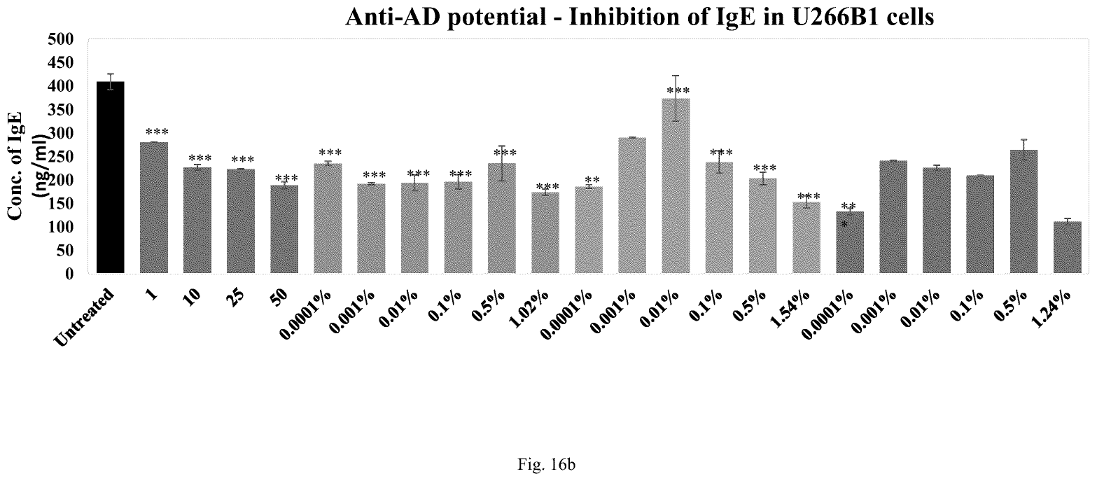

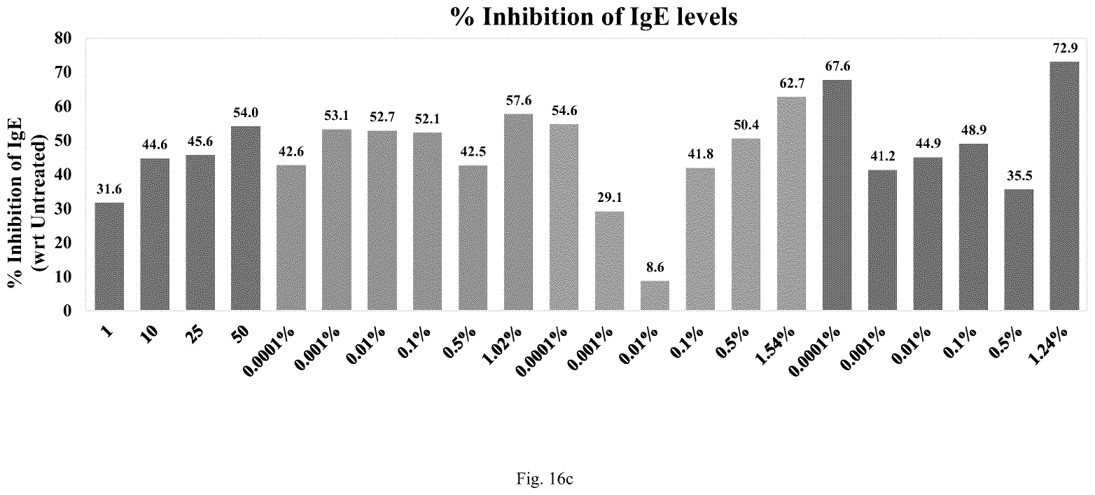

### 2d)—Anti-Allergic Effect by Inhibition of IgE (as Seen in FIGS. 16a-16c)

### Study Design

Test System—U266B1 (Human B-cell Myeloma cell line)
Number of cells plated—40,000 cells/48 well plate
FBS concentration—10%
Test Items—TF-125 (10.2%), TF-133 (15.4%), TF-134 (12.4%)
Time point—48h treatment
Estimation method—ELISA
Positive Control—Dexamethasone

### Procedure

Cells were plated in 10% FBS in 48-well plates and incubated for 24 hours. Cells were then treated with 3 Test Items in 10% FBS at non-cytotoxic concentrations for 72 hours. After 72h, culture supernatants were collected. IgE levels were determined using ELISA as follows: 2-fold dilution of standard was prepared and added to respective wells. 1:20 dilution of samples (supernatants of cells) was prepared and pipetted into the respective wells and incubated for 90 min at RT. After washing away any unbound substances for a total of 3 times, detection antibody was added to each well and incubated for 60 min at 37° C. After washing for 3 times, respective conjugate solution was added to each well and incubated for 45 min at 37° C. Following a wash (5 times), substrate solution was added to the wells and incubated at 37° C. in dark for 30 min. The reaction was stopped by adding Stop solution to each well. The optical density was measured at 450 nm.

### Calculations

Percent Inhibition in each sample was calculated wrt control (Untreated cells).

[(B−A)/B]*100; Where A=Test Item treated cells, B=Control (untreated) cells.

### Acne

Acne is a chronic inflammatory disease of the pilosebaceous unit. Its pathophysiology includes hyper-seborrhea, abnormal follicular keratinization and Propionibacterium acnes proliferation in the pilosebaceous unit. Recent research has shed some new light on the involvement of the sebaceous gland, as well as on the pro-inflammatory activity of the cutaneous microbiome. During puberty, alteration of the sebaceous lipid profile, called dysseborrhoea, stress, irritation, cosmetics and potential dietary factors lead to inflammation and formation of different types of acne lesions.

Altered follicular keratinization, sebum hyperproduction, and Propionibacterium acnes (P. acnes) colonization/growth within the pilo-sebaceous unit results in disruption of the follicular epithelium. P. acnes can then leak out of the pilo-sebaceous unit and get into contact with myeloid cells such as macrophages, thereby triggering molecular events involved in NLRP3-inflammasome activation, subsequent local release of bioactive IL-10, and neutrophil-rich local inflammation. An earlier interaction between P. acnes and myeloid cells, either by migration of the latter into the follicular space or “leakage” of P. acnes into the peri-follicular dermis, leading to inflammasome activation is being explored.

Acne is a chronic inflammatory disease of the pilosebaceous unit. Its pathophysiology includes hyperseborrhoea, abnormal follicular keratinization and Propionibacterium acnes proliferation in the pilosebaceous unit. During puberty, alteration of the sebaceous lipid profile, called dysseborrhoea, stress, irritation, cosmetics and potential dietary factors lead to inflammation and formation of different types of acne lesions. During puberty, alteration of the sebaceous lipid profile, called dysseborrhoea, stress, irritation, cosmetics and potential dietary factors lead to inflammation and formation of different types of acne lesions. Dysbiosis, the process leading to a disturbed skin barrier and disequilibrium of the cutaneous microbiome, resulting in the proliferation of P. acnes strains, is another important process that triggers acne. P. acnes activates the innate immunity via the expression of protease activated receptors (PARs), inflammatory cytokines, resulting in the hyperkeratinisation of the pilosebaceous unit.

The sebocyte culture are well reported as a suitable model to study the pathophysiology of the sebaceous gland in sebostasis, seborrhoea and acne [14-15]. Increase in sebum synthesis, increased colonization of P acnes in comodones, and hyperinflammation by increased secretion of inflammatory cytokines are key hallmark events in acne.

Hence sebostatic activity by inhibition of sebum production in sebocytes, inhibition of inflammatory cytokine in sebocytes and antibacterial activity towards P acnes indicate anti-acne potential of test agents.

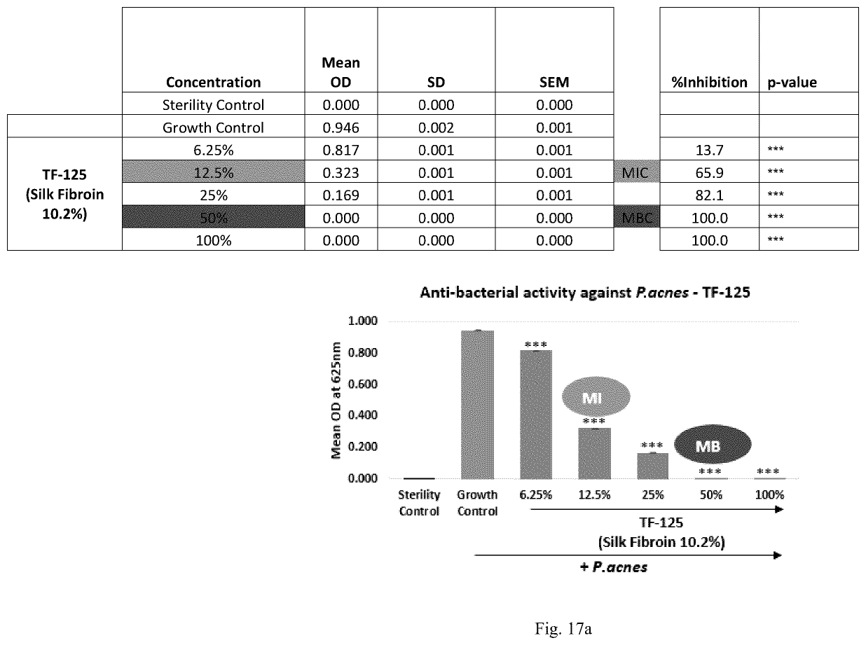

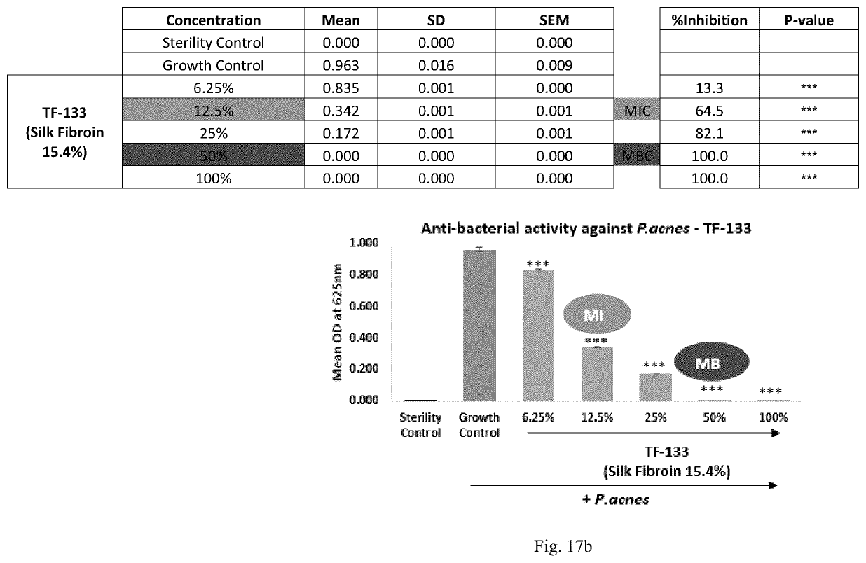

### 3a) Anti-Bacterial Activity Against P. acnes (as Seen in FIGS. 17a-17c)

### Study Design

Test System—Propionibacterium acnes (P. acnes)
Test items—TF-125 (10.2%), TF-133 (15.4%), TF-134 (12.4%)
Estimation method—Broth macro dilution
End point estimation—MIC (Minimum inhibitory concentration)

### MBC (Minimum Bactericidal Concentration)

### Procedure

P. acnes were revived and cultured at sterile anaerobic conditions. Test items stock provided was considered as 100%. Test items (5 dilutions at 1:1) were inoculated with P. acnes suspension (5×105 CFU/ml concentration). Sterility control (only growth medium) was included as Negative control. Growth control (only P. acnes in growth medium) was included as Untreated. Clindamycin at working concentration of 6.7 μg/ml was used as Positive control. All the test tubes were incubated at 37° C., 200 rpm for 48 hours in the BOD incubator. End Point OD625 was taken and analyzed to determine MIC (Minimum Inhibitory Concentration) and MBC (Minimum Bactericidal Concentration). Lowest concentration of TI preventing appearance of turbidity (cloudiness) is considered as MIC. Lowest concentration of TI that kills ≥99.9% bacteria is considered as MBC.

### Calculations

% Inhibition of P. acnes was calculated w.r.t growth control.

% Inhibition of P. acnes=Mean O.D of ((Growth control-test item)/Growth control)*100

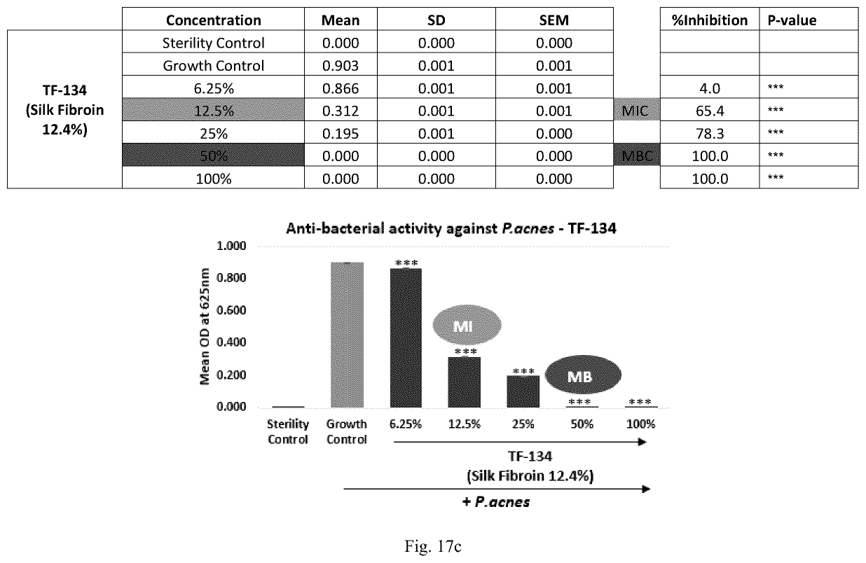

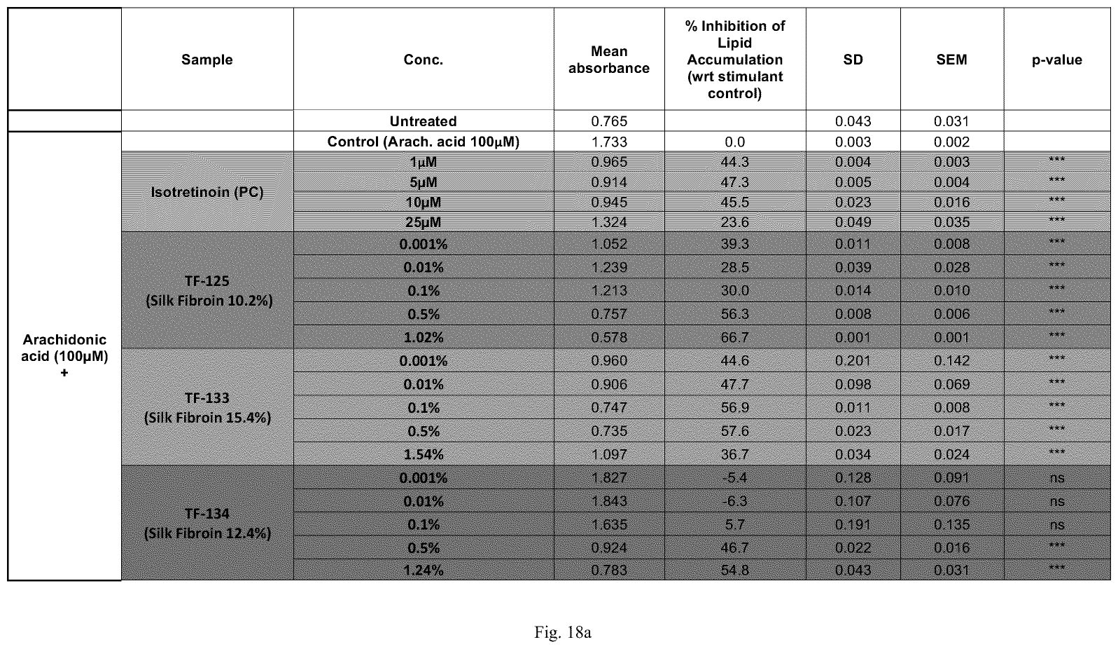

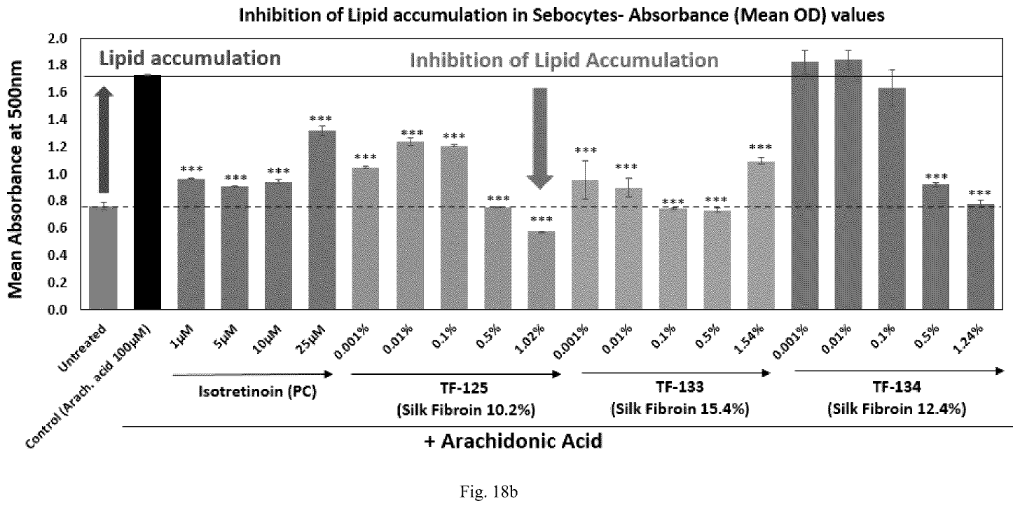

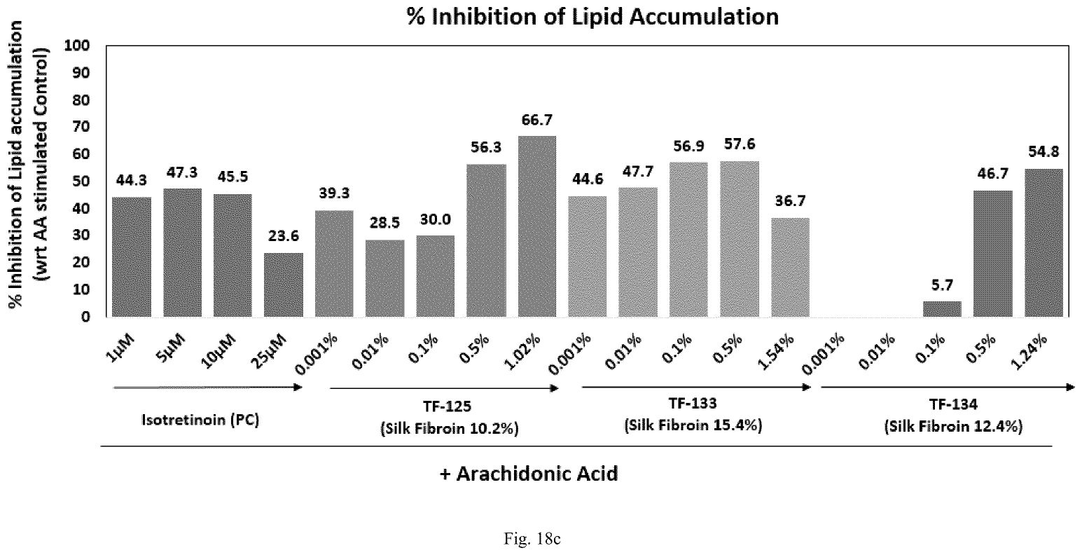

### 3b) Sebostatic Potential by Inhibition of Sebum Synthesis in Sebocytes (as Seen in FIGS. 18a-18c)

### Study Design

Test System—SebE6E7 (Human Sebocytes)
Number of cells plated—50000 cells/12 well plate
FBS concentrations—0.5%
Test items—TF-125 (10.2%), TF-134 (12.4%), TF-133 (15.4%)
Time points—48h Co-treatment, Arachidonic Acid (100 μM) stimulation
Estimation method—Lipid content by Oil-Red-O dye (fat-soluble dye that stains neutral triglycerides and lipids, used for quantitative and qualitative measurement of lipid droplet formation)
Positive Control-Isotretinoin

### Procedure

Cells were plated in 12-well plates in 10% FBS and incubated for 24 hours. Cells were then serum starved with 1% FBS for 24 hours. Cells were treated with 3 Test items at non-cytotoxic concentrations and co-stimulated with Arachidonic Acid (100 μM) for 48 hours. After 48 hours, cells were fixed and lipid content was measured by staining with Oil-O-Red dye. Cell layer was washed with PBS and fixed in 10% formalin. Cells were washed with 60% Isopropanol and air-dried. Air-dried cell layers were stained with Oil-Red-O stain at RT. Cell layers were washed with Milli-Q water to remove any unbound stain. The lipid bound stain was eluted with 100% Isopropanol and optical density was measured at 500 nm. Percent inhibition of sebum synthesis (lipid accumulation) was calculated wrt control (Arachidonic Acid stimulated cells).

### Calculations

Percent Inhibition in each sample was calculated wrt control (Untreated cells).

$$[ ( B - A ) / B ] * 100 ; Where A = Test Item treated cells ,$$

$$B = Control ( untreated ) cells .$$

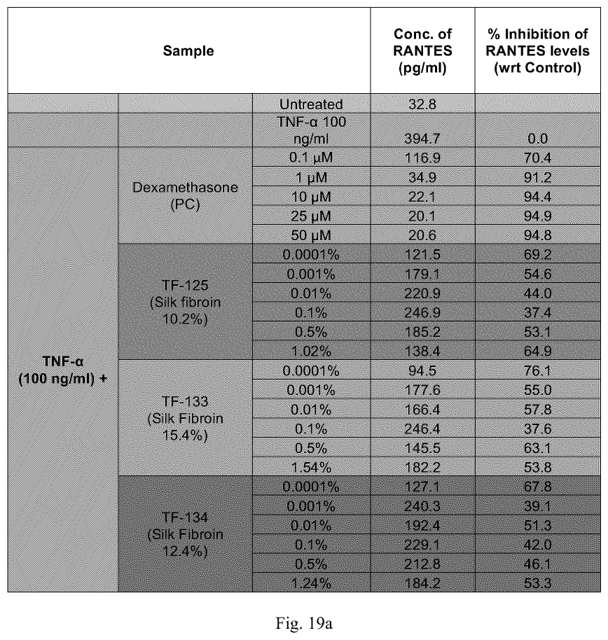

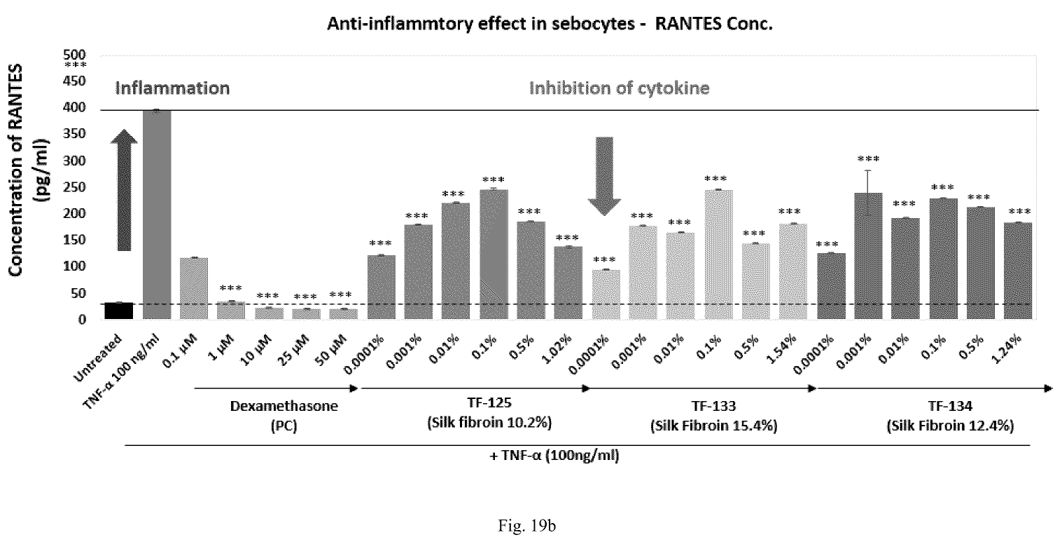

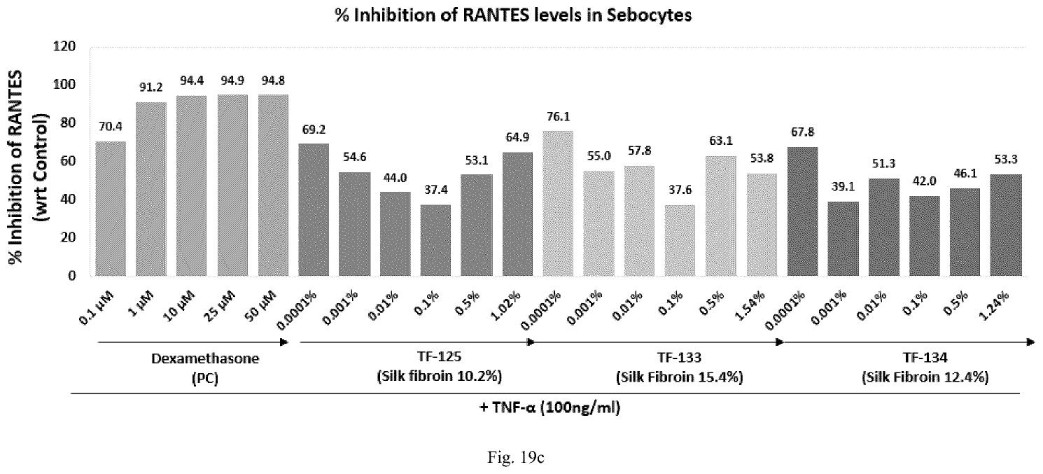

### 3c)—Anti-Inflammatory Activity by Inhibition of Cytokine in Sebocytes (as Seen in FIGS. 19a-19c)

### Study Design

Test System—SebE6E7 (Human Sebocytes)
Number of cells plated—50000 cells/12 well plate
FBS concentration—0.5%
Test items—TF-125 (10.2%), TF-133 (15.4%), TF-134 (12.4%)
Time point—48h Co-treatment, TNF-alpha (100 ng/mL) stimulation
Estimation method—ELISA
Positive Control—examethasone

### Procedure

Cells were plated in 12-well plates 10% FBS and incubated for 24 hours. Cells were then sera starved with 0.5% FBS for 24 hours. Cells were treated with 3 Test items at non-cytotoxic concentrations and co-stimulated with rhTNF-alpha (100 ng/ml) for 48 hours. After 48 hours, SN was collected, and level of cytokine was determined by ELISA. Level of RANTES (supernatants) was determined using ELISA as follows: Assay diluent was added to each well. Respective kit standard and samples (supernatants) were directly pipetted into the wells and incubated for 2 h at RT. After washing away any unbound substances for a total of 3 times, respective conjugate was added to each well and incubated for 1-2 hours at RT. Following a wash (3 times) to remove any unbound conjugate, substrate solution was added to the wells and incubated for 30 min at RT in dark. The reaction was stopped by adding Stop solution to each well. The optical density of the color was measured at 450 nm. Conc of RANTES was calculated from standard curve.

### Calculations

Percent Inhibition in each sample was calculated wrt control (Untreated cells).

$$[ ( B - A ) / B ] * 100 ; Where A = Test Item treated cells ,$$

$$B = Control ( untreated ) cells .$$

### C. Evaluation of Anti-Viral Activity of Fibroin Proteins (Tf-125, Tf-133, Tf-134) by Virucidal Assay Against Sars-Cov2

Severe acute respiratory syndrome coronavirus 2 (SARS-CoV-2) is a strain of coronavirus that causes COVID19 (coronavirus disease 2019), the respiratory illness responsible for the ongoing COVID-19 pandemic. SARS-CoV-2 is a member of a large family of viruses called coronaviruses. These viruses can infect people and some animals. SARS-CoV-2 was first known to infect people in 2019. The spike (S) protein of SARS-CoV-2, which plays a key role in the receptor recognition and cell membrane fusion process, is composed of two subunits, S1 and S2. The spikeS2 binds to ACE2 receptors in lung cells and infests the infection in body. Several virucidal agents are used for disinfecting the virus.

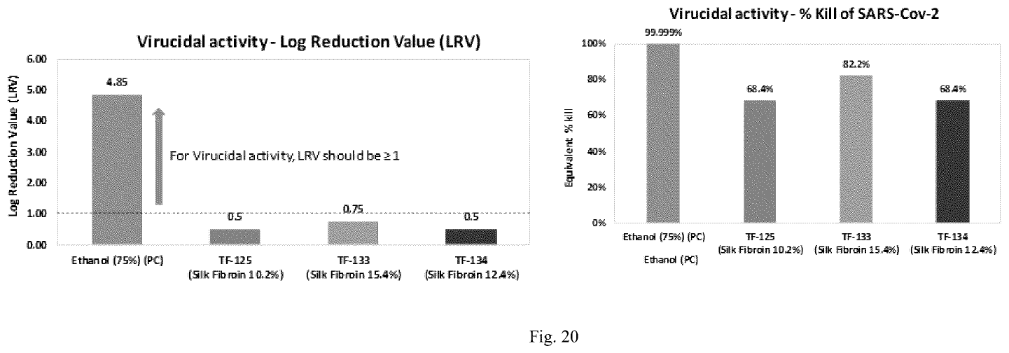

### 1a. Virucidal Activity Against SARS-CoV-2 (as Seen in FIG. 20)

### Study Design—Anti-Viral—Virucidal Activity Against SARS-CoV-2

### Study Design

Test system—SARS-CoV-2 Isolate USA-WA1/2020 (equivalent to Wuhan strain))
Host Cell line—VeroE6 (African green monkey kidney Cell Line)
Study Method—ASTM E1052
Test items—TF-125 (10.2%), TF-133 (15.4%), TF-134 (12.4%)
Contact Time—10 min
Time point—7 days
Positive Control—Ethanol

### Procedure

### A. Evaluation of the Virucidal Activity of the Test Item Against SARS-CoV-2

Test Items were used at neat concentration.
Infection medium containing virus (SARS-CoV-2 Isolate USA-WA1/2020) was prepared at approximately 106 TCID50/mL.
Virus only control (without TI), cytotoxicity control (only TI) and positive control (75% Ethanol), neutralization control and cell only control (without virus) were maintained during the assay.
Test Items were incubated with virus (at 9:1 ratio) for 10 min at RT.
Following the contact period, test Item-virus mixture was neutralized by 1/10 dilution in test media (DMEM).
Surviving virus load in test Item treated samples was quantified by standard end-point dilution assay.

### B. Standard End-Point Dilution Assay and Calculation of TCID50/mL

30,000 Vero E6 cells in a volume of 200 μL/well in DMEM (containing 10% FBS) were plated into 96-well plates and incubated overnight (12-18 h) at 37° C.
Samples were serially diluted using 10-fold dilutions in DMEM and added to host (Vero E6) cells plated in 96 well plates. Plates were incubated at 37° C. with 5% CO2 for 1 hour.
Upon completion of incubation the plate was supplemented with 150 μL test media (DMEM) and incubated at 37° C. in CO2 incubator (5%) for 7 days.
On day 7 post-infection plates were scored for the presence or absence of viral cytopathic effect (CPE).
The end-point titers (50% tissue culture infectious dose, TCID50) of the samples were determined by Spearman-Karber method.
The Virucidal activity of the test items was reflected by log reduction value (LRV) of the test items in comparison to virus-only control.
On basis of log reduction value (LRV), % killing was evaluated
   1 log reduction=90% kill
   2 log reduction=99% kill
   3 log reduction=99.9% kill

### Calculations

### LRV was Calculated Wrt Control (Untreated Cells)

LRV=Log 10 Virus titer in control—Log 10 LOD of the test

### D. Evaluation of Wound Healing Potential of Fibroin Proteins (Tf-125, Tf-133, Tf-134) Using In Vitro Endothelial Cell-Based Assays by Multi-Parametric Analysis

### Endothelial Cells—Wound Healing Process

Wound healing is a complex phenomenon that involves different cell types with various functions, i.e., keratinocytes, fibroblasts, and endothelial cells, all influenced by the action of soluble mediators and rearrangement of the extracellular matrix (ECM). Endothelial cells (Ecs) are involved in various physiological process. Endothelial cells (Ecs), uniquely localized and strategically forming the inner lining of the vascular wall, constitute the largest cell surface by area in the human body. There are multiple types of endothelial cells including, vascular endothelial cells (direct contact with blood) and lymphatic endothelial cells (direct contact with lymph). Vascular endothelial cells line the entire circulatory system, from the heart to the smallest capillaries. Endothelial cells have a critical role in the healing process after wounding or inflammation. Migration and proliferation are both necessary components for wound or gap closure. Effective wound healing in the vasculature, however, relies on stimulation of one cell type (endothelial) and simultaneous inhibition of another (smooth muscle). Acute wounds, involve the four overlapping (but well-defined) phases of hemostasis, inflammation, proliferation, and remodeling; an avascular scar is the final stage of the wound healing process. Endothelial cells migrate during vasculogenesis and angiogenesis but also in a damaged vessel to restore vessel integrity.

### Endothelial Cells—Growth Factors in Wound Healing

Eahy 926 cell line is a continuous, cloneable, human cell line that displays a number of features characteristic of vascular endothelial cells. The scratch wound assay provides a useful, inexpensive and easy-to-perform screening method for testing individual or combinations of growth factors or cytokines. Growth factors and cytokines play an important role in the wound healing process. They are polypeptide molecules that control the growth, differentiation, and metabolism of cells during each of the three phases of wound healing They induce proliferation and migration of cells within the wound environment. The wound healing process is orchestrated by growth factors and cytokines released by a variety of cells that accumulate within the provisional matrix and ECM (e.g., platelets, neutrophils, fibroblasts, endothelial cells, macrophages, lymphocytes). Various kinds of growth factor such as epidermal growth factor (EGF), fibroblast growth factor (FGF), transforming growth factor (TGF), keratinocyte growth factor (KGF), hepatocyte growth factor (HGF), platelet-derived growth factor (PDGF) and insulin like growth factor (IGF) play a key role in corneal wound healing. and these growth factors are expressed in the corneal epithelial cells, keratocytes and endothelial cells.

### Endothelial Cells—TGF-β1 Role in Wound Healing

The success of the wound healing process depends on growth factors, cytokines, and chemokines involved in complex integration of signals that coordinate cellular processes. TGF-β1 is important in inflammation, angiogenesis, re-epithelialization, and connective tissue regeneration. It is shown to have increased expression with the onset of injury. TGF-01 facilitates the recruitment of additional inflammatory cells and augments macrophage-mediated tissue debridement. In vitro studies show that TGF-01 helps initiate granulation tissue formation by increasing the expression of genes associated with ECM formation including fibronectin, the fibronectin receptor, and collagen and protease inhibitors. Hence, Growth factors, cytokines and chemokines are crucial for coordinating multiple cell types during the healing process, making cutaneous wound healing possible.

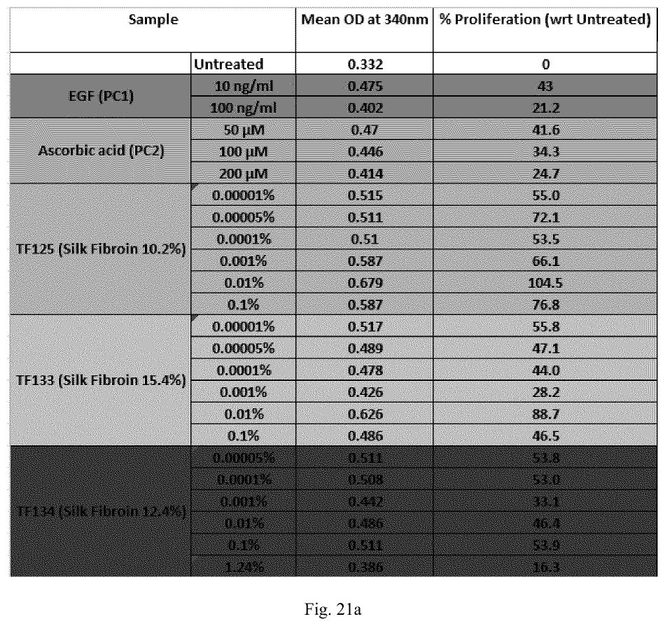

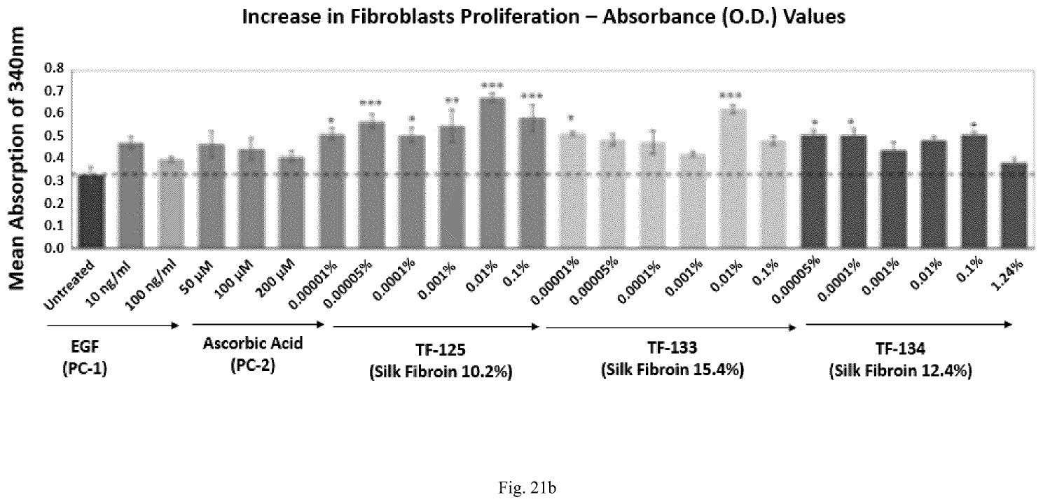

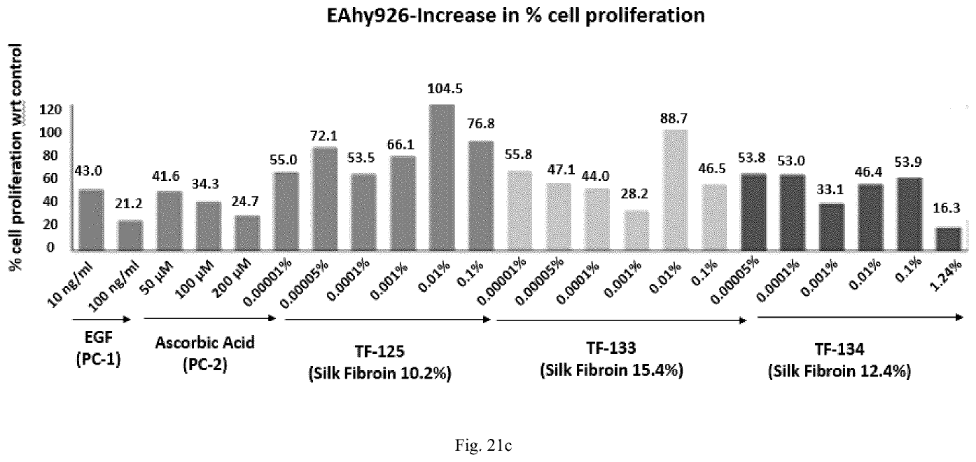

### 1a. Increase in Proliferation of Endothelial Cells (as Seen in FIGS. 21a-21c)

Study Design Assay 1a)—Wound Healing—Increase in Proliferation

### Study Design

Test System—Human Endothelial Cells (Eahy.926)
Number of cells plated—2000 cells/96-well plates
FBS concentrations—0.5%
Test items—TF-125 (10.2%), TF 133 (15.4%), TF-134 (12.4%)
Time points—3 days
Estimation method—BrdU assay
Positive Control/s—Ascorbic acid, EGF

### Procedure

Cells were plated in 10% FBS in 96-well plates and incubated for 24 h.
Cells were serum starved in 0% FBS for 24 h.
Cells were treated with 3 Test items at various concentrations in 0.5% FBS.
After 3 days (72h), the effect on proliferation was determined by BrdU assay.
   10 μl of BrdU solution was added/well and cells were incubated for 90 minutes at 37° C.
      After removing medium, cells were fixed, and the DNA was denatured in one step by adding 200 μl of FixDenat. Cells were incubated for 30 min at RT (15-25° C.).
      The FixDenat solution was removed and 100 μl of Anti-BrdU-POD solution was added to each well. Cells were incubated for 90 min at RT (15-25° C.).
   Wells were washed 3 times using 200 μl of washing solution.
   100 μl/well of substrate solution was added.
   Cells were incubated for 30 min at RT (15-25° C.).
      After incubation, a blue coloured complex was formed. Absorbance of each well was measured at 370 nm.

### Calculations

Increase in proliferation was calculated wrt control (Untreated cells). [(A−B)/B]*100; Where A=Test Item treated cells, B=Control (Untreated) cells

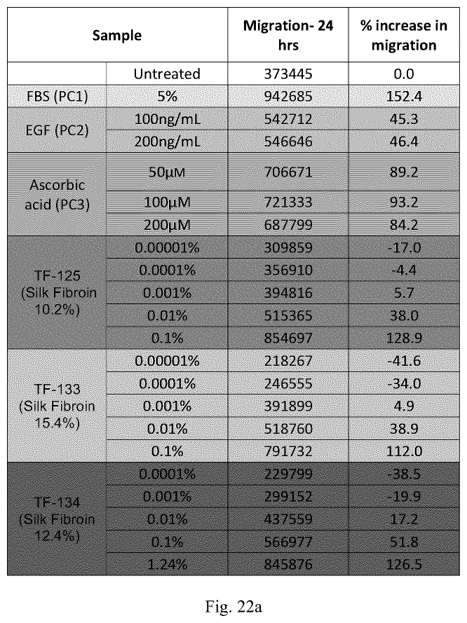

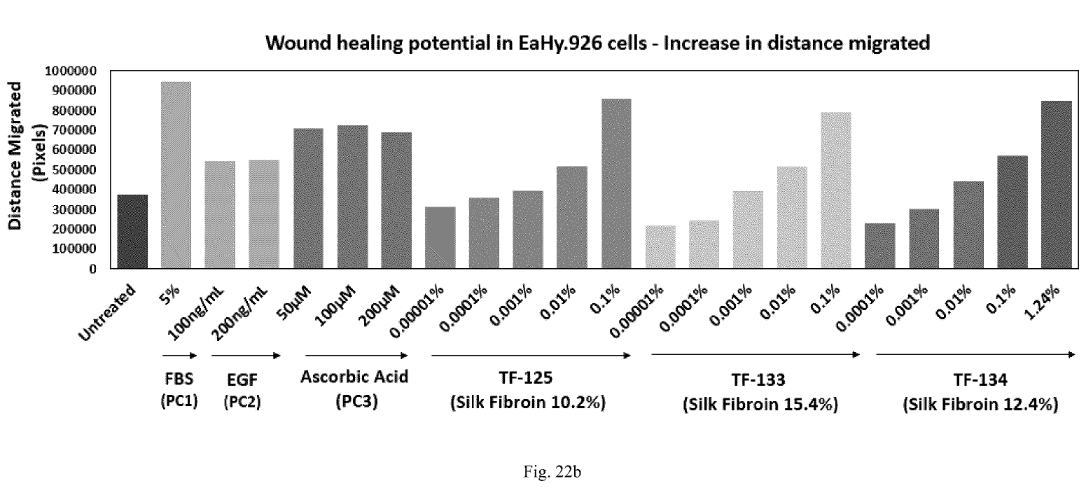

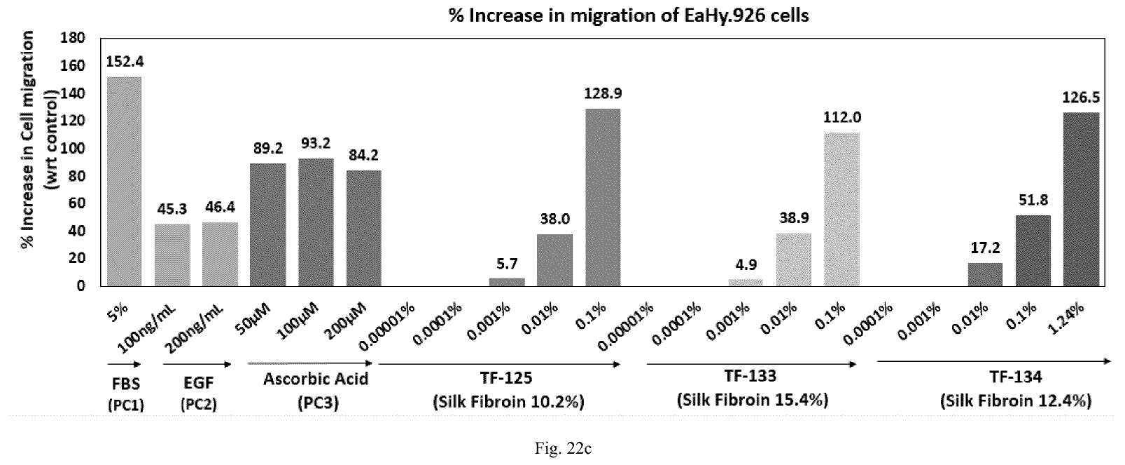

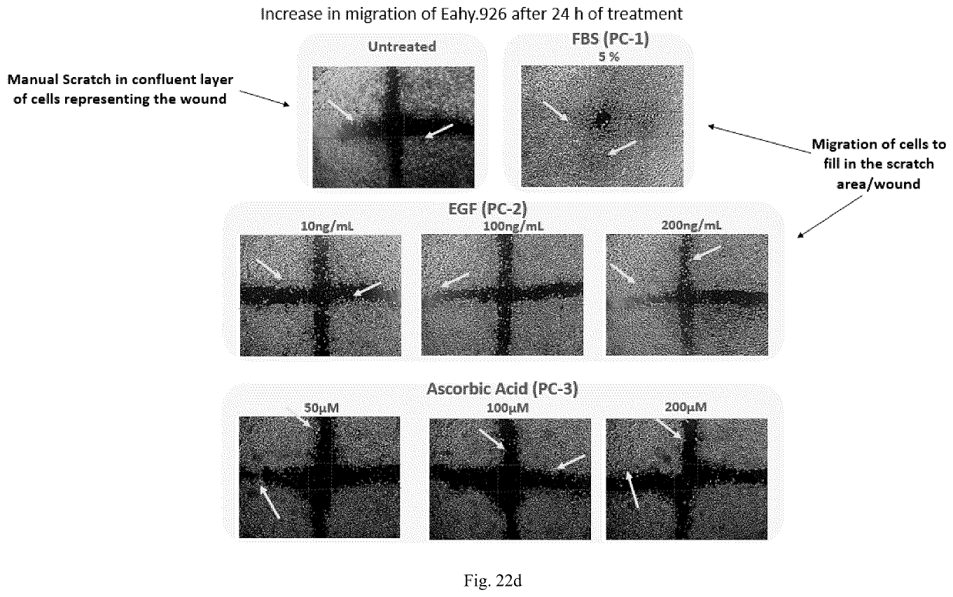

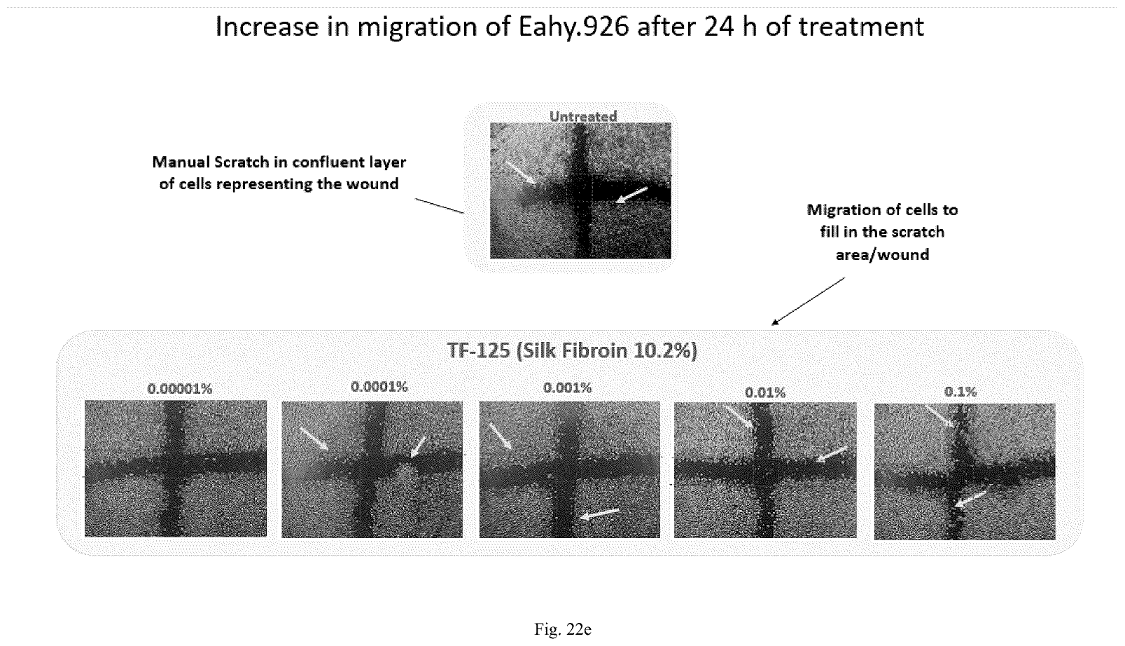

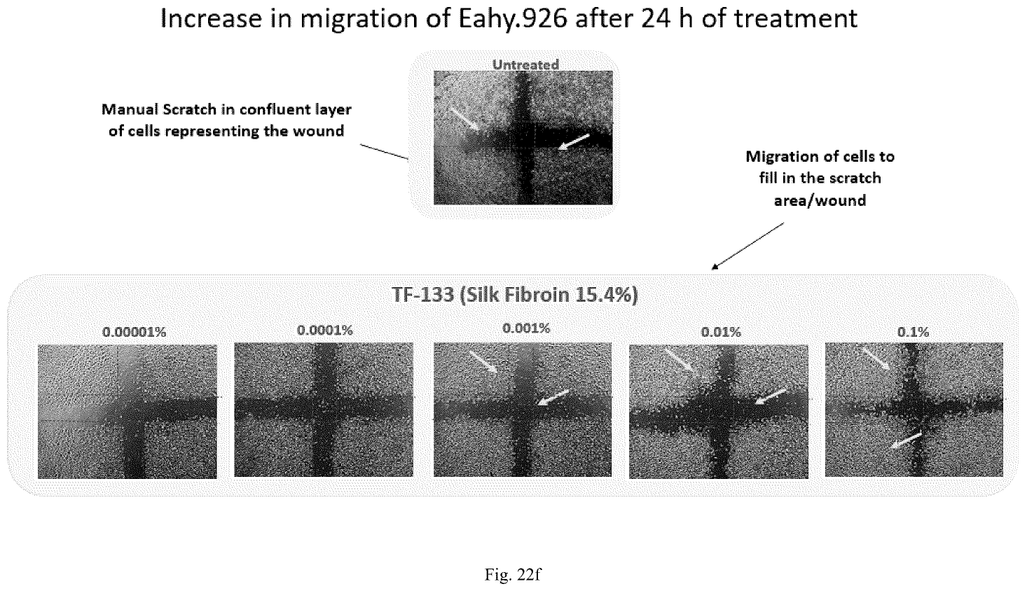

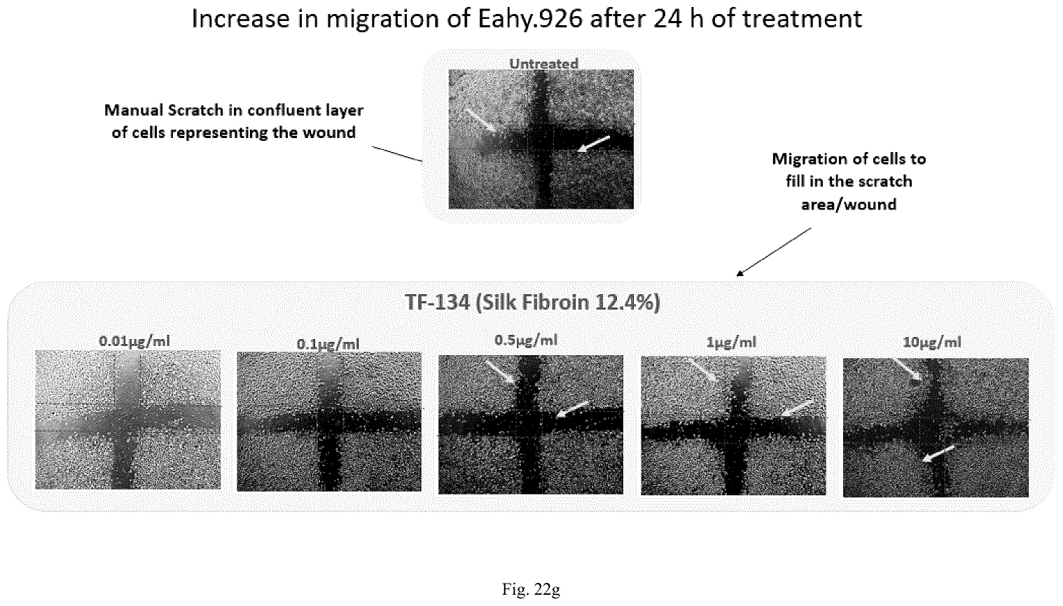

### 1b. Increase in Cell Migration of Endothelial Cells (as Seen in FIGS. 22a-22g)

Study Design Assay 1b)—Wound Healing—Increase in Cell Migration

### Study Design

Test System—Human Endothelial Cells (Eahy.926)
Number of cells plated—0.25 million cells/well
FBS concentration—0.1%
Test Items—TF-125 (10.2%), TF 133 (15.4%), TF-134 (12.4%)
Time point—24h
Estimation method—Scratch assay
Positive Control/s—EGF, FBS, Ascorbic Acid

### Procedure

Cells were plated in 10% FBS in 12-well plate and incubated for 24 h.
Cells were serum starved in 0.1% FBS for 24 h.
Scratch was created in each well using a sterile tip followed by treatment of cells with 3 Test items with non-cytotoxic concentrations of TIs and PCs.
Images were captured (0h).
After 16 h, the images were captured to determine the effect of Test Items on migration.
Increase in migration was calculated using ImageJ software.

### Calculations

Increase in migration was calculated wrt control (Untreated cells).

$$[ ( A - B ) / B ] * 100 ; Where A = Test Item treated cells , B = Control ( Untreated ) cells$$

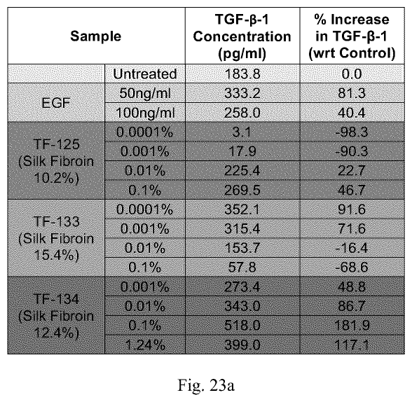

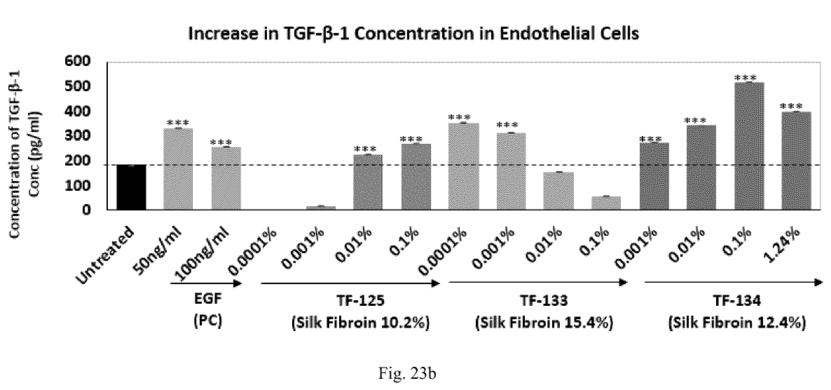

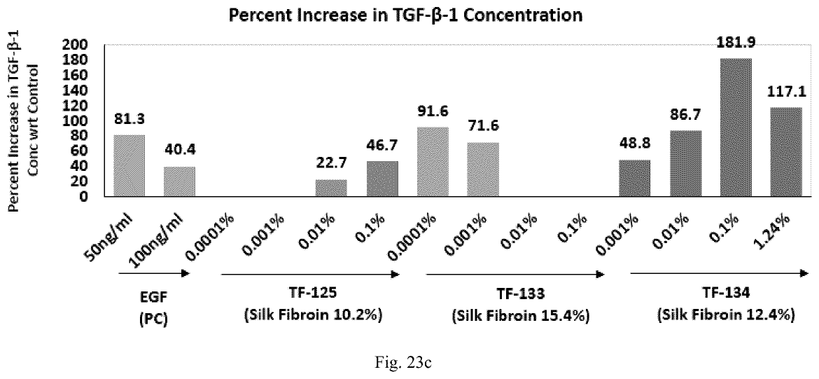

### 1c. Increase in Secretion of Growth Factors in Endothelial Cells (as Seen in FIGS. 23a-23c)

### Study Design Assay 1c) Wound Healing—Increase in Secretion of Growth Factor

### Study Design

Test System—Human Endothelial Cells (Eahy.926)
Number of cells plated—0.4 million cells/6-well plates
FBS concentrations—0%,
Test items—TF-125 (10.2%), TF 133 (15.4%), TF-134 (12.4%)
Time points—24h
Estimation method—ELISA
Positive Control/s—EGF

### Procedure

Cells were plated in 10% FBS in 24-well plates and incubated for 24 h.
Cells were sera Starved with 0% FBS for 24h.
Cells were treated with 3 Test items in 0% FBS at various concentrations for 72h.
After incubation, culture supernatants were collected, and lysates were also prepared.
Levels of TGF-b1 were determined using ELISA as follows—
   Samples for ELISA were activated using 1N HCl followed by neutralization with 1.2N NaOH.
   Assay diluent was added to each well. Respective kit standards and samples (activated) were directly pipetted into the wells and incubated for 2 h at RT.
   After washing away any unbound substances for a total of 3 times, respective conjugate was added to each well and incubated for 1-2 h at RT.
   Following a wash (3 times) to remove any unbound conjugate, substrate solution was added to the wells and incubated for 30 min at RT in dark. The reaction was stopped by adding Stop solution to each well.
   The optical density of the color was measured at 450 nm. Conc of cytokines were calculated from standard curve.

### Calculations

Increase in TGF-b1 levels in each sample was calculated wrt control (Untreated cells).

$$[ ( A - B ) / B ] * 100 ; Where A = Test Item treated cells , B = Control ( Untreated ) cells .$$

### E. Evaluation of Skin-Health Promotion, Anti-Aging and Skin Whitening Potential of Fibroin Proteins (Tf-125, Tf-133, Tf-134) Using in Vitro Cell-Based Assays by Multi-Parametric Analysis

As the body's largest organ, skin protects against germs, regulates body temperature and enables touch (tactile) sensations. The skin's main layers include the epidermis, dermis and hypodermis. The skin and its derivatives (hair, nails, sweat and oil glands) make up the integumentary system. One of the main functions of the skin is protection. It protects the body from external factors such as bacteria, chemicals, and temperature. The epidermis is the top layer of the skin. Keratin, a protein inside skin cells, makes up the skin cells and, along with other proteins, sticks together to form this layer. The epidermis . . .
Acts as a protective barrier:
   The epidermis keeps bacteria and germs from entering the body and bloodstream and causing infections. It also protects against rain, sun and other elements.
Makes new skin:
   The epidermis continually makes new skin cells. These new cells replace the approximately 40,000 old skin cells that the body sheds every day. You have new skin every 30 days.
Protects our body:
   Langerhans cells in the epidermis are part of the body's immune system.

They help fight off germs and infections.
Provides skin color:
   The epidermis contains melanin, the pigment that gives skin its color. The amount of melanin you have determines the color of the skin, hair and eyes. People who make more melanin have darker skin and may tan more quickly.

Skin changes are among the most visible signs of aging. Evidence of increasing age includes wrinkles and sagging skin. Aging slows down the skin regeneration process, the skin can appear thicker, less flexible, and more wrinkled or textured.

Skin is constituted by 4 key cell populations—Fibroblasts, Keratinocytes, Melanocytes and sebocytes.
Fibroblasts are the most common cell type of the connective tissues found throughout the body and the principal source of the extensive extracellular matrix (ECM) characteristic of these tissues.
Keratinocytes form a protective skin barrier and lock the moisture. These cells are also responsible for turnover and renewal of skin.
Sebocytes secret sebum to keep the skin moist.
Melanocytes synthesize melanin that imparts pigmentation to skin.

Skin aging is important medical and social problem in modern world. Fibroblasts synthesize all components of the extracellular matrix of the dermis, including collagen, elastin, proteoglycans, and minor proteins. Consequently, the changes in the size and functional status of these cells may disrupt the formation of intercellular substance, which will contribute to the appearance of outward signs of aging.

During the aging process, the proliferative and metabolic activity of fibroblasts decreases and their functions are impaired, leading to reduction of the synthesis of structural substances such as collagen, elastin, hyaluronic acid, and chondroitin. Researches have shown that there is an age-related decrease in dermal fibroblasts number is associated with diminished proliferation of these cells. Test agents/formulations that stimulate the proliferation of fibroblasts are beneficial in promoting skin health and good for rejuvenation and renewal of skin. Hence in the present study the resultant effect of test items on proliferation of skin fibroblasts and keratinocytes was investigated.

Extracellular matrix (ECM) proteins such as Collagen, Elastin, Hyaluronic acid (HA) are essential building blocks of skin and are vital for the renewal and regeneration of skin. Collagen causes an increase in fibroblasts and extracellular matrix proteins and a decrease in metalloproteinase. These rising fibroblasts found in the various layers of the human dermis produce a plethora of extracellular matrix proteins that enhance skin health and thus slow skin aging. Elastin's main role is to provide stretchiness in our body, and it's approximately 1,000 times stretchier than collagen. Elastin provides benefits to skin function beyond solely mechanical elasticity. By acting as a substrate for cell growth, elastin can support improved regeneration and remodeling of the dermis, which is critical for effective wound healing and scar repair. Skin aging is also associated with loss of skin moisture. The key molecule involved in skin moisture is hyaluronan or hyaluronic acid (HA), a glycosaminoglycan (GAG) with a unique capacity to bind and retain water molecules. It helps to retain and attract water and keeps tissues hydrated and lubricated.” it's a hydrating ingredient that our body already produces—similar to squalene—and it's a must-have to keep mature skin looking plump, hydrated and dewy. Levels of elastin, collagen, and hyaluronic acid gradually decrease due to aging, exposure to sun and environment and other stress. This results in loose, saggy skin and formation of wrinkles and fine lines. Test agents/formulations that promote these ECM markers are good for skin health promotion. Rejuvenation and replenishment of skin by increased synthesis of ECM markers supports skin health and managed aging. Hence in the present study the resultant effect of test items on ECM markers in skin fibroblasts was investigated.

Skin wound healing aims to repair and restore tissue through a multistage process that involves different cells and signaling molecules that regulate the cellular response and the dynamic remodeling of the extracellular matrix. Healing can follow two different mechanisms: regeneration or repair. The sequence of events after skin injury has been extensively studied and involves many cell types and signals to induce wound healing. These stimuli promote the arrival of progenitor cells to the site that will start the regeneration of the damaged tissue. However, this process, which is inherent and highly orchestrated during development, loses its effectiveness in the adult body, resulting in disorganized extracellular matrix (ECM) commonly known as a scar.

Day-to-day external insults, such as radiation and chemical exposure, may cause damage to DNA, but humans are equipped with a powerful DNA repair mechanism that is able to offset the damage. However, as the age increases, the repair mechanism efficiency is reduced, which causes some DNA errors to escape the repair mechanism. Promotion of fibroblasts and keratinocytes migration potential suggests improved repair, renewal and wound healing properties by test agents/formulations. Hence in the present study the resultant effect of test items on migration of skin fibroblasts and keratinocytes was investigated.

Some areas of skin are continuously exposed to a variety of environmental stressors that can inflict direct and indirect damage to skin cell DNA. Skin homeostasis is maintained by mesenchymal stem cells in inner layer dermis and epidermal stem cells (ESCs) in the outer layer epidermis. Reduction of skin stem cell number and function has been linked to impaired skin homeostasis. Chronic exposure to ultraviolet B (UVB) is a major cause of skin aging. Skin is continuously exposed to a variety of environmental stresses, including ultraviolet (UV) radiation. UVB is an inherent component of sunlight that crosses the epidermis and reaches the upper dermis, leading to increased oxidative stress, activation of inflammatory response and accumulation of DNA damage among other effects. UVB is the most dangerous component of sunlight. Due to its high energy, UVB is able to cross the epidermis and reach the upper dermis where is interacts with cellular chromophores, leading to DNA damage and increased oxidative stress. These events activate numerous signaling pathways that lead to decreased collagen production, increased synthesis and activity of matrix metalloproteases (MMPs), increased production of ROS, aggravated inflammatory response and secretion of inflammatory cytokines, enhanced apoptosis and ultimately loss of cell viability. External factors such as ultraviolet radiation (UVR), temperature, air pollutants, smoking, and diet, accelerate skin aging. These factors can cause aging through reactive oxygen species (ROS)-mediated inflammation, as well as aged skin is a source of circulatory inflammatory molecules which accelerate skin aging and cause aging-related diseases. Hence in the present study the resultant effect of test items on UVB induced cell death, apoptosis and inflammation was investigated.

Aging and Matrix Metalloproteinases (MMPs) are a family of ubiquitous endopeptidases that can degrade ECM proteins. Levels of MMPs are elevated in aging skin. Reactive oxygen species (ROS) are a major driving force behind the increase in MMP levels in aged skin. ROS are generated in the skin from both extrinsic and intrinsic sources, such as ultraviolet irradiation and metabolically generated pro-oxidants. Reactive oxygen species (ROS) generated in the aging process activate mitogen-activated protein kinases (MAPKs) and induce transcription factors, including activator protein 1 (AP-1) and nuclear factor-κB (NF-κB). This activation increases matrix metalloproteinase (MMP) expression and inhibits transforming growth factor-β (TGF-β) signaling, which leads to collagen fragmentation and decreased collagen biosynthesis. This hinders the mechanical interaction between fibroblasts and the extracellular matrix (ECM), and consequently reduces the size of dermal fibroblasts. Aged fibroblasts produce a greater amount of ROS that further increases the expression of MMPs and inhibits TGF-β signaling, creating a positive feedback loop that accelerates dermal aging. Test agents/formulations that target MMPs and inhibit the expression in fibroblasts are beneficial for skin health promotion. Hence in the present study the resultant effect of test items on levels of MMPs in skin fibroblasts was investigated.

With aging, the amount of fibroblasts decreases markedly with age and their functional activity is also reduced. Ageing process in cells is associated with oxidative stress. Ultraviolet produces reactive oxygen species responsible for accumulation of DNA and cellular damage. Also DNA damage occurs due to intrinsic aging process. Skin is subject to both an intrinsic ageing process (due to the passage of time-left hand panel) and to an extrinsic ageing processes (principally as a result of exposure to ultraviolet radiation [UVR]-right hand panel). Mitochondrial DNA damage, increased ROS production and telomere shortening are thought to mediate this process. DNA damaging effects of UVR result in activation of cell cycle arrest (checkpoint activation) and DNA repair proteins, damaged stem cell niche, and dermal fibroblasts. These facilitate epidermal stem cells/ESC depletion and loss of extracellular matrix (ECM) integrity, leading to premature skin aging. Test agents/formulations that protect DNA against oxidative stress and UVB have anti-aging properties. Hence in the present study the resultant effect of test items on DNA protection against UVB and oxidative damage was investigated.

Human skin serves as a barrier against multiple environmental insults, including pathogenic microorganisms, pollutants, toxic chemicals, and UV radiation. In the outermost layer of the epidermis, the cornified envelope functions as a mechanical and permeability barrier. The cornified cell envelope structure is formed beneath the plasma membrane in terminally differentiating stratified squamous epithelia. It provides a vital physical barrier to these tissues in mammals and consists of a 10 nm thick layer of highly crosslinked insoluble proteins. In cultured keratinocytes, CEs are developed in 5-7 days of culture in low Calcium medium. CE can be prepared by exhaustive boiling cultured keratinocytes in a solution containing a surfactant such as SDS and recovering the insoluble fraction by removing the soluble components by such means as centrifugation. Formulations/agents that promote the formation of CE strengthen the skin barrier properties, ensure moisture lock inside and minimizes crack formation. Hence in the present study the resultant effect of test items on cornified envelope formation was investigated.

Defective skin barrier formation leads to dry and cracked skin. The skin is them impaired to lock the moisture inside. The barrier dysfunction correlates with the downregulation of barrier-related molecules such as filaggrin (FLG), loricrin (LOR), and involucrin (IVL). IVL is expressed in the upper spinous layer, but mainly in the granular layers, and is involved in the initial step of cornified envelope formation. LOR is the most abundant component of the cornified envelope. It is very hydrophobic, insoluble, and is easily polymerized via disulfide crosslinking in ambient air, making it suitable as a protein that reinforces the cornified envelope. FLG is involved in aggregating the K1 and K10 filaments into higher molecular-weight parallel structures that facilitate the incorporation of K1 and K10 into the cornified envelope and contribute to the thin granular keratinocyte shape. Aquaporins (AQPs), a family of membrane channel proteins that allow the osmotic movement of water and small neutral solutes. They play key role in Keratinocyte early differentiation, keratinocyte proliferation and migration during wound healing; skin hydration (circadian rhythm); maintenance of epidermal water permeability barrier. Agents that promote the synthesis of surface proteins (FLG, IVL, LOR, AQP3) help in maintaining a health skin barrier and keep the skin hydrated by locking the moisture inside. Hence in the present study the resultant effect of test items on proteins of skin barrier was investigated.

Melanogenesis is the process for the production of melanin, which is the primary cause of human skin pigmentation. Melanin is mainly produced by melanocytes that are localized in the epidermis, the outermost layer of the skin; it is also this layer that determines skin color in humans. Skin color is influenced by a number of intrinsic factors, including skin types and genetic background, and extrinsic factors, including the degree of sunlight exposure and environmental pollution. Skin color is determined by the quantity of melanosomes and their extent of dispersion in the skin. Under physiological conditions, pigmentation can protect the skin against harmful UV injury. However, excessive generation of melanin can result in extensive aesthetic problems, including melasma, pigmentation of ephelides and post-inflammatory hyperpigmentation.

Tyrosinase catalyses the rate-limiting step where L-tyrosine is converted to L-3,4-dihydroxyphenylalanine (L DOPA), leading to the eventual formation of the pigment. Abnormal TYR activity leads to pigmentary disorders, such as the abnormal accumulation of melanin (hyperpigmentation) that accounts for most dermatology visits. Skin lighteners can be divided by their mechanisms of action, such as inhibition of tyrosinase transcription, inhibition of melanosome transfer, and accelerated epidermal turnover, with the most common target being tyrosinase (TYR) inhibition. By decreasing the activity and/or expression of TYR, melanogenesis can be inhibited, leading to reduced melanin production Traditional pharmacological agents, including corticosteroids, hydroquinone and amino mercuric chloride, lighten skin tone through the inhibition of either melanocyte maturation or interference with the process of melanogenesis. Naturally occurring skin-whitening agents exert their effects by regulating melanin production through a number of mechanisms, including inhibiting the expression and activity of TYR and suppressing the uptake and distribution of melanosomes. Hence in the present study the resultant effect of test items on melanin synthesis, migration of melanocytes and tyrosinase inhibition was investigated.

### Skin Health—Fibroblast Based Assays

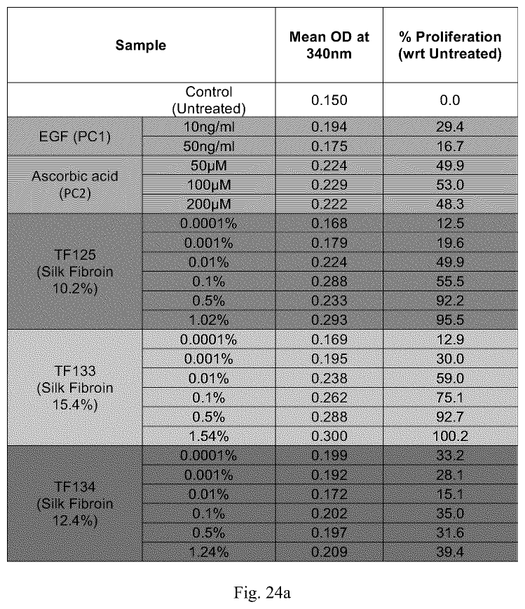

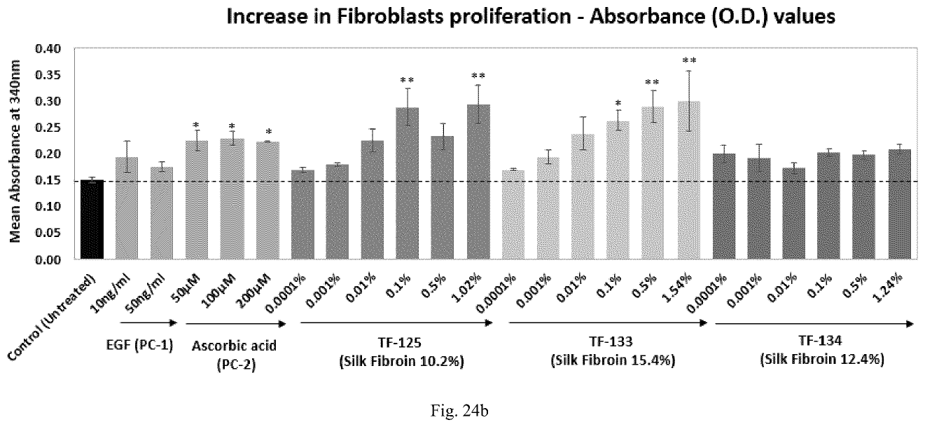

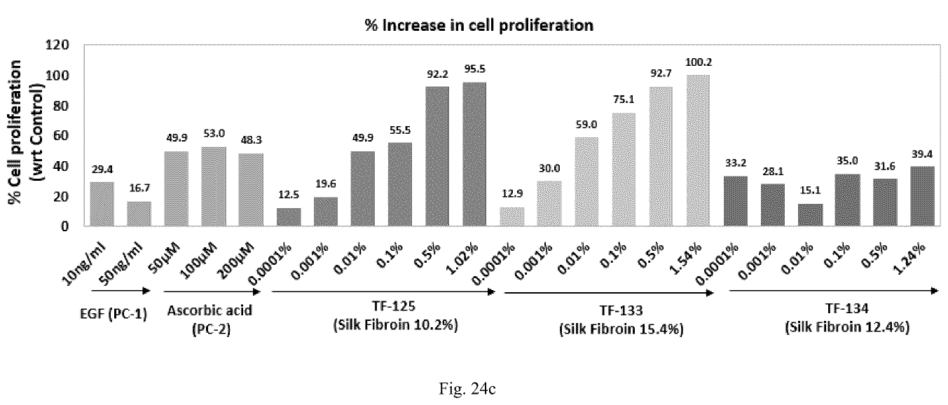

### 1a. Increase in Cell Proliferation of Fibroblasts (as Seen in FIGS. 24a-24c)

Study Design Assay 1a)—Skin Health—Increase in Fibroblasts Proliferation

### Study Design

Test System—Human Fibroblasts (HFF-1)
Number of cells plated—1500 cells/96-well plates
FBS concentrations—0.5%
Test items—TF-125 (10.2%), TF-133 (15.4%), TF-134 (12.4%)
Time points—3 days
Estimation method—BrdU assay
Positive Control/s—Ascorbic acid, EGF

### Procedure

Cells were plated in 10% FBS in 96-well plates and incubated for 24 h.
Cells were serum starved in 0% FBS for 24 h.
Cells were treated with 3 Test items in 0.5% FBS at various concentrations.
After 3 days (72h), the effect on proliferation was determined by BrdU assay.
   10 μl of BrdU solution was added/well and cells were incubated for 90 minutes at 37° C.
   After removing medium, cells were fixed, and the DNA was denatured in one step by adding 200 μl of FixDenat. Cells were incubated for 30 min at RT (15-25° C.).
   The FixDenat solution was removed and 100 μl of Anti-BrdU-POD solution was added to each well. Cells were incubated for 90 min at RT (15-25° C.).
   Wells were washed 3 times using 200 μl of washing solution.
   100 μl/well of substrate solution was added.
   Cells were incubated for 30 min at RT (15-25° C.).
   After incubation, a blue coloured complex was formed. Absorbance of each well was measured at 370 nm.

### Calculations

Increase in proliferation was calculated wrt control (Untreated cells).

$$[ ( A - B ) / B ] * 100 ; Where A = Absorbance in Test Item treated cells , B = Absorbance in Control ( Untreated ) cells$$

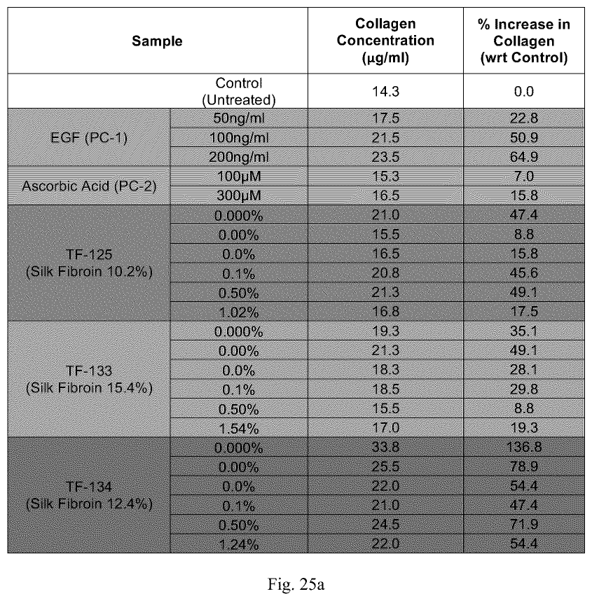

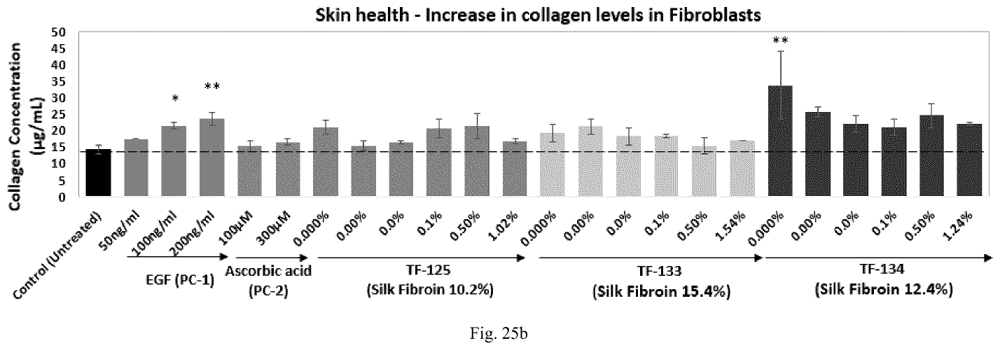

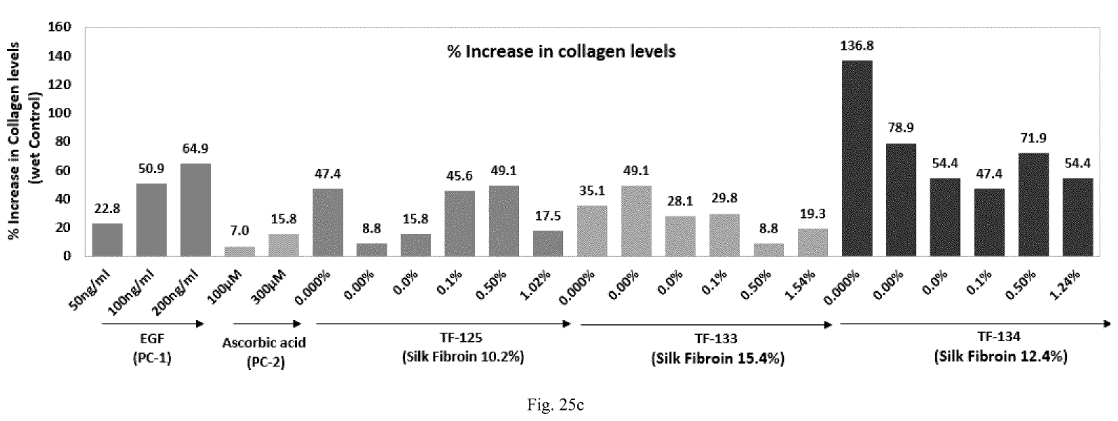

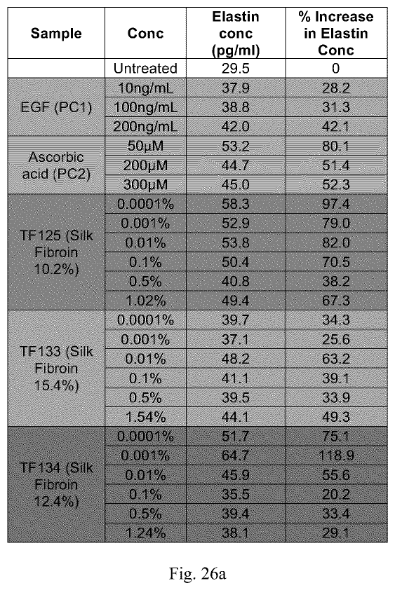

### 1b. Simulation of ECM Markers in Fibroblasts (as Seen in FIGS. 25a-25c)

Study Design Assay 1b)—Skin Health—Increase in ECM (Collagen)

### Study Design

Test System—Human Fibroblasts (HFF-1)
Number of cells plated—50,00 cells/12-well plates
FBS concentration—0.5%
Test items—TF-125 (10.2%), TF-133 (15.4%), TF-134 (12.4%)
Time points—3 days
Estimation method—Direct Red dye Staining (Collagen)
Positive Control/s—Ascorbic acid, EGF

### Procedure

HFF-1 cells were plated in 12-well plate at the density of 50,000 cells/well in DMEM supplemented with 15% FBS.
Following overnight incubation, the cells were subjected to serum starvation for 24 hours in DMEM+0% FBS.
Post serum starvation, cells were treated with non-cytotoxic concentrations of TIs and PCs.
Following 72 hours of treatment with the TIs and PCs, lysates were prepared for collagen estimation and supernatants were collected for determination of HA/Elastin levels.

### Collagen Staining:

i. Cell monolayer was washed with 1×PBS and lysates were prepared in PBS by freeze-thaw method.
ii. The lysates were then stained with Direct red dye and washed with HCl.

The bound dye was eluted with NaOH.
iii. The absorbance was read at 540 nm.

### Calculations

Increase in proliferation was calculated wrt control (untreated cells).

$$[ ( A - B ) / B ] * 100 ; Where A = Absorbance in Test Item treated cells , B = Absorbance in Control ( Untreated ) cells$$

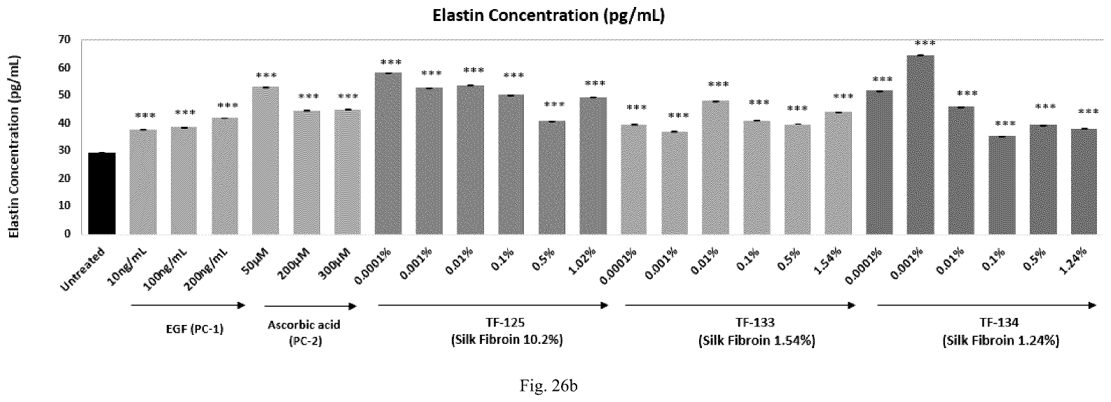

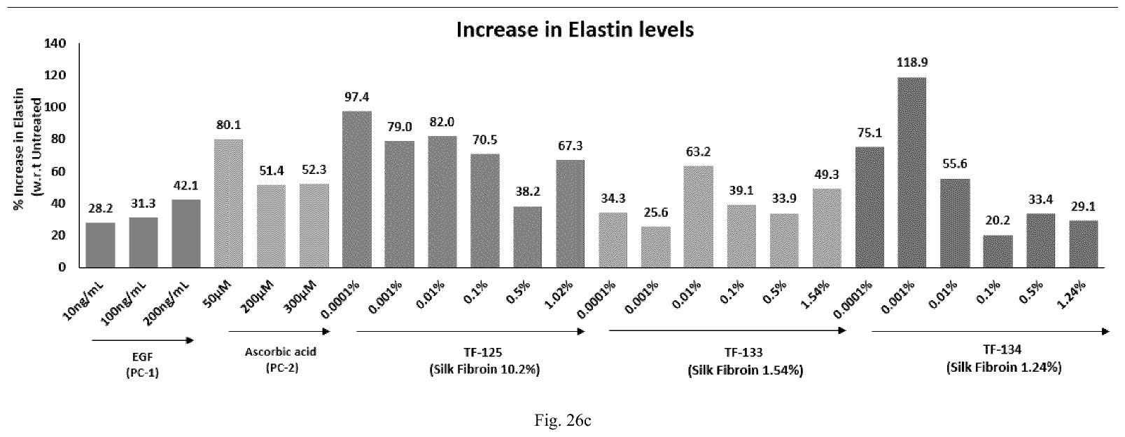

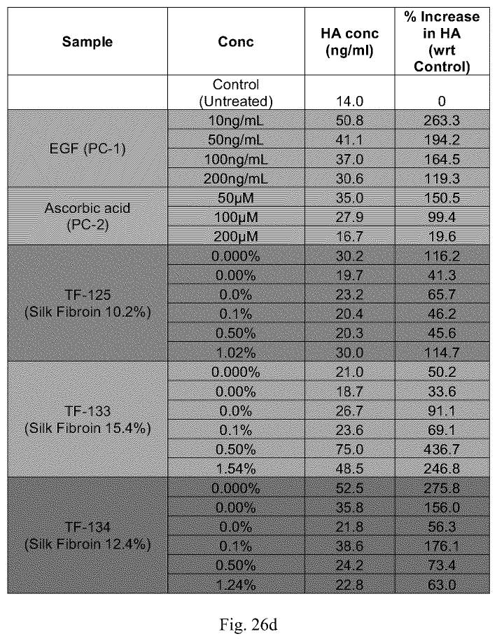

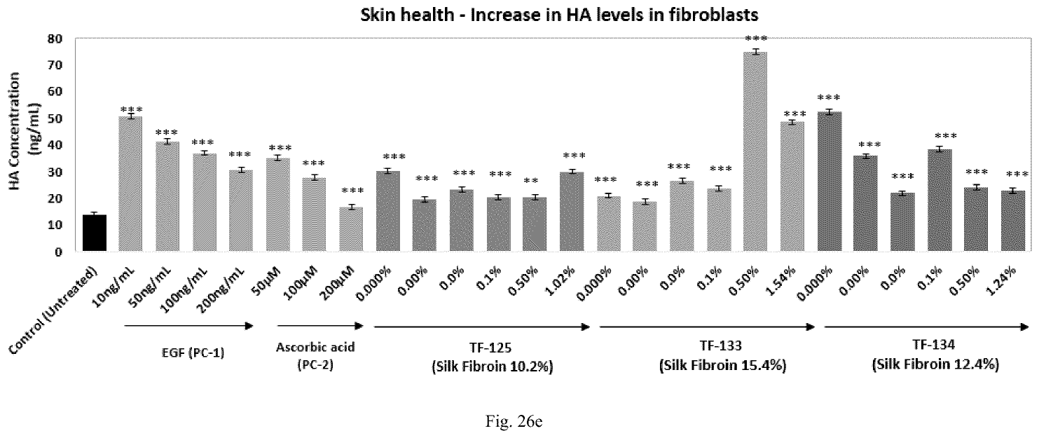

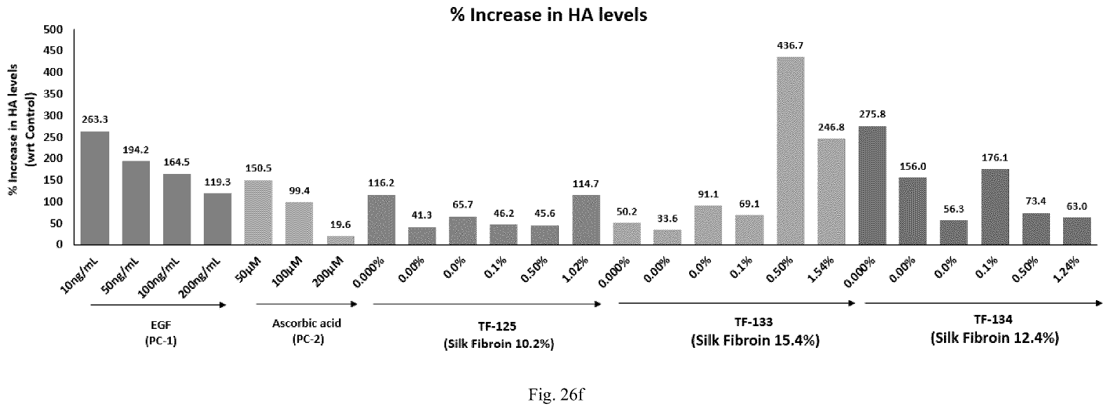

Study Design Assay 1b)—Skin Health—Increase in ECM (HA, Elastin) (as Seen in FIGS. 26a-26f)

### Study Design

Test System—Human Fibroblasts (HFF-1)
Number of cells plated—50,00 cells/12-well plates
FBS concentration—0.5%,
Test items—TF-125 (10.2%), TF-133 (15.4%), TF-134 (12.4%)
Time points—3 days
Estimation method—BrdU assay
Positive Control/s—Ascorbic acid, EGF

### Procedure

HFF-1 cells were plated in 12-well plate at the density of 50,000 cells/well in DMEM supplemented with 15% FBS.
Following overnight incubation, the cells were subjected to serum starvation for 24 hours in DMEM+0% FBS.
Post serum starvation, cells were treated with non-cytotoxic concentrations of TIs and PC.
Following 72 hours of treatment with the TIs and PC, supernatants were collected.
The levels of HA/elastin were determined using ELISA kit as per manufacturer's protocol. Briefly,
   i. Standard and samples (Elastin (Neat) and HA (10× dilution)) were added to respective wells and incubated for 2 hours at 37° C.
   ii. The contents of each well were removed, Biotin-antibody was added to each well and incubated for 1 hour at 37° C.
   iii. Following a wash (3 times) to remove any unbound conjugate, HRP-avidin solution was added to each well and incubated for 1 h at 37° C.
   iv. The plate was again washed 5 times with wash buffer and TMB substrate was added to each well followed by incubation for 30 minutes at 37° C. in the dark.
   v. The reaction was stopped by adding Stop solution to each well.
   vi. The optical density of the color was measured at 450 nm.

### Calculations

Increase in proliferation was calculated wrt control (untreated cells).

$$[ ( A - B ) / B ] * 100 ; Where A = Conc of elastin / HA in Test Item treated cells , B = Conc of elastin / HA in Control ( Untreated ) cells$$

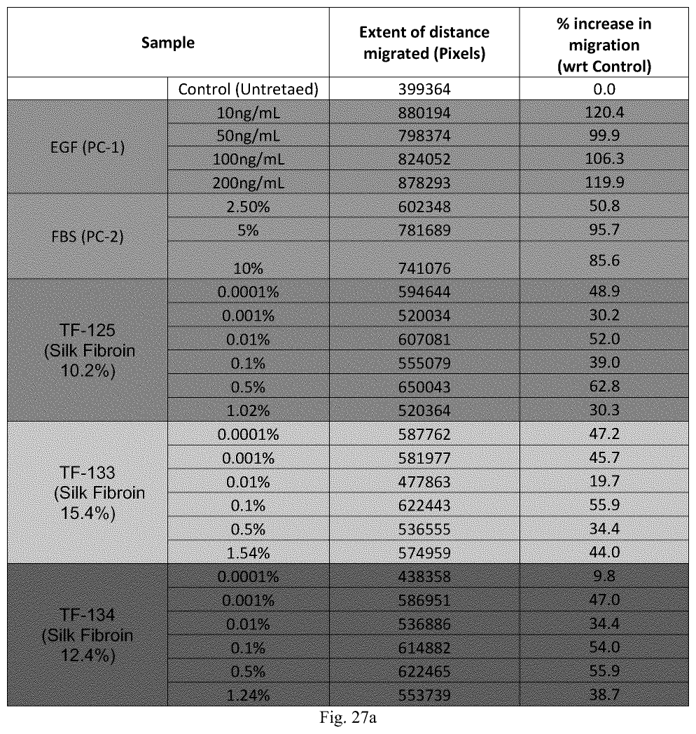

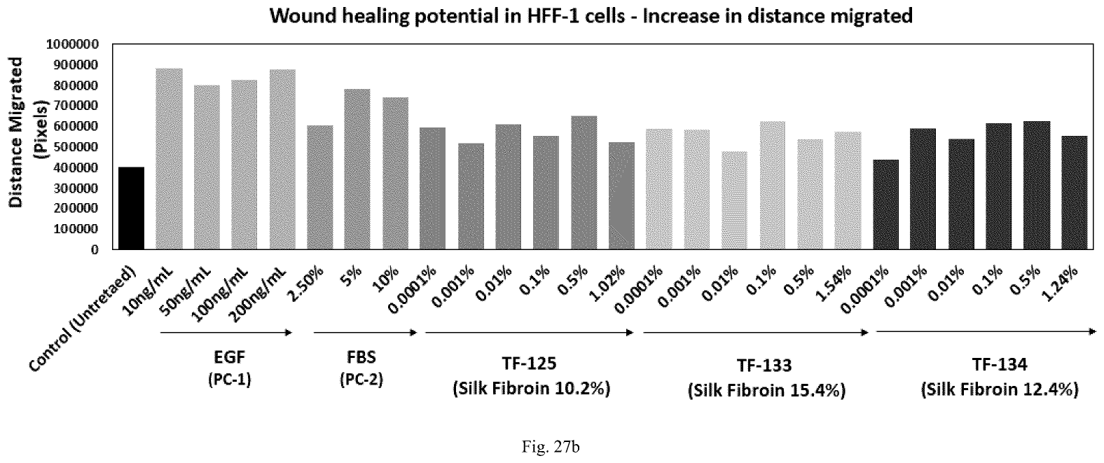

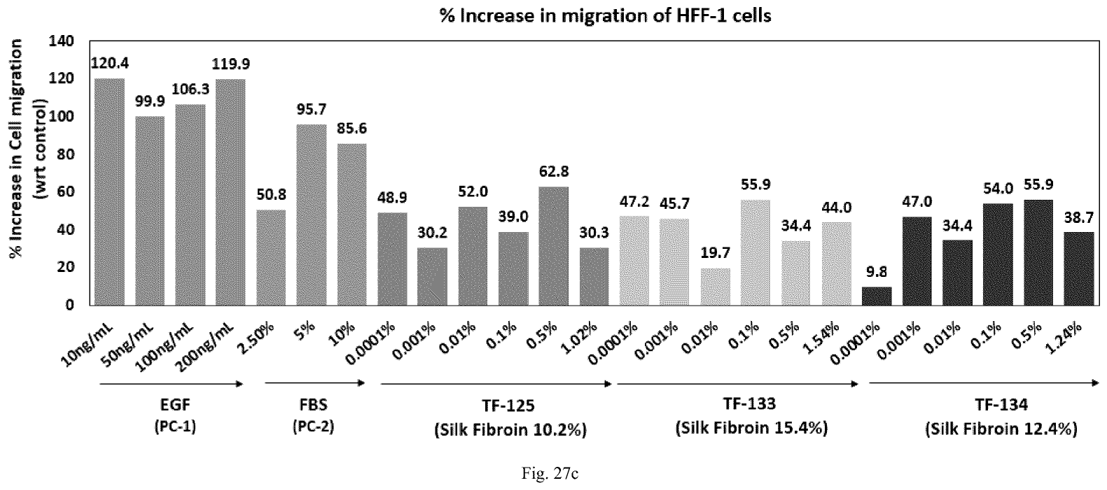

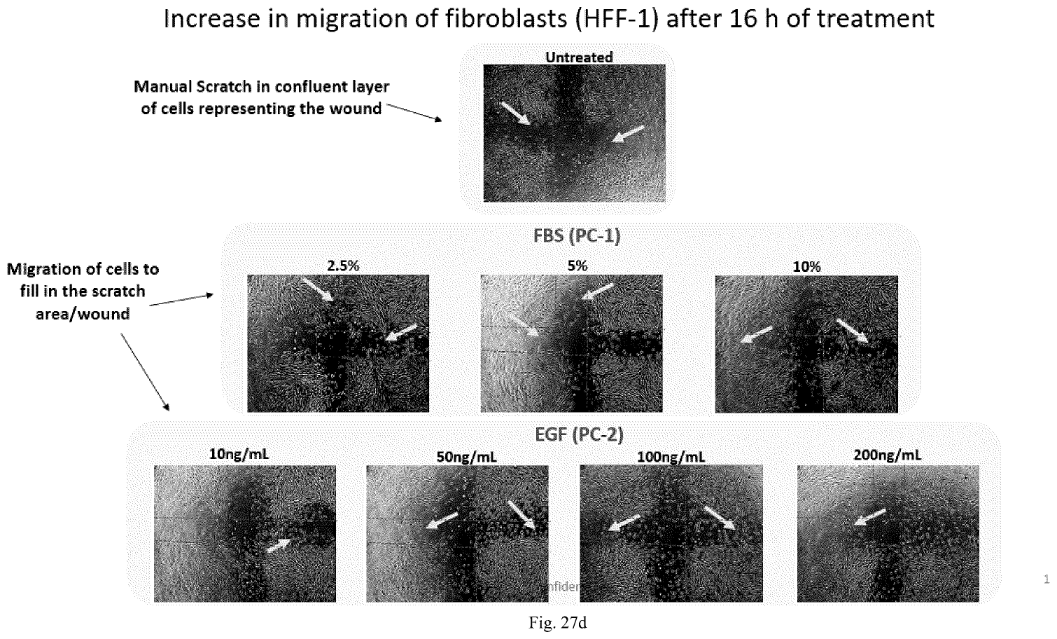

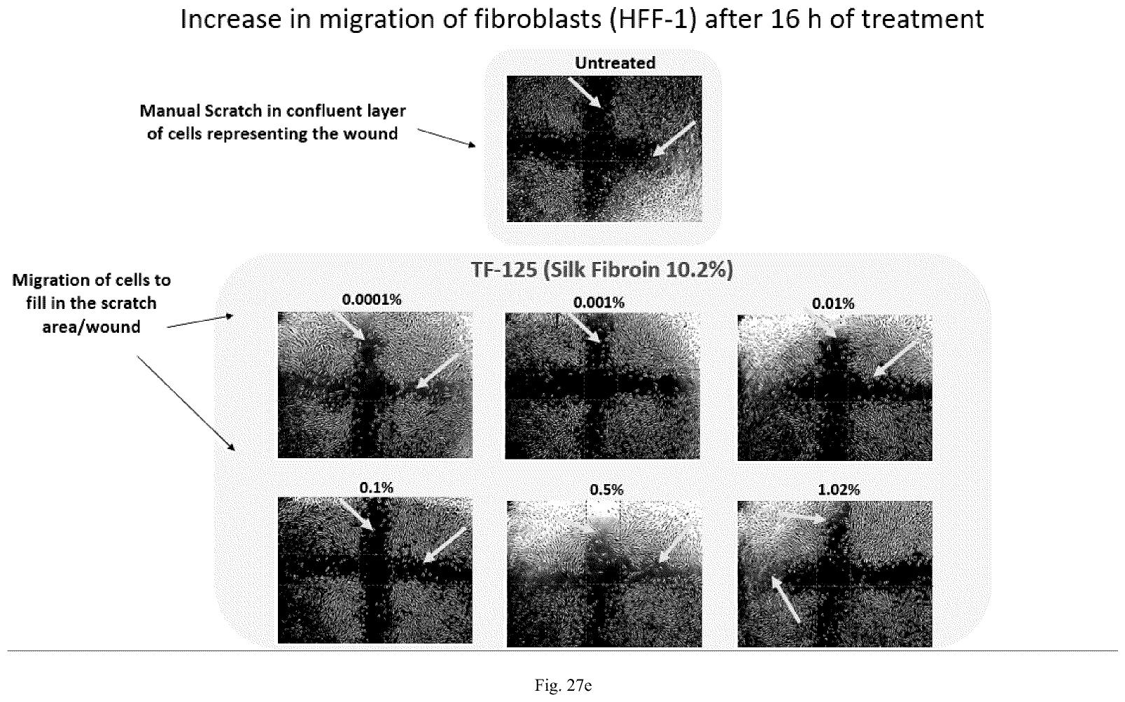

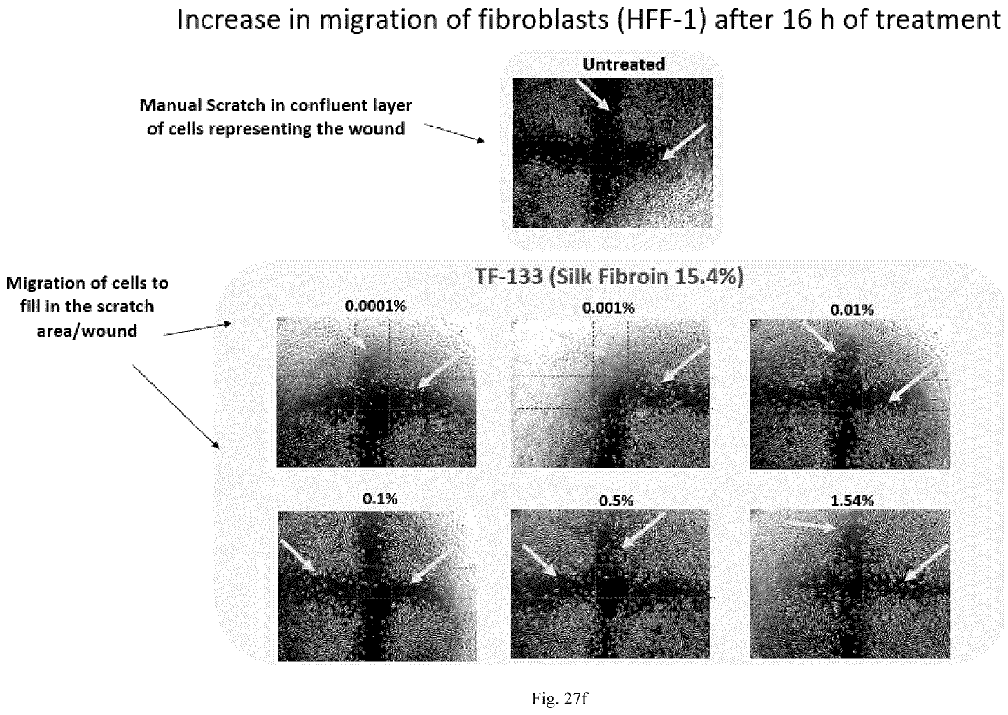

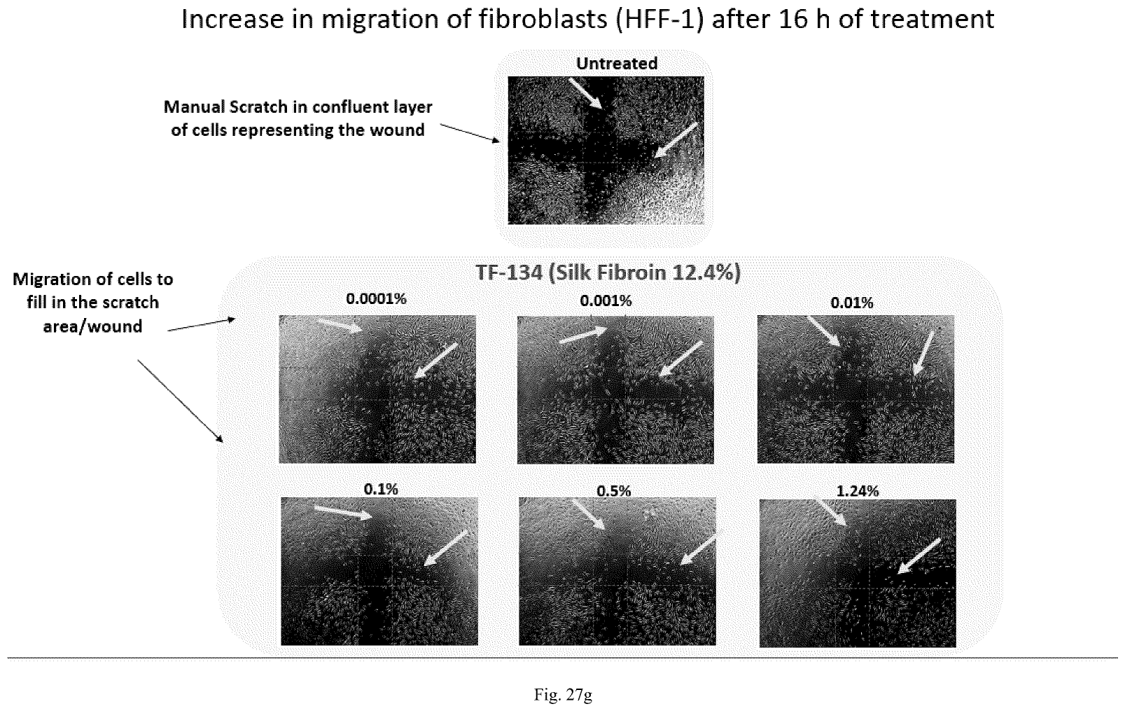

### 1c. Increase in Cell Migration in Fibroblasts (as Seen in FIGS. 27a-27g)

Study Design Assay 1c)—Skin Health—Increase in Migration of Fibroblasts

### Study Design

Test System—Human Fibroblasts (HFF-1)
Number of cells plated—0.1 million cells/well
FBS concentration—0%
Test Items—TF-125 (10.2%), TF-133 (15.4%), TF-134 (12.4%)
Time point—16h
Estimation method—Scratch assay
Positive Control/s—EGF, FBS

### Procedure

Cells were plated in 10% FBS in 12-well plate and incubated for 24 h.
Cells were serum starved in 0% FBS for 24 h.
Scratch was created in each well using a sterile tip followed by treatment of cells with 3 Test items at non-cytotoxic concentrations of TIs and PCs.
Images were captured (0h).
After 16 h, the images were captured to determine the effect of Test Items on migration.
Increase in migration was calculated using ImageJ software.

### Calculations

Increase in migration was calculated wrt control (Untreated cells).

$$[ ( A - B ) / B ] * 100 ; Where A = distance migrated in Test Item treated cells , B = distance migrated in Control ( Untreated ) cells$$

Where Distance migrated=Area in Control (unt) cells−Area in Test Item treated cells

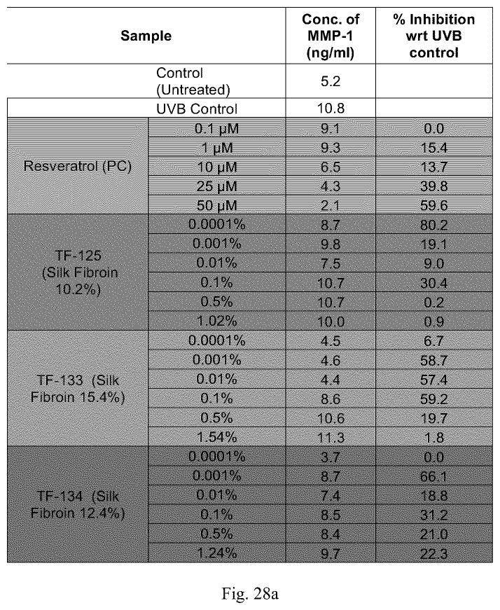

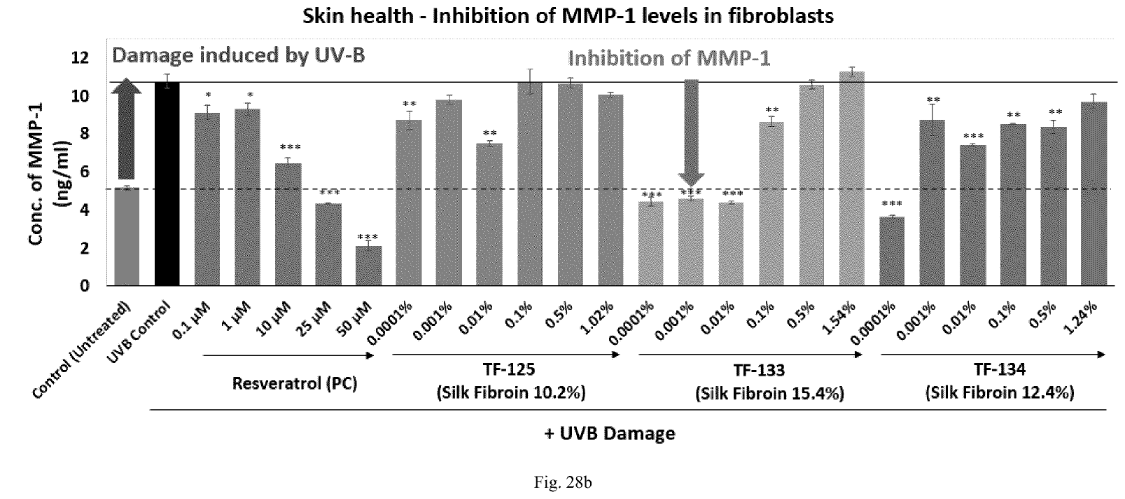

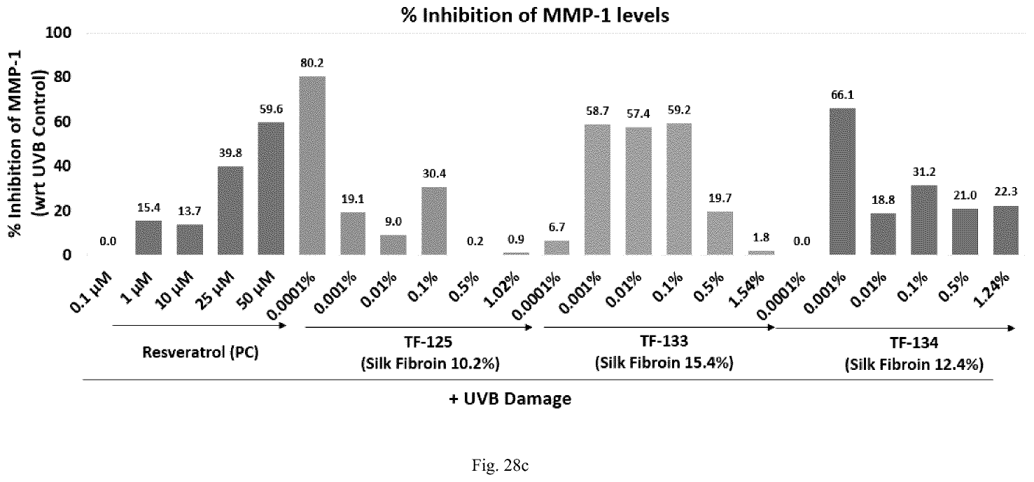

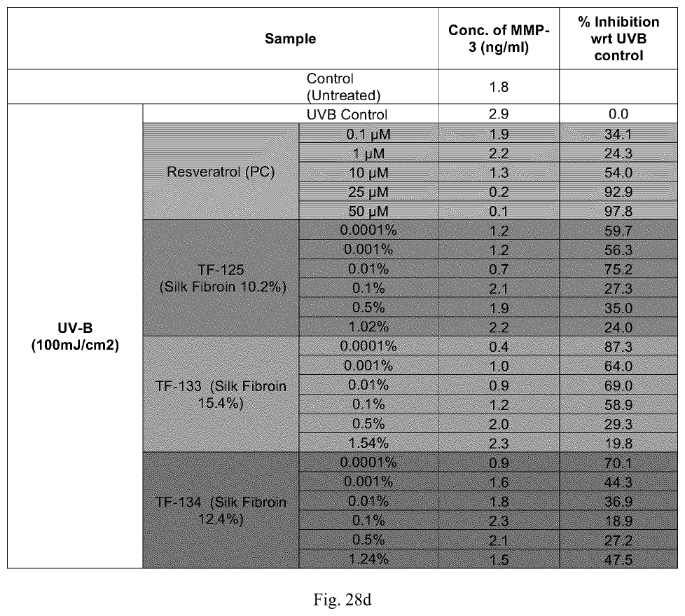

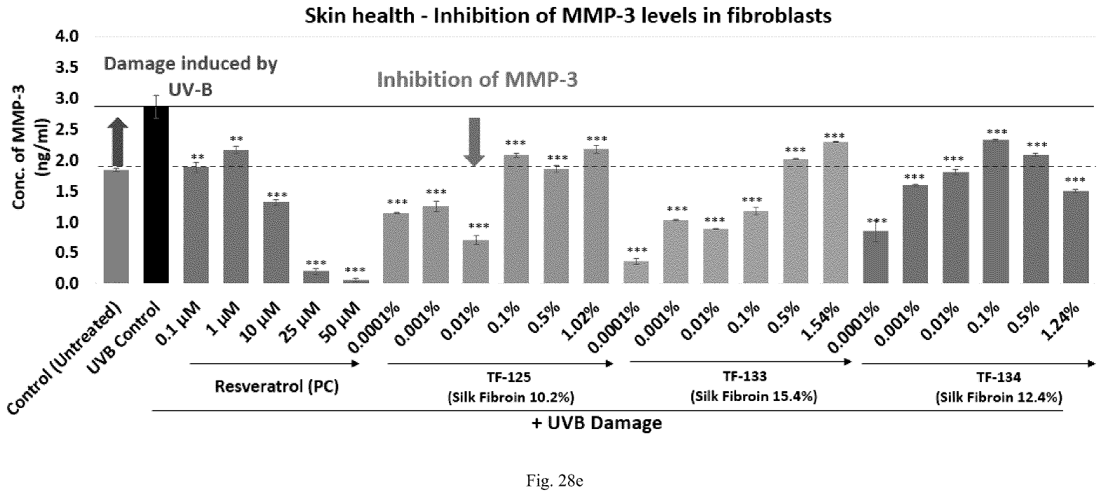

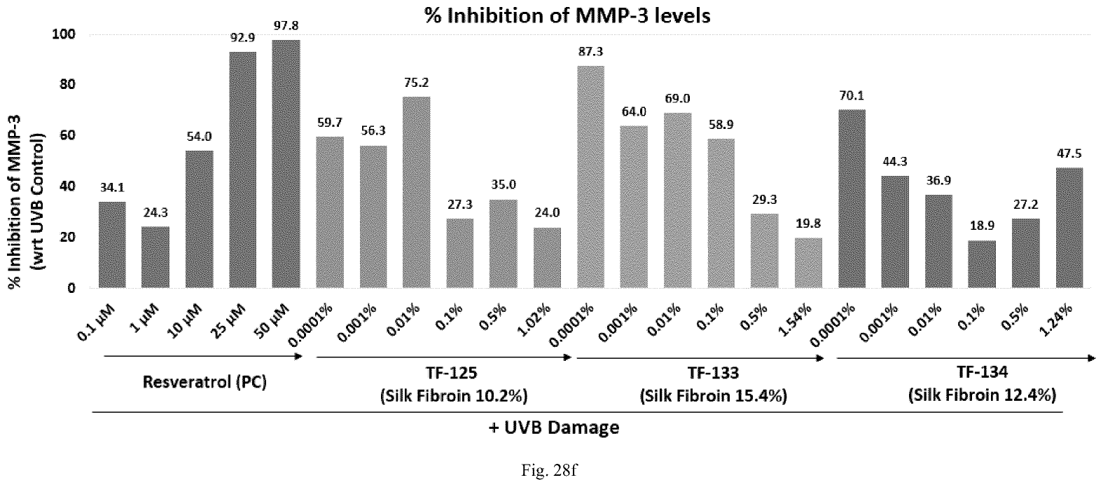

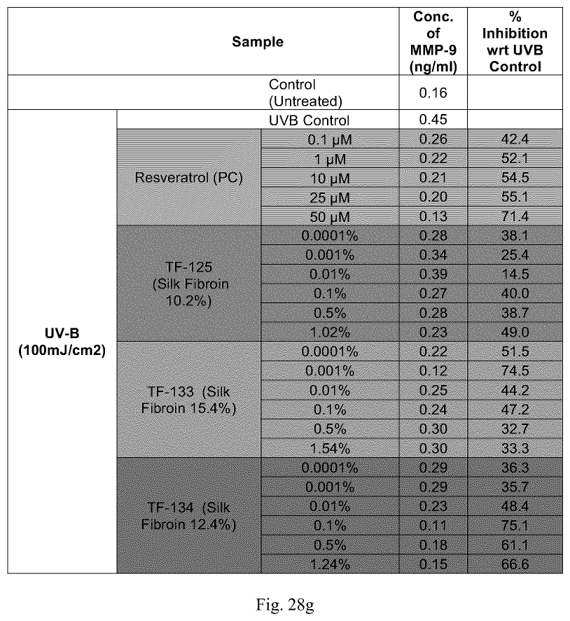

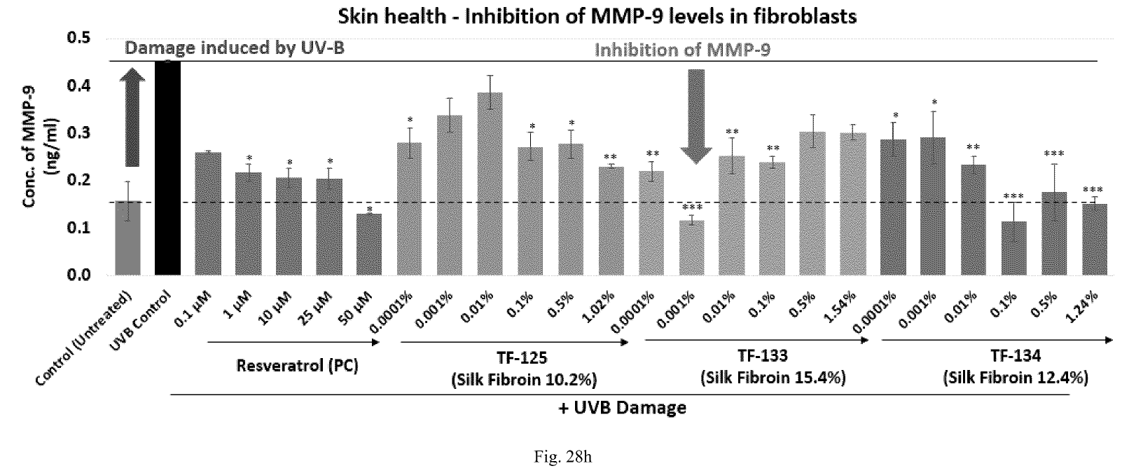

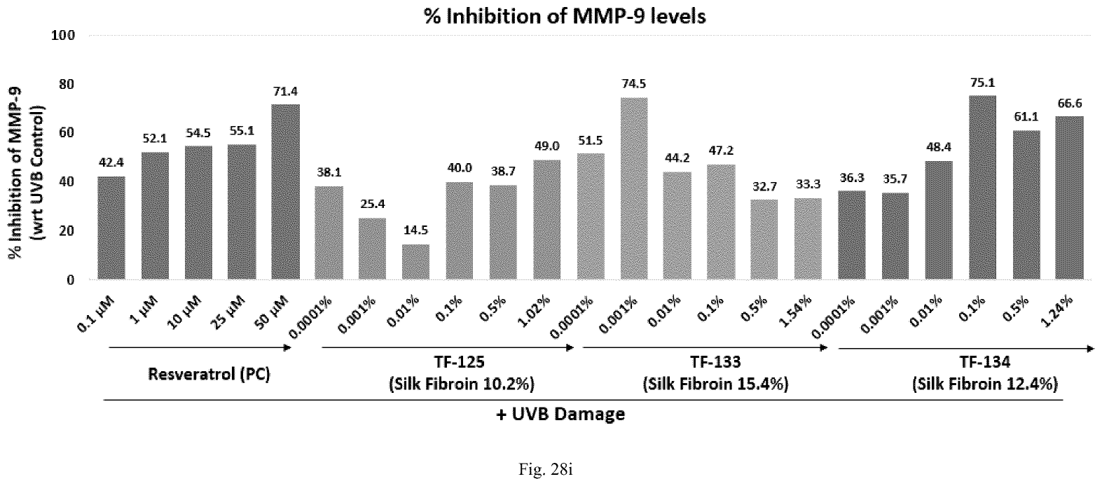

### 1d. Inhibition of MMPs in Fibroblasts (as Seen in FIGS. 28a-28i)

Study Design Assay 1d)—Skin Health—Inhibition of MMPs

### Study Design

Test System—HFF-1 (Skin fibroblasts)
Number of cells plated—0.25 million cells/well
FBS concentration—0%
Test Item—TF-125 (10.2%), TF-133 (15.4%), TF-134 (12.4%)
Time point—24h pretreatment, UVB exposure (100 mJ/cm2), recovery 24h
Estimation method—ELISA
Positive Control/s—Resveratrol

### Procedure

Cells were plated in 6-well plates in 10% FBS and incubated for 24 h. Cells were serum starved in 0.1% FBS for 24 h.
Cells were pre-treated with 3 Test items at non-cytotoxic concentrations for 24 h.
Cells were exposed to UVB irradiation (100 mJ/cm2) for 5.5 min in PBS.
PBS was washed off and replaced with serum-free medium and allowed for recovery for 24 h.
After 24h, culture supernatants were collected. Levels of MMPs were determined by ELISA as follows.
   i. Standard and samples were added to respective wells and incubated for 2 hours at 37° C.
   ii. The contents of each well were removed, Biotin-antibody was added to each well and incubated for 1 hour at 37° C.
   iii. Following a wash (3 times) to remove any unbound conjugate, HRP-avidin solution was added to each well and incubated for 1 h at 37° C.
   iv. The plate was again washed 5 times with wash buffer and TMB substrate was added to each well followed by incubation for 30 minutes at 37° C. in the dark.
   v. The reaction was stopped by adding Stop solution to each well.
   vi. The optical density of the color was measured at 450 nm.

### Calculations

% Inhibition in MMP levels was calculated wrt control (UVB stimulated cells).

$$[ ( B - A ) / B ] * 100 ; Where A = Levels of MMP - 1 / 3 / 9 in Test Item treated cells , B = Levels of MMP - 1 / 3 / 9 in Control ( UVB stimulated cells )$$

### 2. Skin Health—Keratinocytes Based Assays

### 2a. Increase in Cell Proliferation of Keratinocytes

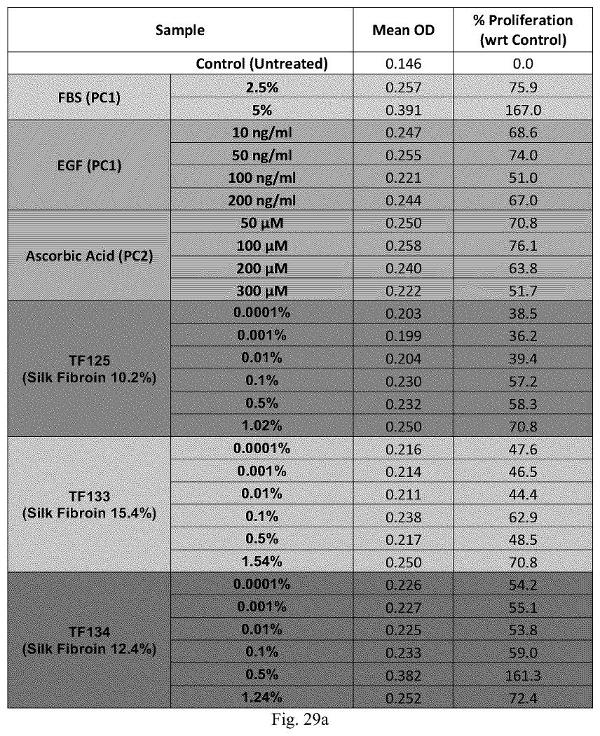

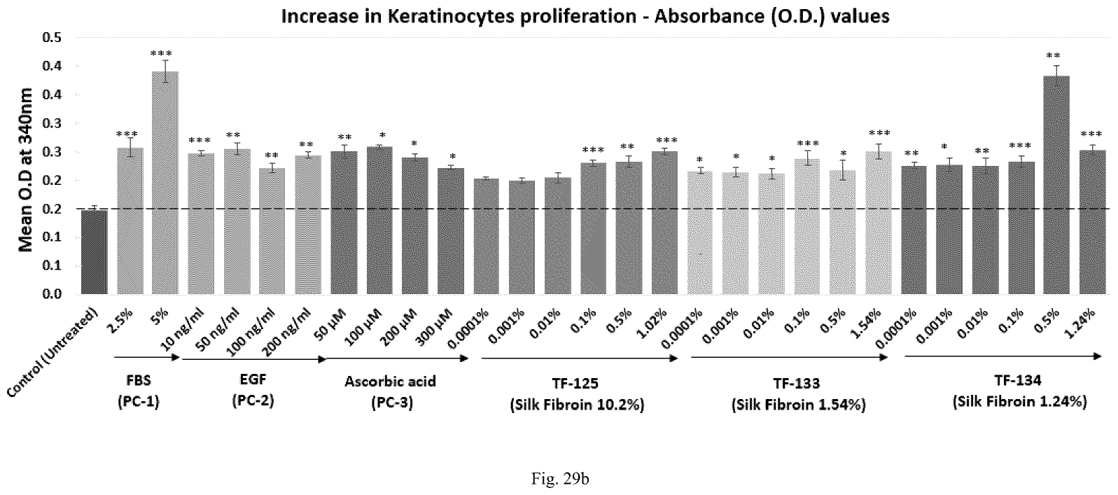

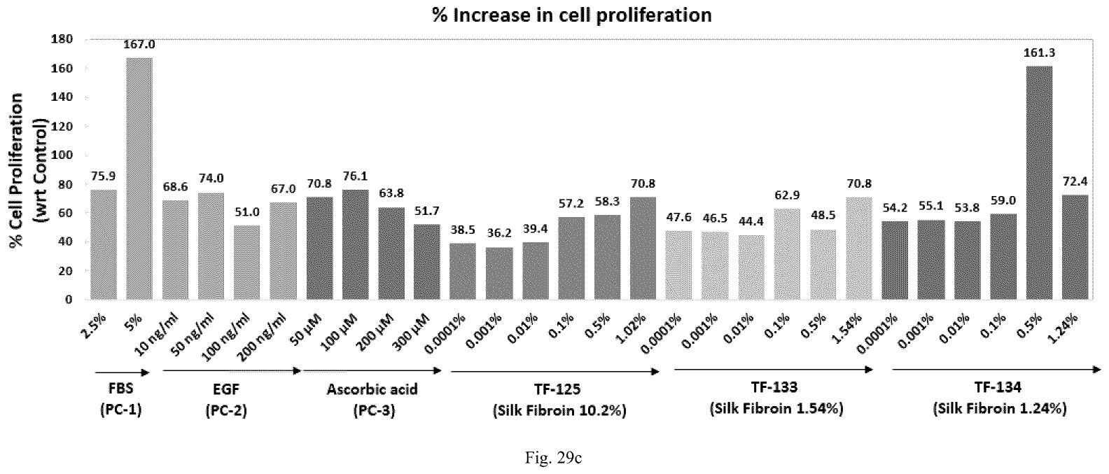

Study Design Assay 2a)—Skin Health—Increase in Keratinocytes proliferation (as seen in FIGS. 29a-29c)

### Study Design

Test System—Human Keratinocytes (HaCaT)
Number of cells plated—1000 cells/well
FBS concentrations—0.1%
Test Items—TF-125 (10.2%), TF-133 (15.4%), TF-134 (12.4%)
Time point—3d, single addition
Estimation method—BrdU assay
Positive Controls—Ascorbic acid, EGF

### Procedure

Cells were plated in 10% FBS in 96-well plates and incubated for 24 h.
Cells were serum starved in 0.1% FBS for 24 h.
Cells were treated with 3 Test items at various concentrations.
After 3 days (72h), the effect on proliferation was determined by BrdU assay.
   i. 10 μl of BrdU solution was added/well and cells were incubated for 90 minutes at 37° C.
   ii. After removing medium, cells were fixed, and the DNA was denatured in one step by adding 200 μl of FixDenat. Cells were incubated for 30 min at RT (15-25° C.).
   iii. The FixDenat solution was removed and 100 μl of Anti-BrdU-POD solution was added to each well. Cells were incubated for 90 min at RT (15-25° C.).
   iv. Wells were washed 3 times using 200 μl of washing solution.
   v. 100 μl/well of substrate solution was added. Cells were incubated for 30 min at RT (15-25° C.).
   vi. After incubation, a blue coloured complex was formed. Absorbance of each well was measured at 370 nm.

### Calculations

Increase in proliferation was calculated wrt control (Untreated cells).

$$[ ( A - B ) / B ] * 100 ; Where A = Absorbance in Test Item treated cells , B = Absorbance in Control ( Untreated ) cells$$

### 2. Increase in Cell Migration of Keratinocytes

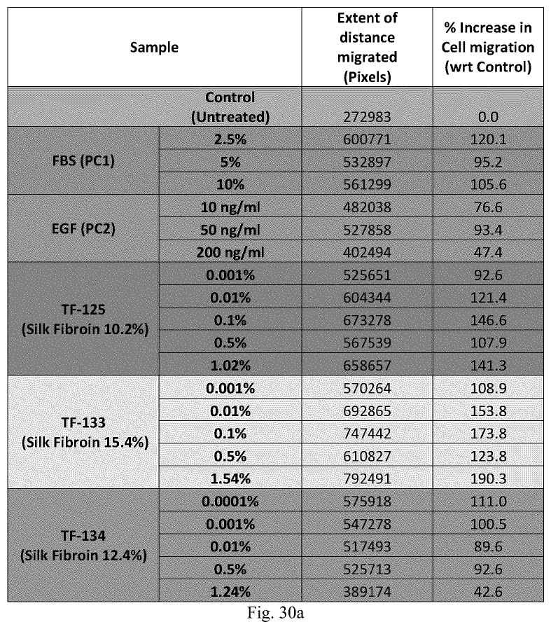

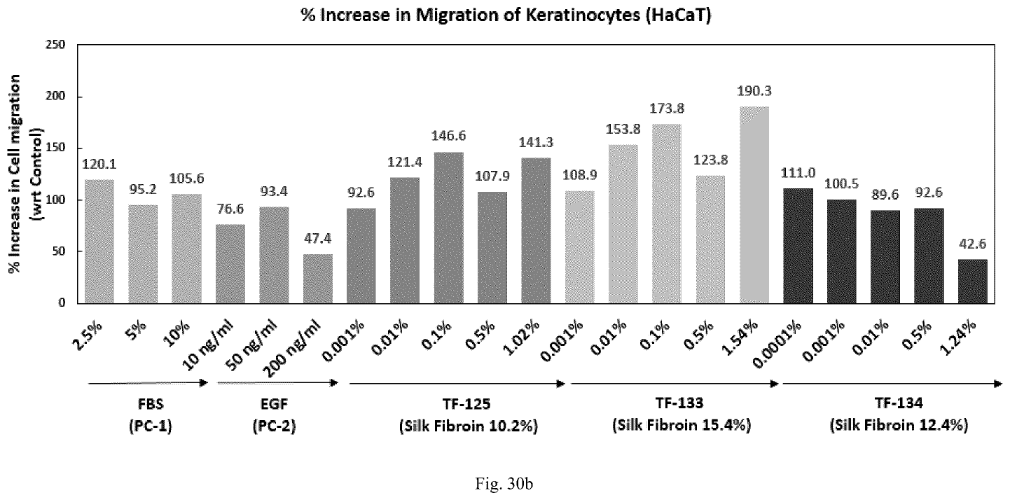

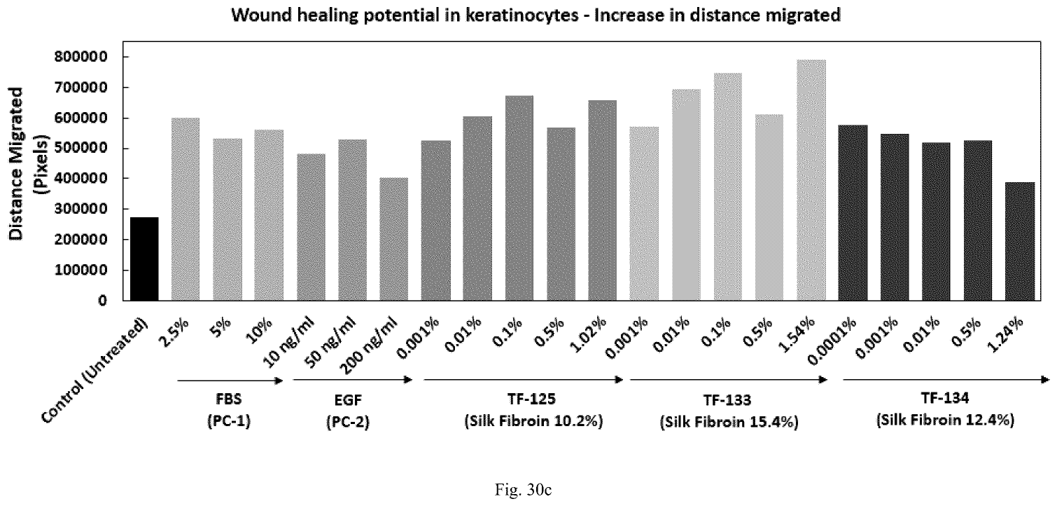

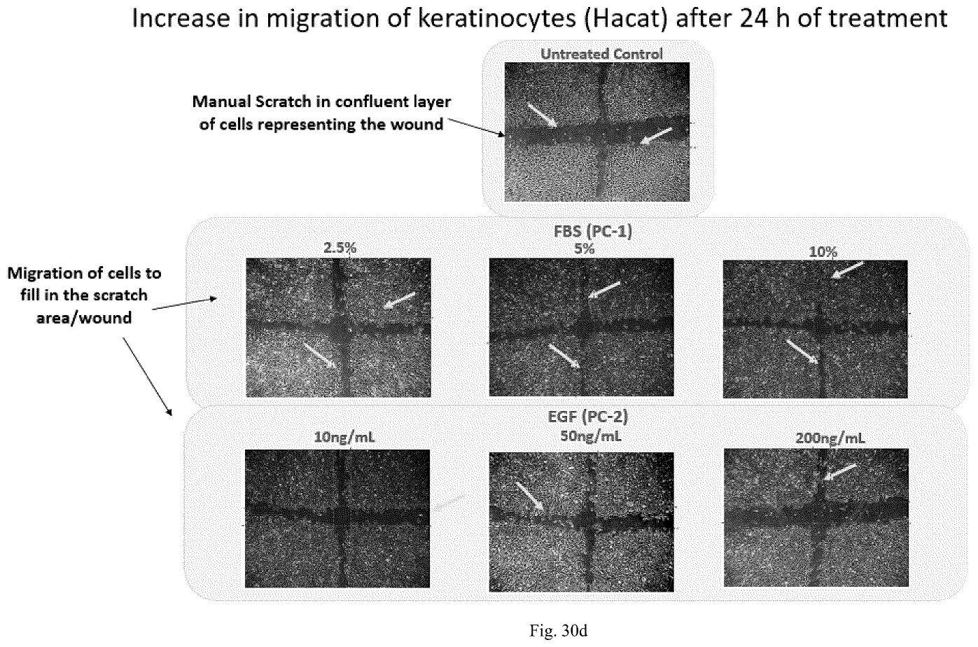

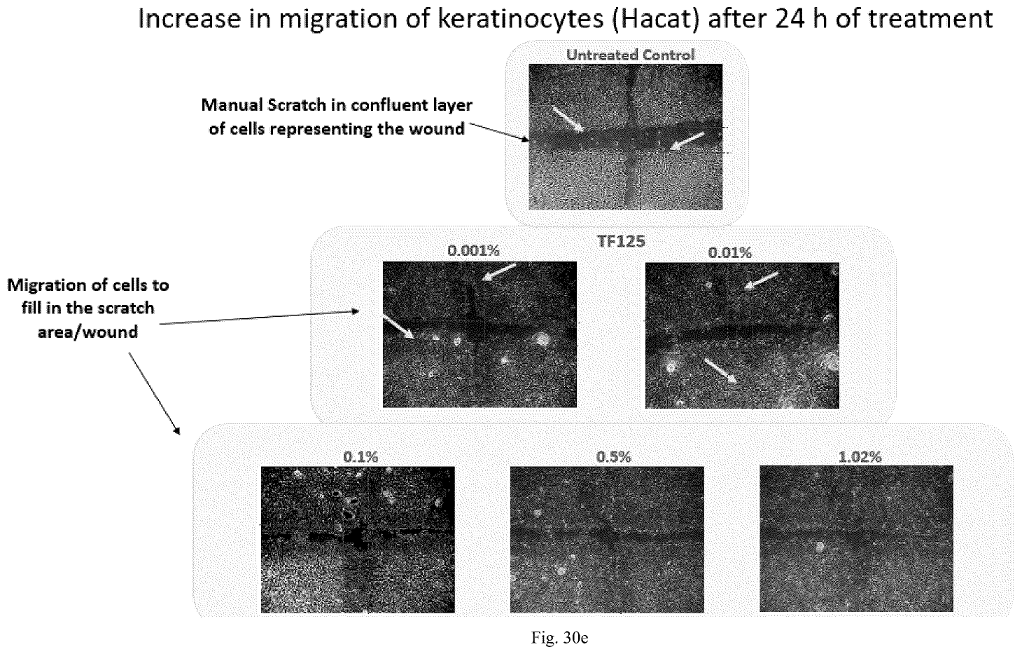

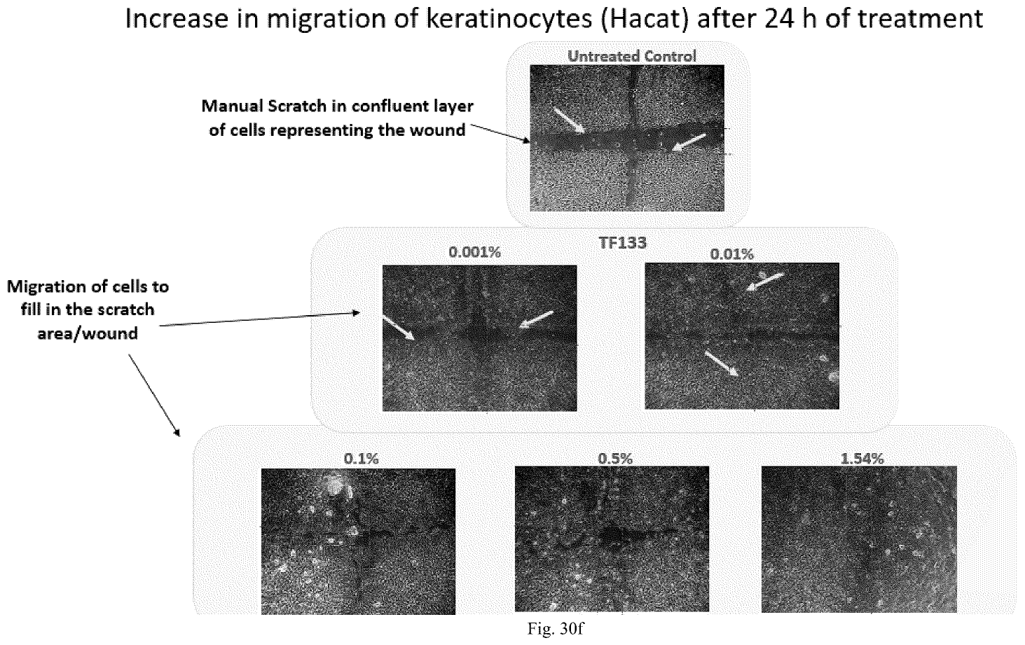

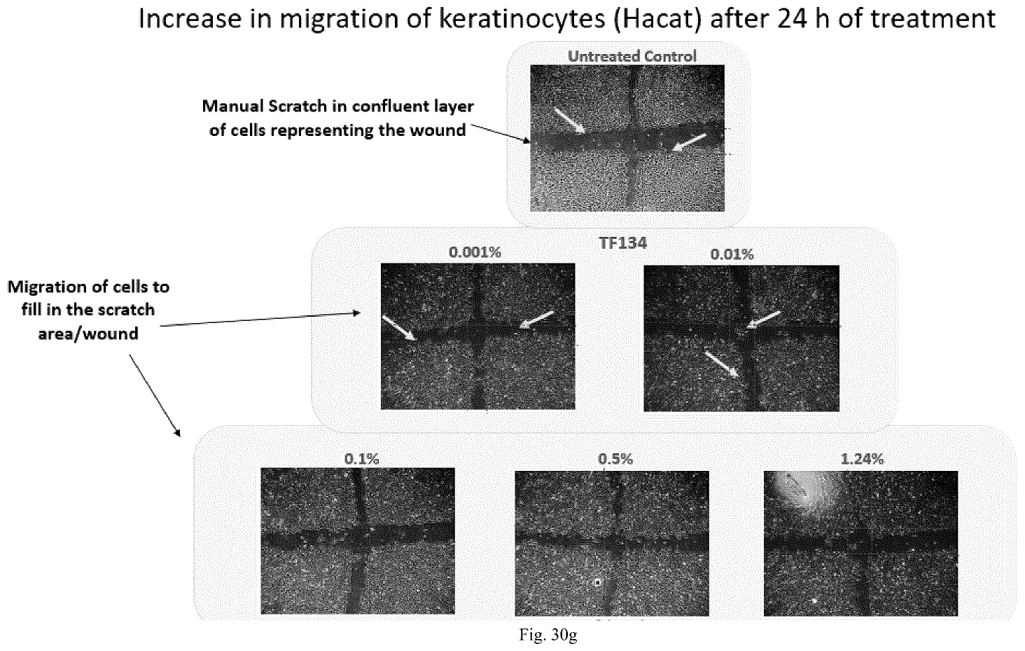

Study Design Assay 2b)—Skin Health—Increase in migration of keratinocytes as seen in FIGS. 30a-30g)

### Study Design

Test System—Human Keratinocytes (HaCaT)
Number of cells plated—0.1 million cells/well
FBS concentration—0%
Test Items—TF-125 (10.2%), TF-133 (15.4%), TF-134 (12.4%)
Time point—24h
Estimation method—Scratch assay

### Procedure

Cells were plated in 10% FBS in 12-well plate and incubated for 24 h.
   Cells were serum starved in 0% FBS for 24 h.
   Scratch was created in each well using a sterile tip followed by treatment of cells with 3 Test items with non-cytotoxic concentrations of TIs and PCs.
   Images were captured (0h).
   After 24 h, the images were captured to determine the effect of Test Items on migration.
   Increase in migration was calculated using ImageJ software.

### Calculations

Increase in migration was calculated wrt control (Untreated cells).

$$[ ( A - B ) / B ] * 100 ; Where A = \% Migration in Test Item treated cells , B = \% Migration in Control ( Untreated ) cells$$

The extent of Migration/Distance migrated was calculated as:

Empty space (pixels) at 0 h—Empty space (pixels) at 24 h
For each sample, respective zero h control was taken into consideration.

### 2c. Increase in Lipid Content in Keratinocytes

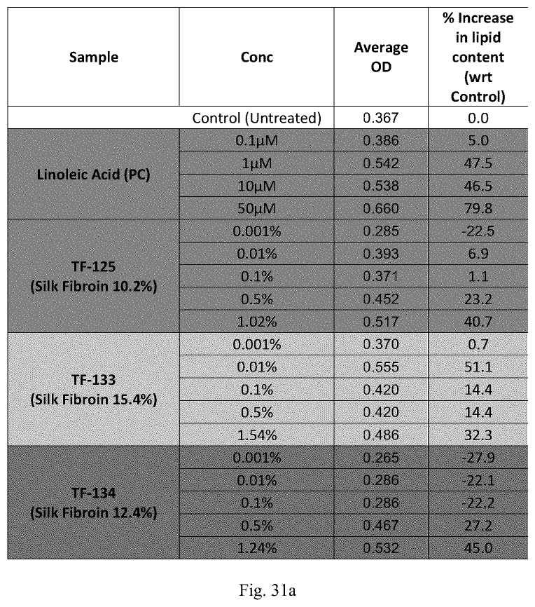

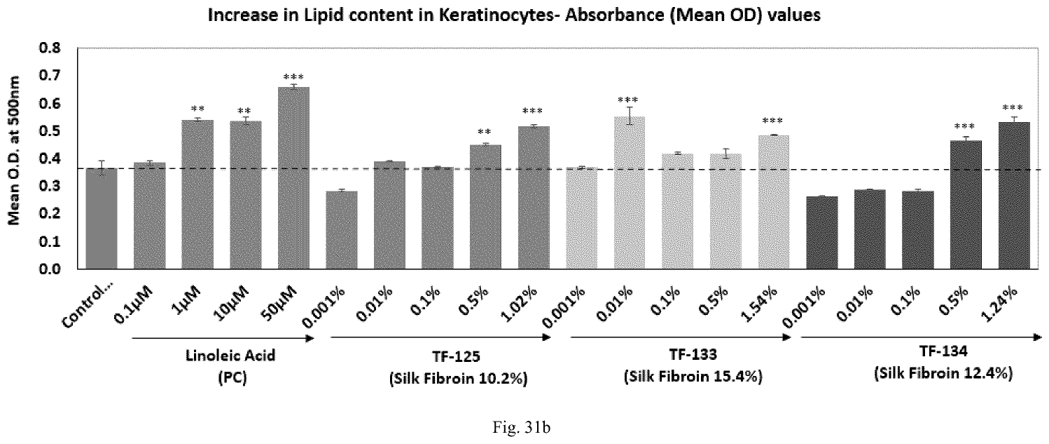

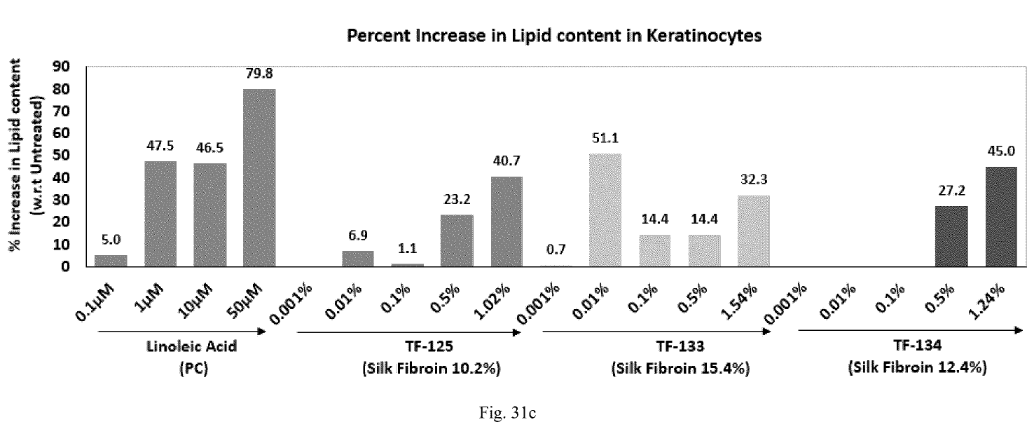

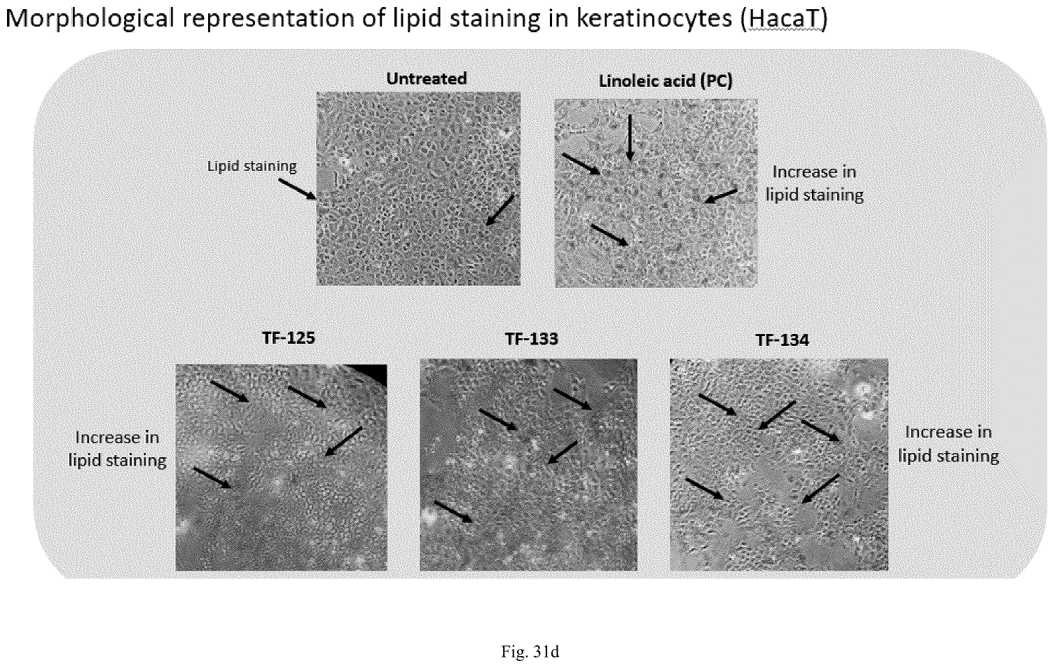

Study Design Assay 2c)—Skin Health—Increase in Lipid Content in Keratinocytes (HaCaT) (as Seen in FIGS. 31a-31d)

### Study Design

Test System—Human Keratinocytes (HaCaT)
Number of cells plated—50,000 cells/well in 12 well plate
FBS concentrations—0% FBS
Test Items—TF-125 (10.2%), TF-133 (15.4%), TF-134 (12.4%)
Time points—48h
Estimation method—Lipid content by Oil-Red-O dye (fat-soluble dye that stains neutral triglycerides and lipids, used for quantitative and qualitative measurement of lipid droplet formation)
Positive Control—Linoleic Acid

### Procedure

Cells were plated in 12-well plates in 10% FBS and incubated for 24 h.
Cells were sera starved with 0% FBS for 24h.
Cells were treated with 3 Test items at non-cytotoxic concentrations for 48 h.
After 48 hours, cells were fixed, and lipid content was measured by staining with Oil-O-Red dye.
   Cell layer was washed with PBS and fixed in 10% formalin. Cells were washed with 60% Isopropanol and air-dried.
   Air-dried cell layers were stained with Oil-Red-O stain at RT.
   Cell layers were washed with Milli-Q water to remove any unbound stain.
   The lipid bound stain was eluted with 100% Isopropanol and optical density was measured at 500 nm.
Percent increase of lipid content was calculated wrt control.

### Calculations

Percent Increase in each sample was calculated wrt control (Untreated cells).

$$[ ( A - B ) / B ] * 100 ; Where A = Absorbance in Test Item treated cells , B = Absorbance in Control ( Untreated ) cells .$$

### 2d. Improvement of Skin Barrier by Formation of Keratinocytes

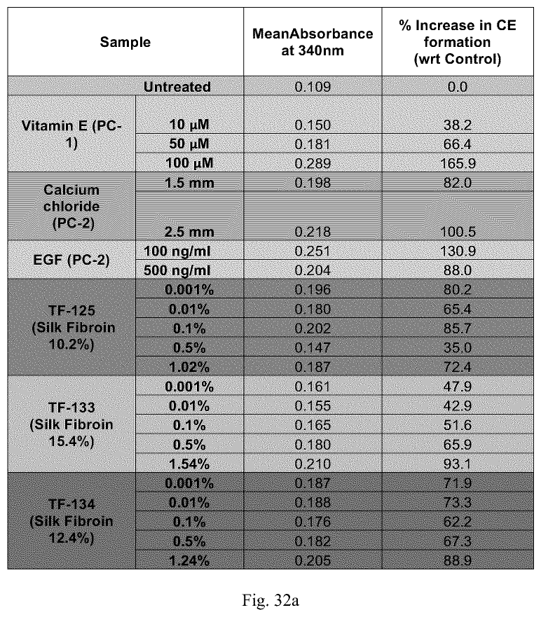

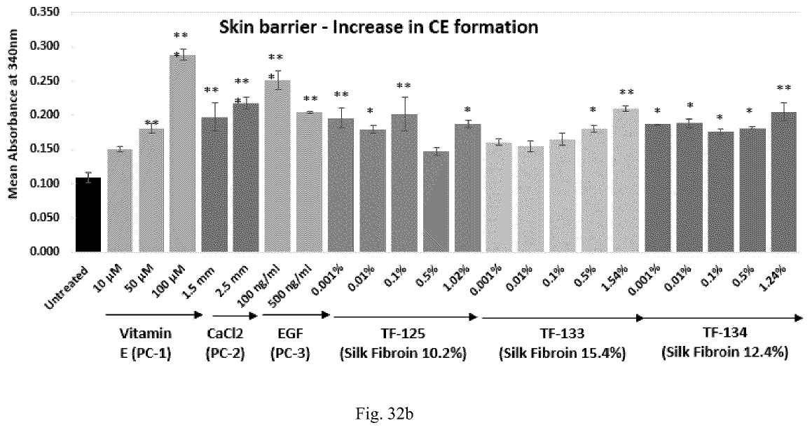

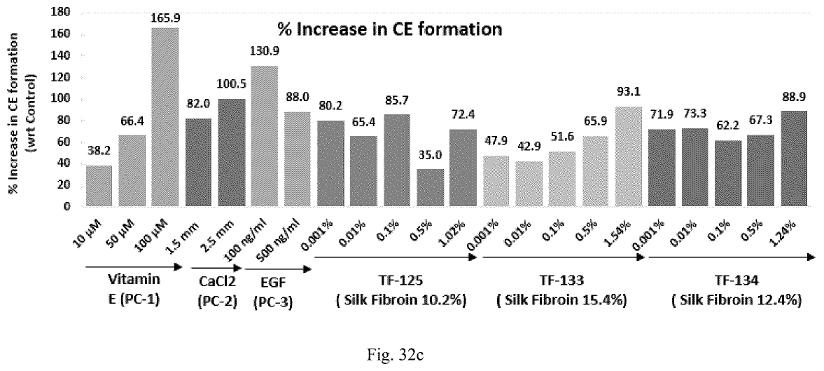

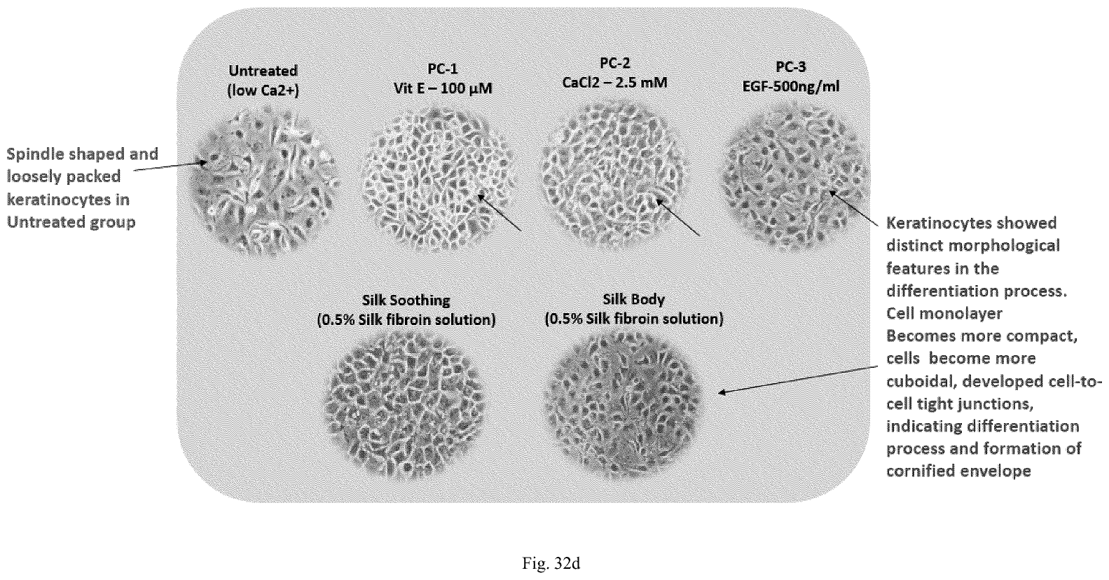

Study Design Assay 2 d)—Skin Health—Improvement of Skin Barrier Formation by Increased Formation of Cornified Envelope (CE) (as Seen in FIGS. 32a-32d)

### Study Design

Test System—Human Keratinocytes (HaCaT)
Number of cells plated—1 million cells/6-well plates
FBS concentrations—0%, Low calcium
Test items—TF-125 (10.2%), TF-133 (15.4%), TF-134 (12.4%)
Time points—7 days, followed by re-addition of Test Item on every 3rd day.
Estimation method—Turbidity of membrane/Absorbance at 340 nm.
Positive Control/s—Vitamin E, Calcium chloride, EGF.

### Procedure

Cells were plated in 10% FBS in 6-well plates and incubated for 24 h.
Cells were treated with 3 Test items at various concentrations in calcium free medium for 7 days, replacing media every 3rd day.
After incubation, cells processed for skin barrier strengthening potential measured as follows—
   Cells were harvested by trypsinization in pre-labelled Eppendorf tubes and centrifuged at 300 g for 5 mins.
   Supernatant was discarded and the cell pellet washed with 1×PBS to remove media residues.
   Cells were washed at 300 g for 5 mins.
   Supernatant was discarded and cell pellet was resuspended in 200 μl CE buffer (2% SDS, 20 mM DTT and 0.1M tris Buffer).
   The mixture was boiled at 95° C. for 5 mins to dissolve crosslinked envelopes.
   90 μl of mixture transferred to 96 well plates in duplicate and absorbance was measured at 340 nm.

### Calculations

Increase in formation of Cornified envelopes in each sample was calculated wrt control (Untreated cells).

$$[ ( A - B ) / B ] * 100 ; Where A = Absorbance in Test Item treated cells , B = Absorbance in Control ( Untreated ) cells .$$

### 2e. Improvement of Skin Barrier by Increased Expression of Surface Proteins in Keratinocytes

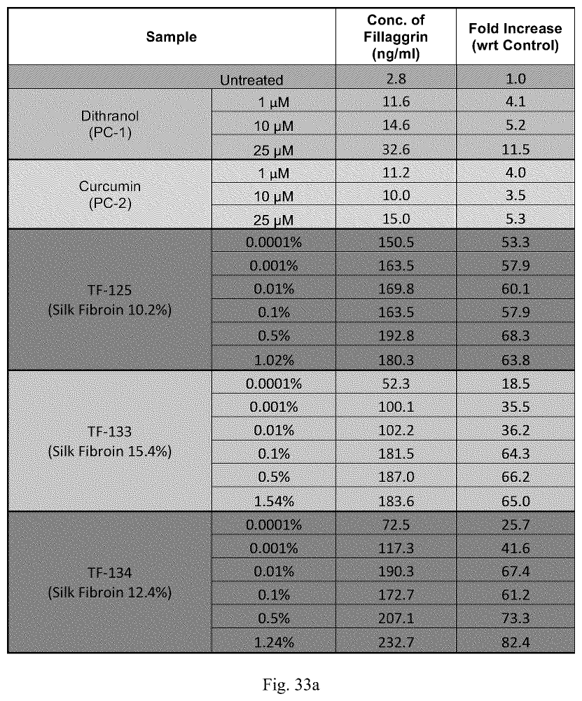

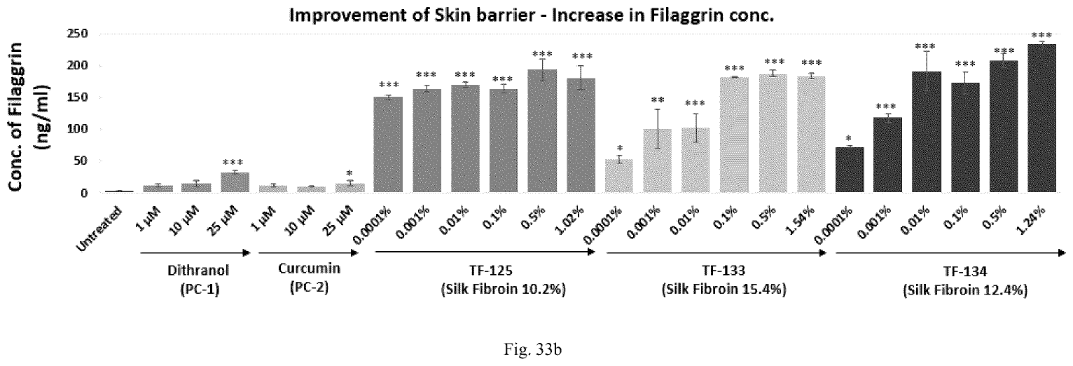

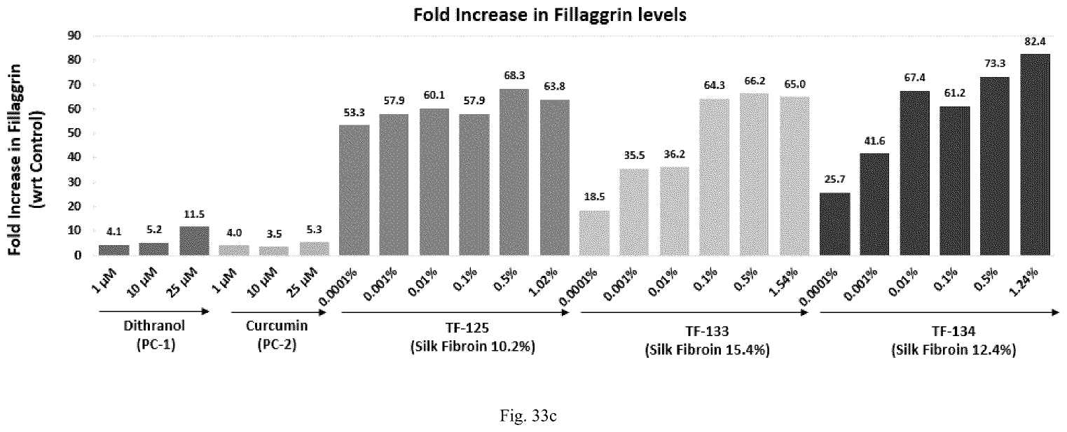

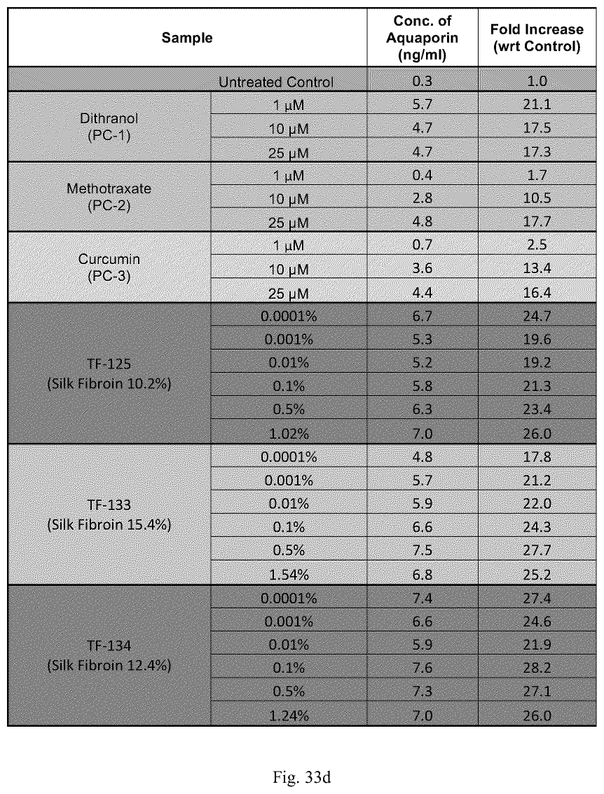

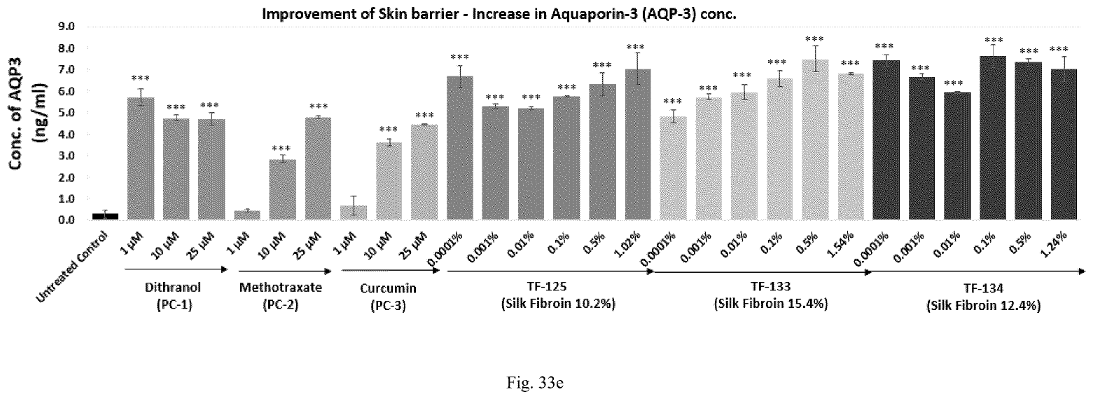

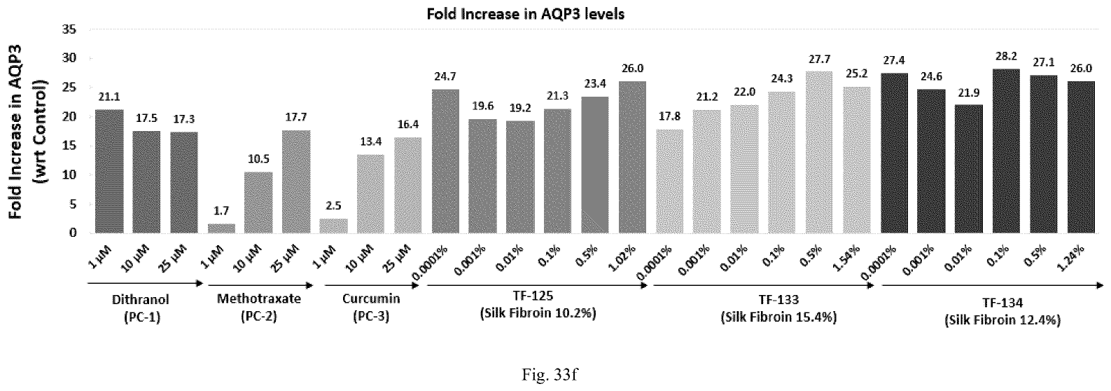

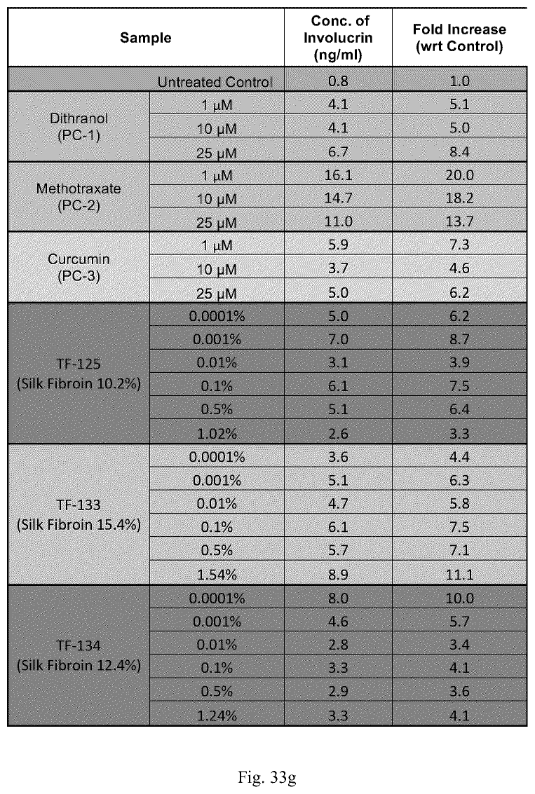

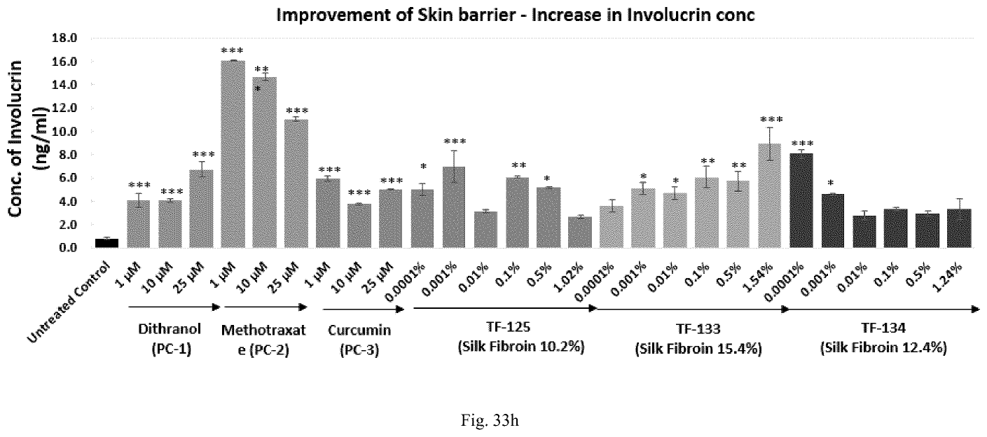

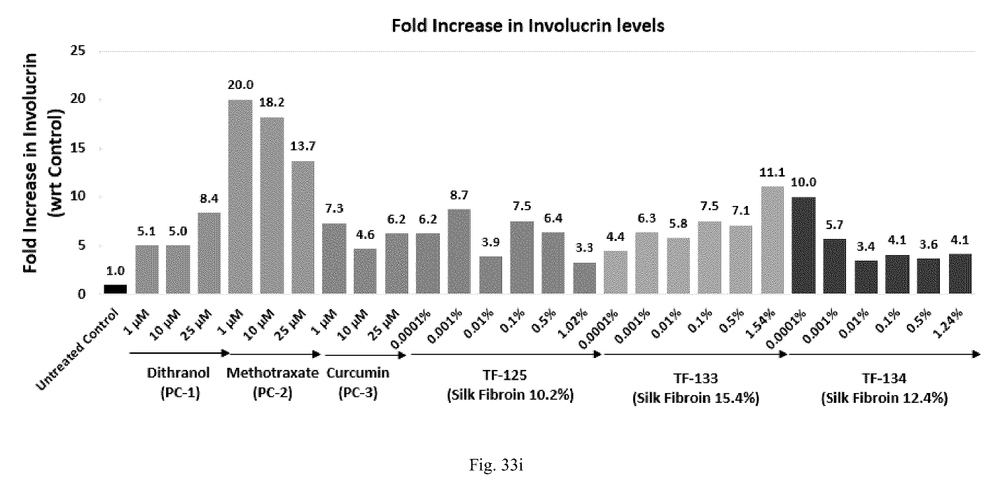

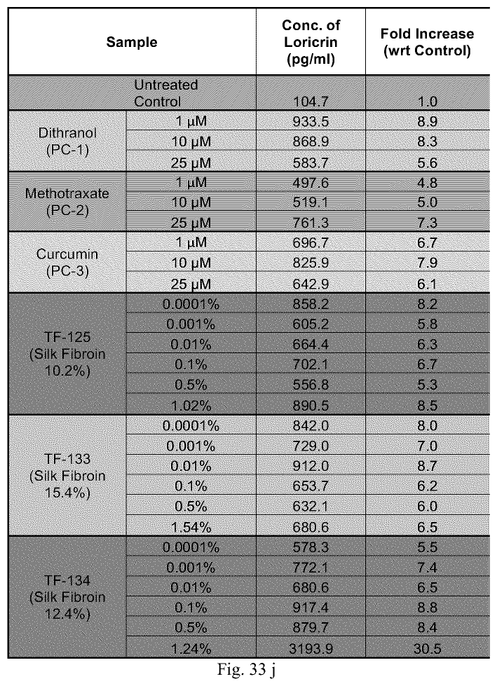

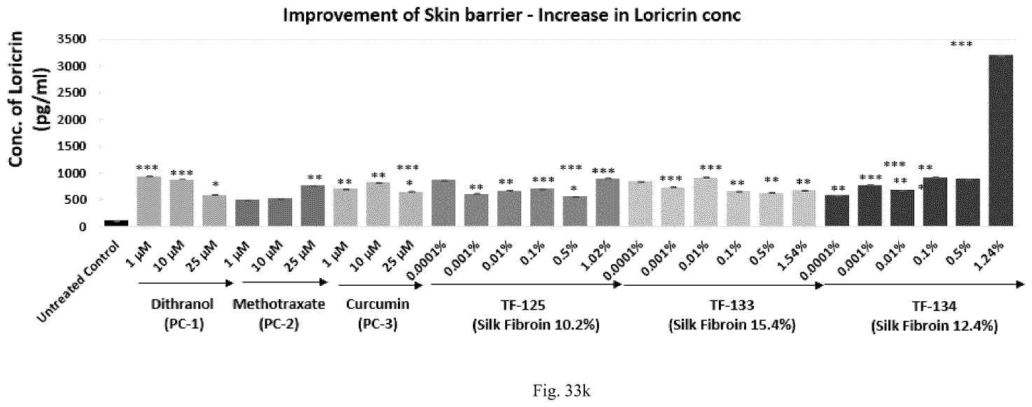

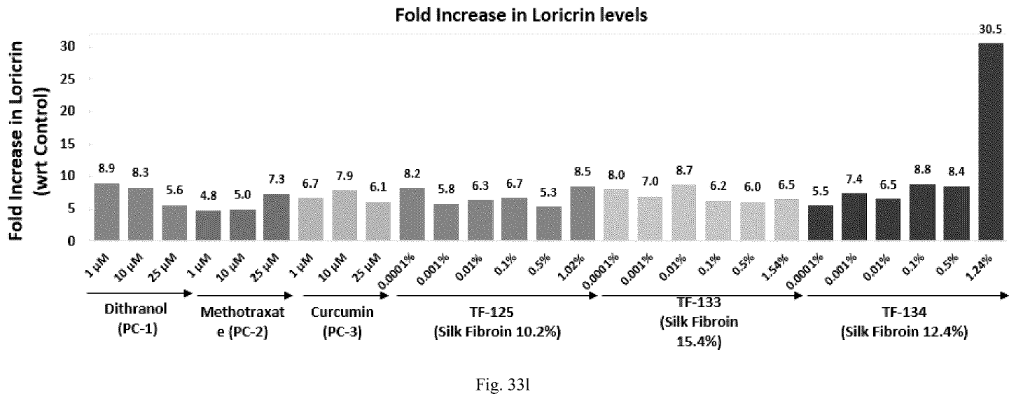

Study Design Assay 2e) Skin Health—Increase in Expression of Surface Markers for Improvement of Skin Barrier (as Seen in FIGS. 33a-331)

### Study Design

Test System—Human Keratinocytes (HaCaT)
Number of cells plated—0.1 million cells/24-well plates
FBS concentrations—0%,
Test items—TF-125 (10.2%), TF-133 (15.4%), TF-134 (12.4%)
Time points—72h
Estimation method—ELISA
Positive Control/s—Methotrexate, Dithranol, Curcumin.

### Procedure

Cells were plated in 10% FBS in 24-well plates and incubated for 24 h.
Cells were sera Starved with 0% FBS for 24h.
Cells were treated with 3 Test items in 0% FBS at various concentrations for 72h.
After incubation, culture supernatants were collected, and lysates were also prepared.
Levels of Filaggrin, AQP3 (supernatants) and Involucrin, Loricrin (lysates) were determined using ELISA as follows—
   Respective kit standards and samples (supernatants of cells) were directly pipetted into the wells and incubated for 1-2 h at 37° C.
   Liquid was removed from each well and plate was incubated at 37° C. for 1 hour after addition of Biotin-antibody.
   Following a wash (3 times) to remove any unbound conjugate, HRP-avidin solution was added to the wells and incubated for 30 min at 37° C.
   Plate was again washed for 3 times.
   TMB substrate was added to each well for 15-30 mins and plate followed by incubation at 37° C.
   The reaction was stopped by adding Stop solution to each well.
   The optical density of the color was measured at 450 nm.

### Calculations

Increase in Filaggrin levels in each sample was calculated wrt control (Untreated cells).

$$[ ( A - B ) / B ] * 100 ; Where A = conc of marker in Test Item treated cells , B = conc of marker in Control ( Untreated ) cells .$$

### 3. Inhibition of ECM Degradation Enzymes Elastase, Hyaluronidase

### 3a. Inhibition of ECM Degradation Enzyme (Elastase)

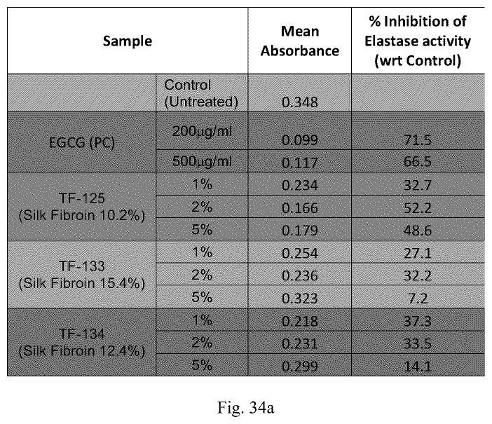

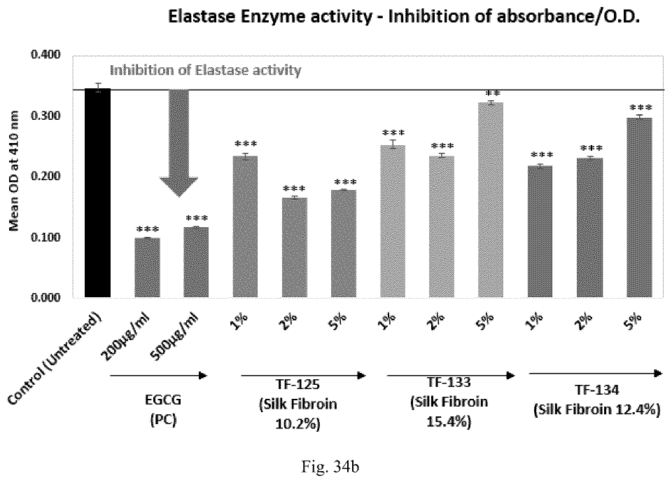

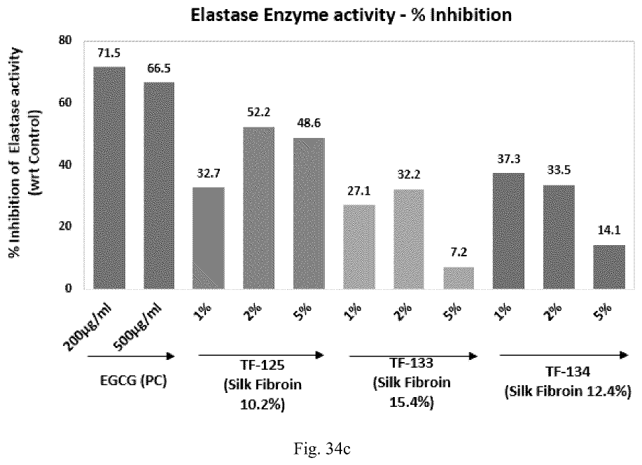

Study Design—Assay 3a)—Skin Health—Inhibition of ECM Degradation Enzyme (Elastase) (as Seen in FIGS. 34a-34c)

### Study Design

Test system—Elastase from porcine pancreas
Test items—TF-125 (10.2%), TF-133 (15.4%), TF-134 (12.4%)
Positive Control—EGCG

### Preparation of Assay Reagents, Enzyme and Substrate

### A. Tris-HCl Buffer 0.2 M, pH 8.0

1.576 g of Tris HCl buffer was weighed and dissolved in 40 ml of milliQ water.
Using 1 N HCl, pH of this solution was adjusted to 8.0.
Final volume of this solution was adjusted to 50 ml with milliQ water.

### B. Elastase Enzyme

The concentration of stock enzyme was 10.1 mg/ml. It was diluted using Tris-HCl buffer to obtain final working stock of 100 μg/ml.
   C. Substrate—N-Succinyl-Ala-Ala-Ala-p-nitroanilide (SANA)
1.42 mg of SANA was weighed and dissolved in 1.967 ml of Tris-HCl buffer respectively to prepare 1.6 mM stock solution.

### Procedure

For pre-incubation, 0.025 ml of elastase was pre-equilibrated with TIS; TF-125, TF-133 and TF-134 at concentrations 1%, 2%, 5% (v/v) for 15 min at 25° C.
Elastase (E) treated with buffer alone was taken as control sample.
SANA alone was included as substrate blank.
Enzyme+Substrate was included as control.
Reaction was initiated by addition of 0.125 ml of SANA and incubated for 20 min at 25° C.
The absorbance of PNA released was measured at 410 nm using Biotek reader.

### Calculations

Percent inhibition of Elastase activity will be calculated as:

$$\% Inhibition = [ ( R - X ) / R ] * 100$$

$$Where , X = Absorbance of Test Item treated sample$$

$$R = Absorbance of Control ( Untreated )$$

### 4. Anti-Aging Potential

### 4a. Anti-Aging Potential—DNA Protection

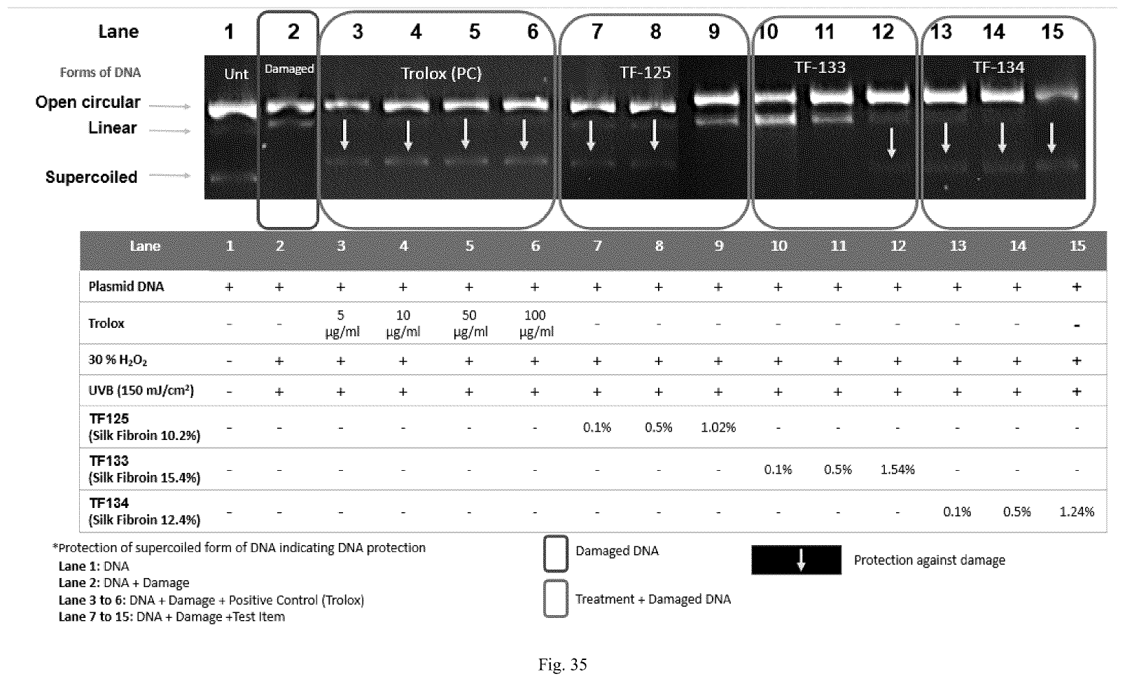

Study Design Assay 4a) Anti-Aging—DNA Protection Against Oxidative+UV-B Damage (as seen in FIG. 35)

### Study Design

Test System—Cell free assay
Test items—TF-125, TF-133, TF-134
Time point—1h
Estimation method—Restoration of Native DNA against oxidative damage measured qualitatively by Gel Electrophoresis
Positive Control—Trolox

### Procedure

15 μl of Plasmid DNA (pBR322 Plasmid DNA from E. coli) was incubated with different conc. of 3 Test Items (2 μl) and Trolox (positive control) for 1 h at 37□C.
Oxidative damage was induced with 30% H₂O₂ (vol 2 μl) for 20 min at 37□C and subsequently exposed to UVB damage (150 mJ/cm2) for 5 min.
After irradiation, samples were run on an electrophoresis gel using 1% agarose gel.
Segregated form of plasmid DNA bands were analyzed and captured using BIORAD Gel documentation System.
Restoration in the plasmid DNA pattern of test items treated DNA, as compared to control untreated DNA was observed.

Intact DNA shows 3 forms of DNA—Open circular, linear and supercoiled,
Damaged DNA showed only 2 bands, Open circular and linear,
Protective effect showed reappearance of supercoiled band.

### Interpretation:

Restoration of Native DNA against oxidative+UVB damage

### 5. Anti-Aging Potential—Fibroblast Based Assays

### 5a. Cytoprotection in Fibroblasts Against UVB

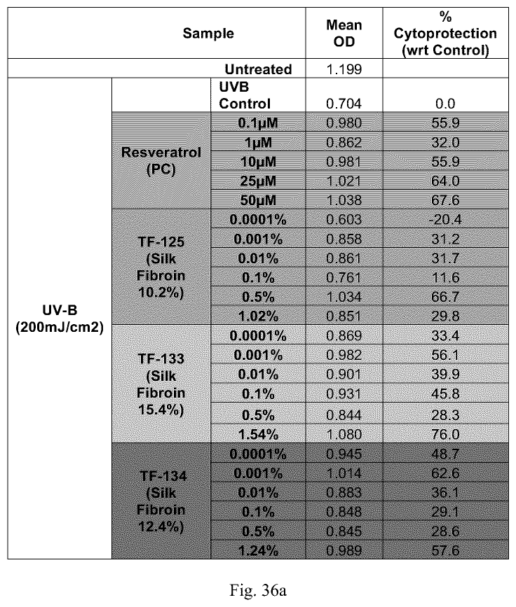

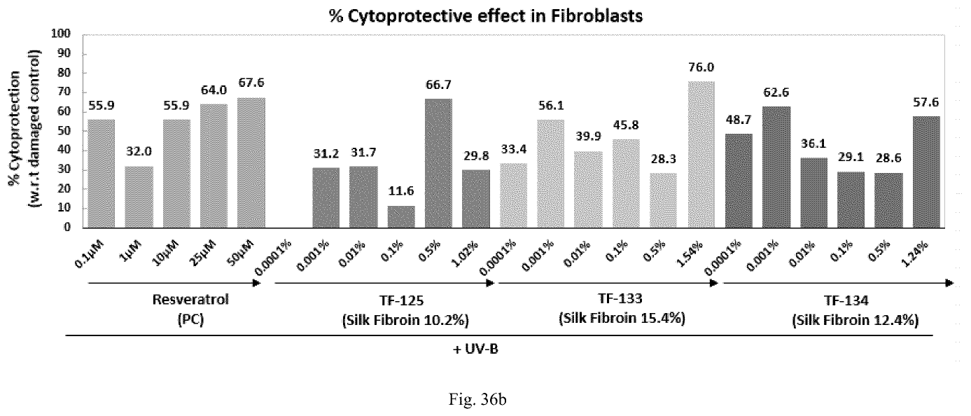

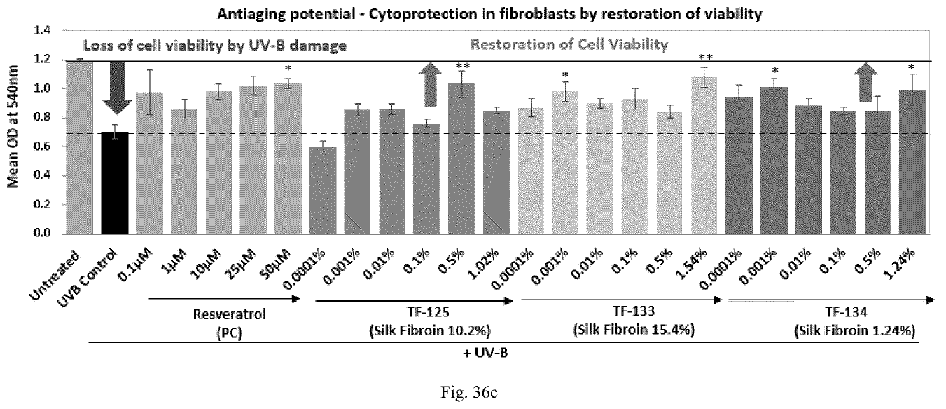

Study Design Assay 5a) Anti-Aging—Cytoprotection Against UV-B Induced Damage (as Seen in FIGS. 36a-36c)

### Study Design

Test System—HFF-1 (Skin fibroblasts)
Number of cells plated—8000 cells/well plate
FBS concentrations—0.1%
Test items—TF-125 (10.2%), TF-133 (15.4%), TF-134 (12.4%)
Time points—24h pretreatment, UVB exposure (200 mJ/cm2), recovery 24h
Estimation method—MTT assay
Positive Control—Resveratrol

### Procedure

Cells were plated in 96-well plates in 10% FBS and incubated for 24 h.
Cells were pre-treated with 3 Test Items at various concentrations for 24 h.
Cells were exposed to UVB irradiation (200 mJ/cm2) for 15 min in PBS.
PBS was washed off and replaced with medium and allowed for recovery for 24 h.
After 24h, cell viability was assessed by MTT assay.
   20 μl of MTT solution was added to each well and cells were incubated for 3 h at 37° C.
   After incubation, supernatants were removed and 150 μl DMSO added to all wells to extract formazan crystals.
   A purple-coloured formazan complex was formed. Absorbance of each well was measured at 540 nm.

### Calculation

Protective effect on cell viability was calculated wrt controls (untreated and UV-B damaged cells).

$$\{ ( Absorbance of TI + UVB ) - ( Absorbance of UVB alone ) / ( Absorbance of Untreated ) - ( Absorbance of UVB alone ) \} * 100$$

### 5b. Anti-Apoptotic Effect in Fibroblasts Against UVB

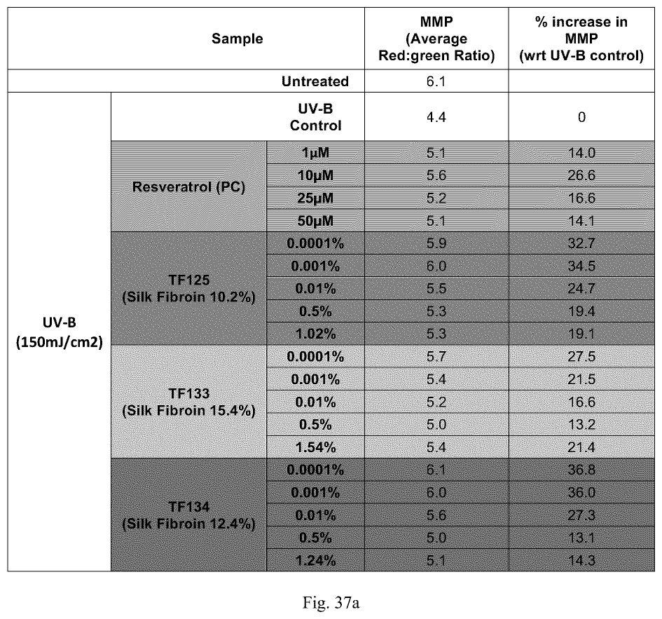

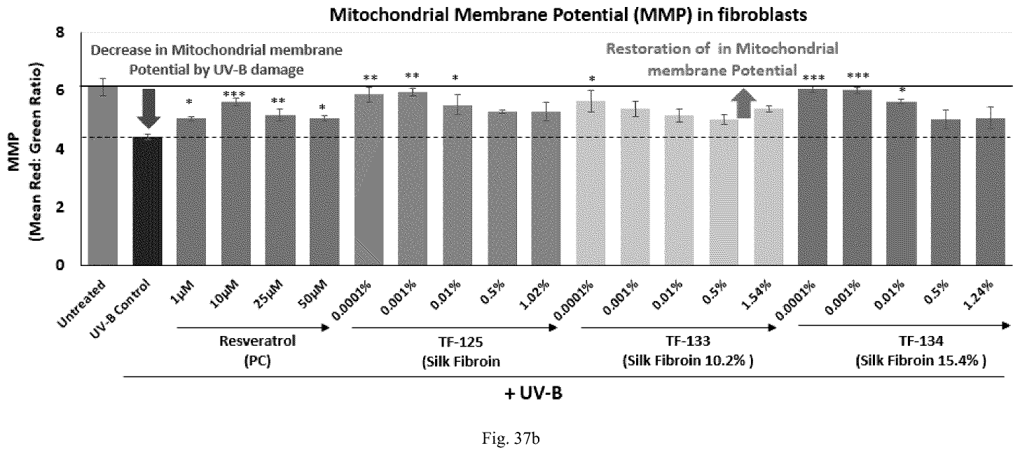

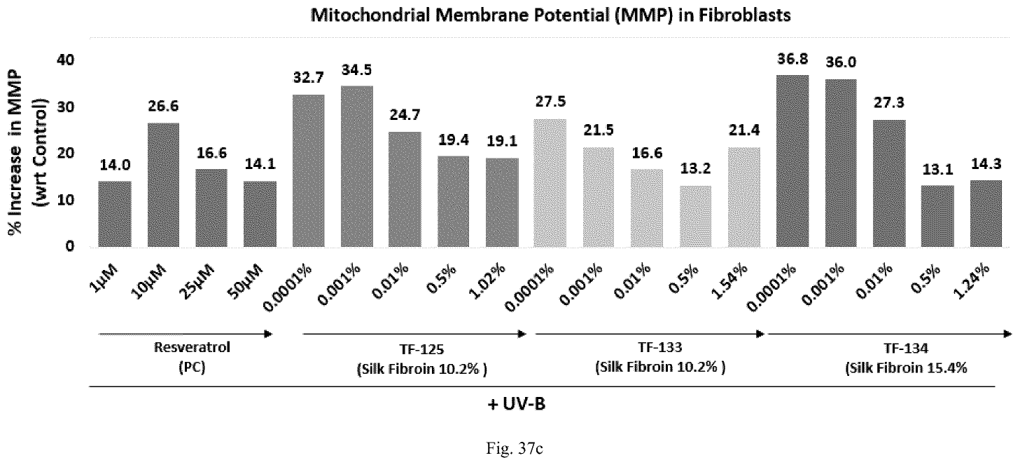

Study Design Assay 5b)—Anti-Aging—Increase in Mitochondrial Membrane Potential (MMP) (as Seen in FIGS. 37a-37c)

### Study Design

Test System—Human Fibroblasts (HFF-1)
Number of cells plated—8,000
FBS concentrations—0.5% FBS
Test items—TF-125 (10.2%), TF-133 (15.4%), TF-134 (12.4%)
Time point—24h pretreatment, UVB exposure (150 mJ/cm2), recovery 24h
End points—Mitochondrial Membrane Potential using JC-1 dye.
Positive Control—Resveratrol

### Procedure

Cells were plated in 10% FBS in 96-well plates and incubated for 24 h.
Cells were treated with 3 Test items in 0.5% FBS at various concentrations for 24h.
After 24h, damage was induced using UVB (150 mJ/cm2) damage in cells.
Cells were washed and incubated for recovery for another 24 h in serum-free medium.
After 24 h, the effect on Mitochondrial membrane Potential was determined by JC-1 assay. Active mitochondria in live cells exhibit brighter red fluorescence signal compared to mitochondria with lower membrane potential in apoptotic cells which fluoresce green with JC-1 dye. Ratio of Red:Green indicates degree of MMP. Inhibition of MMP shows depolarization of MMP.
   After incubation, medium from each well was removed.
   100 μl of 10 μM JC-1 dye in PBS was added to each well and cells were incubated at 37° C. for 25 mins.
   After incubation, cells were rinsed with PBS to remove dye and Fluorescence was measured at 528/590 for red and 485/528 for green.
   Ratio of Red: green (healthy cells) was calculated.

### Calculations

Increase of Mitochondrial membrane potential was calculated wrt UVB damage control.

$$[ ( A - B ) / B ] * 100 ; Where A = Ratio of Red : Green in Test Item treated cells , B = Ratio of Red : Green in Control ( Untreated ) cells$$

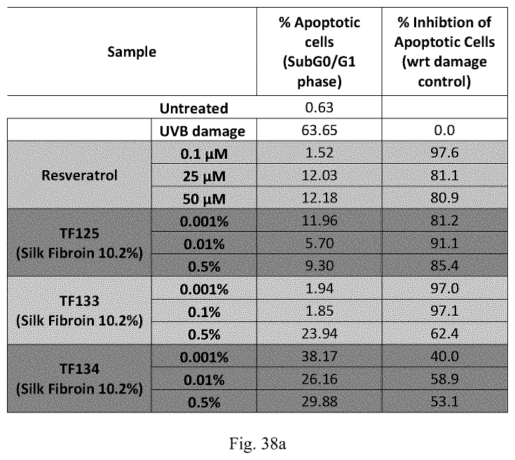

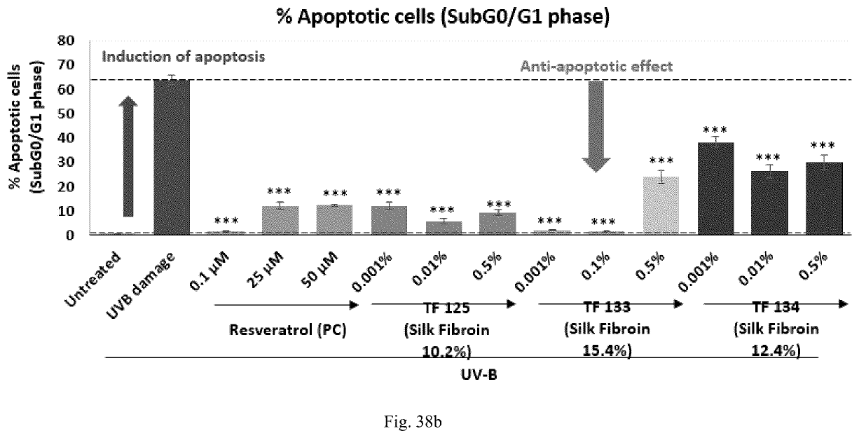

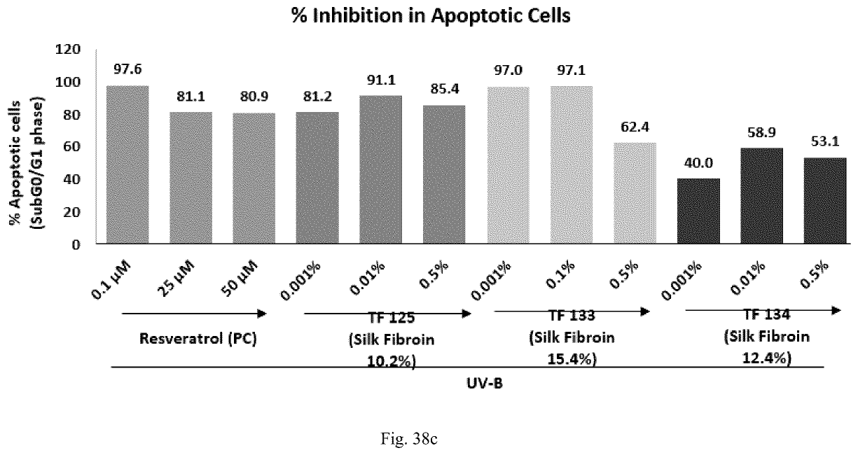

Study Design Assay 5b)—Anti-Aging—Inhibition in subG0/G1 Cells by Cell Cycle Assay (as Seen in FIGS. 38a-38c)

### Study Design

Test System—Human Fibroblast cell line (HFF-1)
Number of cells plated—0.25 million cells/12-well plates
FBS concentrations—0.5%,
Test items—TF-125 (10.2%), TF-133 (15.4%), TF-134 (12.4%)
Time points—24h pretreatment, UVB exposure (100 mJ/cm2), recovery 24h
Estimation method—SubG0/G1 apoptotic cells by Propidium iodide solution
Positive Control—Resveratrol

### Procedure

Cells were plated in 10% FBS in 12-well plates and incubated for 24 h.
Cells were treated with 3 Test items in 0.5% FBS at various concentrations.
After 24h, damage was induced using UVB (150 mJ/cm2) damage in cells.
Cells were washed and incubated for recovery for another 24 h in serum-free medium.
After 24 h, the anti-apoptotic effect was measured by Cell-cycle analysis.
   After incubation, medium from each well was removed and cells were harvested by trypsinization and centrifuged at 450 g for 5 min (low brake).
   Supernatant was discarded and cell pellet washed with 1×PBS at 450 g for 5 min (low brake).
   Cells were fixed with Ice-cold 70% ethanol (500 μl) and stored at 4° C. for 24 h prior to staining.
   For staining, Ethanol fixed cells were centrifuged at low brake and washed with PBS.
   200 μl of cell cycle reagent was added to each sample at kept at RT for staining for 30 min in dark.
   After incubation, cells were acquired using flow cytometer.

### Calculations

Decrease in apoptotic cell population (sub G0/G1) was calculated wrt UVB damage control.

$$[ ( B - A ) / B ] * 100 ; Where A = \% sub G 0 / G 1 cells in Test Item treated cells , B = \% sub G 0 / G 1 cells in UVB damage Control$$

### 5c. Anti-Inflammatory Activity in Fibroblasts

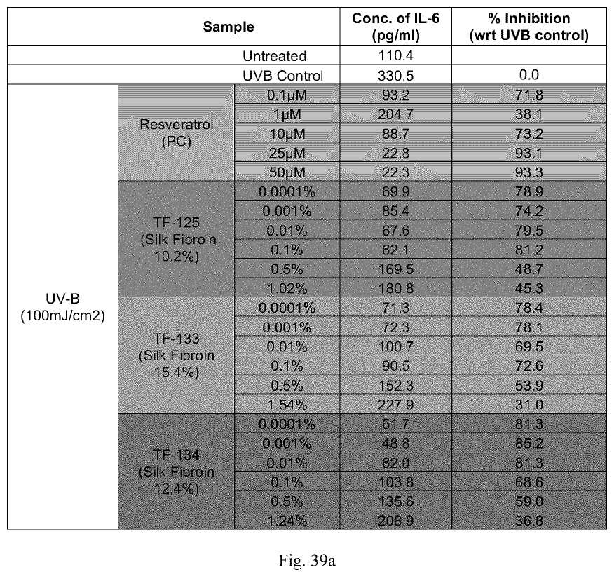

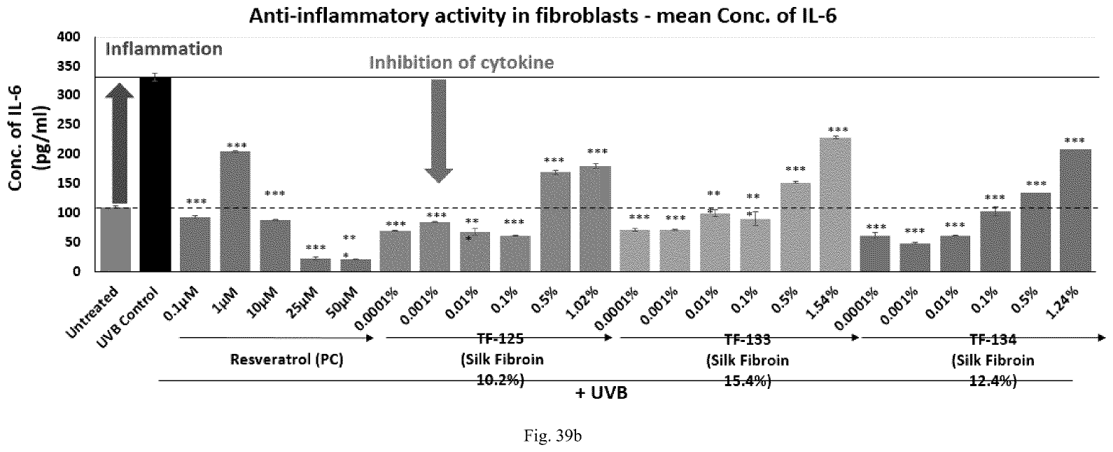

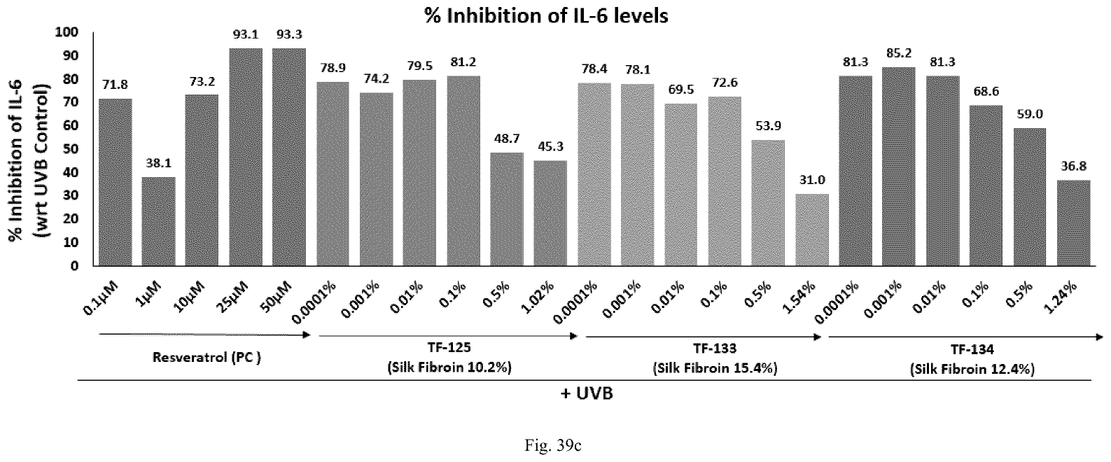

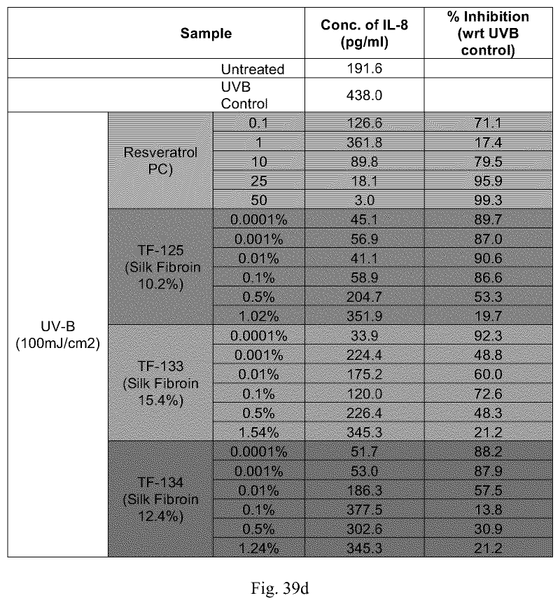

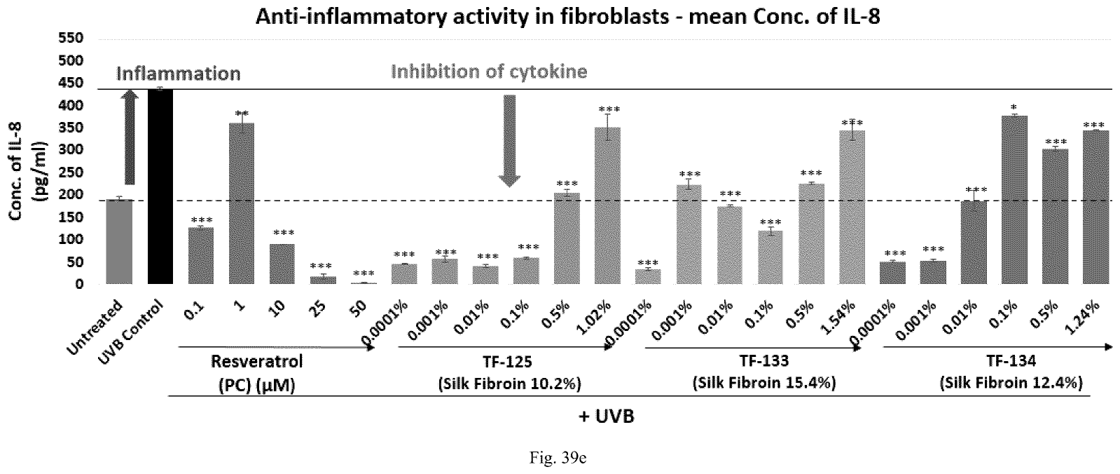

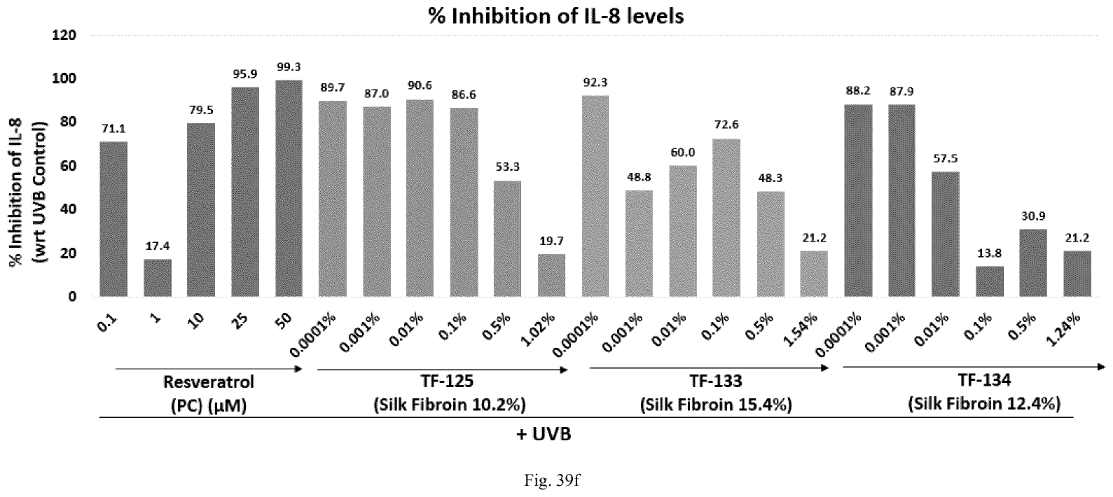

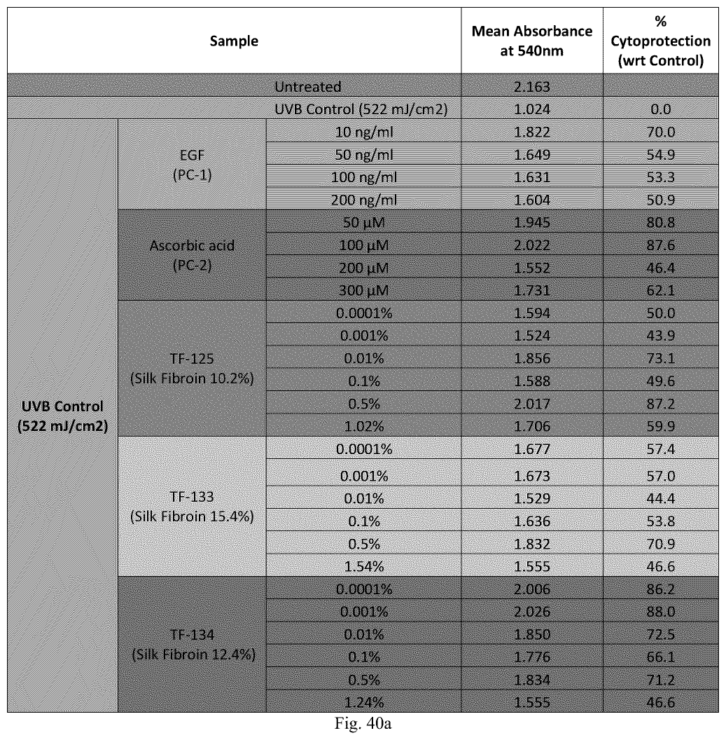

Study Design Assay 5c)—Anti-Aging—Anti-Inflammatory Activity by Inhibition of Cytokines (as Seen in FIGS. 39a-39f)

### Study Design

Test System—Human Fibroblast cell line (HFF-1)
Number of cells plated—0.25 million cells/6-well plates
FBS concentrations—0%,
Test Items—TF-125 (10.2%), TF-133 (15.4%), TF-134 (12.4%)
Time points—24h pretreatment, UVB exposure (100 mJ/cm2), recovery 24h
Estimation method—ELISA
Positive Control—Resveratrol

### Procedure

Cells were plated in 10% FBS in 6-well plates and incubated for 24 h. Cells were serum starved in 0% FBS for 24 h.
Cells were treated with 3 Test Items in 0% FBS at non-cytotoxic concentrations for 24h.
After 24h, damage was induced using UVB (100 mJ/cm2 for 5.5 min) damage in cells.
Cells were washed and incubated for recovery for another 24 h in serum-free medium.
After incubation, culture supernatants were collected.
Levels of IL-6, IL-8, IL-1-α and TNF-α (supernatants) were determined using ELISA as follows—
   Assay diluent was added to each well. Respective kit standards and samples (supernatants) were directly pipetted into the wells and incubated for 2 h at RT.
   After washing away any unbound substances for a total of 3 times, respective conjugate was added to each well and incubated for 1-2 h at RT.
   Following a wash (3 times) to remove any unbound conjugate, substrate solution was added to the wells and incubated for 30 min at RT in dark.

The reaction was stopped by adding Stop solution to each well.
The optical density of the color was measured at 450 nm. Conc of cytokines were calculated from standard curve.

### Calculations

Inhibition in levels of cytokines was calculated wrt UVB control.

$$[ ( B - A ) / B ] * 100 ; \\ Where A = Levels of cytokines in Test Item treated cells , \\ B = Levels of cytokines in UVB Control .$$

### 6. Anti-Aging Potential—Keratinocytes Based Assays

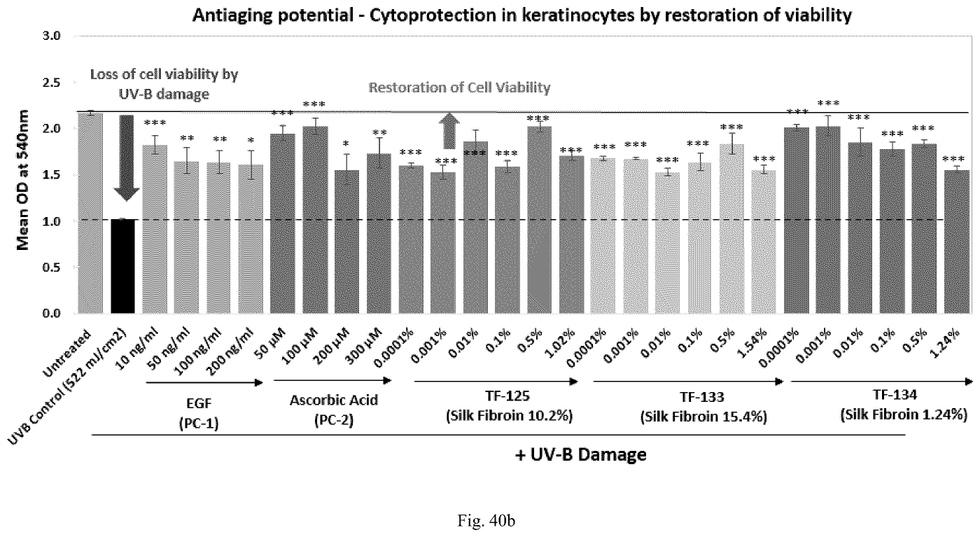

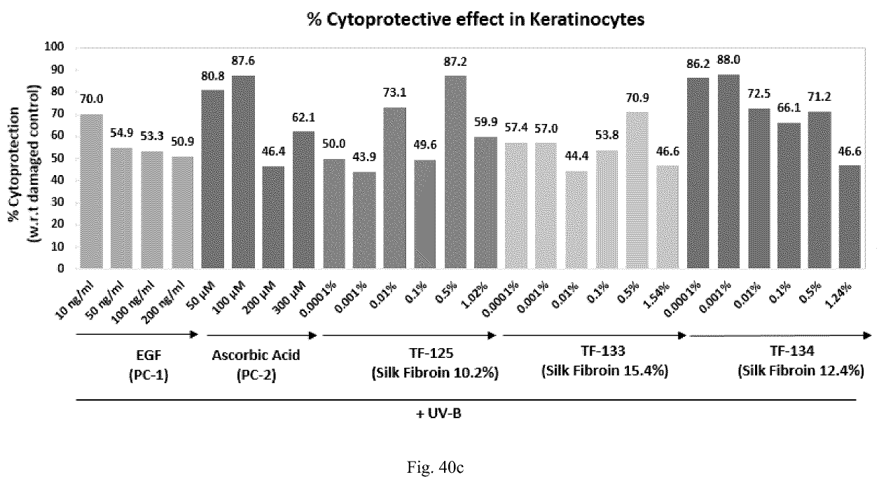

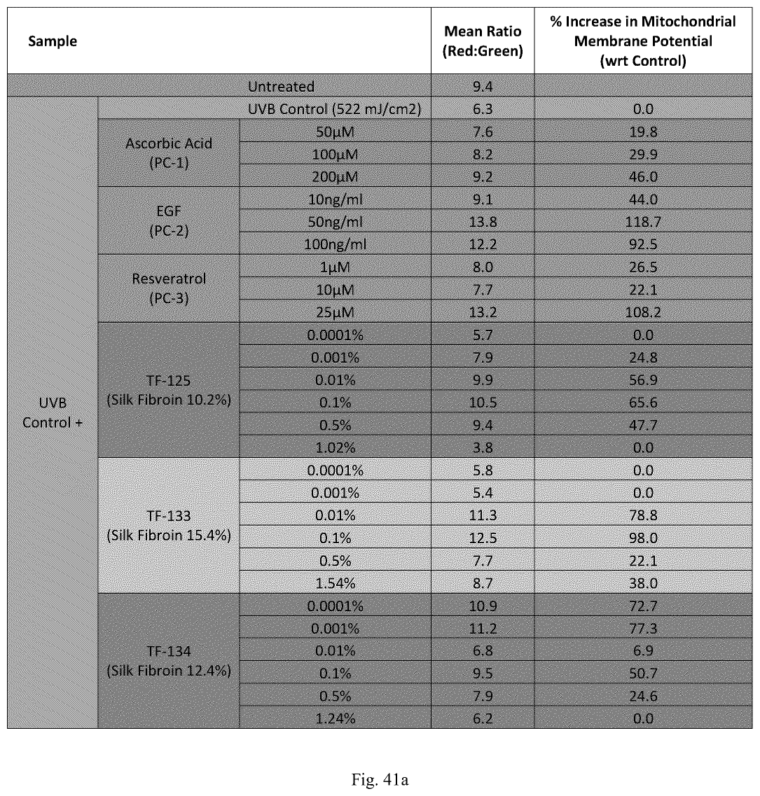

### 6a. Cytoprotection in Keratinocytes Against UVB (as Seen in FIGS. 40a-40c)

### Study Design

Test System—Human Keratinocytes (HaCat)
Number of cells plated—10000 cells/well plate
FBS concentrations—0.1%
Test items—TF-125 (10.2%), TF-133 (15.4%), TF-134(12.4%)
Time points—24h pretreatment, UVB exposure (522 mJ/cm2), recovery 24h
Estimation method—MTT assay
Positive Control—Ascorbic Acid, EGF

### Procedure

Cells were plated in 96-well plates in 10% FBS and incubated for 24 h.
Cells were pre-treated with 3 Test Item at various concentrations for 24 h.
Cells were exposed to UVB irradiation (200 mJ/cm2) for 15 min in PBS.
PBS was washed off and replaced with medium and allowed for recovery for 24 h.
After 24h, cell viability was assessed by MTT assay.
   20 μl of MTT solution was added to each well and cells were incubated for 3 h at 37° C.
   After incubation, supernatants were removed and 150 μl DMSO added to all wells to extract formazan crystals.
   A purple-coloured formazan complex was formed. Absorbance of each well was measured at 540 nm.

### Calculation

Protective effect on cell viability was calculated wrt controls (untreated and UV-B damaged cells).

$$\{ ( Absorbance of TI + UVB ) - ( Absorbance of UVB alone ) / ( Absorbance of Untreated ) - ( Absorbance of UVB alone ) \} * 100$$

### 6b. Anti-Apoptotic Effect in Keratinocytes Against UVB

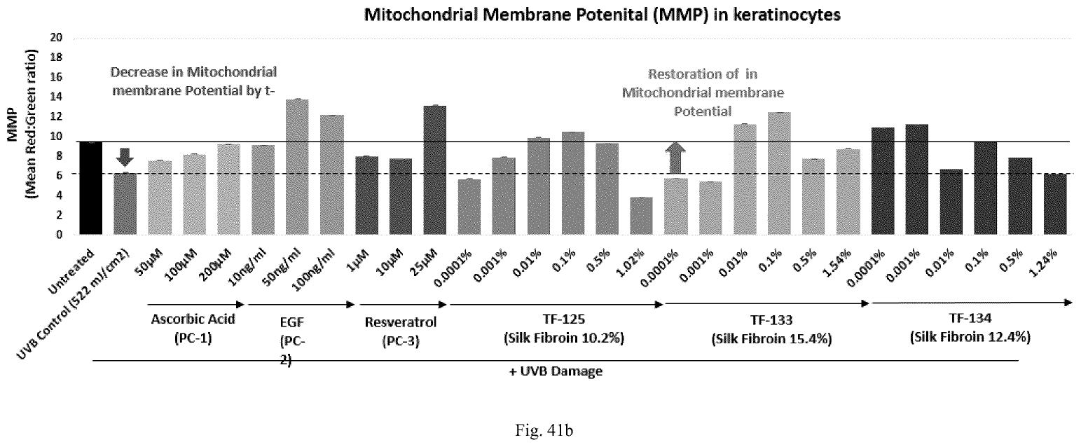

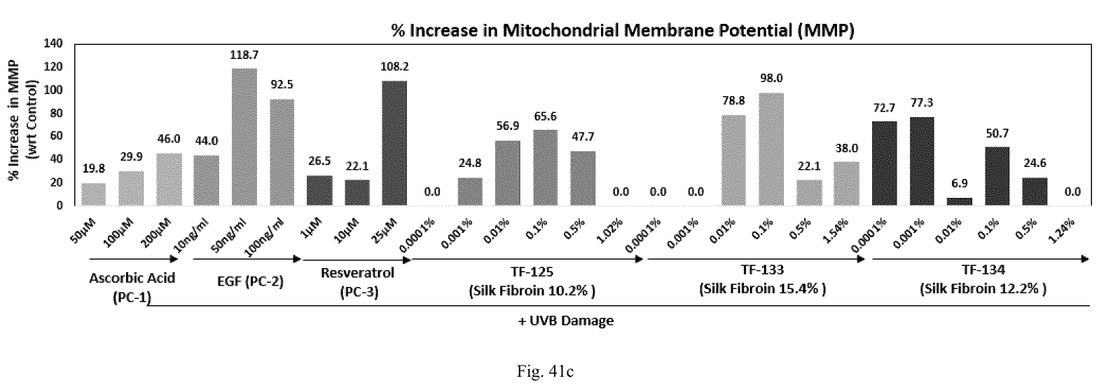

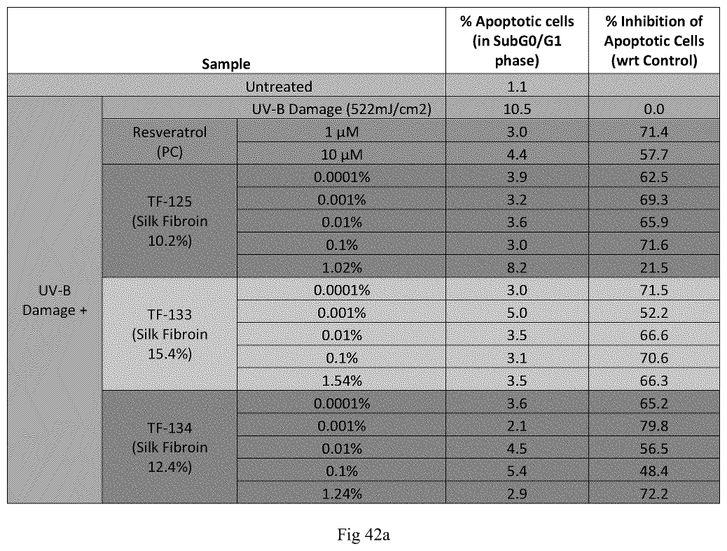

Study Design Assay 6b. Anti-Aging—Increase in Mitochondrial Membrane Potential (MMP) (as Seen in FIGS. 41a-41c)

### Study Design

Test System—Human Keratinocytes (HaCat)
Number of cells plated—10000 cells/well plate
FBS concentrations—0.1%
Test items—TF-125 (10.2%), TF-133 (15.4%), TF-134(12.4%)
Time points—24h pretreatment, UVB exposure (522 mJ/cm2), recovery 24h
End points—Mitochondrial Membrane Potential using JC-1 dye.
Positive Control—Ascorbic Acid, EGF

### Procedure

Cells were plated in 10% FBS in 96-well plates and incubated for 24 h.
Cells were treated with 3 Test items in 0.1% FBS at various concentrations for 24h.
Cells were exposed to UVB irradiation (522 mJ/cm2) in PBS.
PBS was washed off and replaced with serum free medium and allowed for recovery for 24h.
After 24 h, the effect on Mitochondrial membrane Potential was determined by JC-1 assay. Active mitochondria in live cells exhibit brighter red fluorescence signal compared to mitochondria with lower membrane potential in apoptotic cells which fluoresce green with JC-1 dye. Ratio of Red:Green indicates degree of MMP. Inhibition of MMP shows depolarization of MMP.
   After incubation, medium from each well was removed.
   100 μl of 10 μM JC-1 dye in PBS was added to each well and cells were incubated at 37° C. for 25 mins.
   After incubation, cells were rinsed with PBS to remove dye and Fluorescence was measured at 528/590 for red and 485/528 for green.
   Ratio of Red: green (healthy cells) was calculated.

### Calculations

Increase of Mitochondrial membrane potential was calculated wrt UVB damage control.

$$[ ( A - B ) / B ] * 100 ; \\ Where A = Ratio of Red : Green in Test Item treated cells , \\ B = Ratio of Red : Green in UVB damage control cells$$

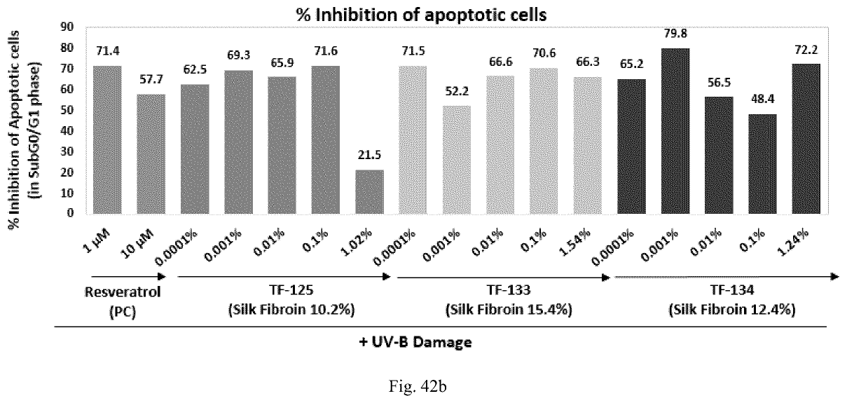

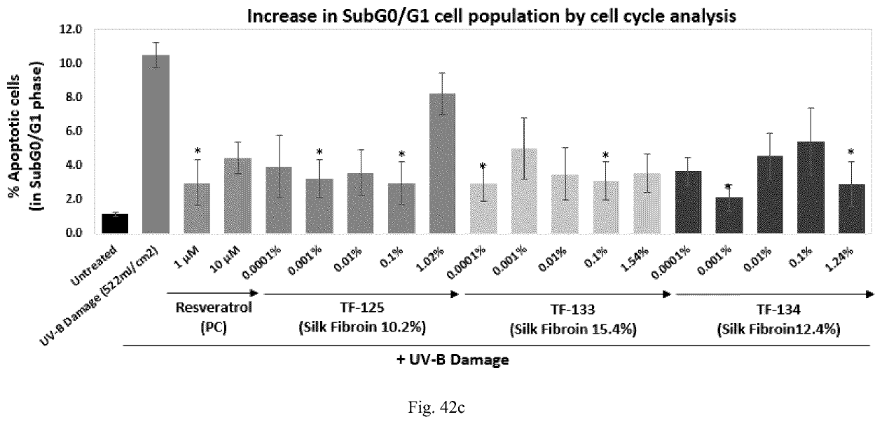

Study Design Assay 6c)—Anti Aging—Inhibition in subG0/G1 Cells by Cell Cycle Assay (as Seen in FIGS. 42a-42c)

### Study Design

Test System—Human Keratinocytes (HaCat)
Number of cells plated—10000 cells/well plate
FBS concentrations—0.1%
Test items—TF-125 (10.2%), TF-133 (15.4%), TF-134(12.4%)
Time points—24h pretreatment, UVB exposure (522 mJ/cm2), recovery 24h
End points—Mitochondrial Membrane Potential using JC-1 dye.
Positive Control—Ascorbic Acid, EGF

### Procedure

Cells were plated in 10% FBS in 96-well plates and incubated for 24 h.
Cells were treated with 3 Test items in 0.1% FBS at various concentrations for 24h.
Cells were exposed to UVB irradiation (522 mJ/cm2) in PBS.
PBS was washed off and replaced with serum free medium and allowed for recovery for 24h.
After 24 h, the effect on Mitochondrial membrane Potential was determined by JC-1 assay. Active mitochondria in live cells exhibit brighter red fluorescence signal compared to mitochondria with lower membrane potential in apoptotic cells which fluoresce green with JC-1 dye. Ratio of Red:Green indicates degree of MMP. Inhibition of MMP shows depolarization of MMP.
   After incubation, medium from each well was removed.
100 μl of 10 μM JC-1 dye in PBS was added to each well and cells were incubated at 37° C. for 25 mins.
   After incubation, cells were rinsed with PBS to remove dye and Fluorescence was measured at 528/590 for red and 485/528 for green.
   Ratio of Red: green (healthy cells) was calculated.

### Calculations

Increase of Mitochondrial membrane potential was calculated wrt UVB damage control.

$$[ ( A - B ) / B ] * 100 ; \\ Where A = Ratio of Red : Green in Test Item treated cells , \\ B = Ratio of Red : Green in UVB damage control cells$$

Study Design Assay 6c)—Anti Aging—Inhibition in subG0/G1 Cells by Cell Cycle Assay 91

### Study Design

Test System—Human Keratinocytes (HaCat)
Number of cells plated—0.1 million cells/12-well plates
FBS concentrations—0.1%
Test items—TF-125 (10.2%), TF-133 (15.4%), TF-134(12.4%)
Time points—24h pretreatment, UVB exposure (522 mJ/cm2), recovery 24h
Estimation method—SubG0/G1 apoptotic cells by Propidium iodide solution
Positive Control—Ascorbic Acid, EGF

### Procedure

Cells were plated in 10% FBS in 12-well plates and incubated for 24 h.
Cells were Serum starved with 0.5% FBS for 24h.
Cells were treated with 3 Test items in 0.1% FBS at various concentrations for 24h.
Cells were exposed to UVB irradiation (522 mJ/cm2) in PBS.
PBS was washed off and replaced with serum free medium and allowed for recovery for 24h.
   After 24 h, the anti-apoptotic effect was measured by Cell-cycle analysis.
   After incubation, medium from each well was removed and cells were harvested by trypsinization and centrifuged at 450 g for 5 min (low brake).
   Supernatant was discarded and cell pellet washed with 1×PBS at 450 g for 5 min (low brake).
   Cells were fixed with Ice-cold 70% ethanol (500 μl) and stored at 4° C. for 24 h prior to staining.
   For staining, Ethanol fixed cells were centrifuged at low brake and washed with PBS.
   200 μl of cell cycle reagent was added to each sample at kept at RT for staining for 30 min in dark.
   After incubation, cells were acquired using flow cytometer.

### Calculations

Decrease in apoptotic cell population (sub G0/G1) was calculated wrt UVB damage control.

$$[ ( B - A ) / B ] * 100 ; Where A = \% subG 0 / G 1 cells in Test Item treated , \\ B = \% subG 0 / G 1 cells in UVB damage Control$$

### 6c. Anti-Inflammatory Activity in Keratinocytes

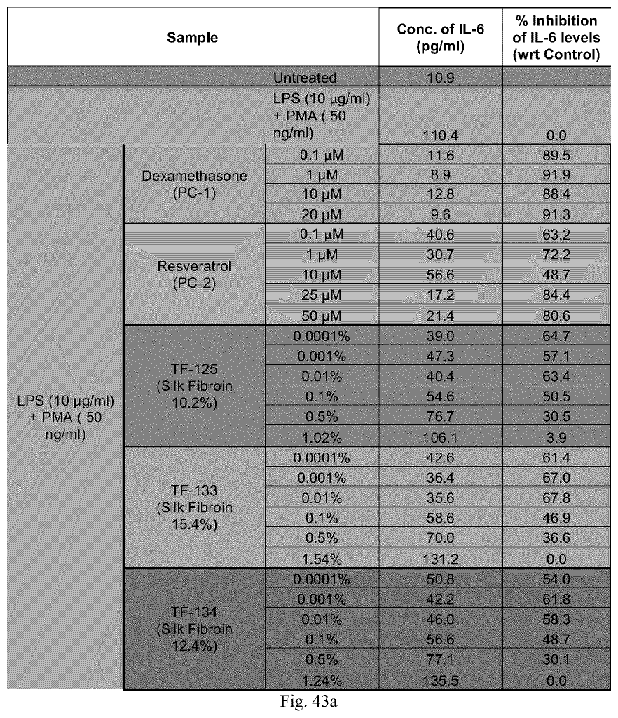

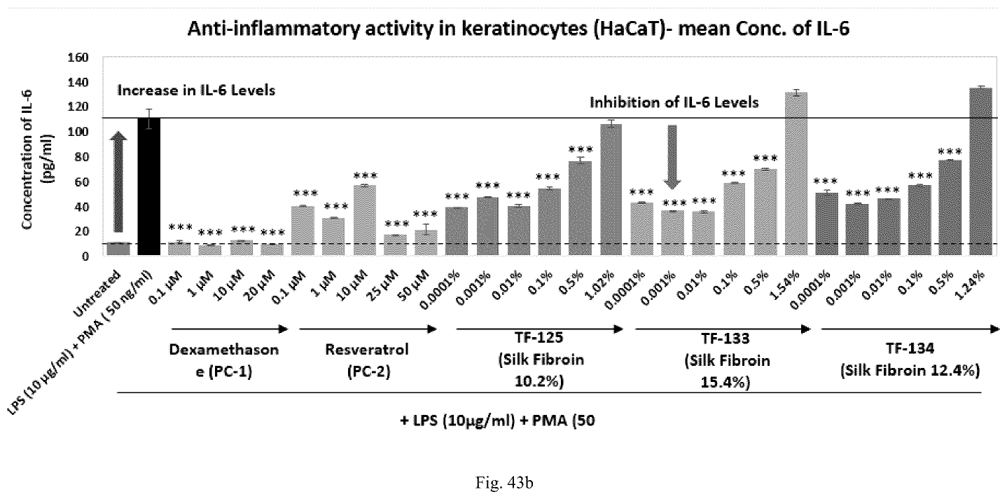

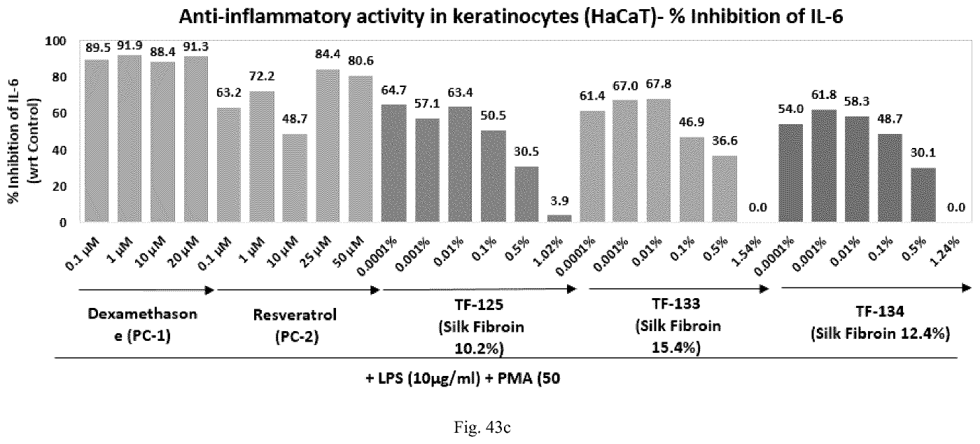

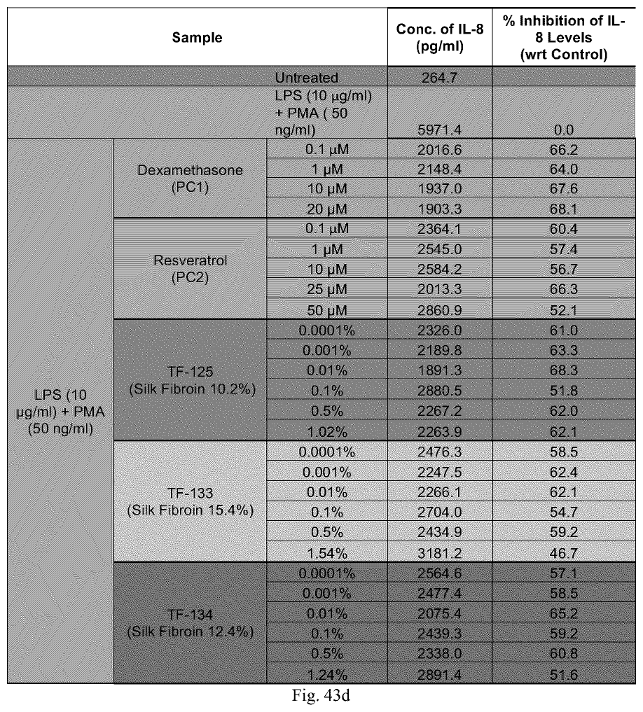

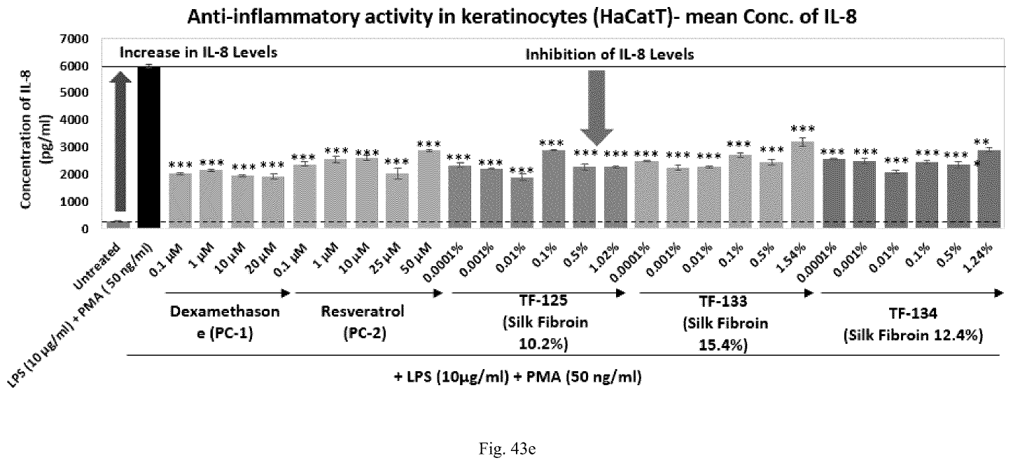

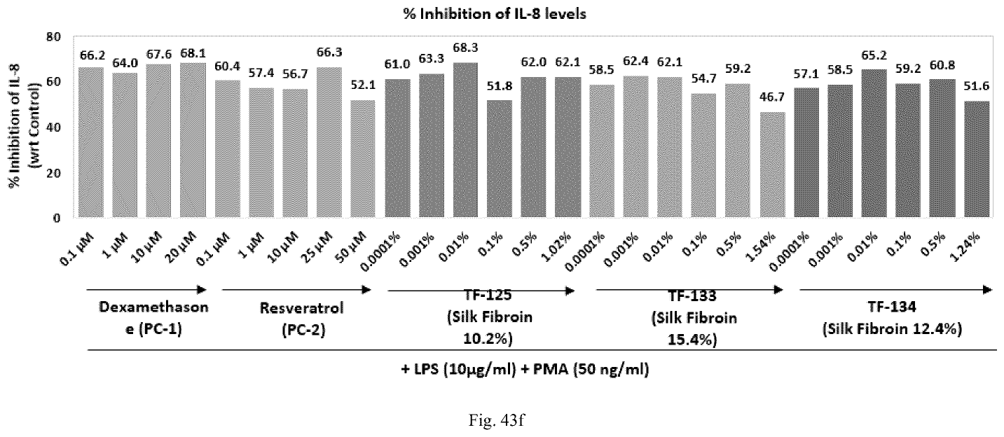

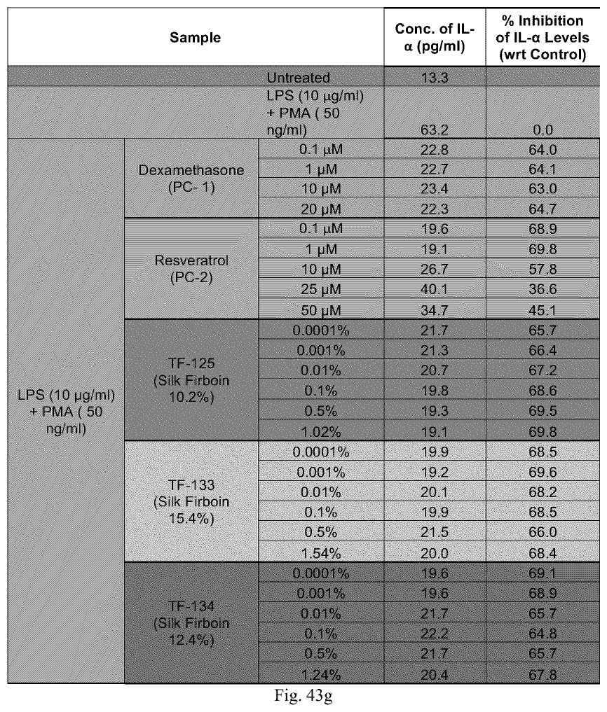

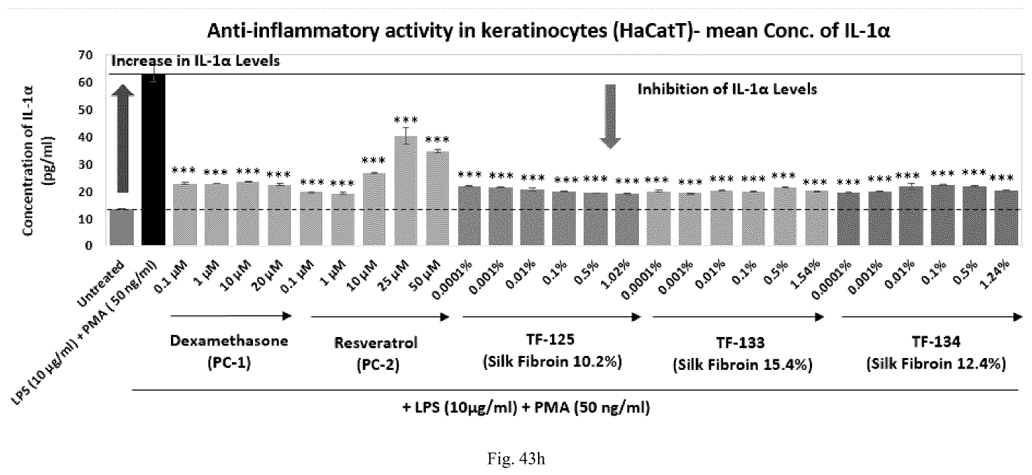

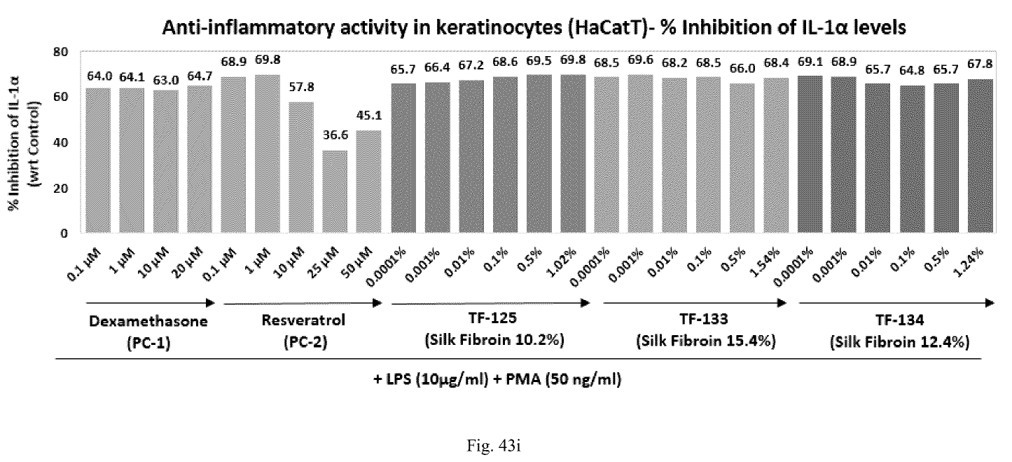

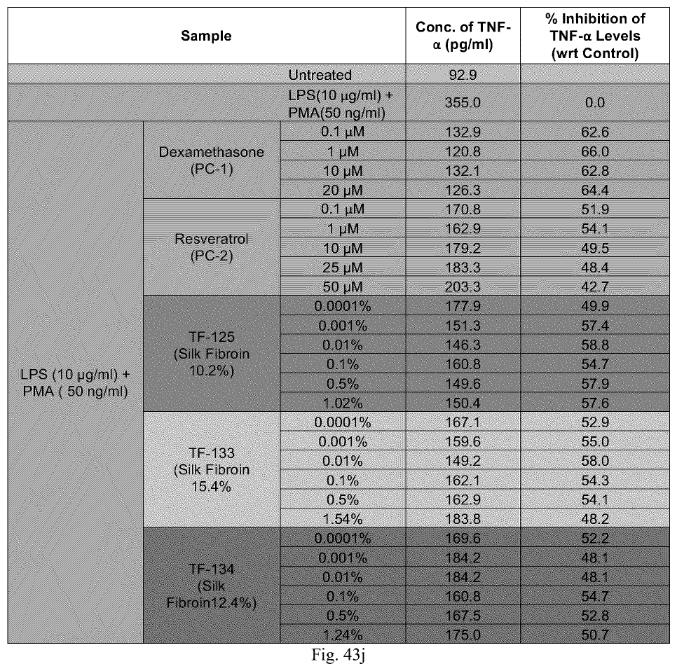

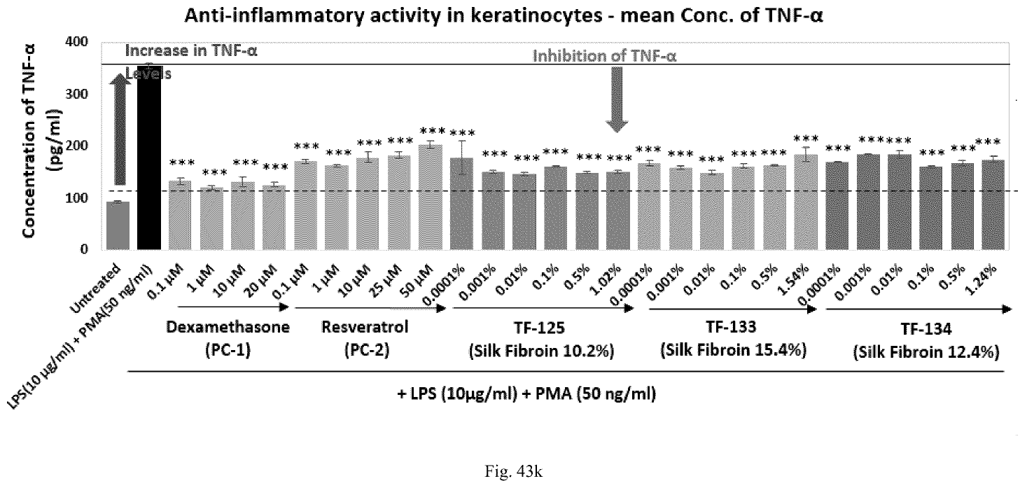

Study Design Assay 6c) Anti-Aging—Anti-Inflammatory Activity by Inhibition of Cytokines (as Seen in FIGS. 43a-43l)

### Study Design

Test System—Human keratinocytes (HaCaT)
Number of cells plated—0.1 million cells
FBS concentrations—0.1% FBS
Test Items—TF-125 (10.2%), TF-133 (15.4%), TF-134(12.4%)
Time point—24h
End point—ELISA
Positive Control/s—Dexamethasone, Resveratrol

### Procedure

Cells were plated in 10% FBS in 6-well plates and incubated for 24 h. Cells were serum starved in 0.1% FBS for 24 h.
Cells were treated with 4 Test Items in 0.1% FBS at non-cytotoxic concentrations along with LPS (10 pg/ml) and PMA (50 ng/ml) for 24h.
After 24h, culture supernatants were collected.
Levels of IL-6, IL-8, TNF-α and IL-1α (supernatants) were determined by ELISA as follows—
Assay diluent was added to each well. Respective kit standards and samples (supernatants) were directly pipetted into the wells and
incubated for 2 h at RT.
After washing away any unbound substances for a total of 3 times, respective conjugate was added to each well and incubated for 1-2 h at
RT.
Following a wash (3 times) to remove any unbound conjugate, substrate solution was added to the wells and incubated for 30 min at RT in
dark. The reaction was stopped by adding Stop solution to each well.
The optical density of the color was measured at 450 nm. Conc of cytokines were calculated from standard curve.

### Calculations

Inhibition in levels of cytokines was calculated wrt Stimulant (LPS+PMA) control.

$$[ ( B - A ) / B ] * 100 ; \\ Where A = Levels of cytokines in Test Item and Stimulant treated cells , \\ B = Levels of cytokines in Stimulant ( LPS + PMA ) Control$$

### 7. Skin Whitening Potential

### 7a. Inhibition of Melanin Content in Melanocytes

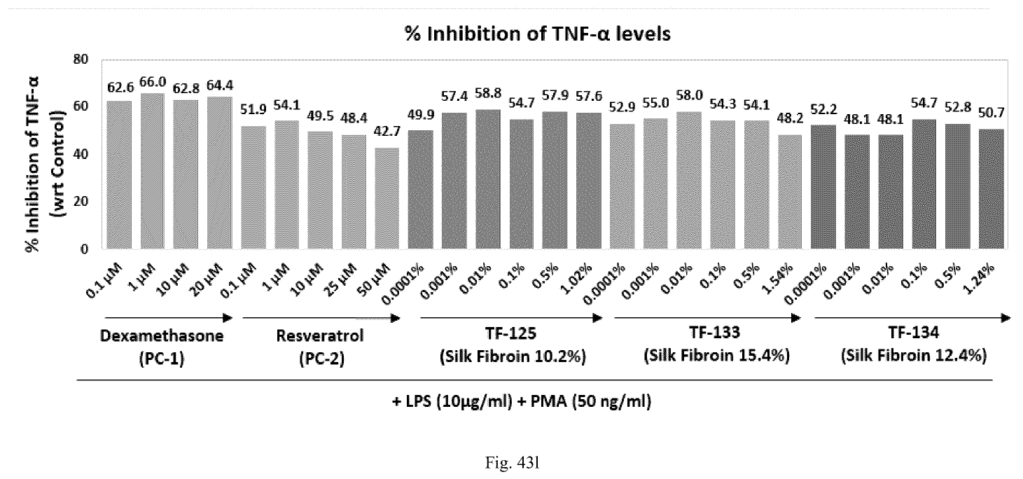

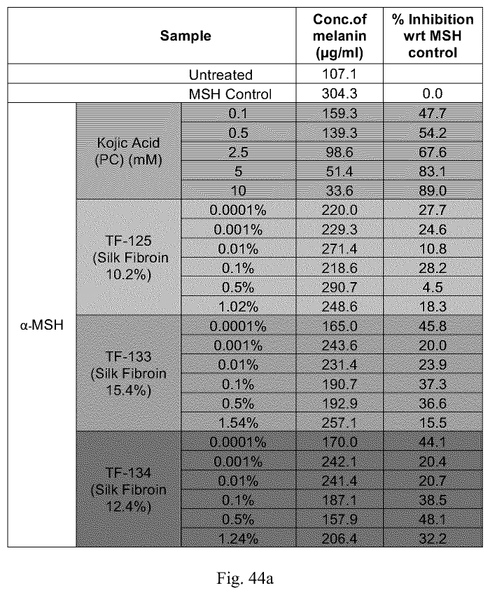

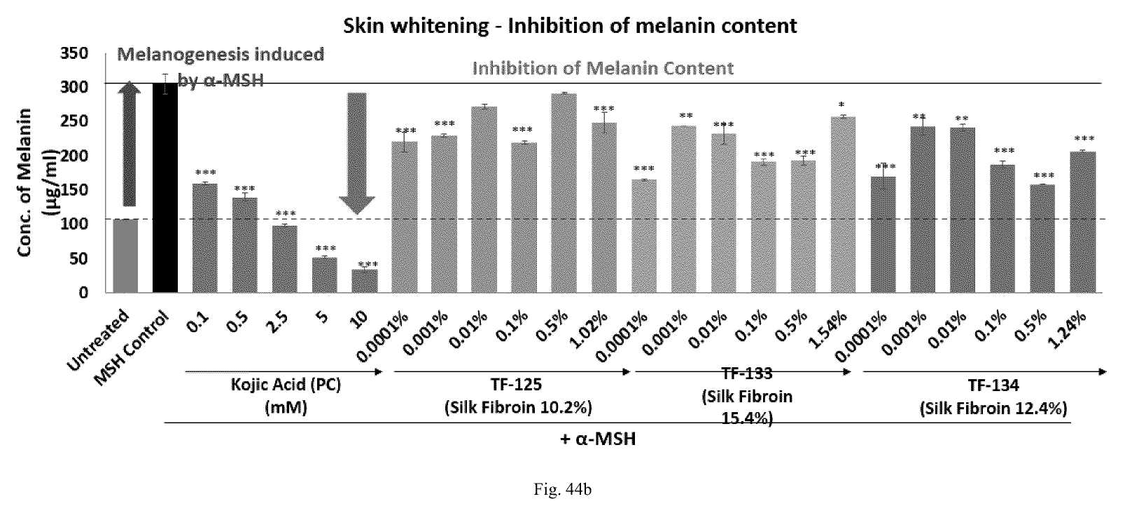

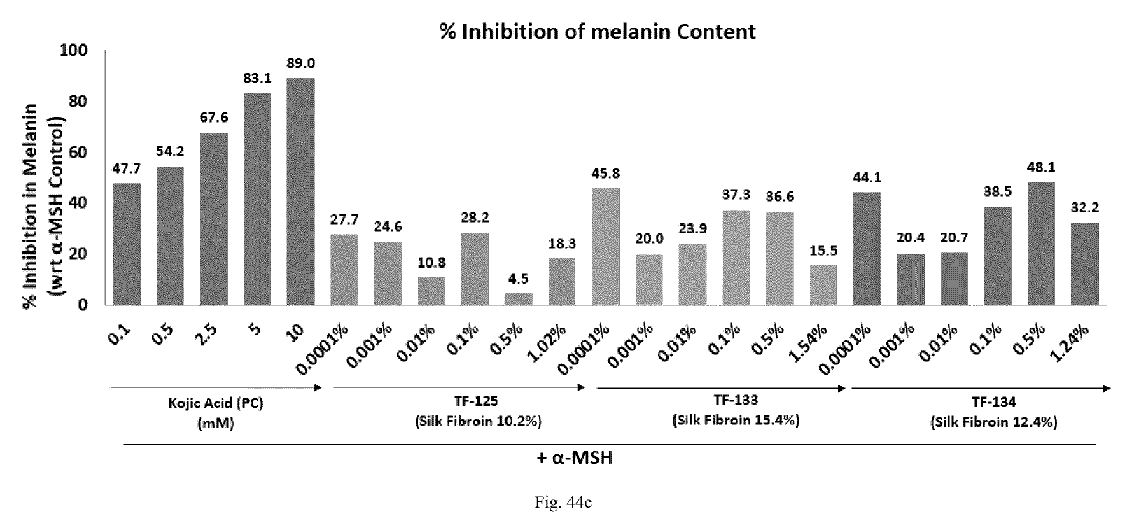

Assay 7a)—Skin Whitening—Inhibition of Melanin Content (as Seen in FIGS. 44a-44c)

### Study Design

Test System—B16F10 (mouse melanoma cell line)
Number of cells plated—40000 cells/well
FBS concentration—10%
Test Items—TF-125 (10.2%), TF-133 (15.4%), TF-134 (12.4%)
Time point—72h
Estimation method—Melanin estimation by NaOH extraction
Positive Control—Kojic Acid

### Procedure

Cells were plated in 6-well plates in 10% FBS and incubated for 24 h.
Cells were co-treated with Test Items at non-cytotoxic concentrations and α-MSH (500 nM) for 72 h.
Cells were washed using PBS and cell lysates were prepared in lysis buffer containing Triton-X and Sodium Phosphate buffer.
Melanin was extracted from cells in 1N NaOH containing 10% DMSO by overnight incubation at 37° C.
Percent Inhibition was calculated wrt Stimulant control (α-MSH stimulated cells).

### Calculations

Inhibition in levels of melanin was calculated wrt Stimulant (α-MSH) control.

$$[ ( B - A ) / B ] * 100$$

$$Where A = Levels of melanin in Test Item treated cells , \\ B = Levels of melanin in Stimulant ( α - MSH ) Control .$$

### 7b. Inhibition of Migration of Melanocytes

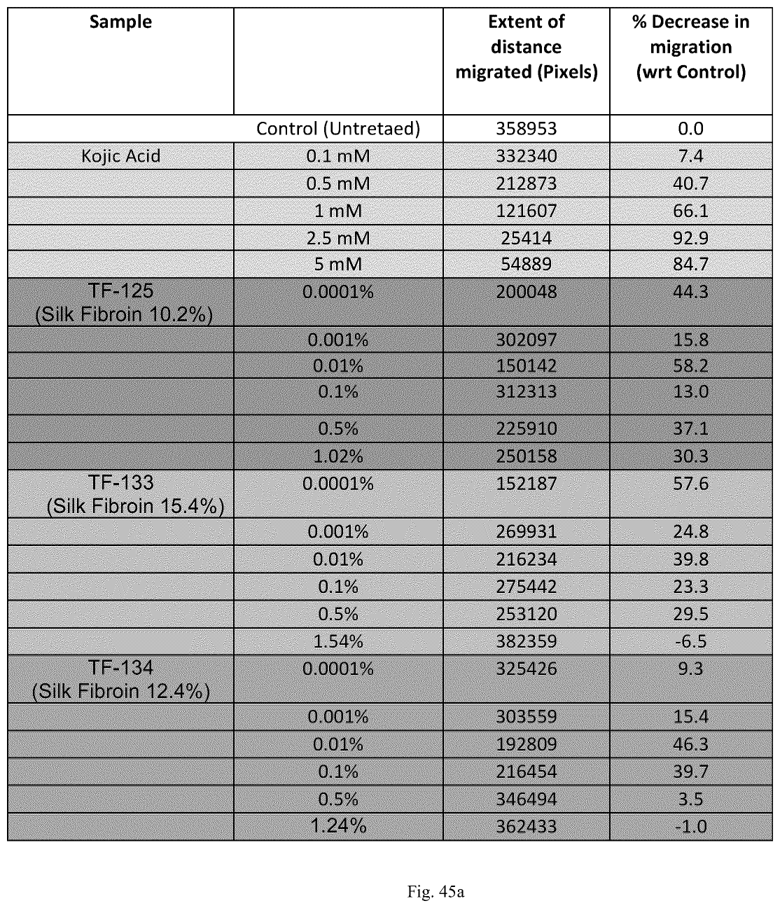

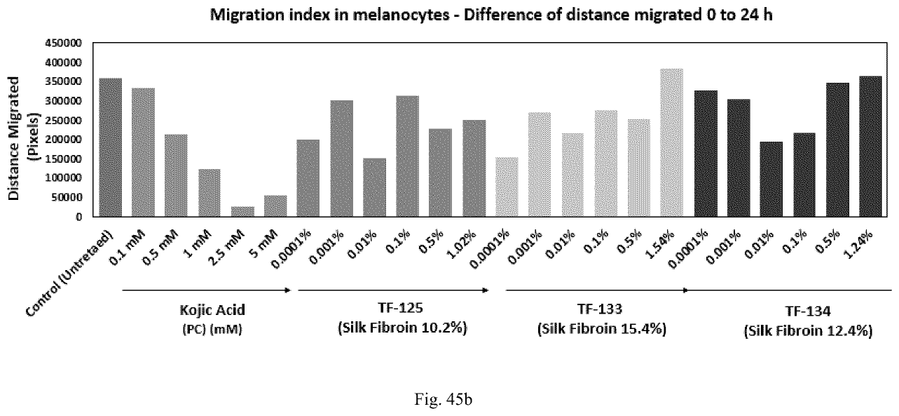

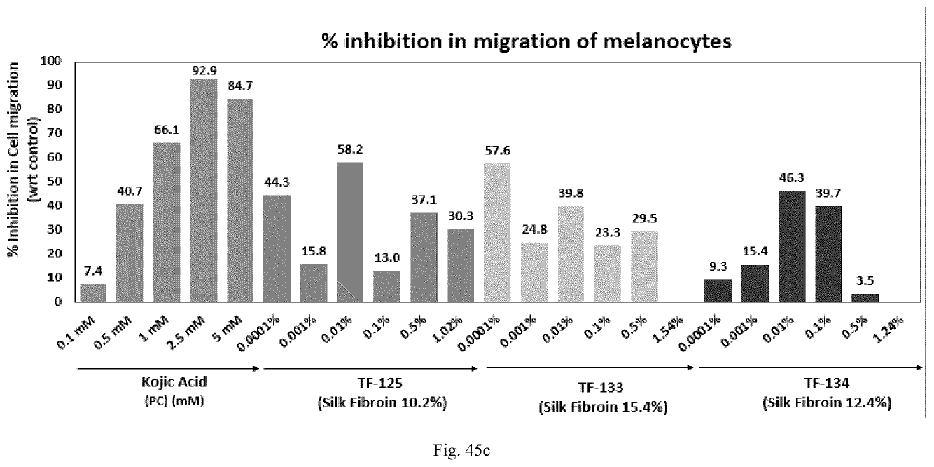

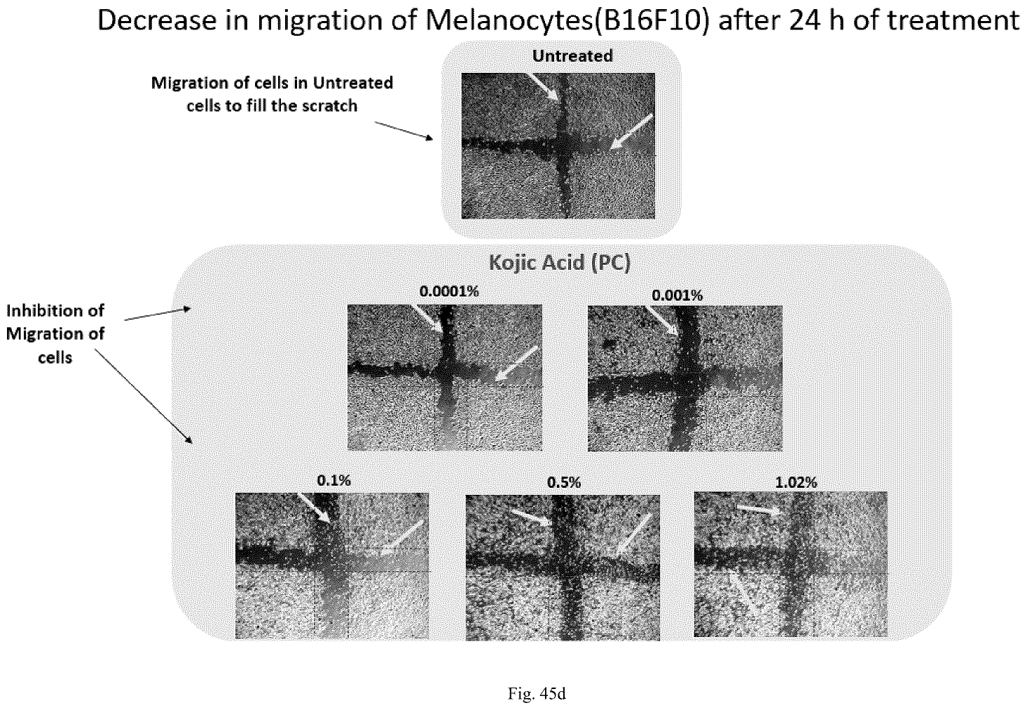

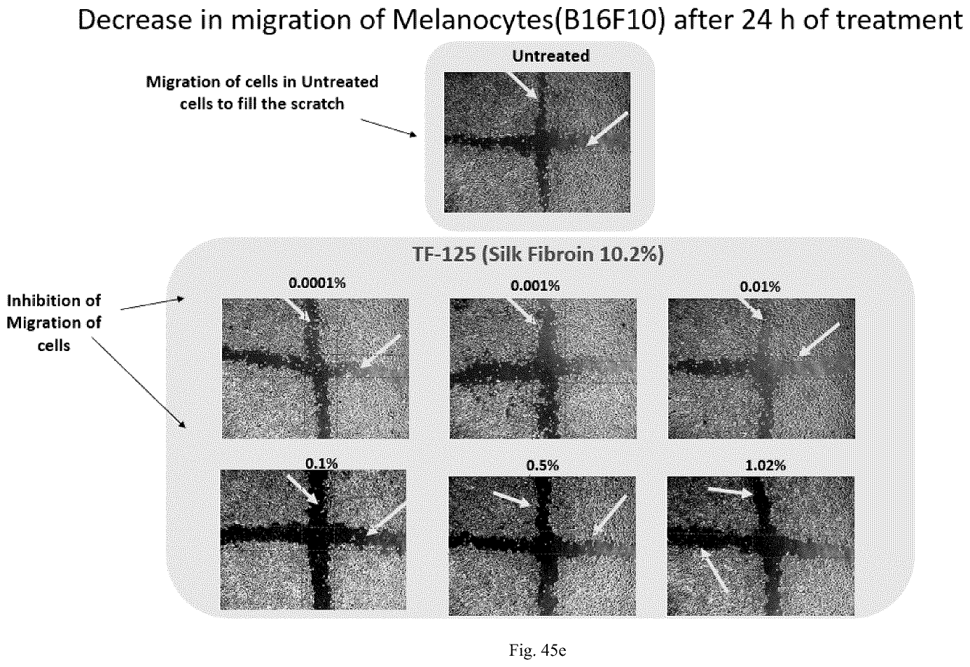

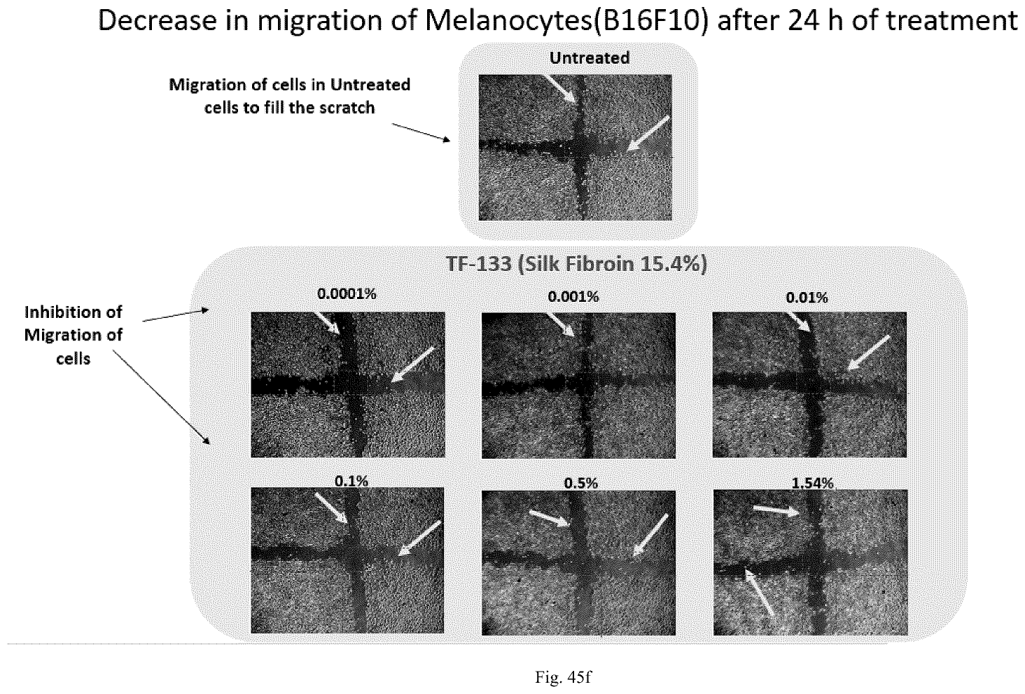

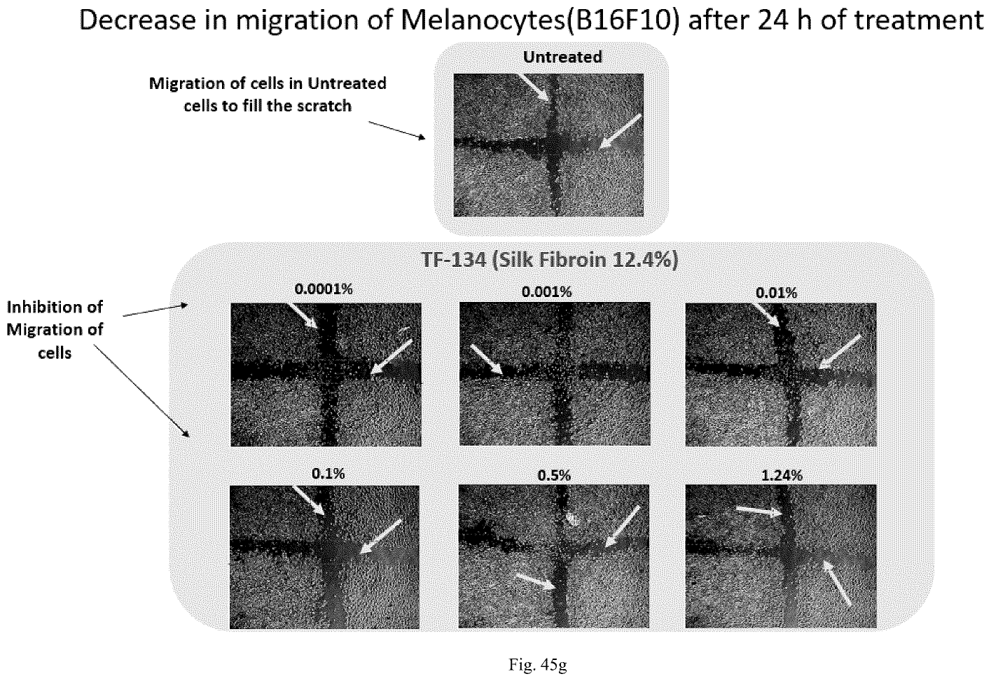

Assay 7b)—Skin Whitening—Inhibition of Melanocytes Migration (as Seen in FIGS. 45a-45g)

### Study Design

Test System—B16F10 (mouse melanoma cell line)
Number of cells plated—0.2×106 cells/well
FBS concentration—1%
Test Items—TF-125 (10.2%), TF-133 (15.4%), TF-134 (12.4%)
Time point—24h
Estimation method—Scratch assay
Positive Control—Kojic Acid

### Procedure

Cells were plated in 12-well plates in 10% FBS and incubated for 24 h.
Scratch was created in each well using a sterile tip followed by treatment of cells with 3 Test items at non-cytotoxic concentrations. Images were captured (0h).
After 24 h, the images were captured to determine the effect of Test Items on migration.
Decrease in migration was calculated using ImageJ software.

### Calculations

Increase in migration was calculated wrt control (Untreated cells).

$$[ ( A - B ) / B ] * 100 ; Where A = \% Migration in Test Item treated cells , \\ B = \% Migration in Control ( Untreated ) cells$$

The extent of Migration/Distance migrated was calculated as:
Empty space (pixels) at 0 h—Empty space (pixels) at 24 h
For each sample, respective zero h control was taken into consideration.

### 7c) Inhibition of Tyrosinase Activity

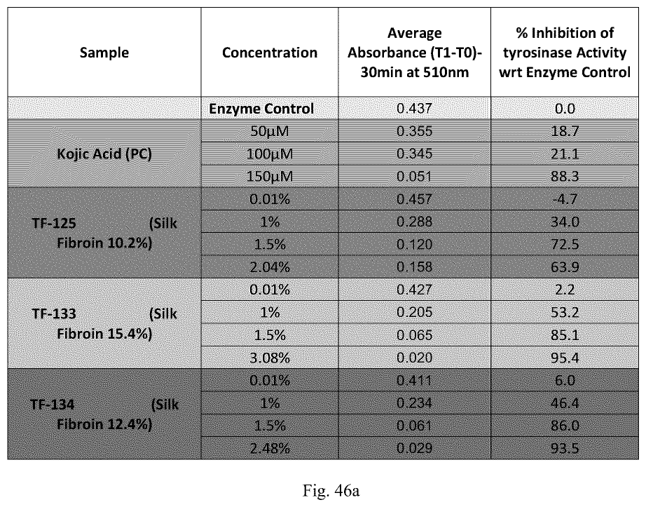

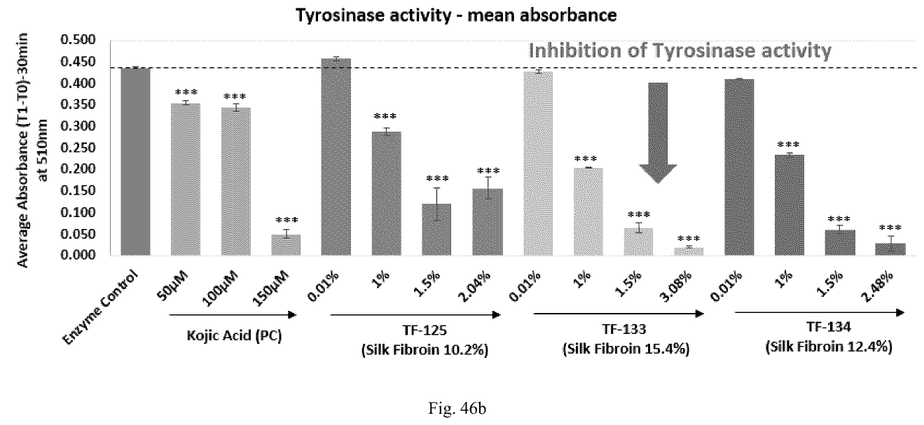

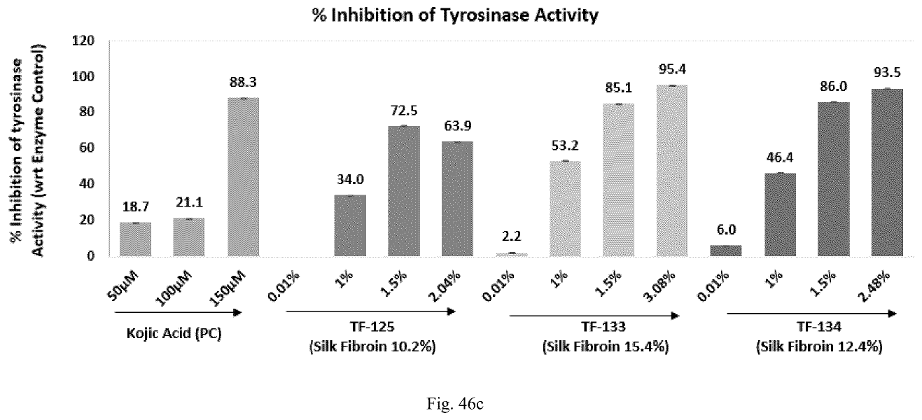

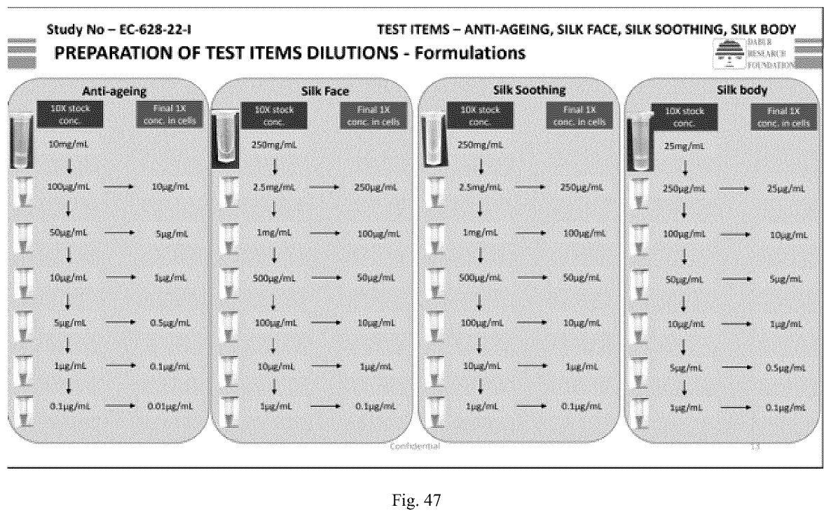

Study Design Assay 7c)—Skin Whitening—Inhibition of Tyrosinase Activity (as Seen in FIGS. 46a-46c)

### Study Design

Test System—Tyrosinase Enzyme
Test Items—TF-125 (10.2%), TF-133 (15.4%), TF-134 (12.4%)
Time points—30 min
Estimation method—Colorimetric cell free assay
Positive Control—Kojic Acid

### Procedure

PC/Test items were added to 96-well plate along with enzyme solution and incubated for 10 min at RT.
After 10 min, the substrate solution was added to each well. The well containing only enzyme and substrate (no inhibitor) was used as Enzyme control.
The absorbance was read at 510 nm at TO
(at 0 min) and T1
(after 30 min).

### Calculations

Inhibition in Tyrosinase activity was calculated wrt Enzyme control.

$$[ ( B - A ) / B ] * 100 ;$$

$$Where A = ( T 1 OD of Test Item treated cells - T 0 OD of Test Item treated cells ) , B = ( T 1 OD of Enzyme Control - T 0 OD of Enzyme Control ) .$$

### Formulations

While the present disclosure has been described at some length and with some particularly with respect to the several described illustrative embodiments set forth below. These illustrative formulations and methods formulating show various suspensions and other preparations that incorporate silk fibroin peptides into various formulations that can be used to apply silk fibroin peptides to affected body parts. These illustrative formulations are for illustrative purposes only and are not intended to limit the disclosure to any such particulars set forth in these formulations or embodiments or any particular embodiment, but it is to be construed with references to the above disclosure as to provide the broadest possible interpretation of therapeutic uses of silk fibroin peptides in view of the prior art and, therefore, to effectively encompass the intended scope of the disclosure. Various formulations are set forth below that provide silk fibroin peptides are as follows:

| Formula# | Silk Ant-Aging Face Lotion; Anti-Ageing; Anti-Aging; | | |
|---|---|---|---|
| CZN-019 | Daily Defense + Sunscreen; Mineral SPF | | |
| Phase | Supplier | Trade Name | INCI |
| WATER | | | |
| PHASE | | | |
| 1 | Crystal | Water | Water |
| 2 | Silklyfe | Silk Fibroin | Hydrolyzed Fibroin |
| | | Solution | (and) Water |
| 1 | Ajinomoto | L- Arginine C- | Arginine |
| | (OR) | Grade (OR) | |
| | Symrise | Biotive L-Arginine | |
| 1 | Evonik | Dermofeel PA | Phytic Acid |
| | | | (and) Aqua |
| 1 | EMD | RonaCare | Magnesium Sulfate |
| | Performance | Magnesium Sulfate | |
| | Materials/ | | |
| | Rona | | |
| 5 | Chemyunion | Hydrolite CG | Caprylyl Glycol |
| 5 | Symrise | Hebeatol Plus | Xylityl |
| | | | Sesquicaprylate |
| 5 | Oh Oh | Organic | Glycerin |
| | Organic | Glycerin, Vegetable | |
| | | Derived (USP) | |
| 5 | CP Kelco | Keltrol CG-SFT | Xanthan Gum |
| OIL | | | |
| PHASE | | | |
| 3 | Kobo | GCP45XZJ | Caprylic/Capric |
| | | | Triglyceride (and) |
| | | | Zinc Oxide (and) |
| | | | Polyhydroxystearic |
| | | | Acid (and) Jojoba |
| | | | Esters |
| 3 | Kobo | GCG50TYSG | Iron Oxides (CI 77492) |
| | | | (And) Caprylic/Capric |
| | | | Triglyceride (And) |
| | | | Polyglyceryl-3 |
| | | | Diisostearate (And) |
| | | | Stearoyl Glutamic |
| | | | Acid |
| 3 | Chemyunion | AM-Melscreen | Mauritia Flexuosa |
| | | Buriti TI FG | Fruit Oil |
| 3 | Chemyunion | Melscreen | Coffea Arabica |
| | | Coffee EL | Seed Oil |
| | | DEO ORG | |
| 3 | Chemyunion | Physavie | Physalis Angulata |
| | | | Extract&Caprylic/ |
| | | | Capric Triglyceride |
| 3 | Kobo | SunBoost ATB | Argania Spinosa |
| | | Natural | Kernel Oil |
| | | | (and) Tocopheryl |
| | | | Acetate (and) |
| | | | Bisabolol |
| 4 | Micro | Naturesoft | Oryza Sativa (Rice) |
| | Powders | 860R | Bran Wax |
| 4 | BASF | Dehymuls | Polyglyceryl-2 |
| | | PGPH | Dipolyhydroxystearate |
| 4 | BASF | Lameform TGI | Polyglyceryl-3 |
| | | | Diisostearate |
| 4 | BASF | Cutina HVG | Hydrogenated |
| | | | Vegetable |
| | | | Glycerides |
| 4 | Kobo | MSS-500W | Silica |
| 4 | Kobo | MSS-500/N | Silica |

| Formula# | | | |
|---|---|---|---|
| CDM-015 | | Silk Body Lotion; Silk Body | |
| Phase | Supplier | Trade Name | INCI |
| WATER | | | |
| PHASE | | | |
| 1 | Crystal | WATER | Water |
| 3 | Silklyfe | Silk Fibroin | Hydrolyzed Silk |
| | | Solution | (and) Water |
| 2 | Evonik | Dermofeel PA | Phytic Acid (and) |
| | | | Aqua |
| 2 | Ajinomoto | L- Arginine | Arginine |
| | (OR) | C-Grade (OR) | |
| | Symrise | Biotive L-Arginine | |
| 2 | Lotioncrafter | Sodium Citrate, | Sodium Citrate |
| | | Dihydrate, | |
| | | Granular, USP | |
| 5 | Lucas Meyer | Siligel | Xanthan gum (and) |
| | Cosmetics | | Lecithin (and) |
| | | | Sclerotium gum |
| | | | (and) Pullulan |
| 5 | Symrise | Hydrolite CG | Caprylyl Glycol |
| 5 | Oh Oh | Organic Glycerin, | Glycerin |
| | Organic | Vegetable | |
| | | Derived (USP) | |
| OIL | | | |
| PHASE | | | |
| 4 | IOI | IMWITOR 372P | Glyceryl Stearate |
| | | | Citrate |
| 4 | Lotioncrafter | Lotioncrafter GMS | Glyceryl stearate |
| 4 | Hallstar | Olivem 1000 | Cetearyl Olivate |
| | | | (and) Sorbitan |
| | | | Olivate |
| 4 | Chemyunion | SENSOVEIL SIL | Orbignya Speciosa |
| | | | (Babassu) Kernel Oil |
| | | | (and) Hydrogenated |
| | | | Soybean Oil (and) |
| | | | Cocos Nucifera |
| | | | (Coconut) Oil (and) |
| | | | Linum Usitatissimum |
| | | | (Linseed) Seed Oil |
| 4 | BASF | Lanette 22 | Behenyl Alcohol |
| 4 | Oh Oh | Organic MCT Oil | CAPRYLIC/CAPRIC |
| | Organic | (Deodorized) from | TRIGLICERIDE |
| | | Coconut Oil | |
| 4 | Oh Oh | Organic Shea | BUTYROSPERMUM |
| | Organic | Butter, Refined | PARKIL |
| | | | SHEA BUTTER |
| 6 | Kobo | MSS-500W | Sillica |
| COOLING | | | |
| DOWN | | | |
| 7 | Kobo | Tocobiol SF | Tocopherol |
| 7 | Chemyunion | Hebeatol Plus | Xylityl |
| | | | Sesquicaprylate |

| Formula# | | Silk Soothing Lotion; | |
|---|---|---|---|
| CAS-009 | | Silk Soothing; Silk Soothing Spray | |
| Phase | Supplier | Trade Name | INCI |
| WATER | | | |
| PHASE | | | |
| 1 | Crystal | WATER | |
| 3 | Silklyfe | Silk Fibroin | Hydrolyzed |
| | | Solution | Fibroin (and) |
| | | | Water |
| 2 | Evonik | Dermofeel PA | Phytic Acid |
| | | | (and) Aqua |
| 2 | Ajinomoto (OR) | L- Arginine C- | Arginine |
| | Symrise | Grade (OR) | |
| | | Biotive L-Arginine | |
| 2 | Lotioncrafter | Sodium Citrate, | Sodium Citrate |
| | | Dihydrate, | |
| | | Granular, USP | |
| 4 | Lucas Meyer | Siligel | Xanthan Gum (and) |
| | | | Lecithin (and) |
| | | | Sclerotium Gum |
| | | | (and) Pullulan |
| 4 | Oh Oh Organic | Organic Glycerin, | Glycerin |
| | | Vegetable | |
| | | Derived (USP) | |
| 4 | Symrise | Hydrolite CG | Caprylyl Glycol |
| 4 | Chemyunion | Hebeatol Plus | Xylityl |
| | | | Sesquicaprylate |
| OIL | | | |
| PHASE | | | |
| 5 | Hallstar | Olivem 1000 | Cetearyl Olivate |
| | | | (and) Sorbitan |
| | | | Olivate |
| 5 | Chemyunion | Physavie | Physalis Angulata |
| | | | Extract&Caprylic/ |
| | | | Capric Triglyceride |
| 5 | Lotioncrafter | Neossance | Squalane |

| | |
|---|---|
| Formula# | Silklyfe Face Serum; |
| CFS-021 | Silk Face; Active Silk Serum |

| Phase | Supplier | Trade Name | INCI |
|---|---|---|---|
| 1 | Crystal | Water | Water |
| 2 | Evonik | Dermofeel | Phytic Acid |
| | | PA | (and) Aqua |
| 2 | Spectrum | Citric Acid | Citric Acid |
| 2 | Lotioncrafter | Sodium | Sodium |
| | | Citrate, | Citrate |
| | | Dihydrate, | |
| | | Granular, USP | |
| 3 | BASF | Rheocare | Xanthan Gum |
| | | XGN | |
| 3 | Genomatica/ | Brontide | Butylene |
| | Essential | | Glycol |
| | Ingredients | | |
| 4 | Ajinomoto | Ajidew NL-50 | Sodium PCA |
| | | | (and) Aqua |
| 4 | Schulke | Euxyl K 712 | Aqua (and) |
| | | | Sodium Benzoate |
| | | | (and) Potassium |
| | | | Sorbate |
| 5 | Silklyfe | Silk Fibroin | Hydrolyzed |
| | | Solution | Fibroin (and) |
| | | | Water |

| | |
|---|---|
| Face and Body Bar | Hydrogenated Vegetable Oil, Sodium |
| | Palmate, Sodium Cocoyl Isethionate, |
| | Water/Aqua/Eau, Sodium Cocoate, |
| | Polyglyceryl-4 Laurate, Glycerin, Helianthus |
| | Annuus (Sunflower) Seed Oil, Citrus Aurantium |
| | Bergamia (Bergamot) Fruit Oil, Jasminum |
| | Grandiflorum (Jasmine) Flower Extract, |
| | Cananga Odorata Flower Oil, Citrus Limon |
| | (Lemon) Peel Oil, Lavandula Hybrida Oil, Coco- |
| | Glucoside, Hydrolyzed Fibroin, Sodium |
| | Astrocaryum Murumuruate, Guar |
| | Hydroxypropyltrimonium Chloride, Citric Acid, |
| | Sodium Chloride Tocopheryl Acetate, Ascorbic |
| | Acid, Camellia Sinensis Leaf Extract |
| Silk Soothing Lotion | Water/Aqua/Eau, Glycerin, Squalane, |
| | Cetearyl Olivate, Xylityl Sesquicaprylate, |
| | Hydrolyzed Fibroin, Physalis Angulata Extract, |
| | Tocopherol, Capric Caprylic Triglyceride, |
| | Arginine, Phytic Acid, Sodium Citrate, Xanthan |
| | Gum, Sclerotium Gum, Lecithin, Pullulan, |
| | Caprylyl Glycol, Anhydroxylitol, Silica, |
| | Sorbitan Olivate. |
| Silk Face Serum | Water/Aqua/Eau, Butylene Glycol, |
| | Hydrolyzed Fibroin, Sodium Citrate, Citric Acid, |
| | Phytic Acid, Xanthan Gum, Sodium PCA, |
| | Sodium Benzoate, Potassium Sorbate |
| Silk Body Lotion | Water, Glycerin, Butyrospermum Parkii |
| | (Shea) Butter, Orbignya Speciosa (Babassu) |
| | Kernal Oil, Glyceryl Stearate Citrate, |
| | Hydrogenated Soybean Oil, Glyceryl Stearate, |
| | Caprylic/Capric Triglyceride, Xylityl |
| | Sesquicaprylate, Silica, Hydrolized Fibroin, |
| | Tocopherol, Cocos Nucifera (Coconut) Oil, |
| | Linum Usitatissimum (Linseed) Oil, Helianthus |
| | Annuus (Sunflower) Seed Oil, Arginine, Beta- |
| | Sitosterol, Squalene, Phytic Acid, Sodium |
| | Citrate, Xanthan Gum, Sclerotium Gum, |
| | Lecithin, Pullulan, Cetearyl Olivate, Caprylyl |
| | Glycol, Anhydroxylitol, Behenyl Alcohol, |
| | Sorbitan Olivate |
| Silk Anti-Aging | Active Ingredients: Zinc oxide 19.3% |
| Lotion | Inactive Ingredients: Caprylic/Capric |
| | Triglycerides, Zinc Oxide, Water/Aqua/Eau, |
| | Mauritia Flexuosa Fruit Oil, Coffea Arabica |
| | (Coffee) Seed Oil, Polyglyceryl-2 |
| | Dipolyhydroxystearate, Glycerin, Silica, |
| | Argania Spinosa Kernal Oil, Tocopheryl Acetate, |
| | Polyglyceryl-3 Diisostearate, Oryza Sativa |
| | (Rice) Bran Wax, Polyhydroxystearic Acid, |
| | Xylityl Sesquicaprylate, Jojoba Esters, Physalis |
| | Angulata Extract, Hydrolyzed Fibroin, |
| | Tocopherol, Arginine, Hydrogenated Vegetable |
| | Glycerides, Xanthan Gum, Magnesium Sulfate, |
| | Phytic Acid, Anhydroxylitol, Iron Oxides (CI |
| | 77492), Stearoyl Glutamic Acid, Caprylyl |
| | Glycol, Bisabolol |
| Body Sunscreen | Active Ingredients: Zinc Oxide 21.7% |
| | Inactive ingredients Caprylic/Capric |
| | Triglyceride, Water, Helianthus Annuus |
| | (Sunflower) Seed Oil, Carthamus Tinctorius |
| | (Safflower) Seed Oil, Argania Spinosa Kernel |
| | Oil, Tocopheryl Acetate, Methyl |
| | Dihydroabietate, Polyhydroxystearic Acid, |
| | Jojoba Esters, Hydrolyzed Fibroin, Tocopherol, |
| | Bisabolol, Cellulose Gum, Microcrystalline |
| | Cellulose, Bentonite, Cetearyl Alcohol, Coco- |
| | Glucoside, Caprylhydroxamic Acid, Caprylyl |
| | Glycol, Glycerin. |
| Shampoo Bar | Sodium cocoyl isethionate, Coco |
| | glucoside, butyrospermum parkii (Shea) butter, |
| | Sodium astrocaryum murmurate, |
| | Water/aqua/Eau, Arrowroot starch, hydrolyzed |
| | fibroin, physalis angulata extract, caprylic/capric |
| | triglyceride, guar hydroxypropyltrimonium |
| | chloride, Helianthus Annuus (Sunflower) Seed |
| | Oil, Citrus Aurantium Bergamia (Bergamot) |
| | Fruit Oil, Cananga Odorata Flower Oil, Citrus |
| | Limon (Lemon) Peel Oil, Lavandula Hybrida |
| | Oil, |

### Shampoo Bar

### Silklyfe Shampoo Bar SLSB-013

INGREDIENTS

Sodium Cocoyl Isethionate
Coco Glucoside
Guar Hydroxypropyltrimonium Chloride
Sodium Astrocaryum Murumuruate
Arrowroot Starch
Physalis Angulata Extract
Caprylic/Capric Triglyceride
Butyrospermum Parkii (Shea Butter)
Hydrolyzed Fibroin
Water
Helianthus Annuus Seed Oil,
Citrus Aurantium Bergamia Fruit Oil
Cananga Odorata Flower Oil
Citrus Limon Peel Oil
Lavandula Hybrida Oil

### F. Evaluation of Skin Health Promoting and Anti-Aging Potential of Skin Formulations Using In Vitro Cell Based Assays by Multiparametric Analysis

### 1. Skin Health—Fibroblasts Based Assays

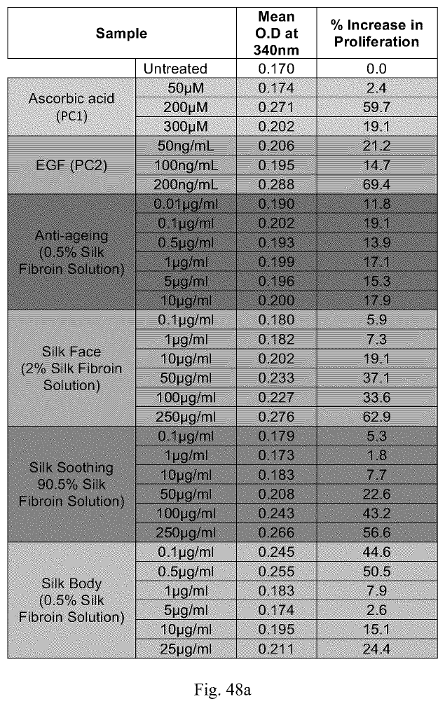

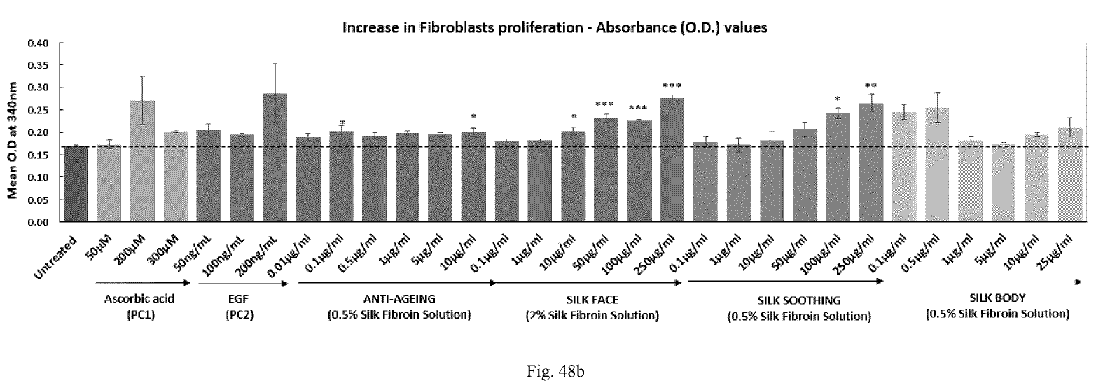

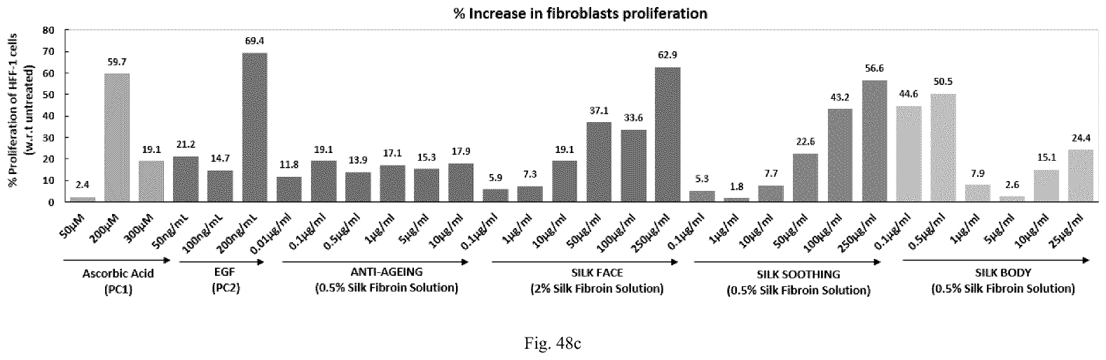

### 1a. Increase in Cell Proliferation in Fibroblasts (as Seen in FIGS. 48a-48c)

### Study Design

Test System—Human Fibroblasts (HFF 1)
Number of cells plated—1500 cells/96 well plates
FBS concentrations—0.5%
Test items—Anti Ageing/Mineral SPF, Silk Face/Silk serum, Silk Soothing/Soothing lotion, Silk Body/Body lotion
Time points—3 days
Estimation method—BrdU assay
Positive Control/s—Ascorbic acid, EGF

### Procedure

Cells were plated in 10% FBS in 96-well plates and incubated for 24 h.
Cells were serum starved in 0% FBS for 24 h.
Cells were treated with 4 Test items in 0.5% FBS at various concentrations.
After 3 days (72h), the effect on proliferation was determined by BrdU assay.
   1. 10 μl of BrdU solution was added/well and cells were incubated for 90 minutes at 37° C.
   2. After removing medium, cells were fixed, and the DNA was denatured in one step by adding 200 μl of FixDenat. Cells were incubated for 30 min at RT (15 25° C.).
   3. The FixDenat solution was removed and 100 μl of Anti-BrdU-POD solution was added to each well. Cells were incubated for 90 min at RT (15 25° C.).
   4. Wells were washed 3 times using 200 μl of washing solution.
   5. 100 μl/well of substrate solution was added.
   6. Cells were incubated for 30 min at RT (15-25° C.).
   7. After incubation, a blue coloured complex was formed.

Absorbance of each well was measured at 370 nm.

### Calculations

Increase in proliferation was calculated wrt control (Untreated cells).

$$[ ( A - B ) / B ] * 100 ; Where A = Absorbance in Test Item treated cells , \\ B = Absorbance in Control ( Untreated ) cells$$

### 1b. Stimulation of ECM Markers in Fibroblasts

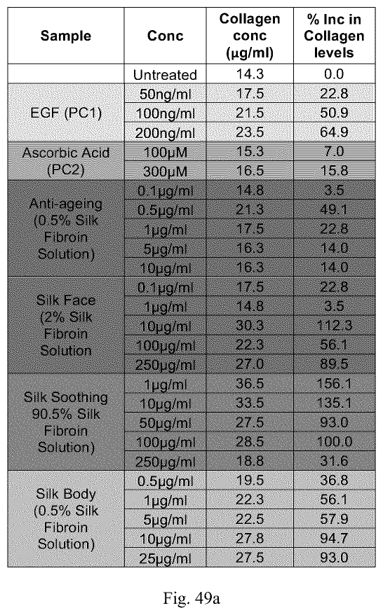

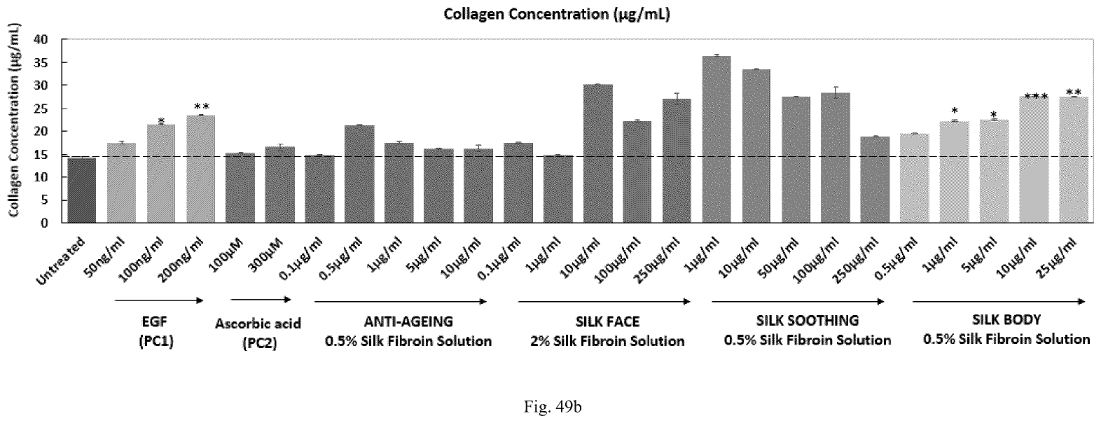

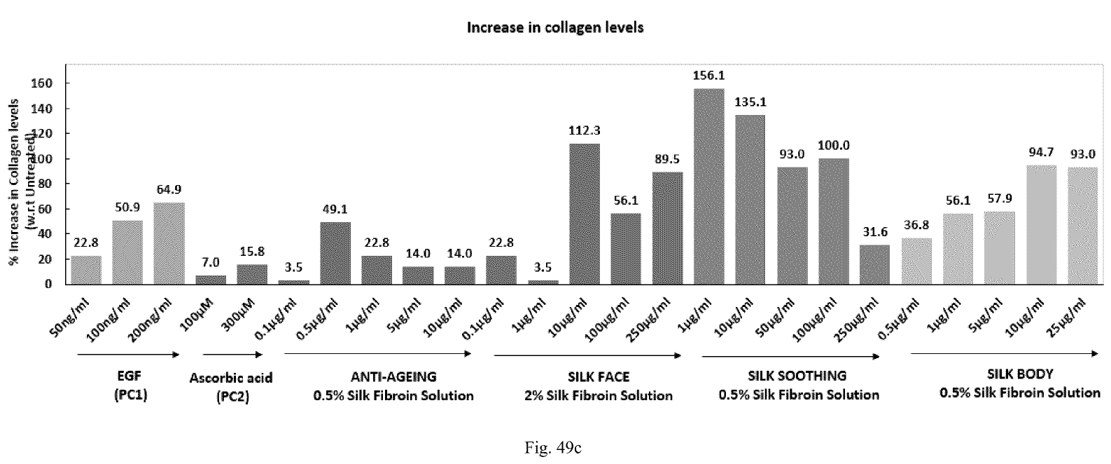

Study Design Assay 1b)—Skin Health—Increase in ECM (Collagen) (as seen in FIGS. 49a-49c)

### Study Design

Test System—Human Fibroblasts (HFF 1)
Number of cells plated—50,00 cells/12 well plates
FBS concentration—0.5%
Test items—Anti Ageing/Mineral SPF, Silk Face/Silk serum, Silk Soothing/Soothing lotion, Silk Body/Body lotion
Time points—3 days
Estimation method—Direct Red dye Staining (Collagen)
Positive Control/s—Ascorbic acid, EGF

### Procedure

HFF-1 cells were plated in 12 well plate at the density of 50,000 cells/well in DMEM supplemented with 15% FBS.
Following overnight incubation, the cells were subjected to serum starvation for 24 hours in DMEM+0% FBS.
Post serum starvation, cells were treated with non
cytotoxic concentrations of TIs and PCs.
Following 72 hours of treatment with the TIs and PCs, lysates were prepared for collagen estimation and supernatants were collected for determination of HA/Elastin levels.

### Collagen Staining:

i. Cell monolayer was washed with 1×PBS and lysates were prepared in PBS by freeze thaw method
ii. The lysates were then stained with Direct red dye and washed with HCl The bound dye was eluted with NaOH
iii. The absorbance was read at 540 nm

### Calculations

Increase in proliferation was calculated wrt control (untreated cells).

$$[ ( A - B ) / B ] * 100 ; Where A = Absorbance in Test Item treated cells , \\ B = Absorbance in Control ( Untreated ) cells$$

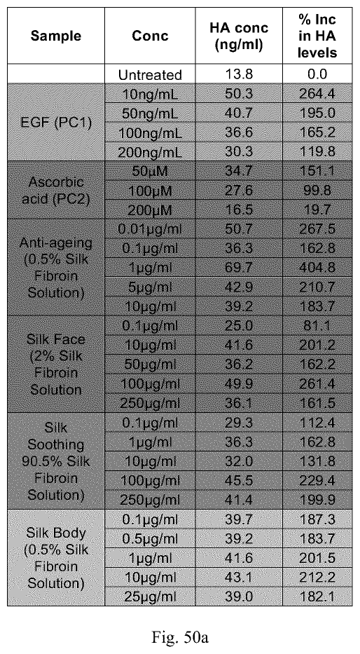

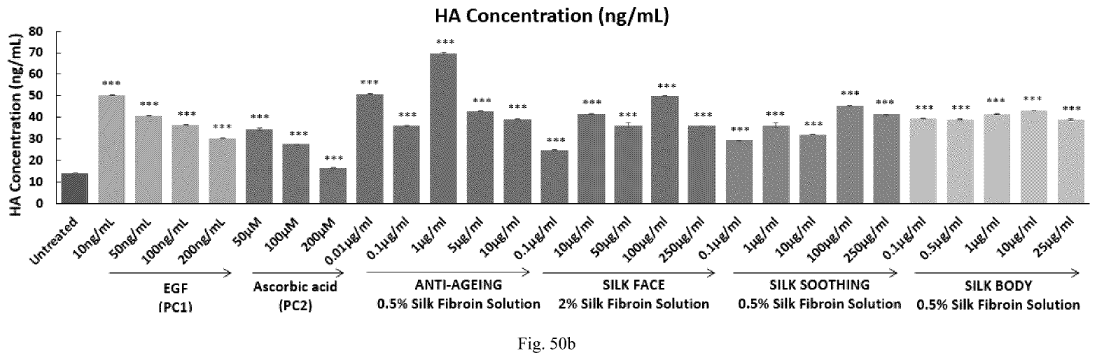

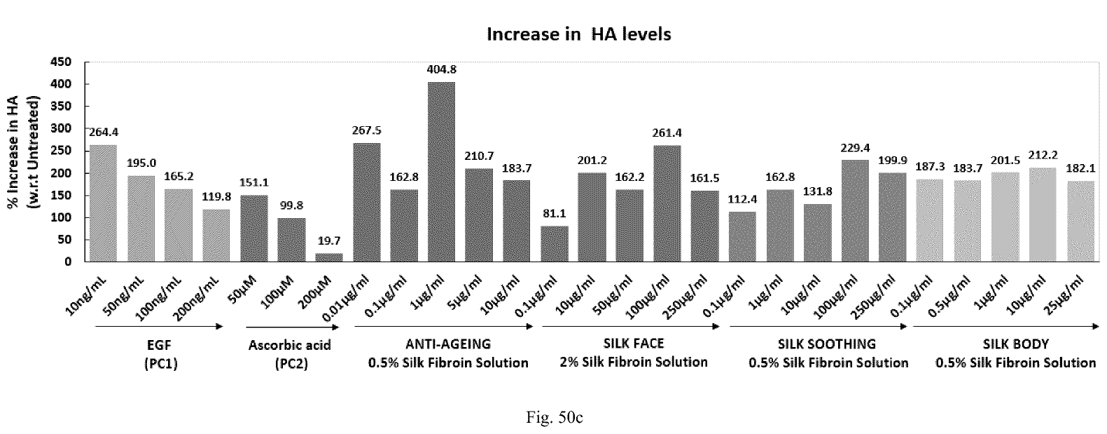

### Study Design Assay 1b) Skin Health Increase in ECM (HA, Elastin) (as Seen in FIGS. 50a-50c)

### Study Design

Test System—Human Fibroblasts (HFF 1)
Number of cells plated—50,00 cells/12 well plates
FBS concentration—0.5%,
Test items—Anti Ageing/Mineral SPF, Silk Face/Silk serum, Silk Soothing/Soothing lotion, Silk Body/Body lotion
Time points—3 days
Estimation method—BrdU assay
Positive Control/s—Ascorbic acid, EGF

### Procedure

HFF-1 cells were plated in 12 well plate at the density of 50,000 cells/well in DMEM supplemented with 15% FBS.
Following overnight incubation, the cells were subjected to serum starvation for 24 hours in DMEM+0% FBS.
Post serum starvation, cells were treated with non-cytotoxic concentrations of TIs and PC.
Following 72 hours of treatment with the TIs and PC, supernatants were collected.
The levels of HA/elastin were determined using ELISA kit as per manufacturer's protocol. Briefly,
   i. Standard and samples (Elastin (and HA 10× dilution)) were added to respective wells and incubated for 2 hours at 37 C
   ii. The contents of each well were removed, Biotin antibody was added to each well and incubated for 1 hour at 37 C
   iii. Following a wash 3 times) to remove any unbound conjugate, HRP avidin solution was added to each well and incubated for 1 h at 37 C
   iv. The plate was again washed 5 times with wash buffer and TMB substrate was added to each well followed by incubation for 30 minutes at 37 C in the dark
   v. The reaction was stopped by adding Stop solution to each well
   vi. The optical density of the color was measured at 450 nm

### Calculations

Increase in proliferation was calculated wrt control (untreated cells).

$$[ ( A - B ) / B ] * 100 ; Where A = Conc in Test Item treated cells , \\ B = Conc in Control ( Untreated ) cells$$

### 1c) Increase in Cell Migration of Fibroblasts

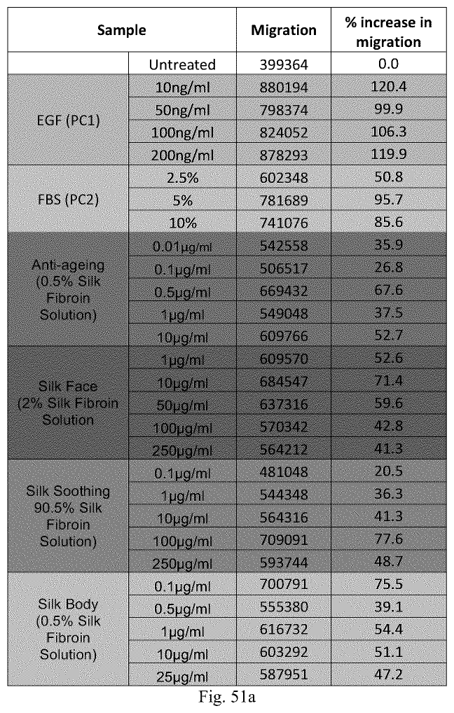

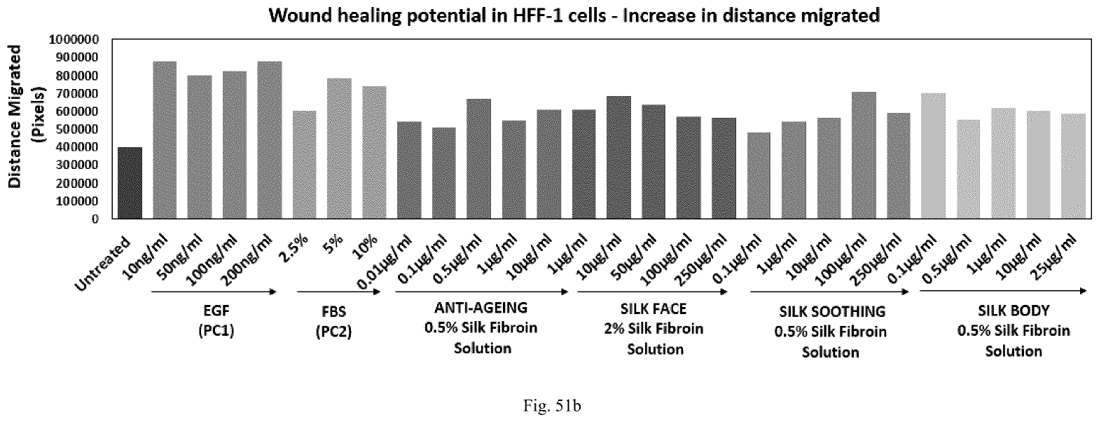

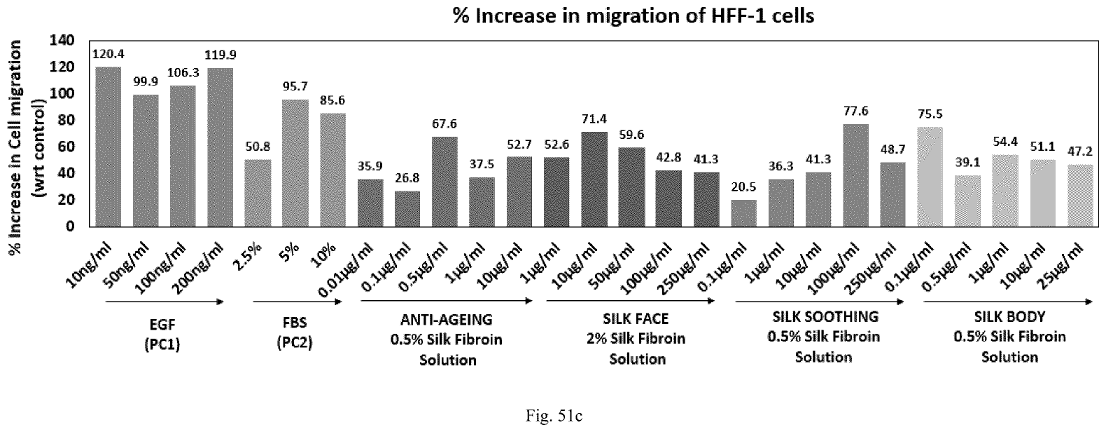

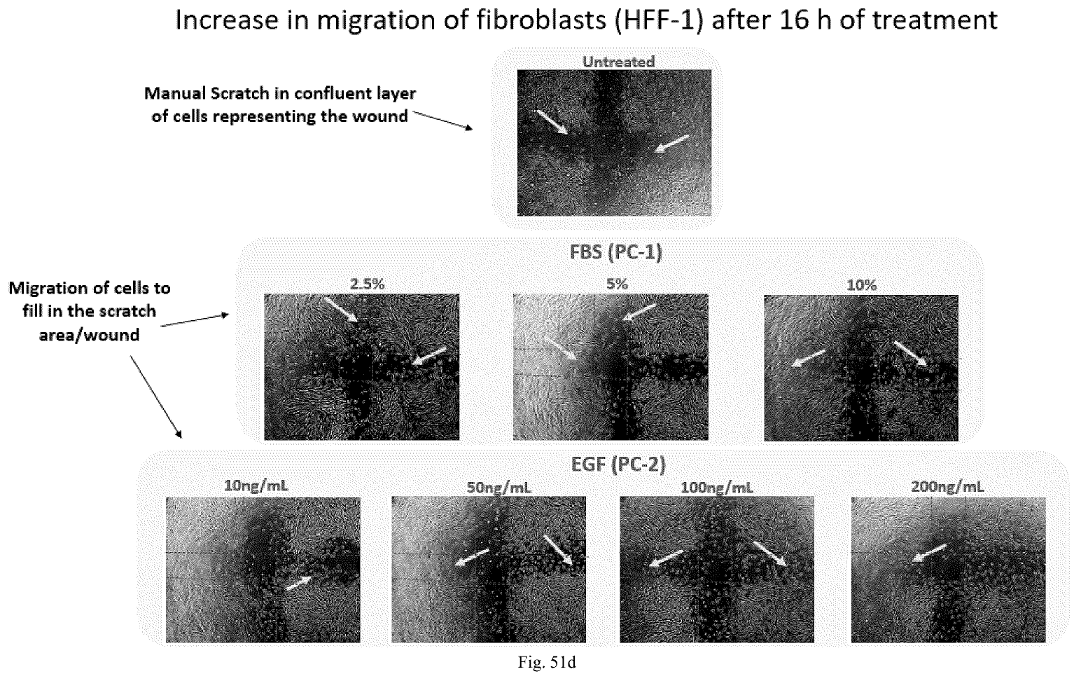

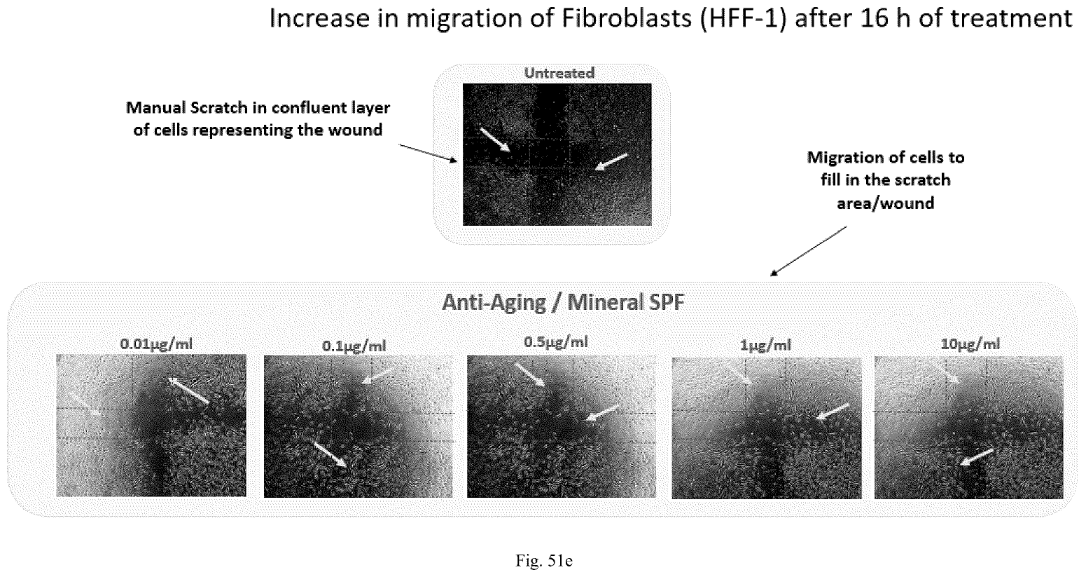

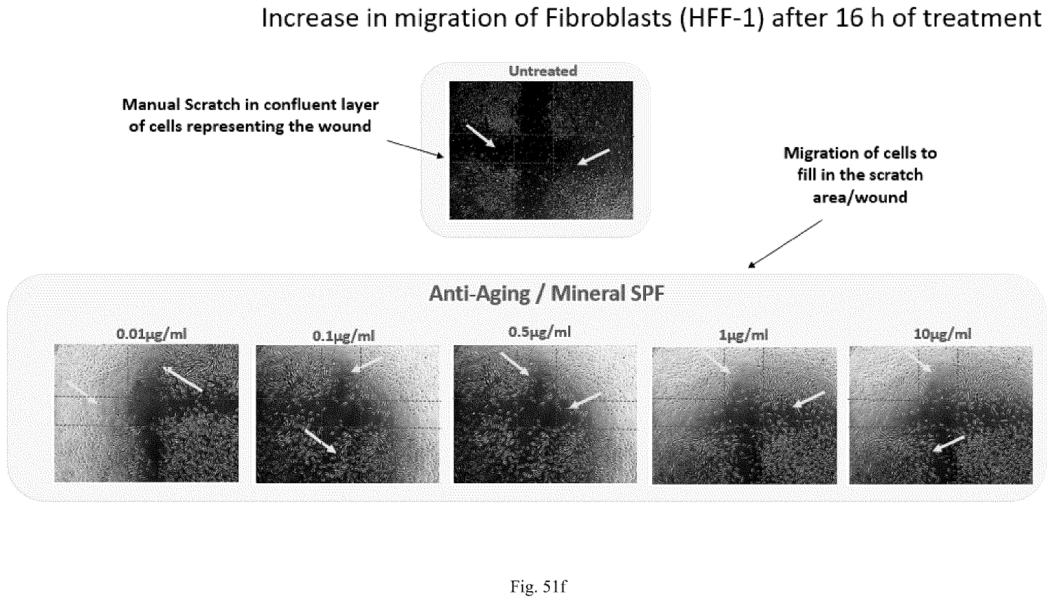

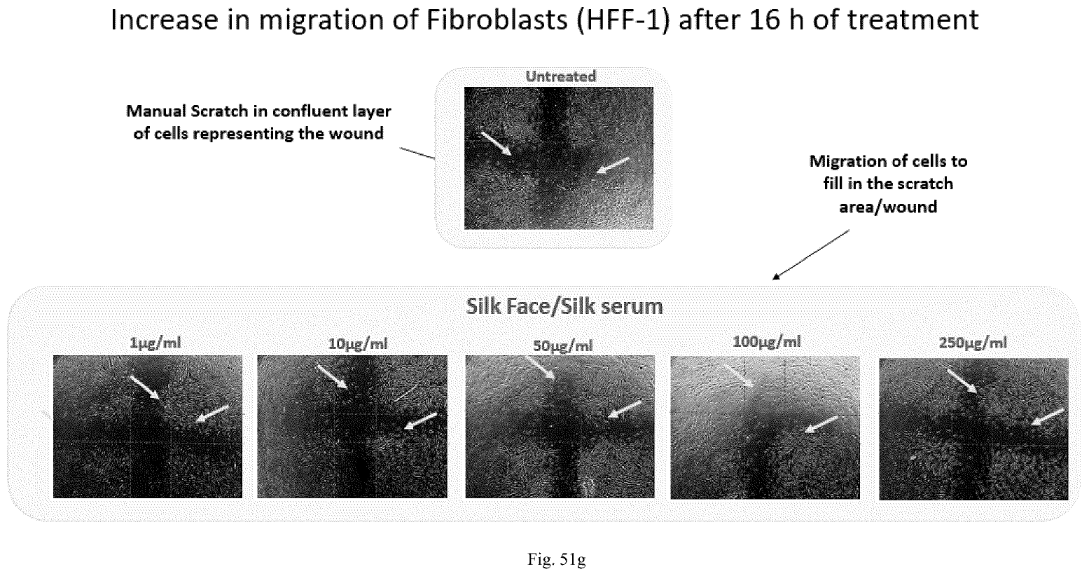

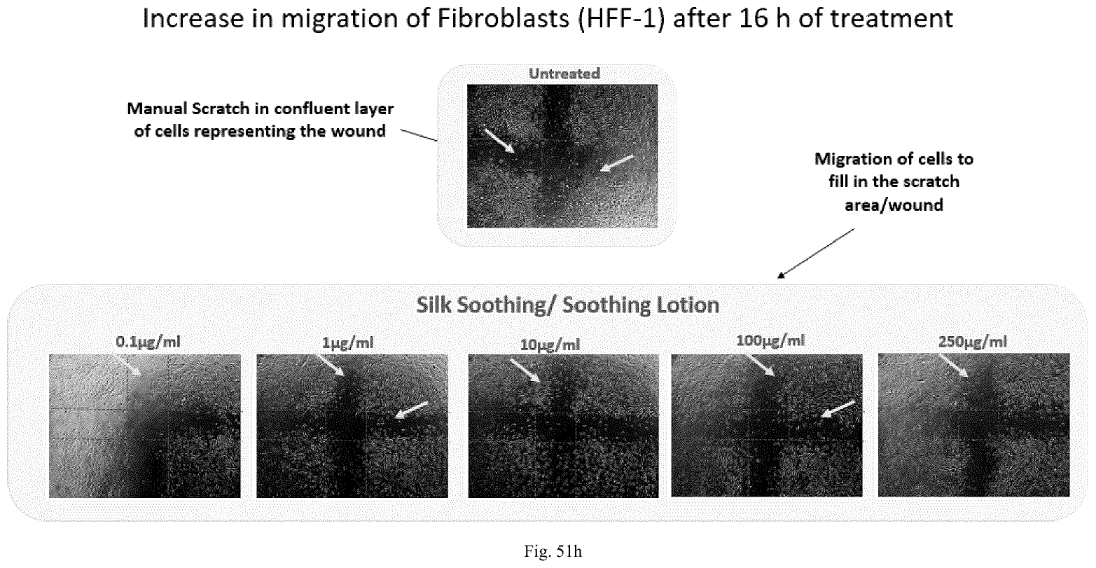

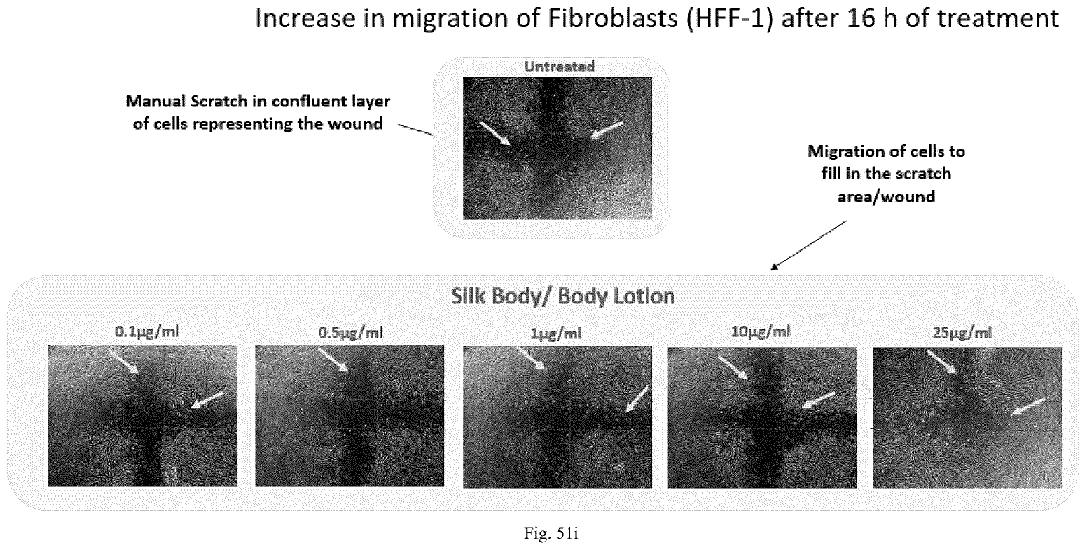

Study Design Assay 1c) Skin Health Increase in Cell Migration (as Seen in FIGS. 51a-51i)

### Study Design

Test System—Human Fibroblasts (HFF 1)
Number of cells plated—0.1 million cells/well
FBS concentration—0%
Test Items—Anti Ageing/Mineral SPF, Silk Face/Silk serum, Silk Soothing/Soothing lotion, Silk Body/Body lotion
Time point—16h
Estimation method—Scratch assay
Positive Control/s—EGF, FBS

### Procedure

Cells were plated in 10% FBS in 12-well plate and incubated for 24 h.
Cells were serum starved in 0% FBS for 24 h.
Scratch was created in each well using a sterile tip followed by treatment of cells with 4 Test items with non
cytotoxic concentrations of TIs and PCs.
Images were captured (0h).
After 16 h, the images were captured to determine the effect of Test Items on migration.
Increase in migration was calculated using ImageJ software.

### Calculations

Increase in migration was calculated wrt control (Untreated cells).

$$[ ( A - B ) / B ] * 100 ; Where A = \% Migration in Test Item treated cells , \\ B = \% Migration in Control ( Untreated ) cells$$

The extent of Migration/Distance migrated was calculated as: Empty space (pixels) at 0 h—Empty space (pixels) at 24 h
For each sample, respective zero h control was taken into consideration.

### 1d) Inhibition of MMPs in Fibroblasts

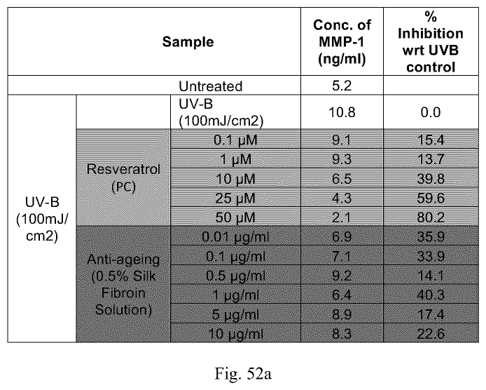

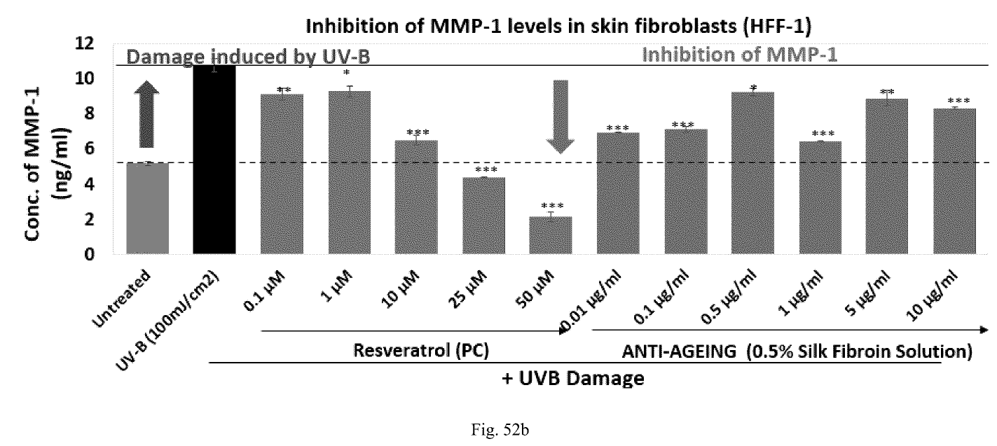

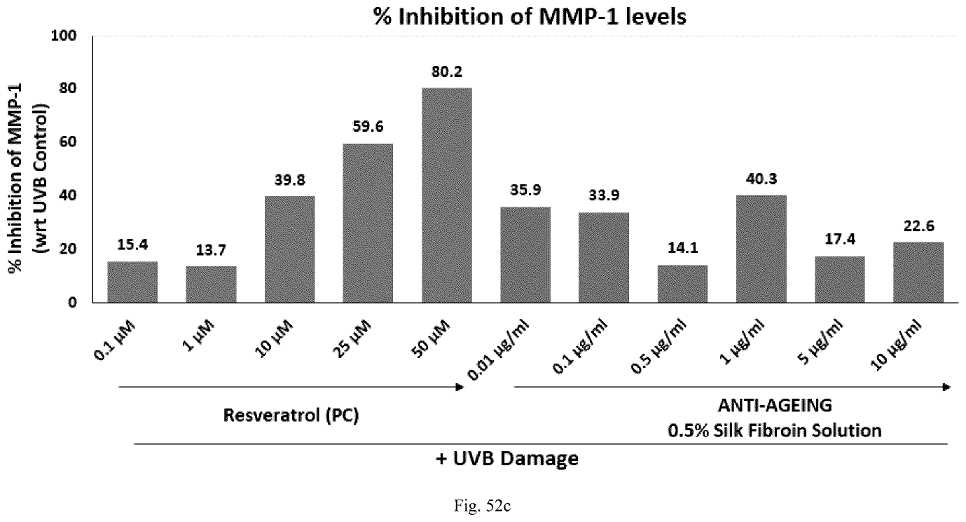

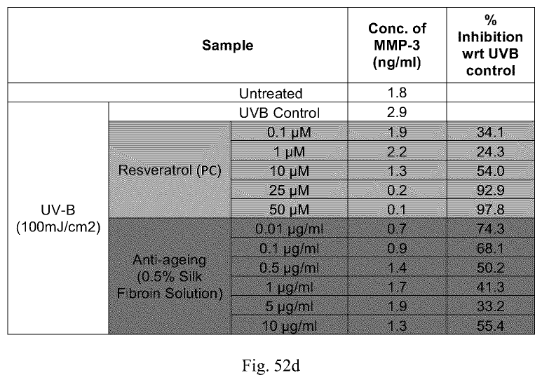

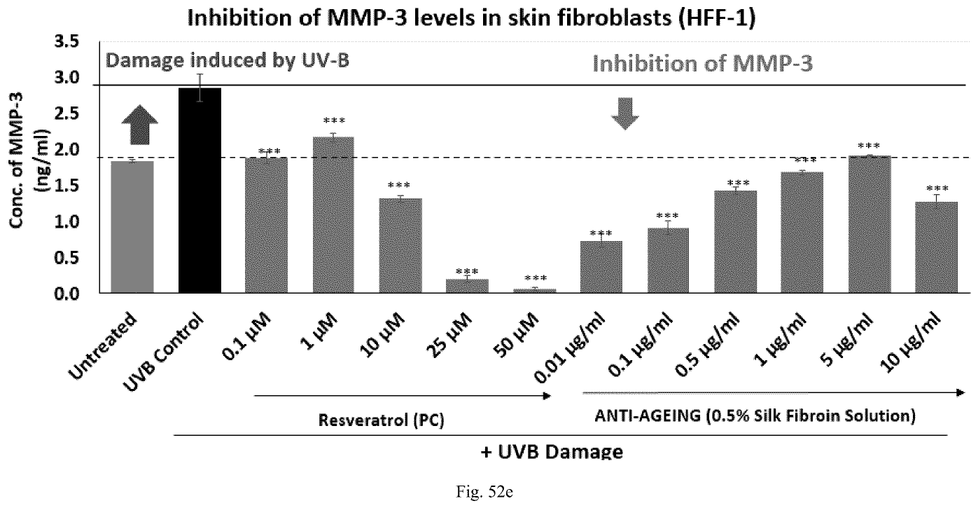

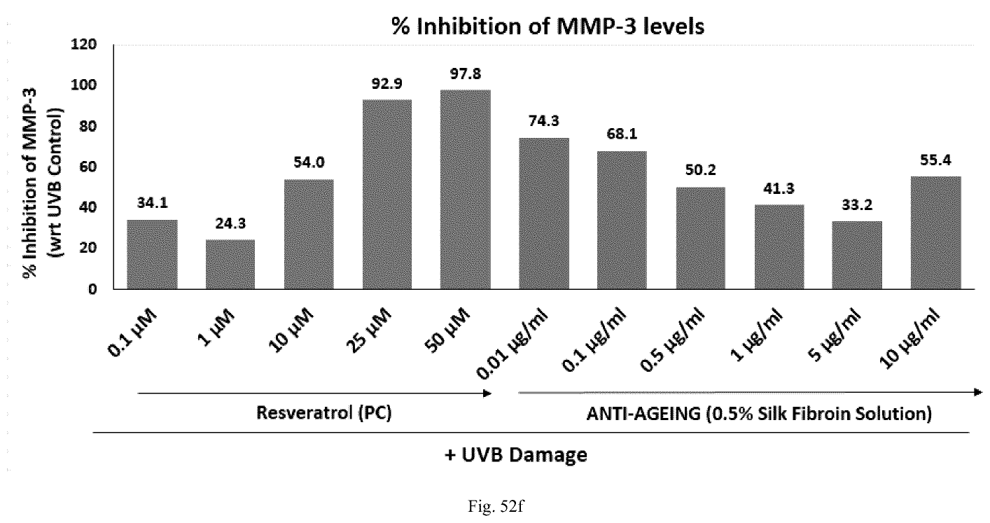

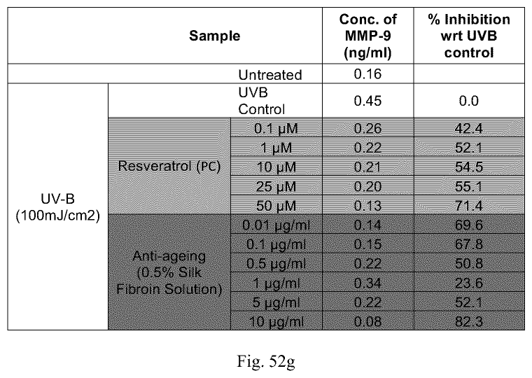

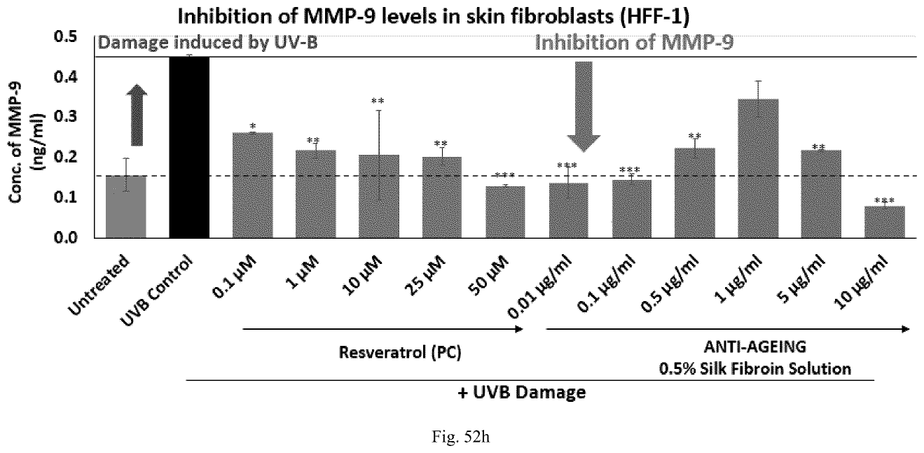

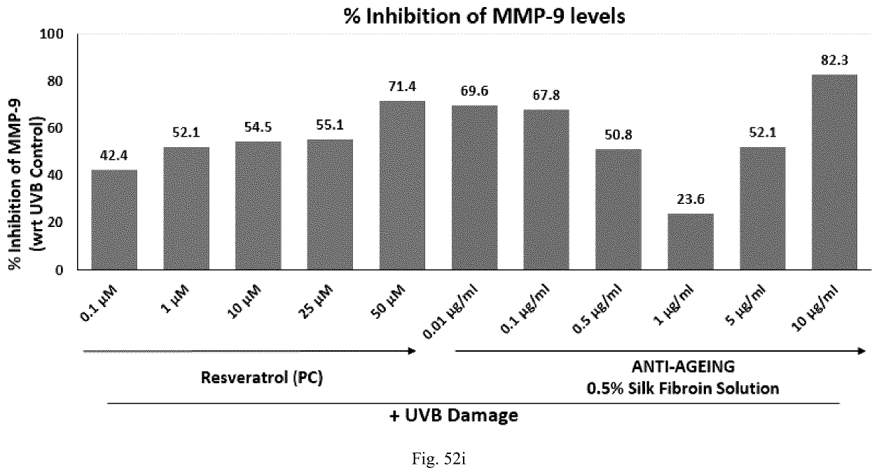

Study Design Assay 1d) Skin Health Inhibition of MMPs (as seen in FIGS. 52a-52i)

### Study Design

Test System—HFF 1 (Skin
Number of cells plated—0.25 million cells/well
FBS concentration—0%
Test Item—Anti Ageing/Mineral SPF
Time point—24h pretreatment, UVB exposure (100 mJ/cm2), recovery 24h
Estimation method—ELISA
Positive Control/s—Resveratrol

### Procedure

Cells were plated in 6-well plates in 10% FBS and incubated for 24 h.

Cells were serum starved in 0.10% FBS for 24 h.
Cells were pre-treated with 4 Test items at non cytotoxic concentrations for 24 h.
Cells were exposed to UVB irradiation (100 mJ/cm2) for 5.5 min in
PBS was washed off and replaced with serum-free medium and allowed for recovery for 24 h.
After 24h, culture supernatants were collected. Levels of MMPs were determined by ELISA as follows
i. Standard and samples were added to respective wells and incubated for 2 hours at 37C.
ii. The contents of each well were removed, Biotin antibody was added to each well and incubated for 1 hour at 37 C.
iii. Following a wash (3 times) to remove any unbound conjugate, HRP avidin solution was added to each well and incubated for 1 h at 37 C.
iv. The plate was again washed 5 times with wash buffer and TMB substrate was added to each well followed by incubation for 30 minutes at 37 C in the dark.
v. The reaction was stopped by adding Stop solution to each well.
vi. The optical density of the color was measured at 450 nm.

### Calculations

% Inhibition in MMP levels was calculated wrt control (UVB stimulated cells).

$$[ ( B - A ) / B ] * 100 ;$$

$$Where A = Levels of MMP 1 / 3 / 9 in Test Item treated cells , \\ B = Levels of MMP 1 / 3 / 9 in Control ( UVB stimulated cells )$$

### 2. Skin Health—Keratinocytes Based Assays

### 2a) Increase in Cell Proliferation of Keratinocytes

### Study Design Assay 2a) Skin Health Increase in Keratinocytes Proliferation

### Study Design

Test System—Human Keratinocytes (HaCaT)
Number of cells plated—1000 cells/well
FBS concentrations—0.1%
Test Items—Anti Ageing/Mineral SPF, Silk Face/Silk serum, Silk Soothing/Soothing lotion, Silk Body/Body lotion
Time point—3d, single addition
Estimation method—BrdU assay
Positive Controls—Ascorbic acid, EGF

### Procedure

Cells were plated in 10% FBS in 96-well plates and incubated for 24 h.
Cells were serum starved in 0.10% FBS for 24 h.
Cells were treated with 4 Test items at various concentrations.
After 3 days (72h), the effect on proliferation was determined by BrdU assay.
i. 10 μl of BrdU solution was added/well and cells were incubated for 90 minutes at 37° C.
ii. After removing medium, cells were fixed, and the DNA was denatured in one step by adding 200 μl of FixDenat Cells were incubated for 30 min at RT 15 25° C.)
iii. The FixDenat solution was removed and 100 μl of Anti BrdU POD solution was added to each well Cells were incubated for 90 min at RT 15-25° C.)
iv. Wells were washed 3 times using 200 μl of washing solution
v. 100 μl/well of substrate solution was added Cells were incubated for 30 min at RT 15 25° C.)
vi. After incubation, a blue coloured complex was formed Absorbance of each well was measured at 370 nm

### Calculations

Increase in proliferation was calculated wrt control (Untreated cells).

$$[ ( A - B ) / B ] * 100 ; Where A = Absorbance in Test Item treated cells , \\ B = Absorbance in Control ( Untreated ) cells$$

### 2b) Increase in Cell Migration of Keratinocytes

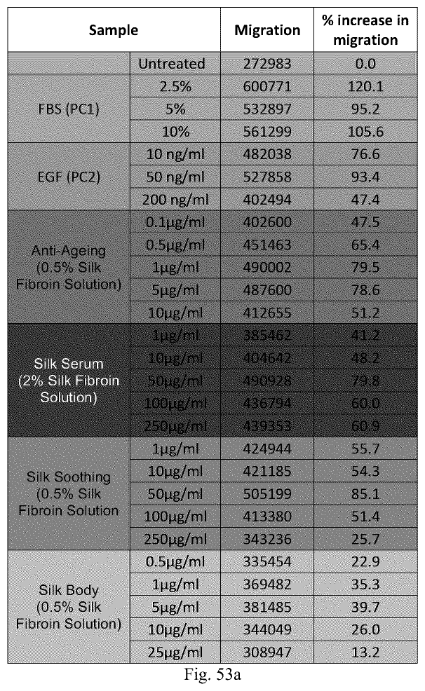

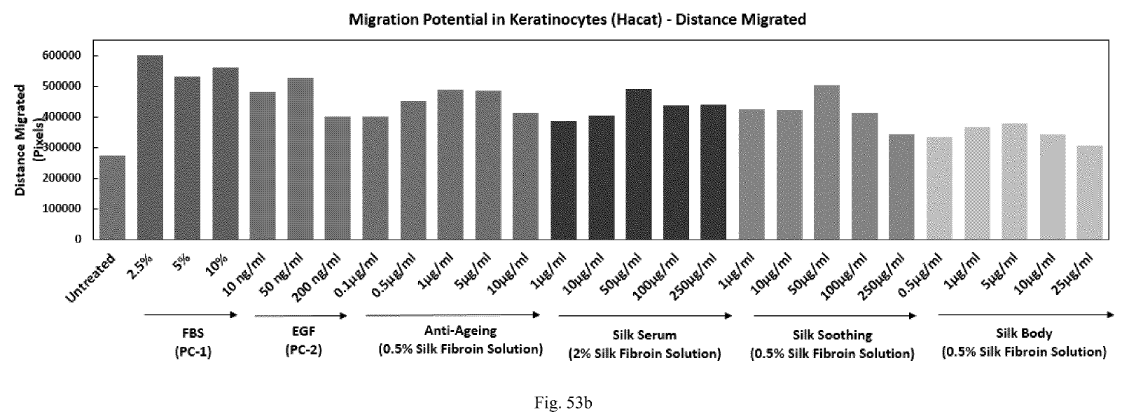

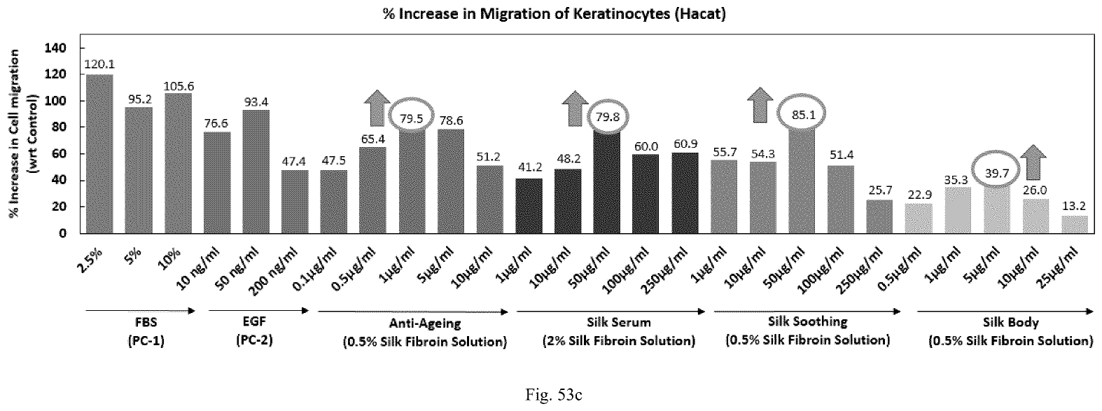

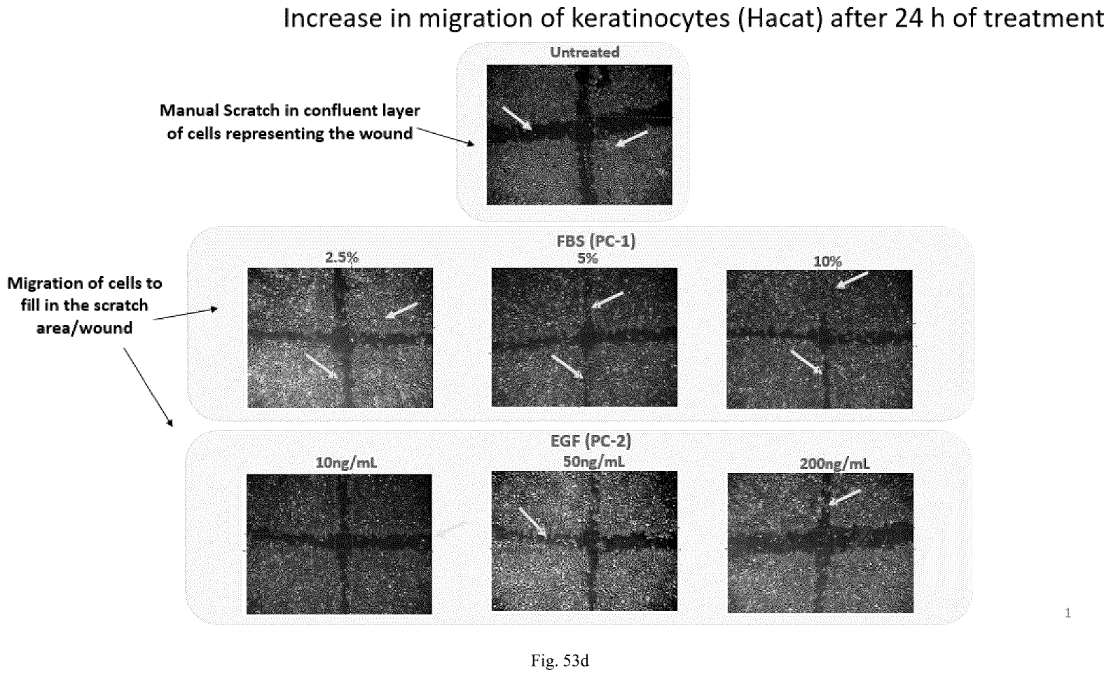

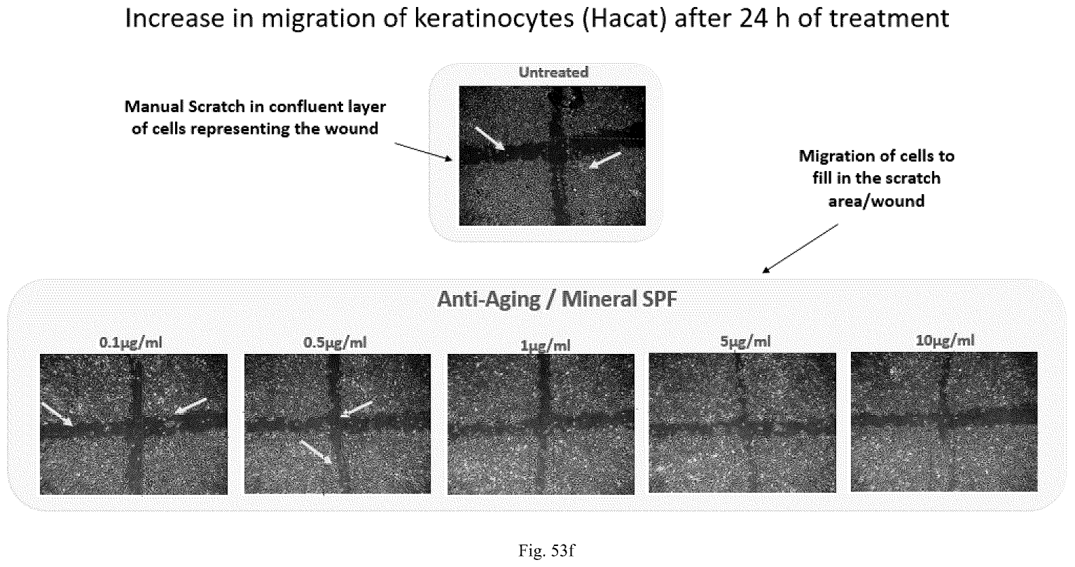

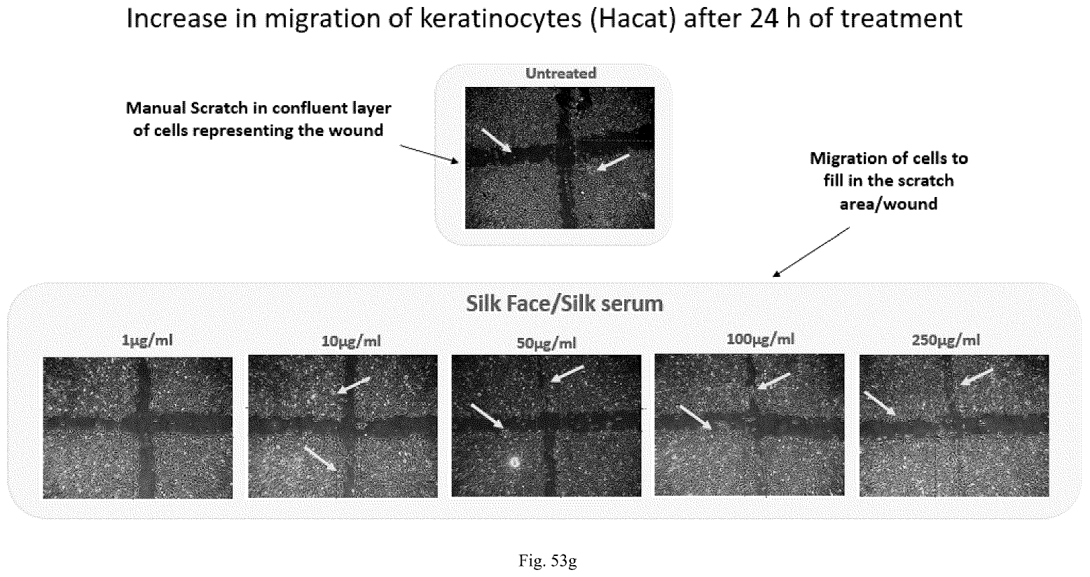

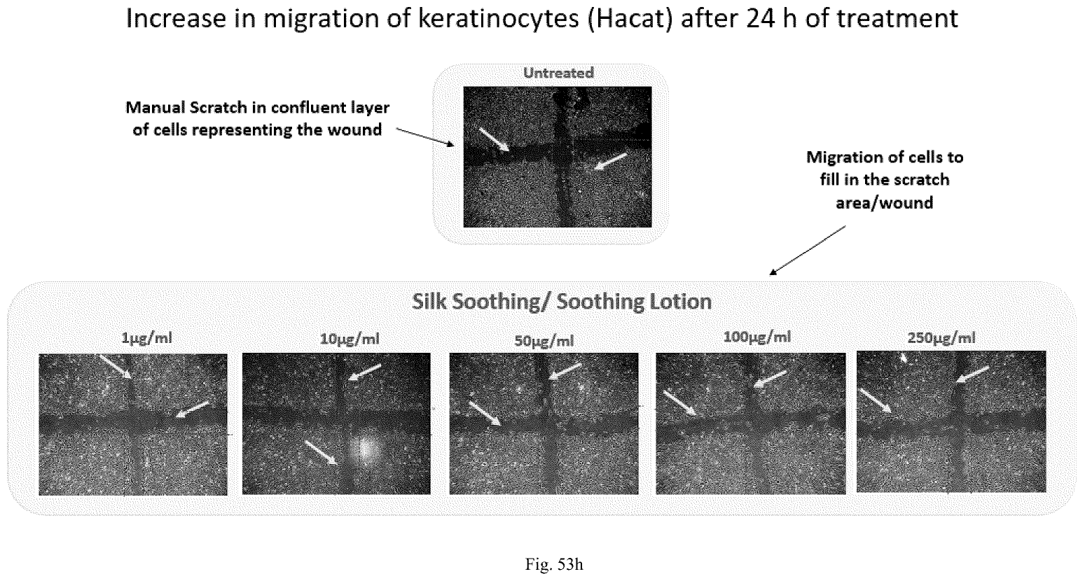

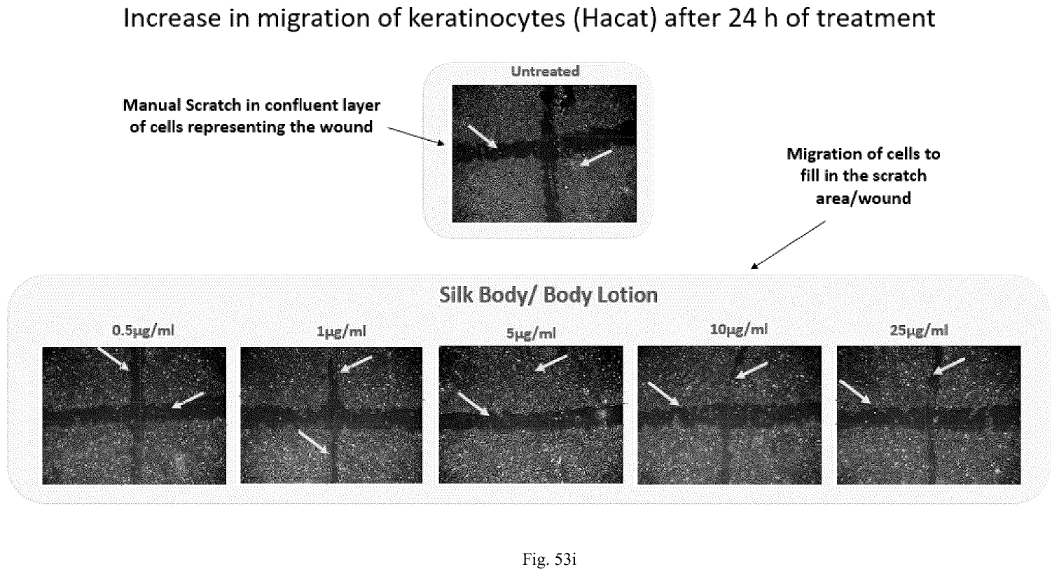

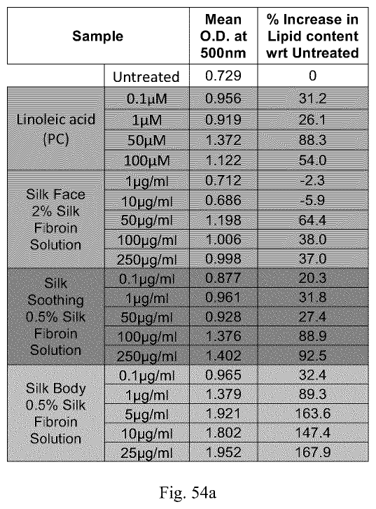

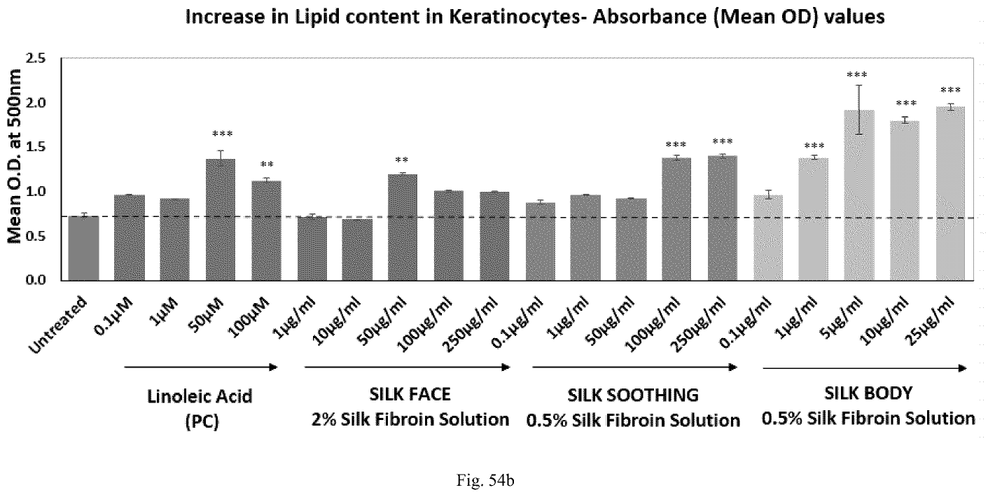

Study Design Assay 2b) Skin Health Increase in Cell Migration (as Seen in FIGS. 53a-53i)

### Study Design

Test System—Human Keratinocytes (HaCaT)
Number of cells plated—0.1 million cells/well
FBS concentration—0%
Test Items—Anti Ageing/Mineral SPF, Silk Face/Silk serum, Silk Soothing/Soothing lotion, Silk Body/Body lotion
Time point—24h
Estimation method—Scratch assay

### Procedure

Cells were plated in 10% FBS in 12-well plate and incubated for 24 h.
Cells were serum starved in 0% FBS for 24 h.
Scratch was created in each well using a sterile tip followed by treatment of cells with 4 Test items with non-cytotoxic
concentrations of TIs and PCs.
Images were captured (0h).
After 24 h, the images were captured to determine the effect of Test Items on migration.
Increase in migration was calculated using ImageJ software.

### Calculations

Increase in migration was calculated wrt control (Untreated cells).

$$[ ( A - B ) / B ] * 100 ;$$

$$Where A = \% Migration in Test Item treated cells , B = \% Migration in Control ( Untreated ) cells$$

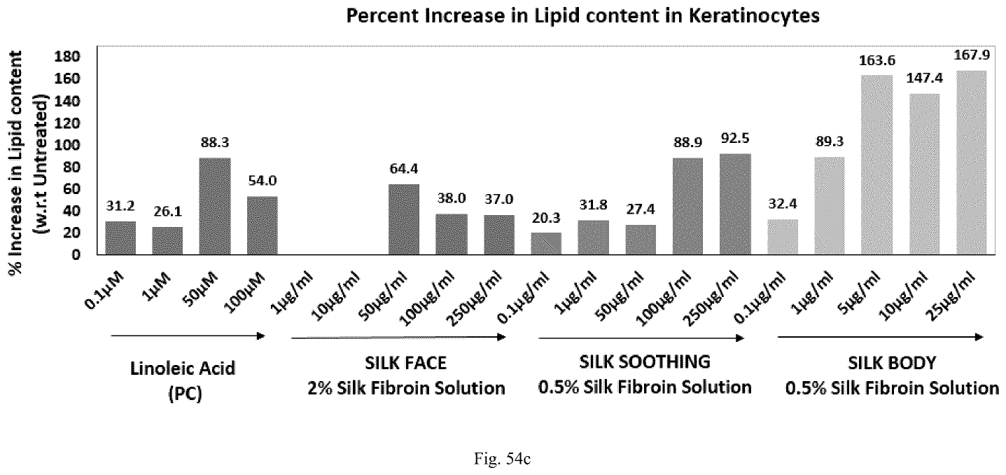

The extent of Migration/Distance migrated was calculated as: Empty space (pixels) at 0 h Empty space (pixels) at 24 h
For each sample, respective zero h control was taken into consideration. 2c) Increase in lipid content in keratinocytes
   Study Design Assay 2c) Skin Health Increase in Lipid Content in Keratinocytes (HaCaT) (as Seen in FIGS. 54a-54c)

### Study Design

Test System—Human Keratinocytes (HaCaT)
Number of cells plated—50,000 cells/well in 12 well plate
FBS concentrations—0% FBS
Test Items—Silk Face/Silk serum, Silk Soothing/Soothing lotion, Silk Body/Body lotion
Time points—48h
Estimation method—Lipid content by Oil Red O dye (fat soluble dye that stains neutral triglycerides and lipids, used for
quantitative and qualitative measurement of lipid droplet formation)
Positive Control—Linoleic Acid

### Procedure

Cells were plated in 12-well plates in 10% FBS and incubated for 24 h.
Cells were sera starved with 0% FBS for 24h.
Cells were treated with 3 Test items at non-cytotoxic concentrations for 48 h.
After 48 hours, cells were fixed and lipid content was measured by staining with Oil-O Red dye.
   1. Cell layer was washed with PBS and fixed in 10% formalin. Cells were washed with 60% Isopropanol and air dried.
   2. Air-dried cell layers were stained with Oil Red O stain at RT.
   3. Cell layers were washed with Milli-Q water to remove any unbound stain.
   4. The lipid bound stain was eluted with 100% Isopropanol and optical density was measured at 500 nm.
   5. Percent increase of lipid content was calculated wrt control.

### Calculations

Percent Increase in each sample was calculated wrt control (Untreated cells).

$$[ ( A - B ) / B ] * 100 ;$$

$$Where A = Absorption in Test Item treated cells , B = Absorbance in Control ( untreated ) cells$$

### 2d) Improvement of Skin Barrier by CE Formation in Keratinocytes

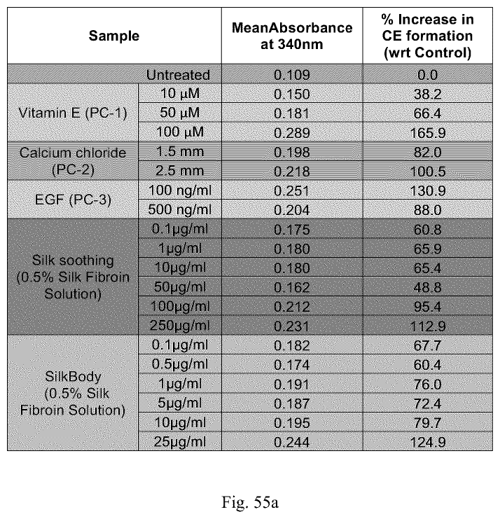

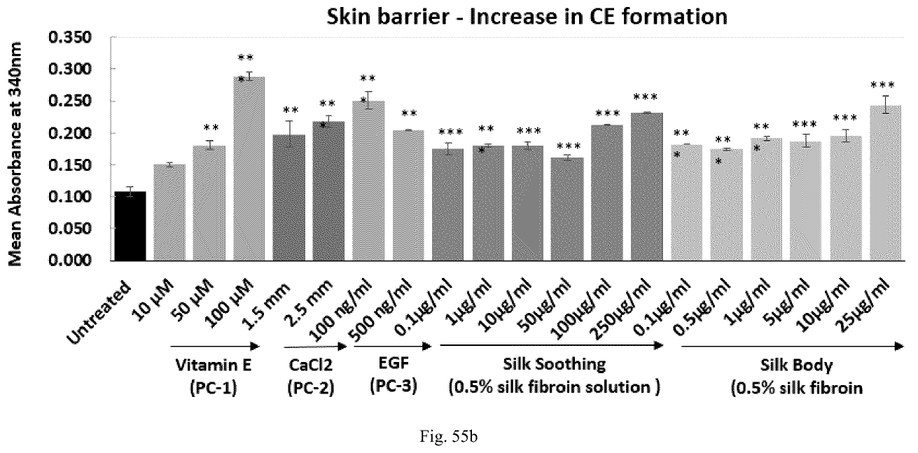

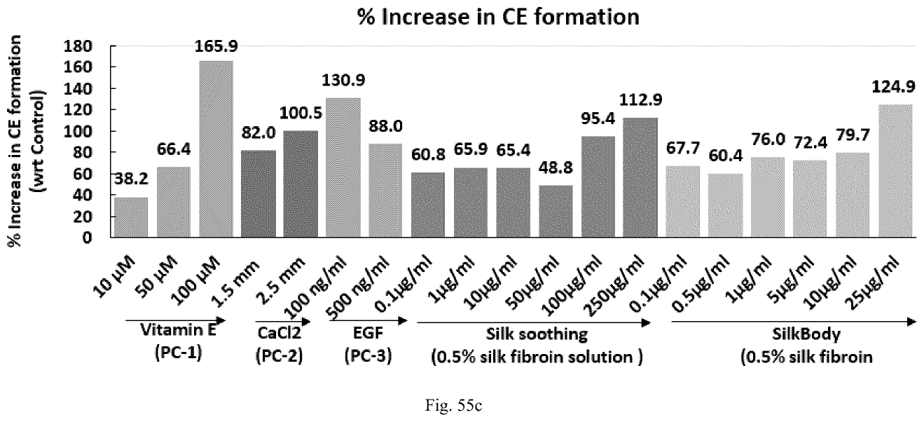

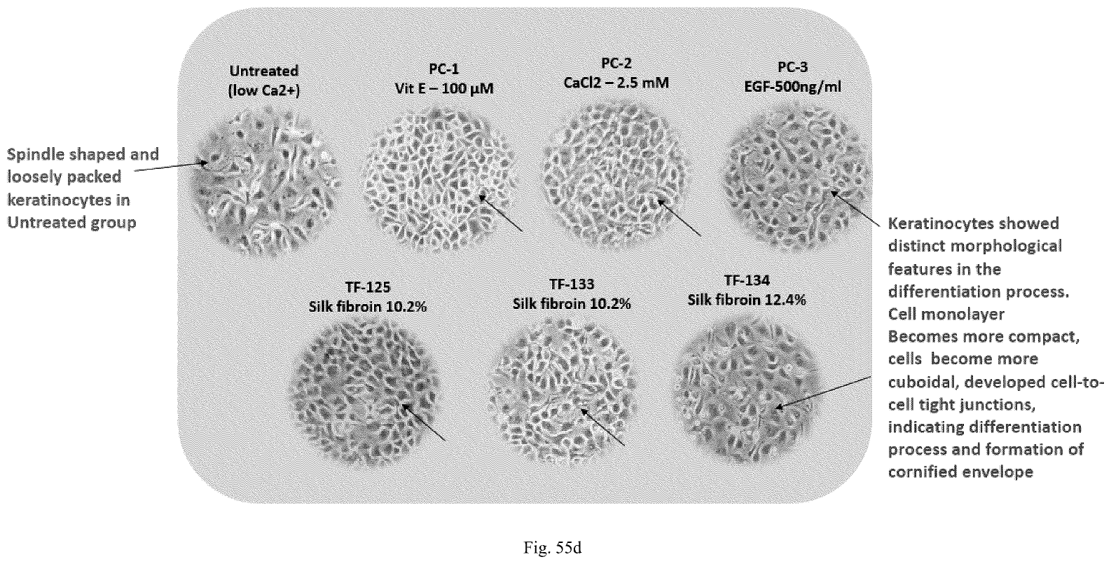

Study Design Assay 2 d) Skin Health Improvement of Skin Barrier Formation by Increased Formation of Cornified Envelope (CE) (as Seen in FIGS. 55a-55d)

### Study Design

Test System—Human Keratinocytes (HaCaT)
Number of cells plated—1 million cells/6 well plates
FBS concentrations—0%, Low calcium
Test items—Silk Soothing/Soothing lotion, Silk Body/Body lotion
Time points—7 days, followed by re addition of Test Item on every 3 rd day.
Estimation method—Turbidity of membrane/Absorbance at 340 nm.
Positive Control/s—Vitamin E, Calcium chloride, EGF.

### Procedure

Cells were plated in 10% FBS in 6-well plates and incubated for 24 h.
Cells were treated with 2 Test items at various concentrations in calcium free medium for 7 days, replacing media every 3rd day.
After incubation, cells processed for skin barrier strengthening potential measured as follows
Cells were harvested by trypsinization in pre-labelled Eppendorf tubes and centrifuged at 300 g for 5 mins.
Supernatant was discarded and the cell pellet washed with 1×PBS to remove media residues.
Cells were washed at 300 g for 5 mins.
Supernatant was discarded and cell pellet was resuspended in 200 μl CE buffer (2% SDS, 20 mM DTT and 0.1M tris Buffer).
The mixture was boiled at 95° C. for 5 mins to dissolve crosslinked envelopes.
90 μl of mixture transferred to 96 well plates in duplicate and absorbance was measured at 340 nm.

### Calculations

Increase in formation of Cornified envelopes in each sample was calculated wrt control (Untreated cells).

$$[ ( A - B ) / B ] * 100 ;$$

$$Where A = Absorption in Test Item treated cells , B = Absorbance in Control ( Untreated ) cells$$

### 2e) Improvement of Skin Barrier by Increased Expression of Surface Proteins in Keratinocytes

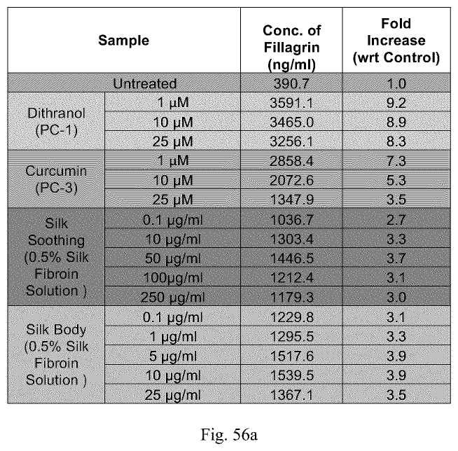

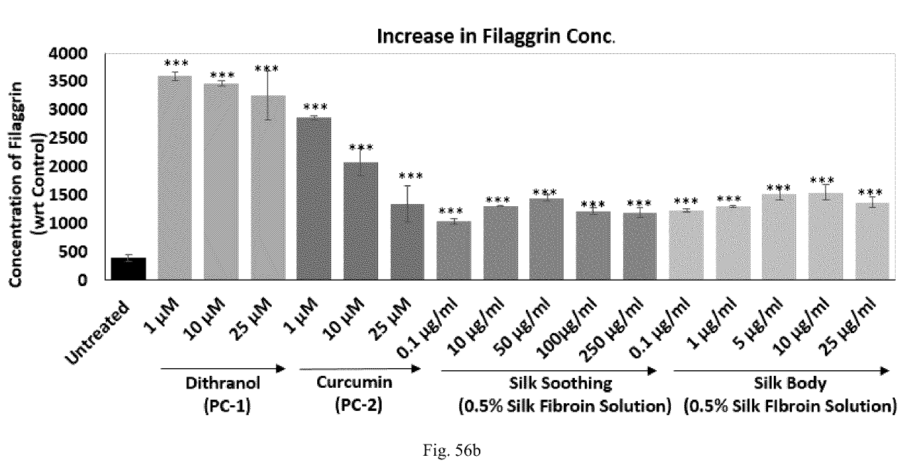

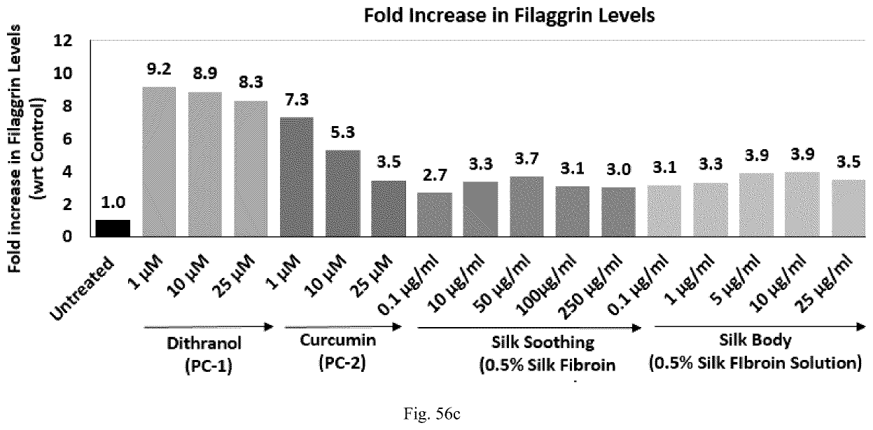

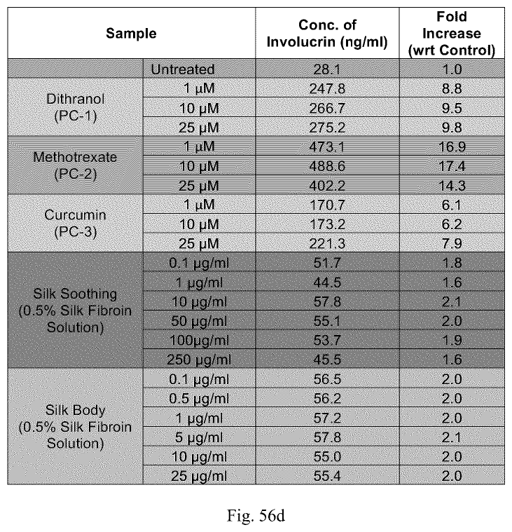

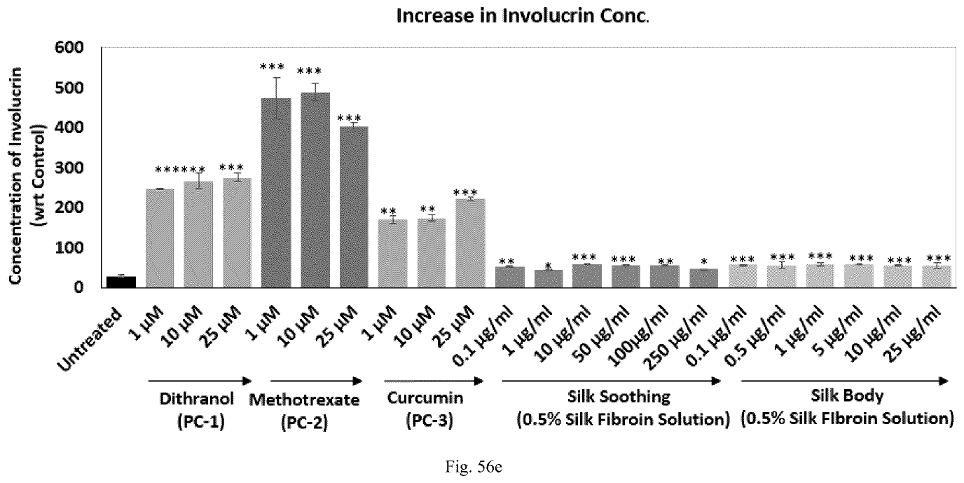

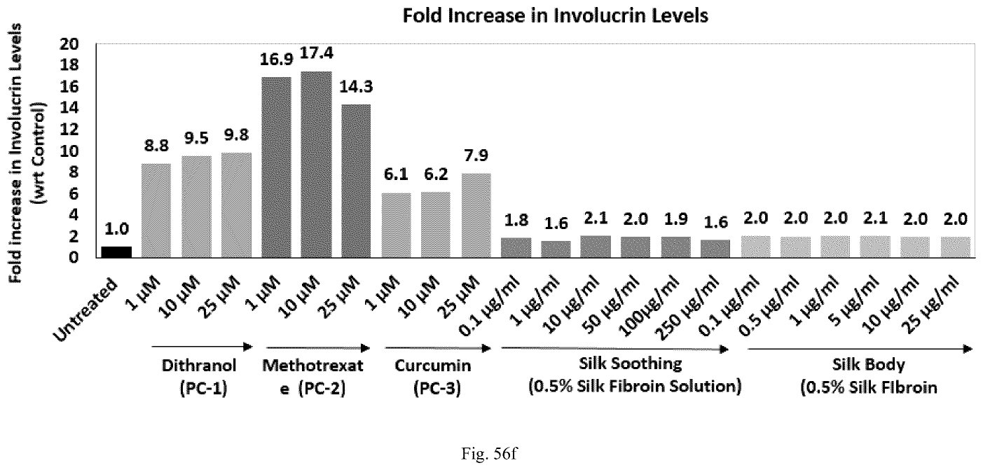

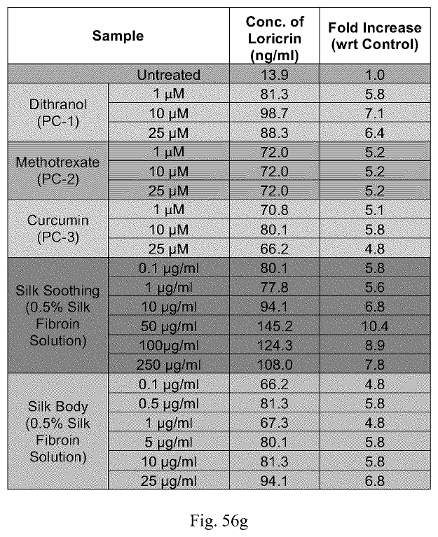

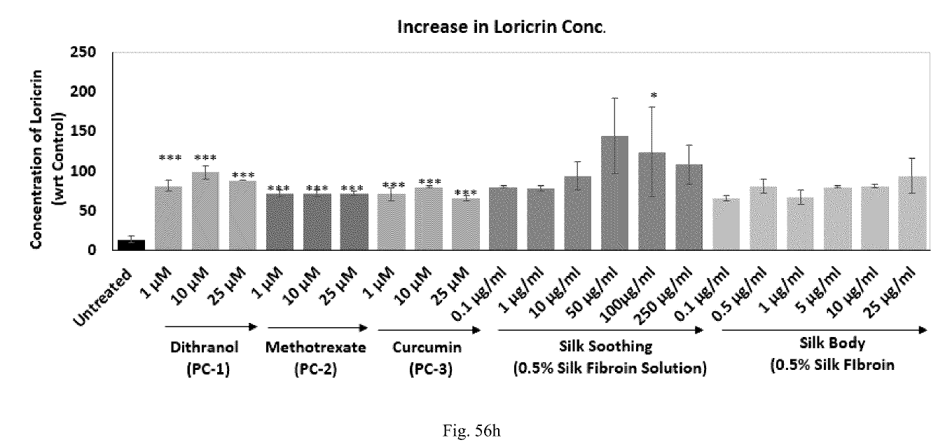

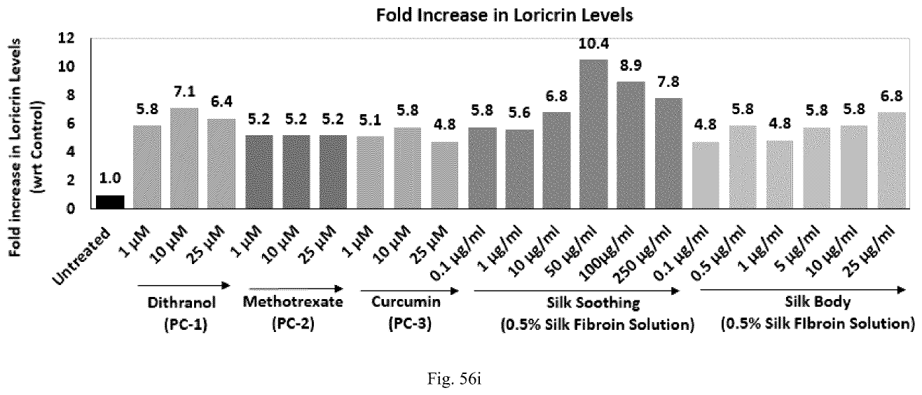

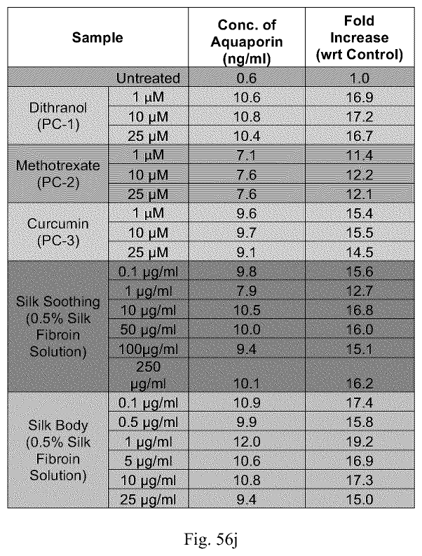

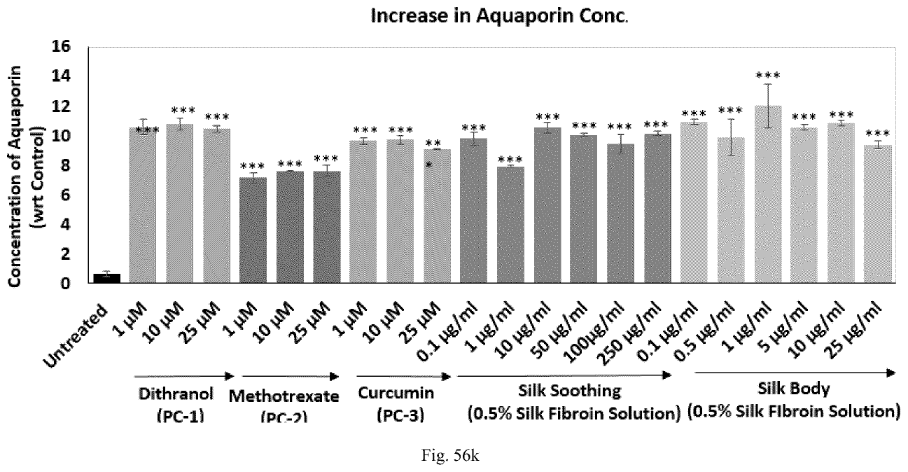

Study Design Assay 2e) Skin Health Increase in Expression of Surface Markers for improvement of Skin Barrier (as Seen in FIGS. 56a-56l)

### Study Design

Test System—Human Keratinocytes (HaCaT)
Number of cells plated—0.1 million cells/24 well plates
FBS concentrations—0%
Test items—Silk Soothing/Soothing lotion, Silk Body/Body lotion
Time points—72h
Estimation method—ELISA
Positive Control/s—Methotrexate, Dithranol, Curcumin.

### Procedure

Cells were plated in 10% FBS in 24-well plates and incubated for 24 h.
Cells were sera Starved with 0% FBS for 24h.
Cells were treated with 2 Test items in 0% FBS at various concentrations for 72h.
After incubation, culture supernatants were collected, and lysates were also prepared.
Levels of Filaggrin, AQP3 (supernatants) and Involucrin, Loricrin (lysates) were determined using ELISA as follows
   Respective kit standards and samples (supernatants of cells) were directly pipetted into the wells and incubated for 1 2 h at 37° C.
   Liquid was removed from each well and plate was incubated at 37° C. for 1 hour after addition of Biotin antibody.
   Following a wash (3 times) to remove any unbound conjugate, HRP avidin solution was added to the wells and incubated for 30 min at 37° C.
   Plate was again washed for 3 times.
   TMB substrate was added to each well for 15
   30 mins and plate followed by incubation at 37° C.
   The reaction was stopped by adding Stop solution to each well.
   The optical density of the color was measured at 450 nm.

### Calculations

Increase in Filaggrin levels in each sample was calculated wrt control (Untreated cells).

$$[ ( A - B ) / B ] * 100 ;$$

$$Where A = conc in Test Item treated cells , B = con c in Control ( Untreated ) cells .$$

### Anti-Aging Potential 3a) DNA Protection Against Oxidative Damage

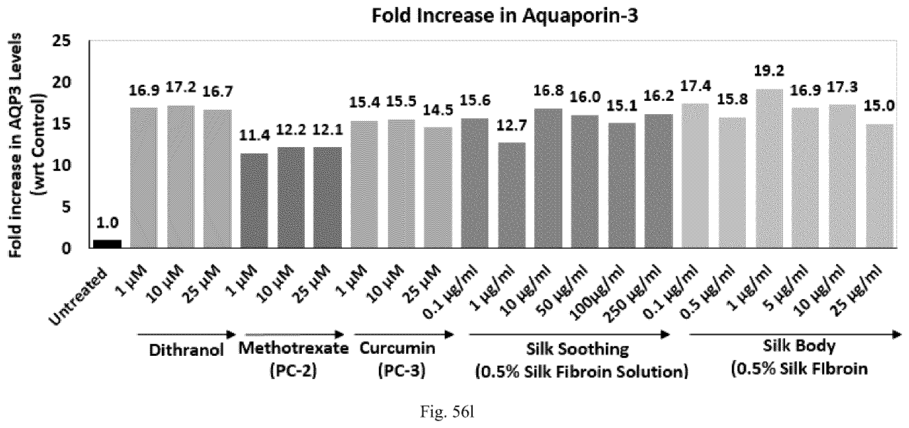

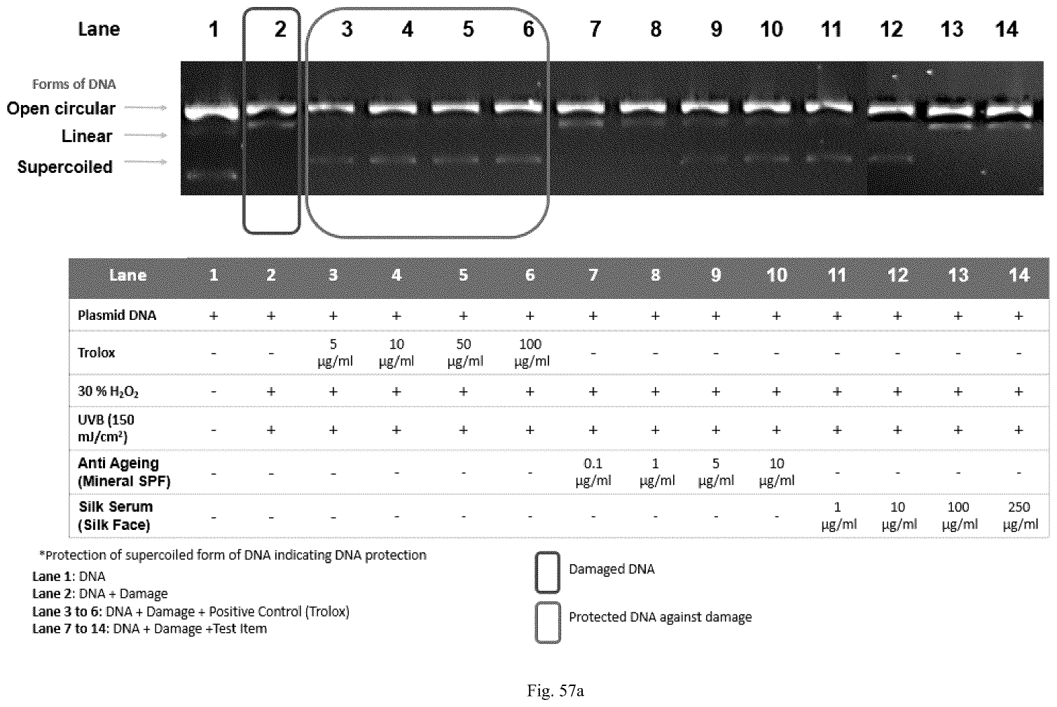

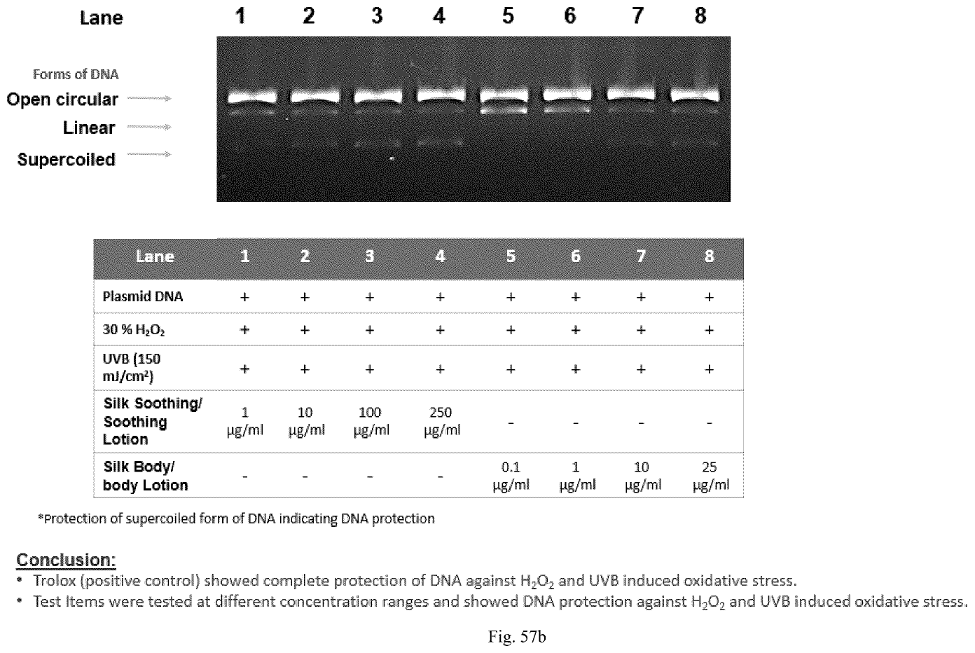

Results Assay 3a) Anti-Aging DNA Protection Against Oxidative+UV-B Damage (as Seen in FIGS. 57a-57b)

### Study Design

Test System—Cell free assay
Test items—Anti Ageing/Mineral SPF, Silk Face/Silk serum, Silk Soothing/Soothing lotion, Silk Body/Body lotion
Time point—1h
Estimation method—Restoration of Native DNA against oxidative damage measured qualitatively by Gel Electrophoresis
Positive Control—Trolox

### Procedure

15 μl of Plasmid DNA (pBR 322 Plasmid DNA from E coli) was incubated with different conc of 4 Test Items 2 μl) and Trolox (positive control) for 1 h at 37 □C
Oxidative damage was induced with 30H 2 O 2 (vol 2 μl) for 20 min at 37 □C and subsequently exposed to UVB damage
150 mJ/cm 2 for 5 min
After irradiation, samples were run on an electrophoresis gel using 1 agarose gel
Segregated form of plasmid DNA bands were analyzed and captured using BIORAD Gel documentation System
Restoration in the plasmid DNA pattern of test items treated DNA, as compared to control untreated DNA was observed

### Intact DNA Shows 3 Forms of DNA Open Circular, Linear and Supercoiled, Damaged DNA Showed Only 2 Bands, Open Circular and Linear, Protective Effect Showed Reappearance of Supercoiled Band

### Interpretation

Restoration of Native DNA against oxidative damage

### Anti-Aging Potential—4. Fibroblasts Based Assays

### 4a) Cytoprotection Against UVB in Fibroblasts

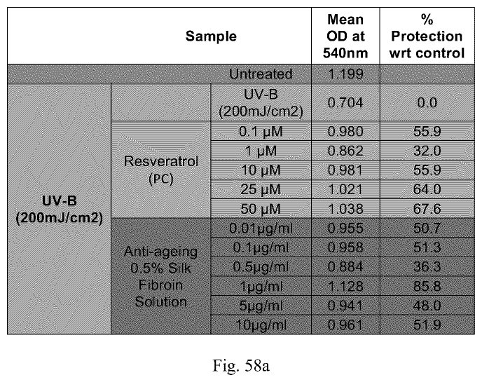

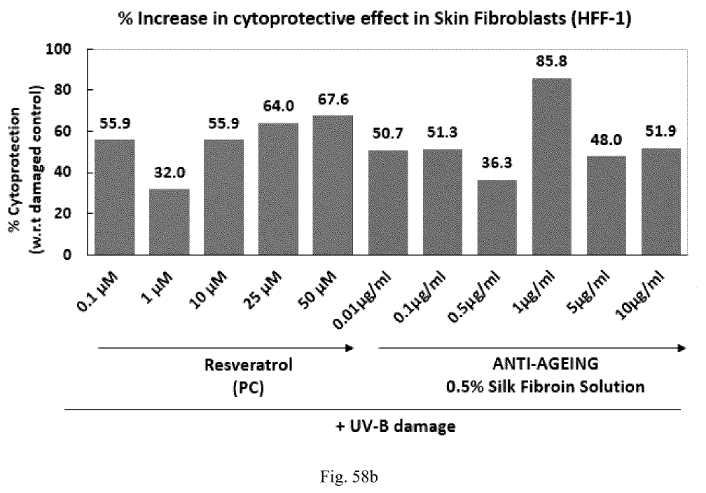

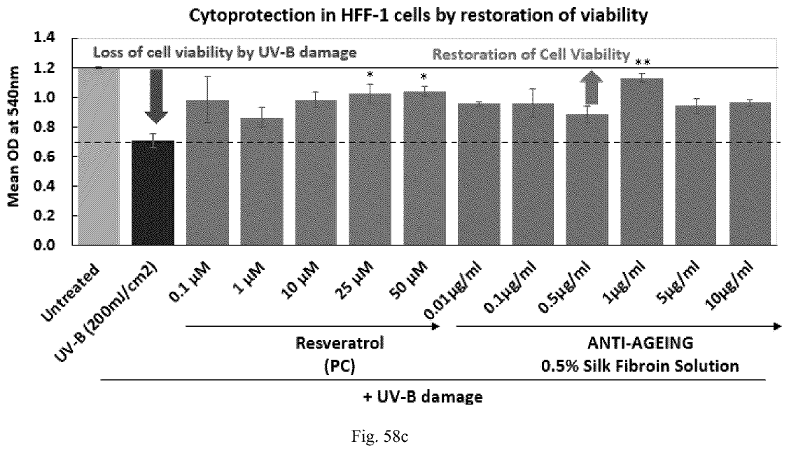

Study Design Assay 4a) Anti-Aging Cytoprotection Against UV B Induced Damage (as Seen in FIGS. 58a-58c)

### Study Design

Test System—HFF 1 (Skin
Number of cells plated—8000 cells/well plate
FBS concentrations—0.1%
Test items—Anti Ageing/Mineral SPF
Time points—24h pretreatment, UVB exposure (200 mJ/cm2), recovery 24h
Estimation method—MTT assay
Positive Control—Resveratrol

### Procedure

Cells were plated in 96-well plates in 10% FBS and incubated for 24 h.
Cells were pre-treated with Test Item at various concentrations for 24 h.
Cells were exposed to UVB irradiation (200 mJ/cm2) for 15 min in PBS.
PBS was washed off and replaced with medium and allowed for recovery for 24 h.
After 24h, cell viability was assessed by MTT assay.
   1. 20 μl of MTT solution was added to each well and cells were incubated for 3 h at 37° C.
   2. After incubation, supernatants were removed and 150 μl DMSO added to all wells to extract formazan crystals.
   3. A purple coloured formazan complex was formed. Absorbance of each well was measured at 540 nm.

### Calculation

Protective effect on cell viability was calculated wrt controls (untreated and UV B damaged cells).

$$\{ ( Absorbance of TI + UVB ) - ( Absorbance of UVB alone ) / ( Absorbance of Untreated ) -- ( Absorbance of UVB alone ) \} * 100$$

### 4b) Cytoprotection Against Oxidative Stress in Fibroblasts

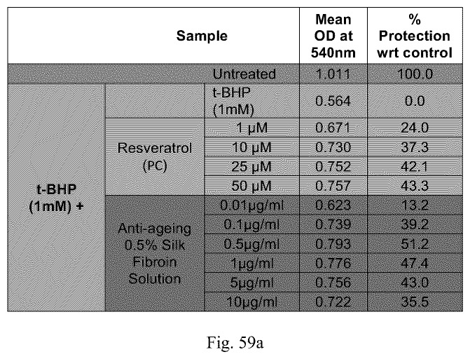

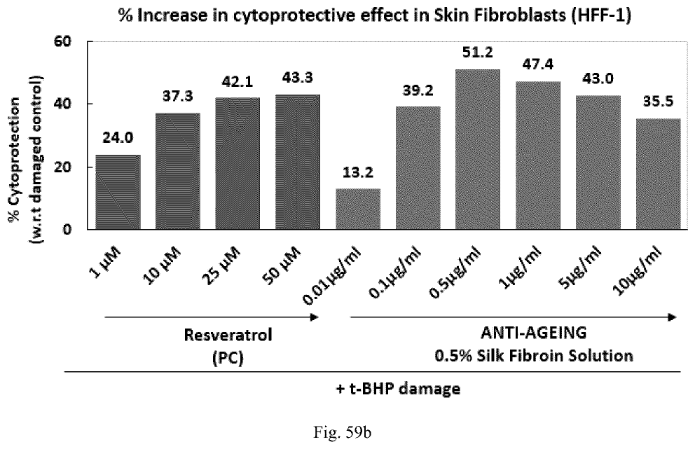

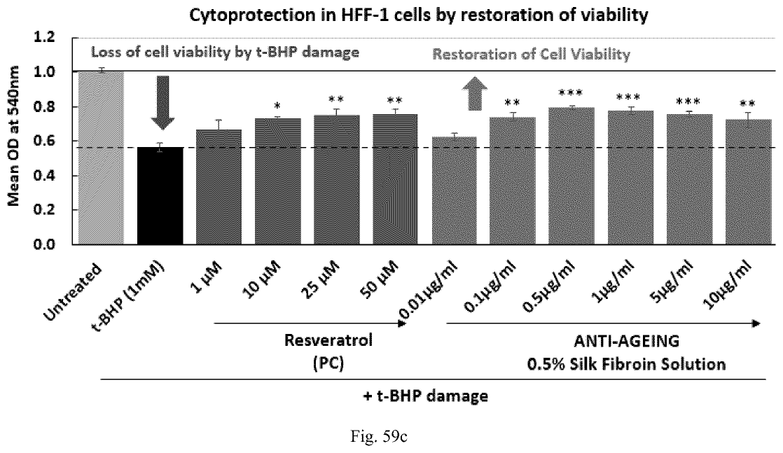

Study Design Assay 4b) Anti-Aging Cytoprotection Against t BHP Induced Damage (as Seen in FIGS. 59a-59c)

### Study Design

Test System—HFF 1 (Skin fibriblasts)
Number of cells plated—8000 cells/well plate
FBS concentrations—0.1%
Test items—Anti Ageing/Mineral SPF
Time points—24h pretreatment, t BHP (1 mM)
Estimation method—MTT assay
Positive Control—Resveratrol

### Procedure

Cells were plated in 96-well plates in 10% FBS and incubated for 24 h.
Cells were pre-treated with Test Item at various concentrations for 24 h.
Cells were exposed to tBHP (1 mM) for 2 hours.
After 2h, cell viability was assessed by MTT assay.
20 μl of MTT solution was added to each well and cells were incubated for 3 h at 37° C.
After incubation, supernatants were removed and 150 μl DMSO added to all wells to extract formazan crystals.
A purple coloured formazan complex was formed. Absorbance of each well was measured at 540 nm.

### Calculation

Protective effect on cell viability was calculated wrt controls (untreated and t BHP damaged cells).

$$\{ ( Absorbance of TI + tBHP ) - ( Absorbance of t BHP alone ) / ( Absorbance of Untreated ) Untreated ) -- ( Absorbance of t BHP alone ) \} * 10$$

### 4c) Anti-Apoptotic Effect Against UVB in Fibroblasts

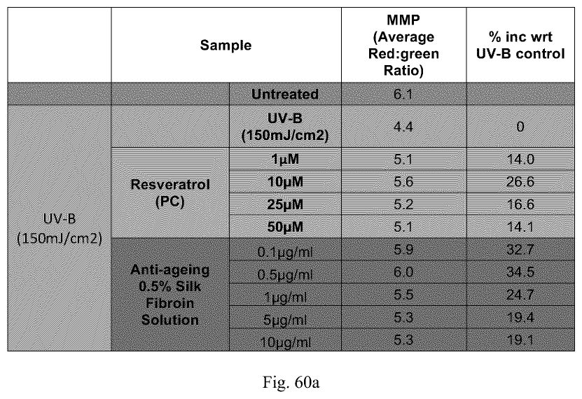

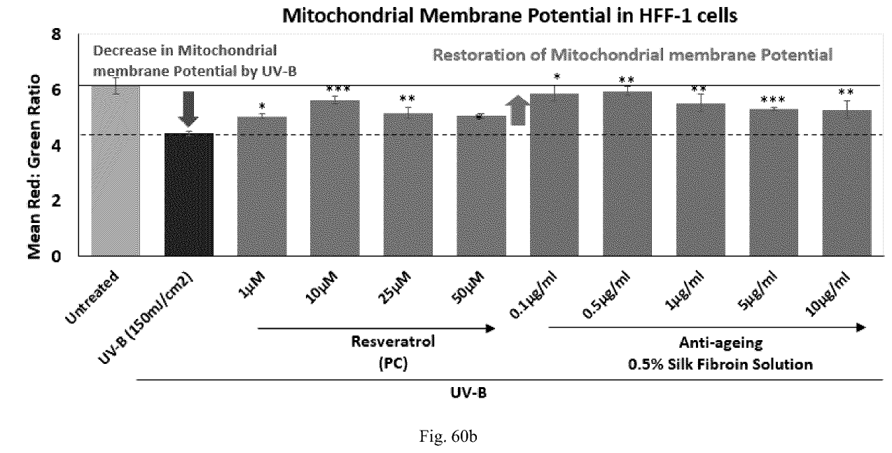

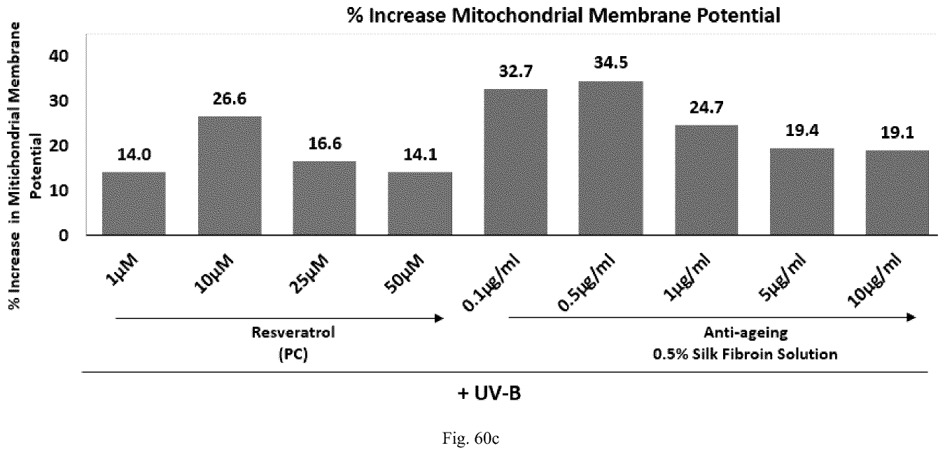

Study Design Assay 4c) Anti-aging Restoration of Mitochondrial Membrane Potential (MMP) (as Seen in FIGS. 60a-60c)

### Study Design

Test System—Human Fibroblasts (HFF 1)
Number of cells plate—8,000
FBS concentrations—0.5% FBS
Test items—Anti Ageing/Mineral SPF
Time point—24h pretreatment, UVB exposure (150 mJ/cm2), recovery 24h
End points—Mitochondrial Membrane Potential using JC 1 dye.
Positive Control—Resveratrol

### Procedure

Cells were plated in 10% FBS in 96-well plates and incubated for 24 h.
Cells were treated with Test item in 0.5% FBS at various concentrations for 24h.
After 24h, damage was induced using UVB (150 mJ/cm2) damage in
Cells were washed and incubated for recovery for another 24 h in serum free medium.
After 24 h, the effect on Mitochondrial membrane Potential was determined by JC 1 assay. Active mitochondria in live cells exhibit
brighter red fluorescence signal compared to mitochondria with lower membrane potential in apoptotic cells which fluoresce
green with JC 1 dye. Ratio of Red:Green indicates degree of MMP. Inhibition of MMP shows depolarization of MMP.
   1. After incubation, medium from each well was removed.
   2. 100 μl of 10 μM JC
   3. 1 dye in PBS was added to each well and cells were incubated at 37° C. for 25 mins.
   4. After incubation, cells were rinsed with PBS to remove dye and Fluorescence was measured at 528/590 for red and 485/528 for green.
   5. Ratio of Red: green (healthy cells) was calculated.

### Calculations

Increase of Mitochondrial membrane potential was calculated wrt UVB damage control.

$$[ ( A - B ) / B ] * 100 ;$$

$$Where A = Ratio of Red : Green in Test Item treated cells , B = Ratio of Red : Green in UVB damage control cells$$

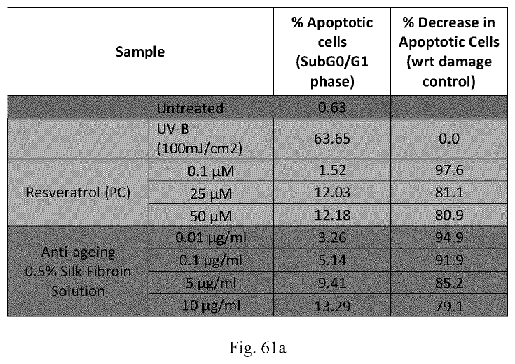

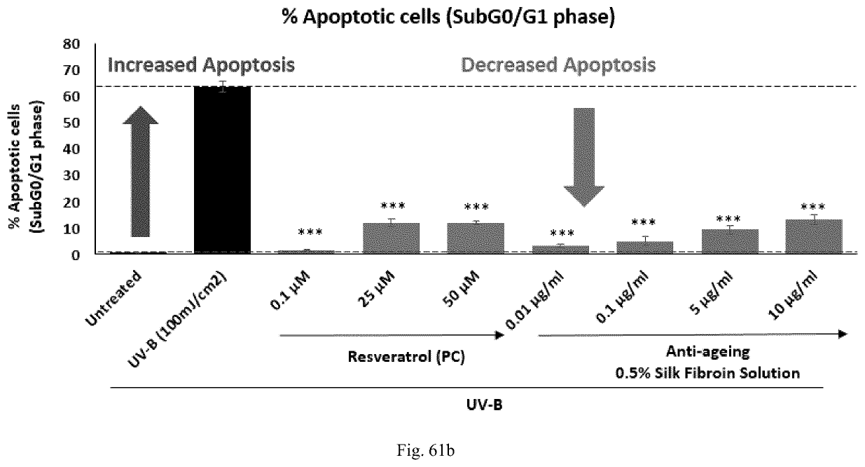

Study Design Assay 4c) Anti-Aging Increase in subG0/G1 Cells by Cell Cycle Assay (as Seen in FIGS. 61a-61c)

### Study Design

Test System—Human Fibroblast cell line (HFF 1)
Number of cells plated—0.25 million cells/12 well plates
FBS concentrations—0.5%,
Test items—Anti Ageing/Mineral SPF
Time points—24h pretreatment, UVB exposure (100 mJ/cm2), recovery 24h
Estimation method—SubG0/G1 apoptotic cells by Propidium iodide solution
Positive Control—Resveratrol

### Procedure

Cells were plated in 10% FBS in 12-well plates and incubated for 24 h.
Cells were treated with Test item in 0.5% FBS at various concentrations.
After 24h, damage was induced using UVB (150 mJ/cm2) damage in
Cells were washed and incubated for recovery for another 24 h in serum free medium.
After 24 h, the anti-apoptotic effect was measured by Cell cycle analysis.
   1. After incubation, medium from each well was removed and cells were harvested by trypsinization and centrifuged at 450 g for 5 min (low
   2. brake).
   3. Supernatant was discarded and cell pellet washed with 1×PBS at 450 g for 5 min (low brake).
   4. Cells were fixed with Ice cold 70% ethanol (500 μl) and stored at 4° C. for 24 h prior to staining.
   5. For staining, Ethanol fixed cells were centrifuged at low brake and washed with PBS.
   6. 200 μl of cell cycle reagent was added to each sample at kept at RT for staining for 30 min in dark.
   7. After incubation, cells were acquired using flow cytometer.

### Calculations

Decrease in apoptotic cell population (sub G0/G1) was calculated wrt UVB damage control.

$$[ ( B - A ) / B ] * 100 ;$$

$$Where A = \% subG 0 / G 1 cells in Test Item treated , B = \% subG 0 / G 1 cells in UVB damage Control$$

### 4d) Inhibition of ROS Generation in Fibroblasts

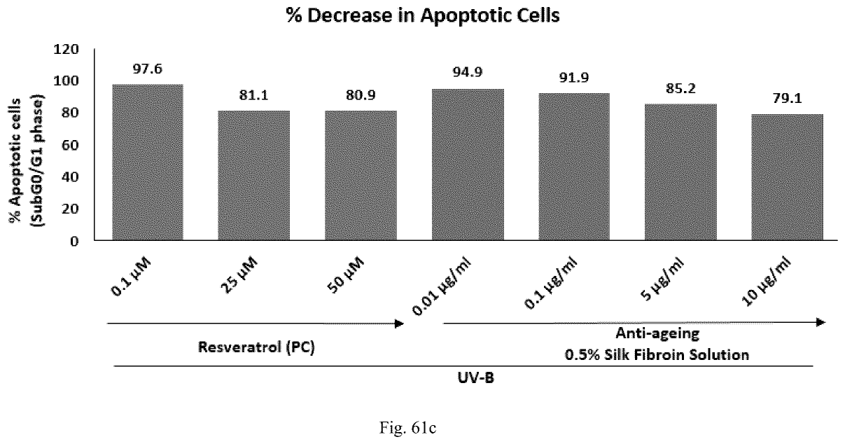

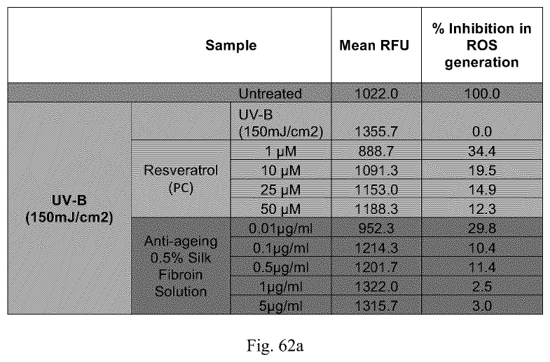

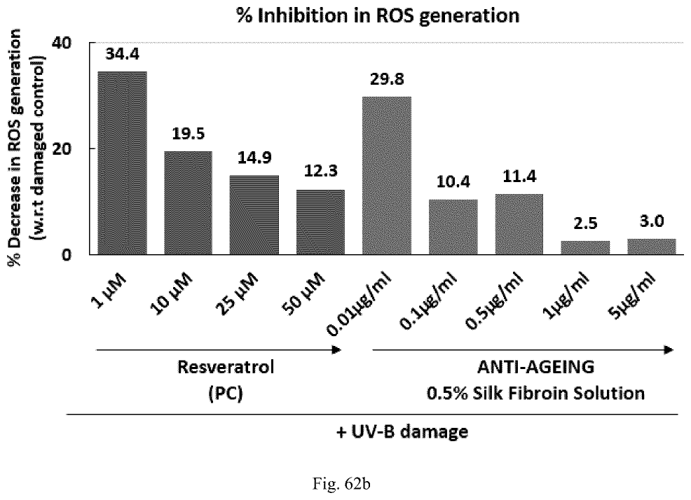

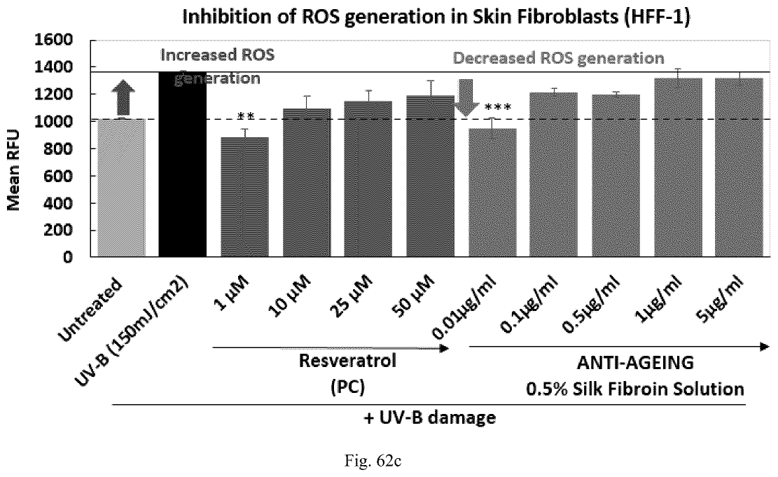

Study Design Assay 4d) Anti aging Decrease in ROS Generation (as Seen in FIGS. 62a-62c)

### Study Design

Test System—Human Fibroblasts (HFF 1)
Number of cells plated—8,000
FBS concentrations—0.5% FBS
Test items—Anti Ageing/Mineral SPF
Time point—24h pretreatment, UVB exposure (150 mJ/cm2), recovery 24h
End points—ROS levels by DCFDA dye.
Positive Control—Resveratrol

### Procedure

Cells were plated in 10% FBS in 96-well plates and incubated for 24 h.
Cells were treated with Test item in 0.5% FBS at various concentrations for 24h.
After 24h, damage was induced using UVB (150 mJ/cm2) damage in
Cells were washed and incubated for recovery for another 24 h in serum free medium.
After 24 h, the effect on ROS generation was determined by DCFDA assay.
   1. After incubation, medium from each well was removed.
   2. 100 μl of 10 μM DCFDA dye in phenol free medium was added to each well and cells were incubated at 37° C. for 45 mins.
   3. After incubation, cells were rinsed with PBS to remove dye and Fluorescence was measured at 485/528.
   4. Decrease in ROS generation was calculated wrt UVB damage control.

### Calculations

Decrease in ROS generation was calculated wrt UVB damage control.

$$[ ( B - A ) / B ] * 100 ;$$

$$Where A = RFU of Test Item treated cells , B = RFU of UVB damage control cells$$

### 4e) Anti-Inflammatory Activity in Fibroblasts

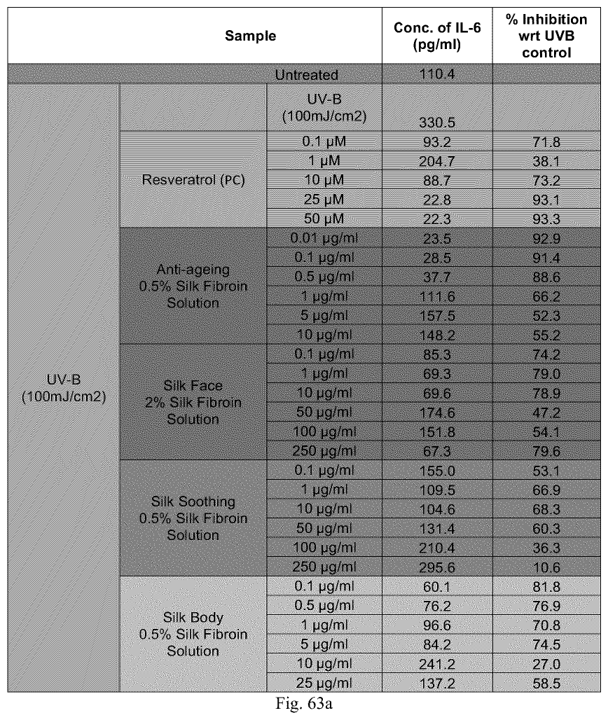

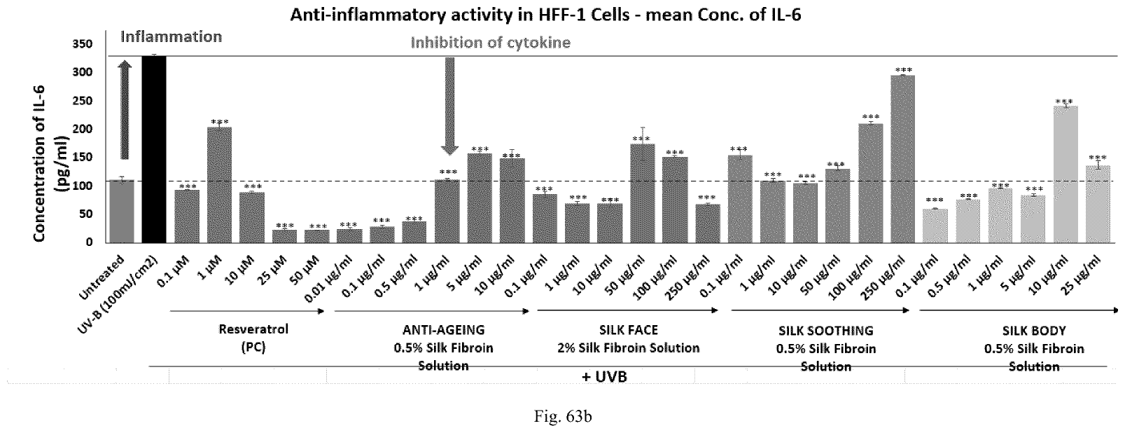

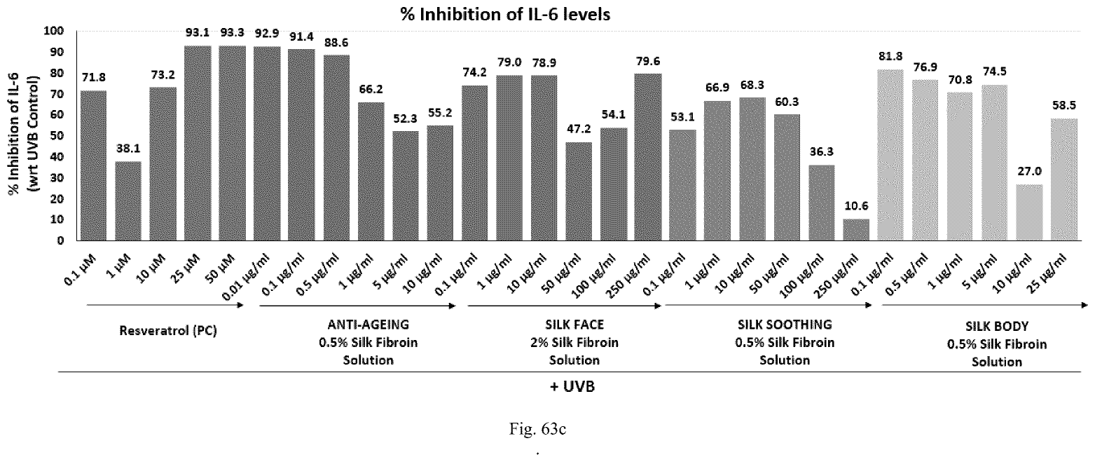

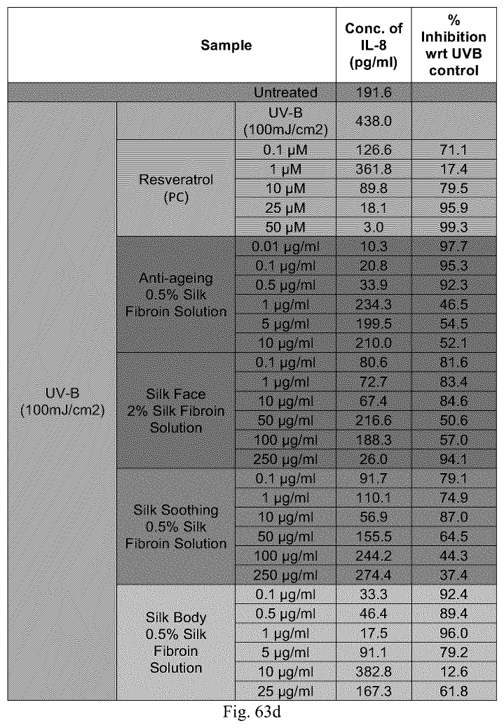

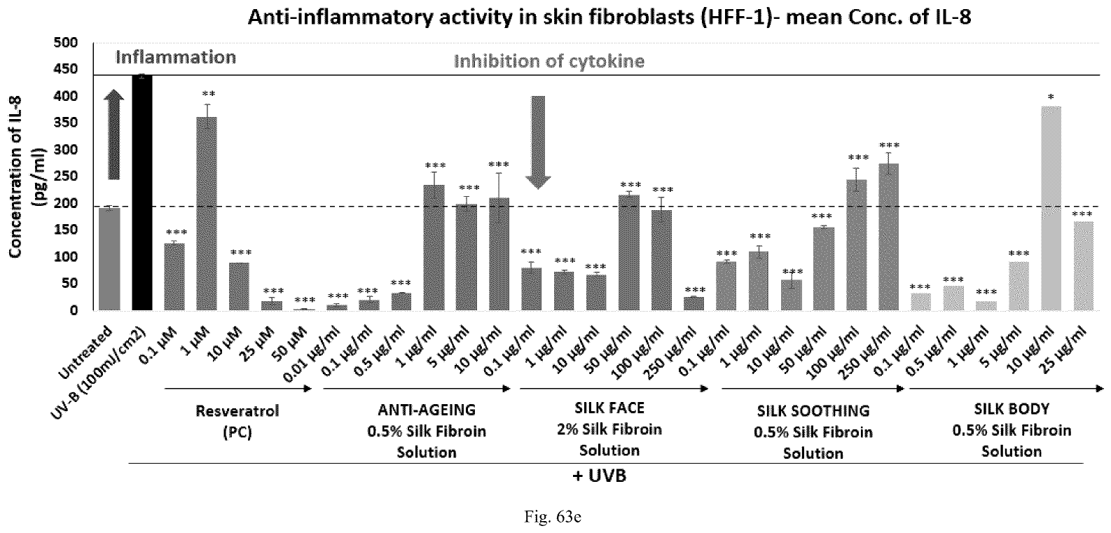

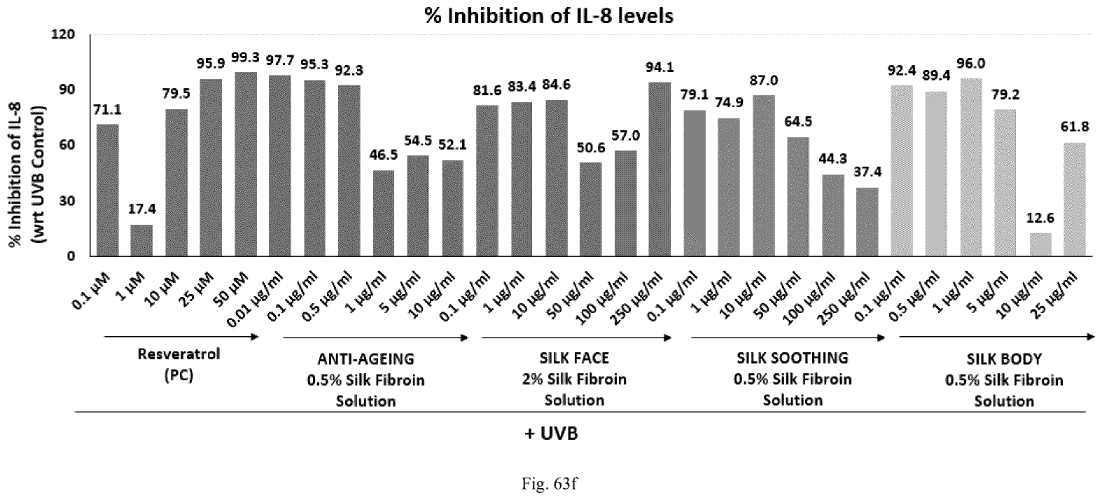

Study Design Assay 4e) Anti-Aging-Anti-inflammatory activity by Inhibition of Cytokines (as Seen in FIGS. 63a-63f)

### Study Design

Test System—Human Fibroblast cell line (HFF 1)
Number of cells plated—0.25 million cells/6 well plates
FBS concentrations—0%,
Test Items—Anti Ageing/Mineral SPF, Silk Face/Silk serum, Silk Soothing/Soothing lotion, Silk Body/Body lotion
Time points—24h pretreatment, UVB exposure (100 mJ/cm2), recovery 24h
Estimation method—ELISA
Positive Control—Resveratrol

### Procedure

Cells were plated in 10% FBS in 6-well plates and incubated for 24 h.

Cells were serum starved in 0% FBS for 24 h.
Cells were treated with 4 Test Items in 0% FBS at non-cytotoxic concentrations for 24h.
After 24h, damage was induced using UVB (100 mJ/cm2 for 5.5 min) damage in
Cells were washed and incubated for recovery for another 24 h in serum-free medium.
After incubation, culture supernatants were collected
Levels of IL6, IL 8, IL 1 α and TNF α (supernatants) were determined using ELISA as follows
   1. Assay diluent was added to each well. Respective kit standards and samples (supernatants) were directly pipetted into the wells and incubated
   2. for 2 h at RT.
   3. After washing away any unbound substances for a total of 3 times, respective conjugate was added to each well and incubated f
   4, or 12 h at RT.
   5. Following a wash (3 times) to remove any unbound conjugate, substrate solution was added to the wells and incubated for 30 mi
   6. n a t RT in dark.
   7. The reaction was stopped by adding Stop solution to each well.
   8. The optical density of the color was measured at 450 nm
   9. Conc of cytokines were calculated from standard curve.

### Calculations

Inhibition in levels of cytokines was calculated wrt UVB control.

$$[ ( B - A ) / B ] * 100 ;$$

$$Where A = Levels of cytokines in Test Item treated cells , B = Levels of cytokines in UVB Control .$$

$$* Levels of IL - 1 - α and TNF - α remain unaffected$$

### 4f) NRF2, NF-κB

### Study Design Assay 4f) Anti-Aging Modulation of NRF 2, NFκB

### Study Design

Test System—Human Fibroblast cell line (HFF 1)
Number of cells plated—0.25 million cells/6 well plates
FBS concentrations—0%,
Test Items—Anti Ageing/Mineral SPF
Time points—24h pretreatment, UVB exposure (150 mJ/cm2), recovery 24h
Estimation method—ELISA
Positive Control—Resveratrol

### Procedure

Cells were plated in 10% FBS in 6-well plates and incubated for 24 h. Cells were serum starved in 0% FBS for 24 h.
Cells were treated with Test Item in 0% FBS at non-cytotoxic concentrations for 24h.
After 24h, damage was induced using UVB (100 mJ/cm2) damage in
Cells were washed and incubated for recovery for another 24 h in serum-free medium.
After incubation, culture supernatants were collected
Levels of NRF 2, NFκB—(lysates) were determined using ELISA as follows
   1. Assay diluent was added to each well. Respective kit standards and samples (supernatants) were directly pipetted into the wells and incubated
   2. for 2 h at RT.
   3. After washing away any unbound substances for a total of 3 times, respective conjugate was added to each well and incubated for 1-2 h at RT.
   4. Following a wash (3 times) to remove any unbound conjugate, substrate solution was added to the wells and incubated for 30 min a t RT in dark.
   5. The reaction was stopped by adding Stop solution to each well. Q
   6. The optical density of the color was measured at 450 nm. Conc of cytokines were calculated from standard curve.

### Calculations

Inhibition in levels of cytokines was calculated wrt UVB control.

$$[ ( B - A ) / B ] * 100 ;$$

$$Where A = Levels of cytokines in Test Item treated cells , B = Levels of cytokines in UVB Control .$$

Levels of NRF-2, NF-κB remained unaffected

### Anti-Aging Potential 5. Keratinocytes Based Assays

### 5a) Anti-Inflammatory Activity in Keratinocytes

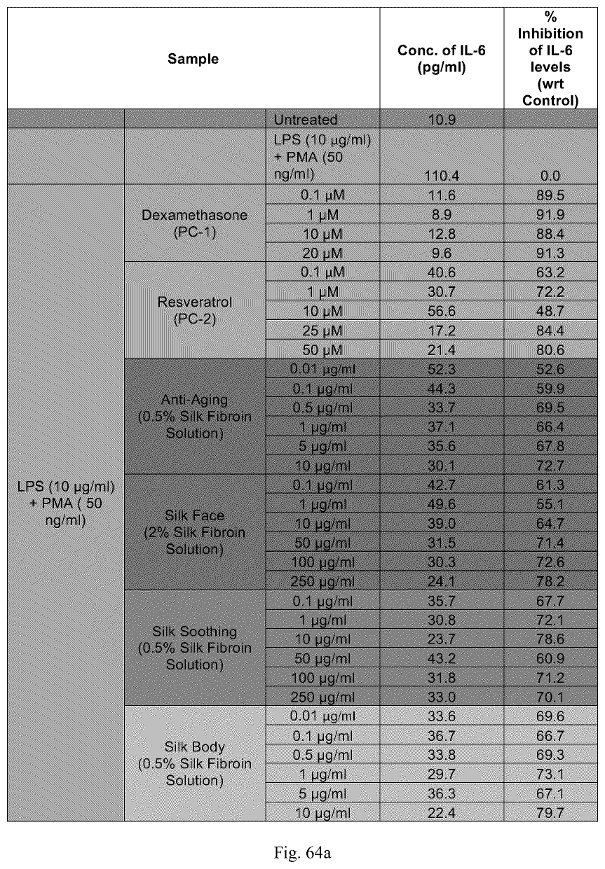

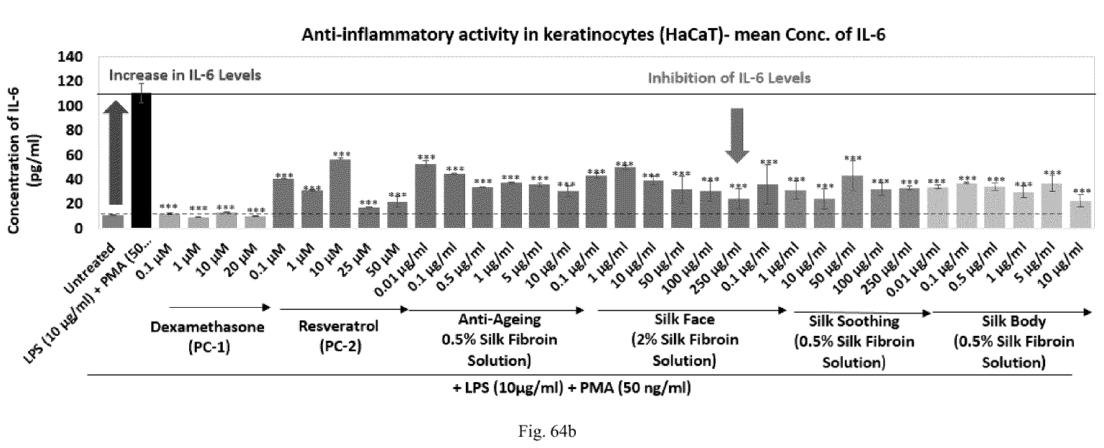

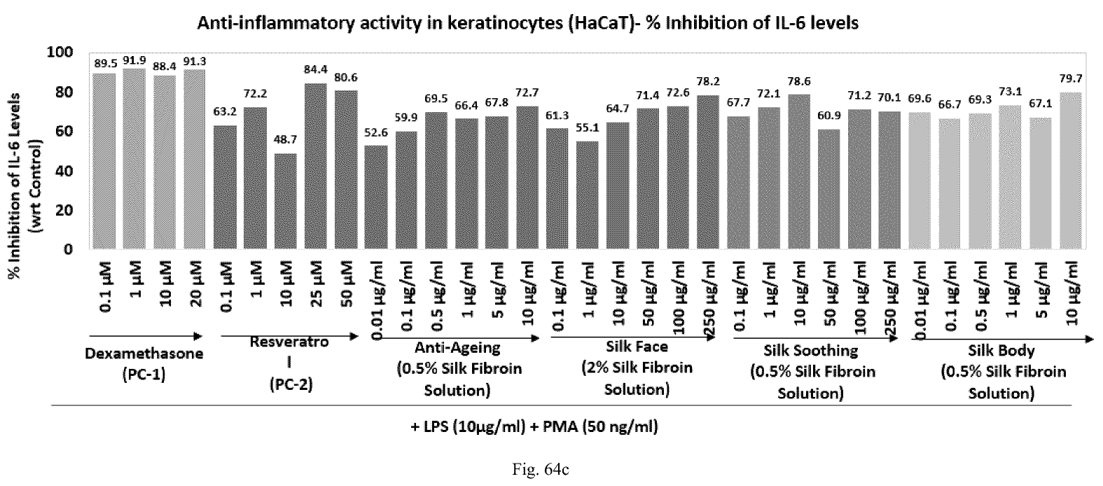

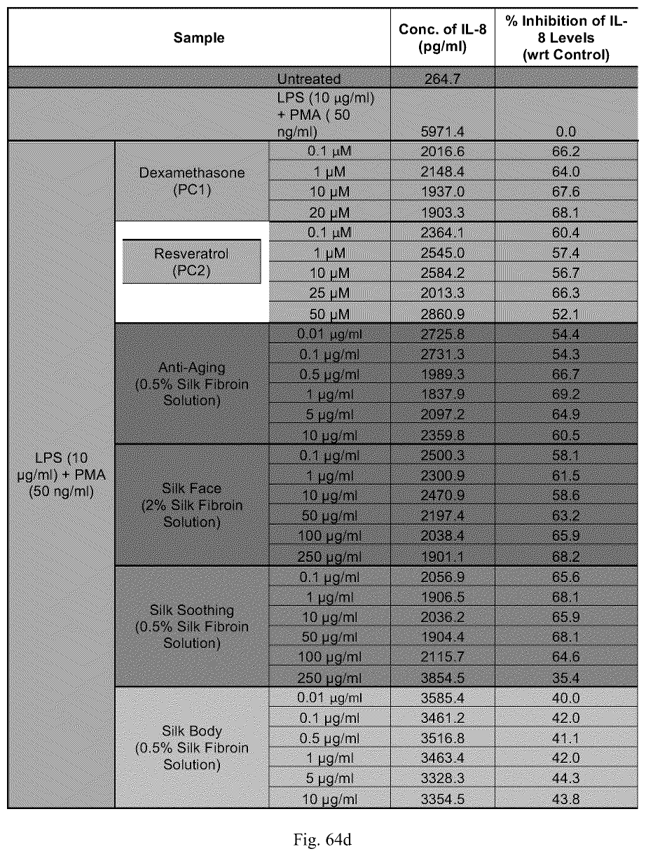

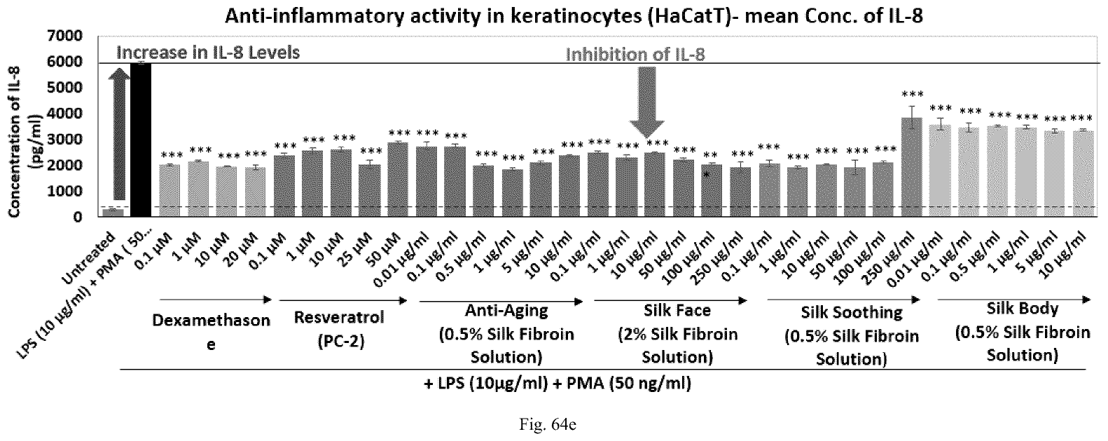

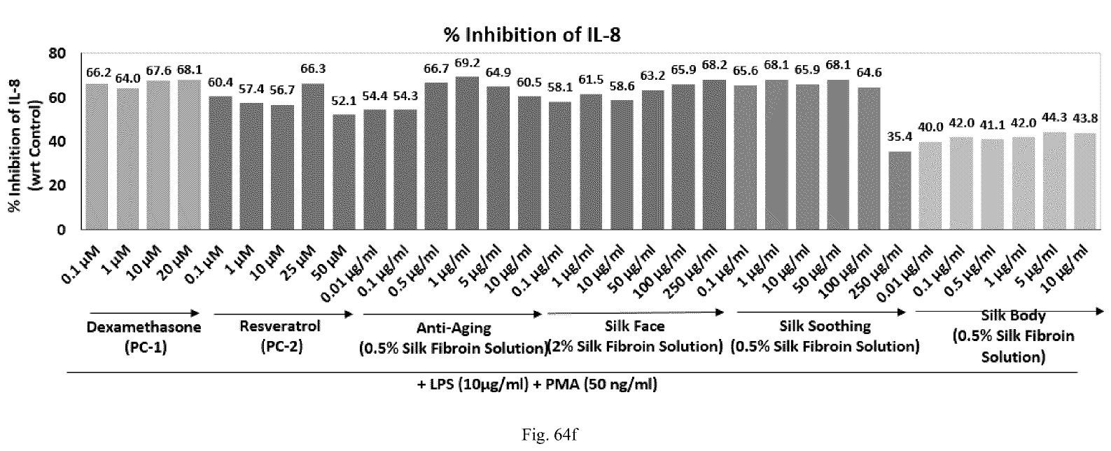

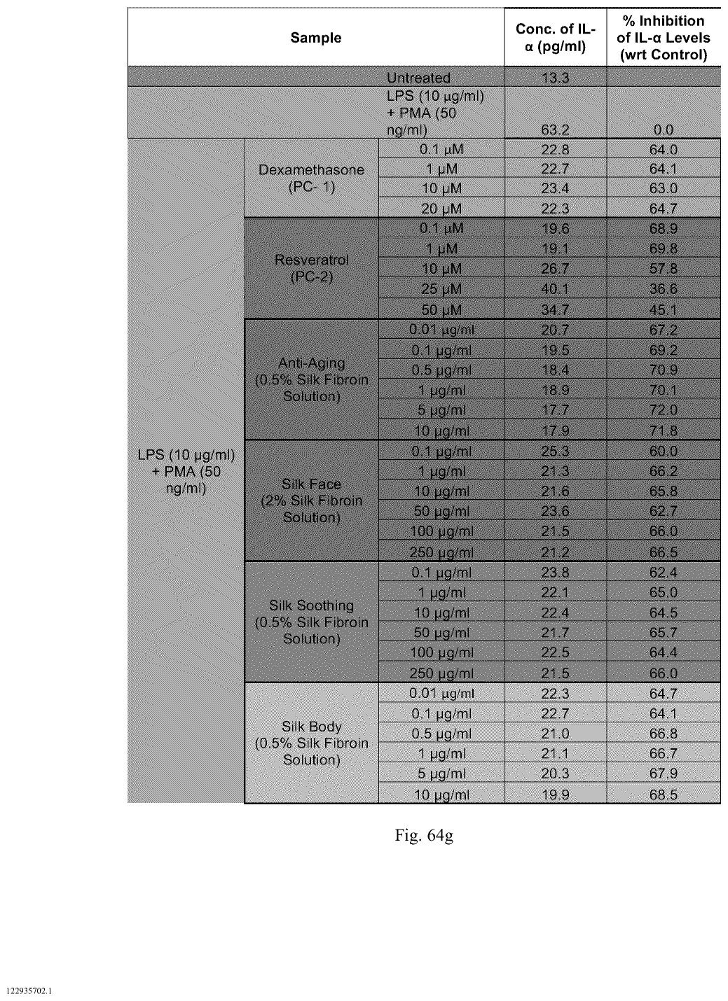

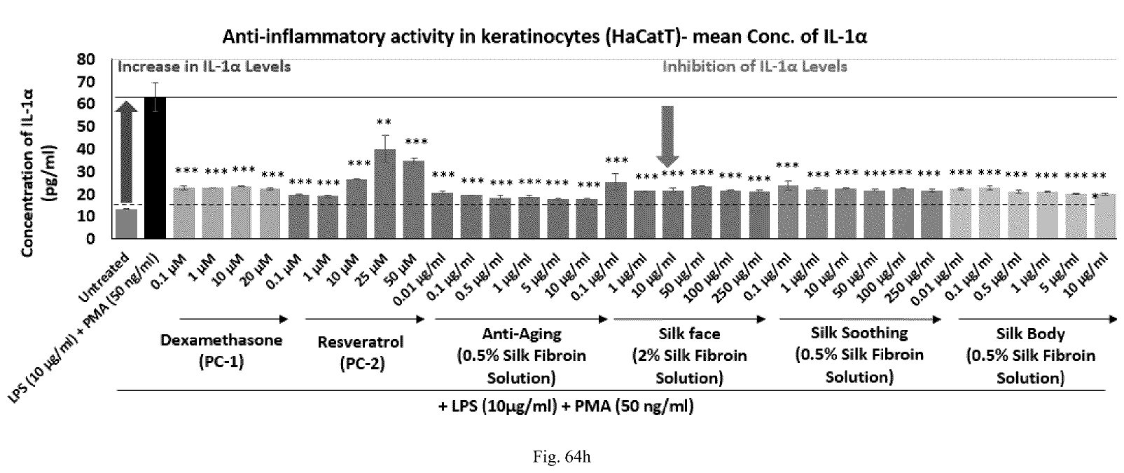

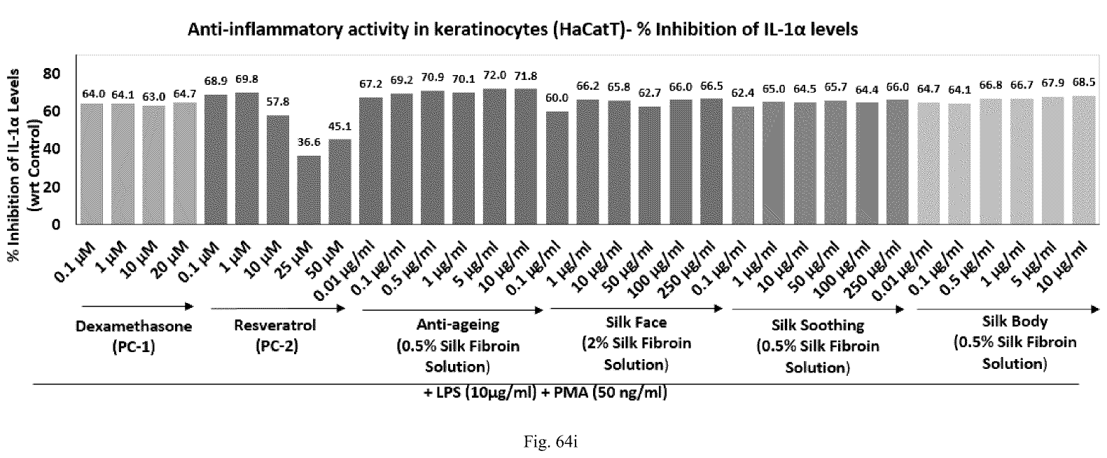

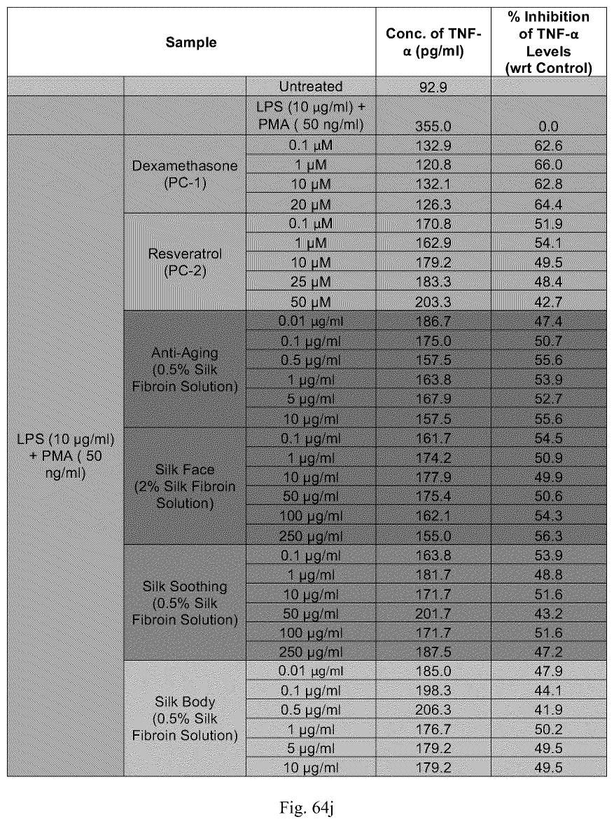

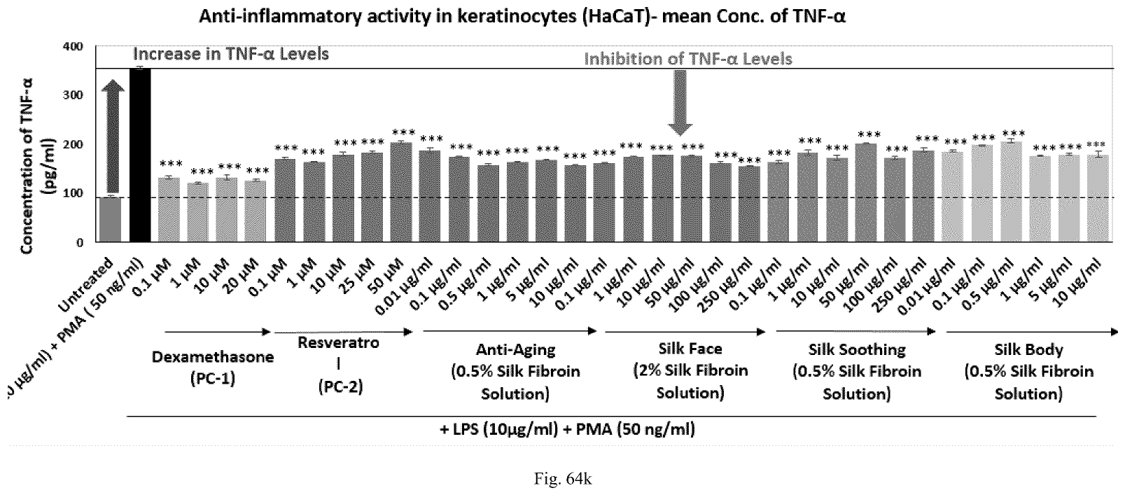

Study Design Assay 5a) Anti-Aging Inhibition of Cytokines (as Seen in FIGS. 64a-64l)

### Study Design

Test System—Human keratinocytes (HaCaT)
Number of cells plated—0.1 million cells
FBS concentrations—0.1% FBS
Test Items—Anti Ageing/Mineral SPF, Silk Face/Silk serum, Silk Soothing/Soothing lotion, Silk Body/Body lotion
Time point—24h
End point—ELISA
Positive Control/s—Dexamethasone, Resveratrol

### Procedure

Cells were plated in 10% FBS in 6-well plates and incubated for 24 h.

Cells were serum starved in 0.10% FBS for 24 h.
Cells were treated with 4 Test Items in 0.1% FBS at non-cytotoxic concentrations along with LPS (10 pg/ml) and PMA (50 ng/ml) for 24h.
After 24h, culture supernatants were collected
Levels of IL6, IL 8, TNF α and IL 1 α (supernatants) were determined by ELISA as follows
   1. Assay diluent was added to each well. Respective kit standards and samples (supernatants) were directly pipetted into the wells and
   2. incubated for 2 h at RT.
   3. After washing away any unbound substances for a total of 3 times, respective conjugate was added to each well and incubated for 1-2 h at RT.
   4. Following a wash (3 times) to remove any unbound conjugate, substrate solution was added to the wells and incubated for 30 min a t RT in
   5. dark. The reaction was stopped by adding Stop solution to each well.
   6. The optical density of the color was measured at 450 nm. Conc of cytokines were calculated from standard curve.

### Calculations

Inhibition in levels of cytokines was calculated wrt Stimulant (LPS+PMA) control.

$$[ ( B - A ) / B ] * 100 ;$$

$$Where A = Levels of cytokines in Test Item and Stimulant treated cells , B = Levels of cytokines in Stimulant ( LPS + PMA )$$

### Anti-Aging Potential 6. Mast Cells-Based Assay

### 6a) Inhibition of Histamine Release

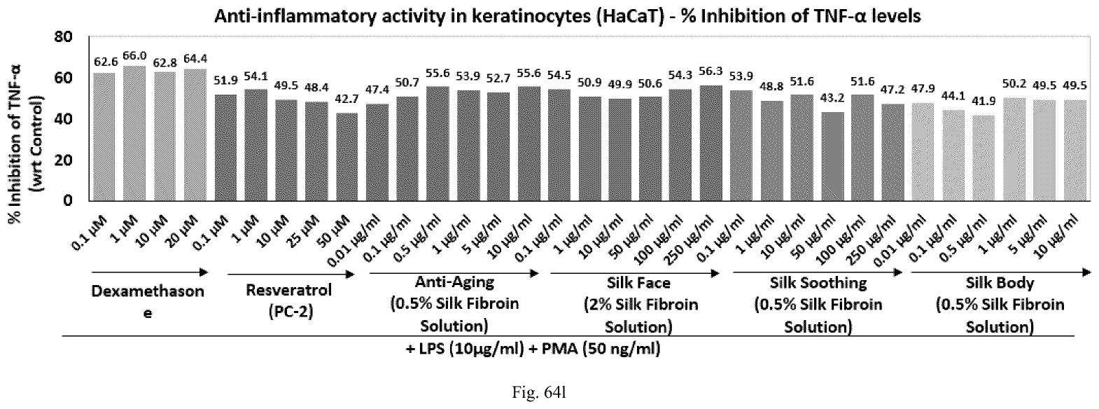

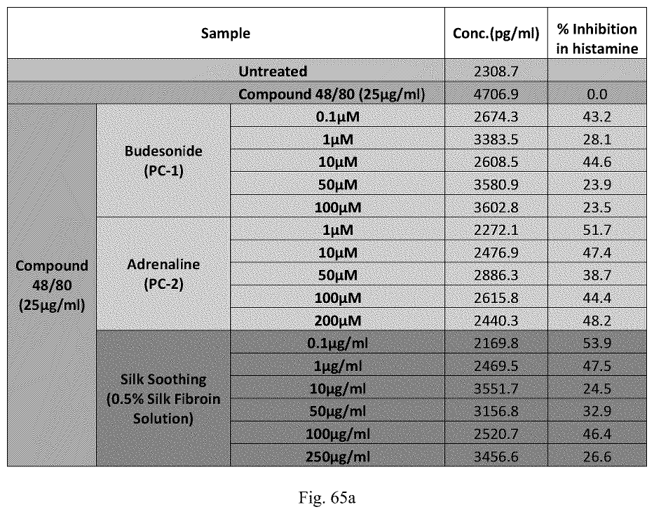

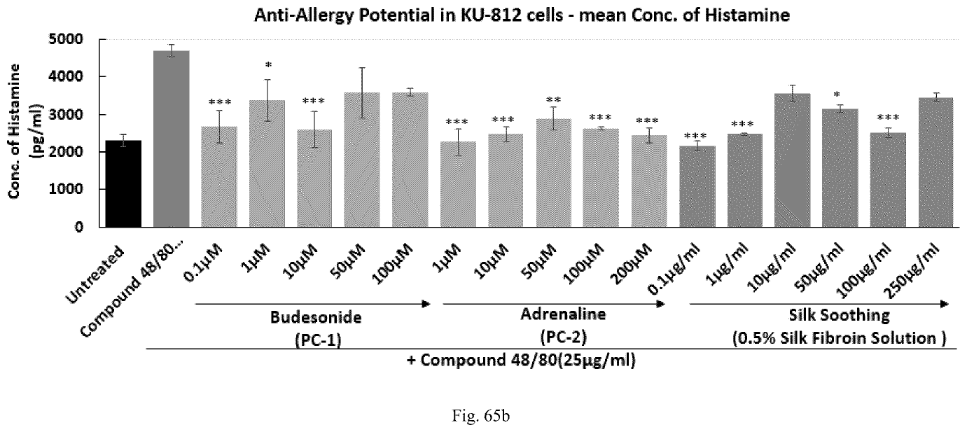

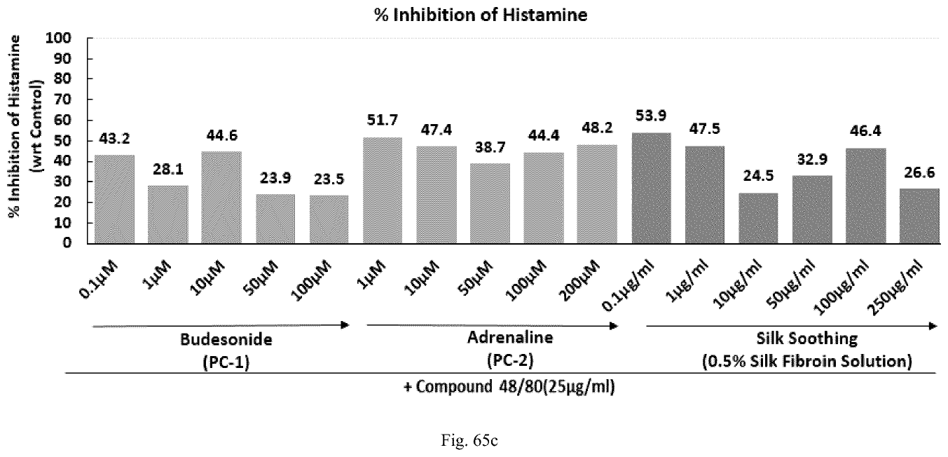

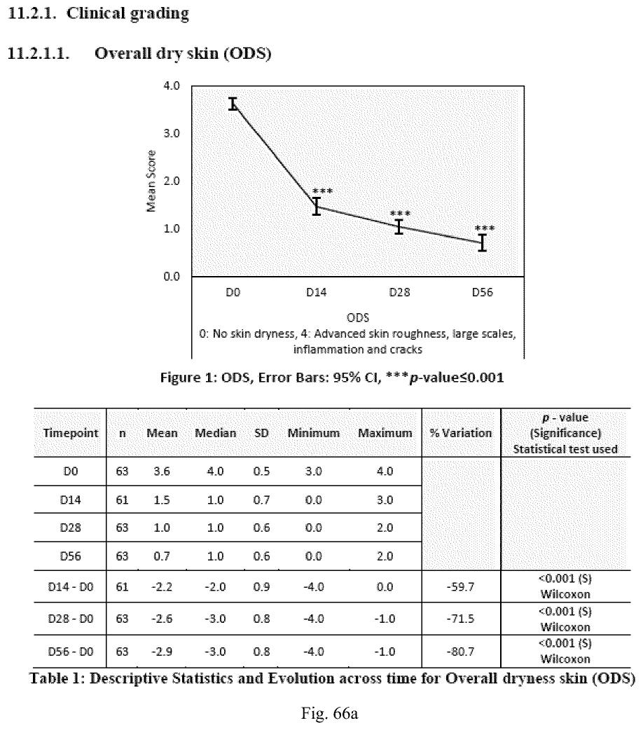

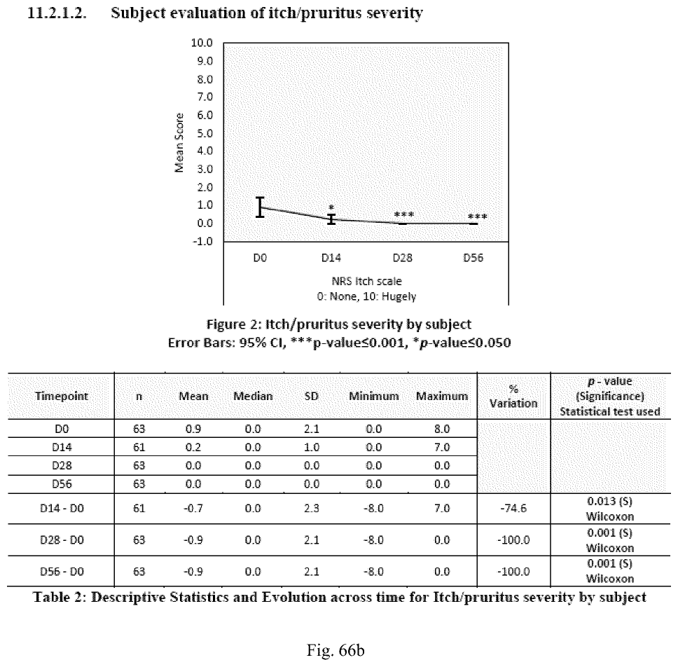

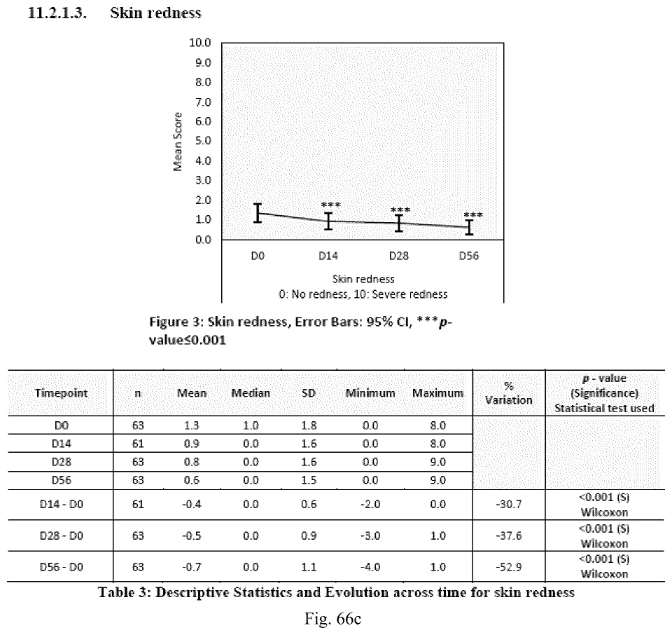

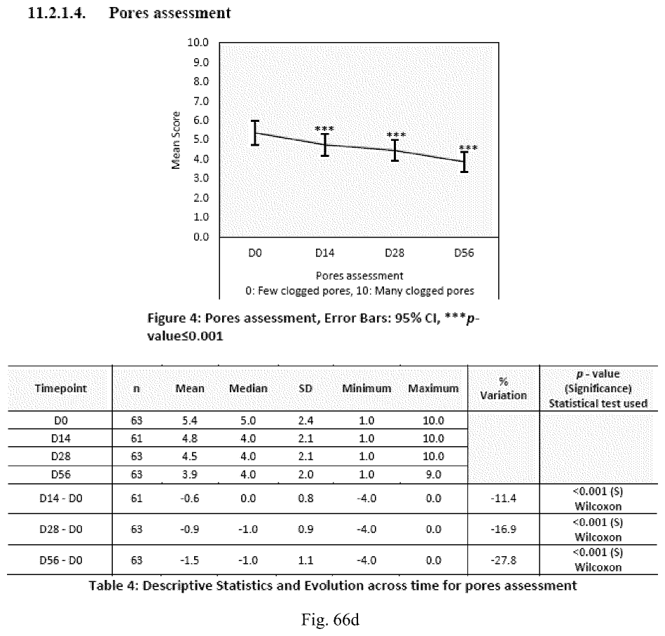

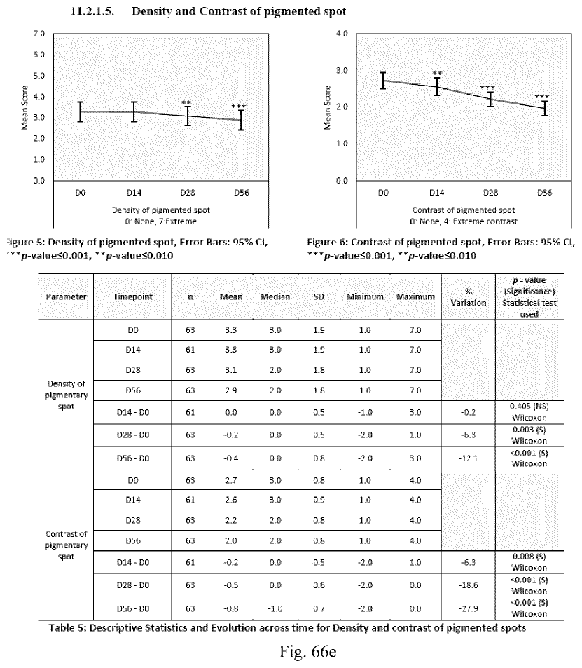

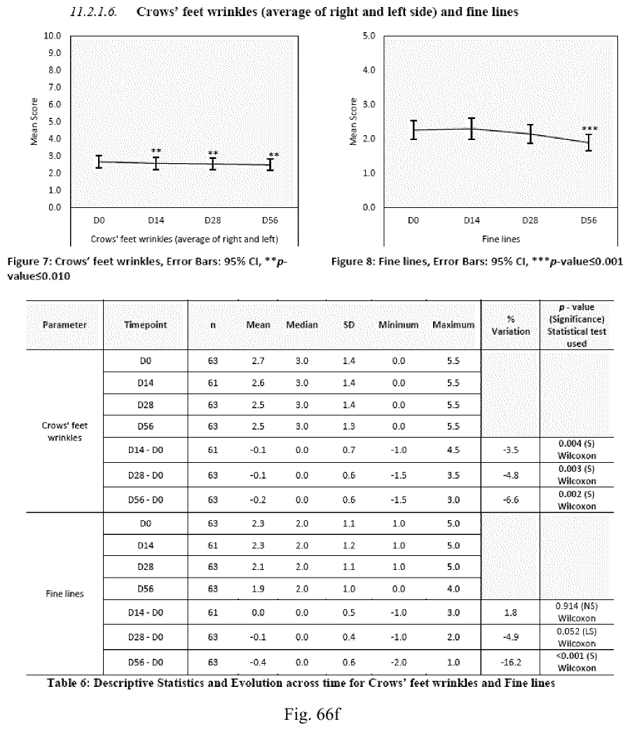

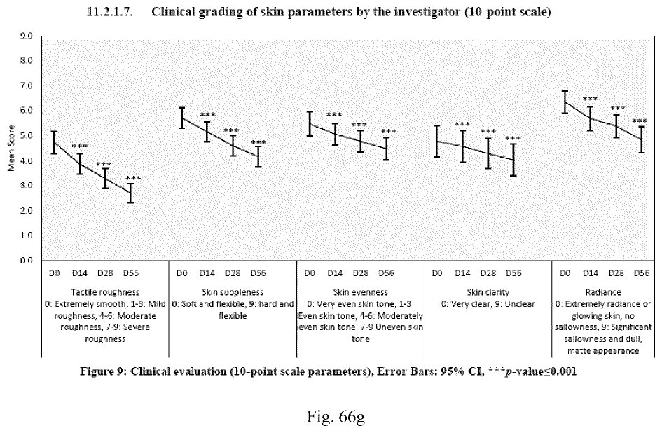

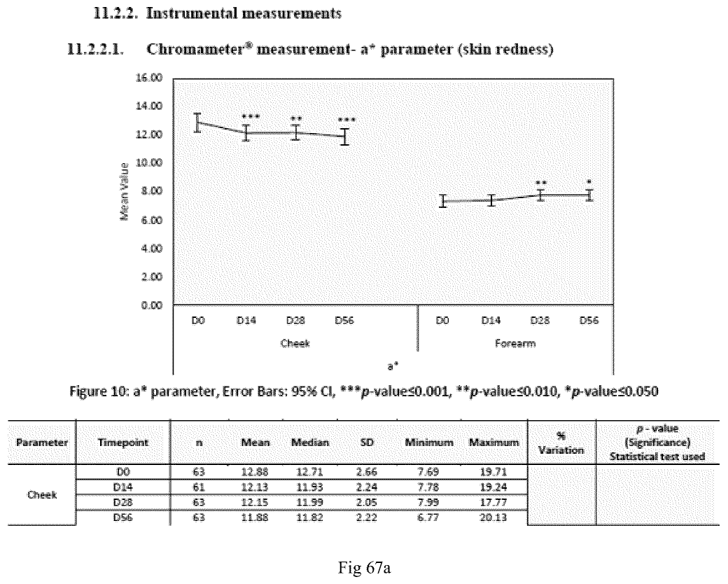

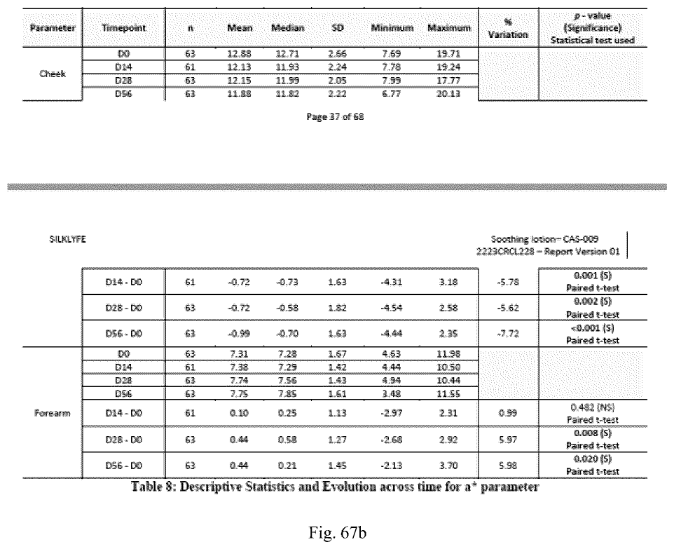

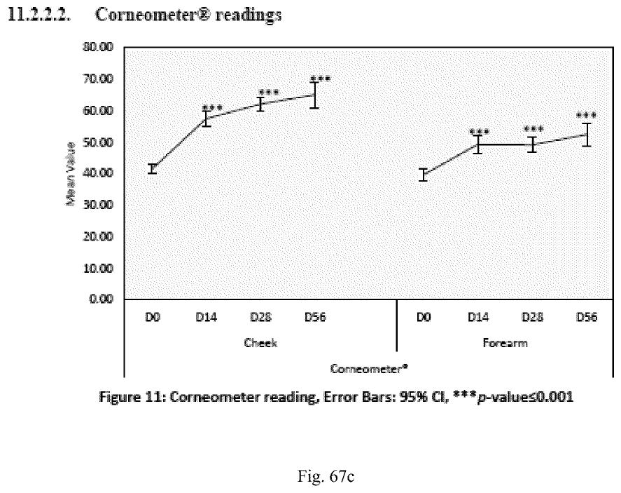

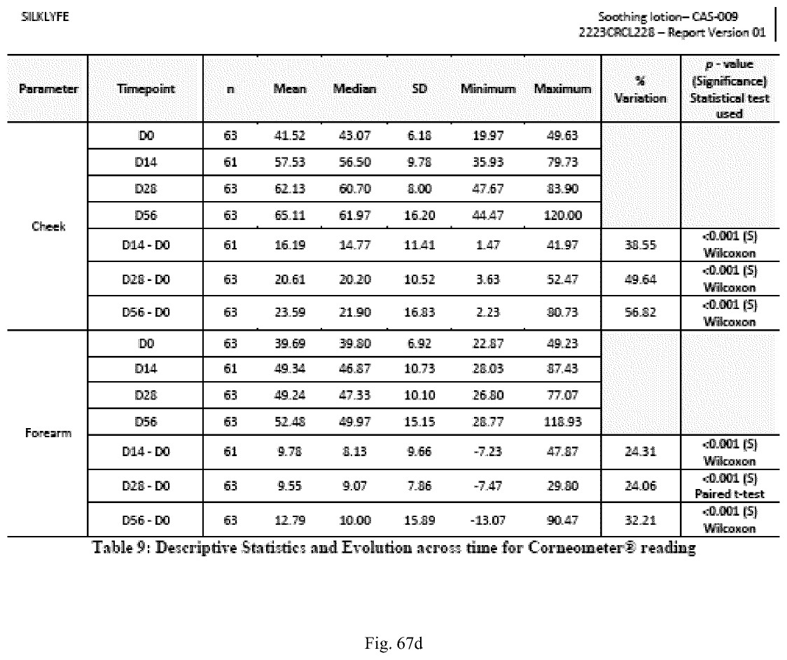

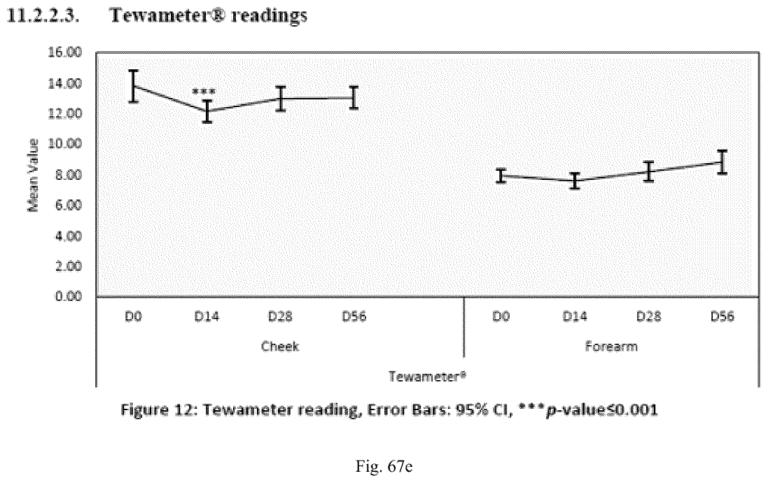

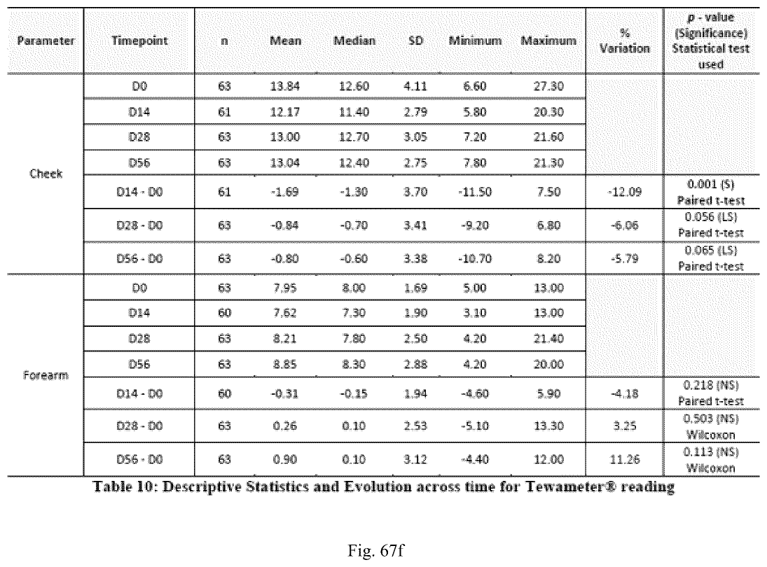

Study Design Assay 6a)—Anti-Aging—Inhibition of Histamine Release (as Seen in FIGS. 65a-65c)

### Study Design

Test System—Human Basophilic Leukemia Cell line (KU812)
Number of cells plated—10×103 cells/well
FBS concentrations—10% FBS
Test Items—Silk Soothing/Soothing lotion
Time point—24h
End point—Histamine Assay
Positive Control/s—Budesonide, Adrenaline

### Procedure

Cells were plated in 10% FBS in 96-well plates and incubated for 24 h.
Cells were treated with Test Item in 10% FBS at non-cytotoxic concentrations for 24h.
After 24 h, cells were pelleted and resuspended in Tyrode buffer (30 mM TrisHCl, 120 mM NaCl, 5 mM KCl, 1 mM CaCl₂), 1 mM MgCl2, 5.6
mM glucose and 0.03% bovine serum albumin).
Cells were stimulated with Compound 48/80.
After stimulation reaction was terminated by incubating the plate on ice for 15 min.
The cell suspension was centrifuged, and supernatant was collected from each well carefully for estimation of the levels of hist amine released
from KU 812 cells.
Supernatant obtained above was mixed with NaOH and ophthalaldehyde dye as following:
Supernatant (160 μl)+1N NaOH (32 μl)+OPT (8 μl) (Ratio 20:4:1)
Samples were incubated for 5 min at RT.
The fluorescence of the above mixture was read at 360 nm excitation/460 nm emission at sensitivity 50.

### Calculations

The percentage inhibition in histamine release in comparison with Control cells (48/80 alone stimulated cells) was calculated using the following formula:

$$\% Inhibition of histamine release = [ ( AB ) / A ] * 100 ; where A = Concentration of histamine in Control cells ( stimulated with Compound 48 / 80 alone ) ; B = Concentration of histamine in treated cells + Compound 48 / 80$$

### Additional Testing Data

Study: a Single-Centre Clinical Trial to Evaluate the Efficacy and Safety of Silk Soothing Lotion for Smoothing, Hydrating and Radiant Skin Along with Smoothing Fine Lines, Reducing Blemishes and Anti-Aging Benefits Claim

### Study Objective:

The principal objective of the study was to evaluate the efficacy and safety of silk soothing lotion for smoothing, hydrating and radiant skin along with smoothing fine lines, reducing blemishes and anti-aging benefits claim.

The secondary objectives were:
To assess the skin dryness by clinical scoring through dermatological evaluation using the Overall dry skin score (ODS)
To assess the density and contrast of pigmentary spots by clinical grading through dermatological evaluation
To assess the skin redness by clinical grading through dermatological evaluation
To assess the tactile roughness, skin suppleness, skin evenness, clarity of skin tone and skin radiance by clinical grading through dermatological evaluation
Assessment of the forehead fine lines and wrinkles and Crow's feet wrinkles Using skin aging atlas through dermatological evaluation
Assessment of pores through dermatological evaluation
Self-Assessment Questionnaire for efficacy, safety, and organoleptic properties of test product
Subject evaluation of itch/pruritus severity using the 11—points itch NRS scale
Evaluation of skin hydration using Corneometer CM 825@/MPA on forearm
Evaluation of skin barrier repair properties by Trans-epidermal water loss measurements over face and forearm using Tewameter
Evaluation of skin redness using the a* parameter of Chromameter measurements on face and forearms
Illustrative photography VISIA

### Methodology:

Clinical scoring of Skin Dryness using the Overall dry skin score (ODS) was done by dermatologist at visits D0, D14, D28 and D56.
Clinical grading of density and contrast of pigmentary spots was done by dermatologist at visits D0, D14, D28 and D56.
Clinical grading of skin redness was done by dermatologist at visits D0, D14, D28 and D56.
Clinical grading of tactile roughness, skin suppleness, skin evenness, clarity of skin tone and skin radiance was done by dermatologist at visits D0, D14, D28 and D56.
Assessment of the forehead fine lines and wrinkles; and Crow's feet wrinkles by Dermatologist using Skin ageing atlas was done by dermatologist at visits D0, D14, D28 and D56.
Assessment of pores was done by dermatologist at visits D0, D14, D28 and D56.
Subject evaluation of itch/pruritus severity using the 11-point NRS itch scale was done by dermatologist at visits D0, D14, D28 and D56.
Evaluation of Skin barrier repair properties by Trans-epidermal water loss measurements—face and forearm at visits D0, D14, D28 and D56
Skin Hydration level measurement using Corneometer CM® 825/MPA6 at visits D0, D14, D28 and D56
Evaluation of skin redness using the a*parameter of Chromameter over face & forearm at visits D0, D14, D28 and D56
Standardized photos were taken with VISIA CR® system at visits D0, D14, D28 and D56
Self-assessment questionnaire at visits D0, D14, D28 and D56

### Experimental Plan:

This was an open single-centre clinical trial study and intra-individual study; each subject was her own control.

### Mode of Use of the Investigational Product:

The investigational product was applied twice per day, in the morning and in the evening. 2-3 sprays for face and 1-3 sprays for forearm were applied.

### Main Inclusion Criteria:

Healthy men and women subject,
Subject aged from 30 to 65 years old,
Caucasian subjects,
Subject with very dry skin condition using the Overall dry skin score (subjects with scores of 3 and 4 will be included)
Ability of the subject to read and understand documents transmitted (consent and information form),
Subject willing to cooperate and participate by the following requirements for the duration of the study: o to use only the investigational product on the face in replacement of their usual cream for dry skin;
Subject willing to report immediately any adverse symptoms,
Subjects willing and capable to follow the study rules and a fixed schedule,
Subjects willing and capable to sign an informed consent document (including the language),
Subject with health insurance and/or social security (according to local legislation).

### Panel Description:

66 healthy female and male subjects were included in the study.

The analyzed panel consisted of:
64 female subjects and 2 male subjects,
Aged between 30 to 65 years old (Mean age: 51±1 years).
Caucasian subjects (66 subjects)
Subject with very dry skin condition (66 subjects)

### Summary of Statistical Analysis Method:

### Efficacy Analysis Population

The efficacy analysis was performed on 63 out 66 subjects. They have completed the study, without any major protocol deviation.

### Descriptive Statistics

We let Tx represent the values observed at time x, for each parameter. Quantitative variables, or those that could reasonably be treated as such, were summarized using the minimum, maximum, measures of central tendency such as the mean and median & measures of dispersion such as the standard deviation (SD). Qualitative variables were summarized in the form of counts and percentages.

### Evolution Across Time

The evolution across time with respect to the baseline, was investigated for each parameter, by using either the Student's Paired t-test or the Wilcoxon Signed Rank Test depending on normality of the difference data.

The latter will be tested using a Shapiro Wilk test at 1% level of significance.

The null and alternative hypotheses are defined as follows:
H0: There is no difference between the two timepoints compared.
H1: There is a difference between the two timepoints compared.

### Significance Level

The null hypothesis was generally rejected if a p-value less than 0.05 (5% significance level) was produced by the statistical procedure.

### Abbreviation Used:

D: Day
S: Significant
NS: Not significant
CI: Confidence Interval
SD: Standard deviation

### Statistical Software Used:

Microsoft Excel 2010 or above
IBM SPSS version 19.0

### Number of Subjects

Number screened: 66 subjects
Number randomised: 66 subjects
Number analyzed: 63 subjects Inclusion criteria

They were defined in the study protocol as the followings:
Healthy men and women subject,
Subject aged from 30 to 65 years old,
Caucasian subjects,
Subject with very dry skin condition using the Overall dry skin score (subjects with scores of 3 and 4 will be included)
Ability of the subject to read and understand documents transmitted (consent and information form),
Subject willing to cooperate and participate by the following requirements for the duration of the study:
to use only the investigational product on the face in replacement of their usual cream for dry skin;
Subject willing to report immediately any adverse symptoms,
Subjects willing and capable to follow the study rules and a fixed schedule,
Subjects willing and capable to sign an informed consent document (including the language),
Subject with health insurance and/or social security (according to local legislation).

### Non-Inclusion Criteria

They were defined in the study protocol as the followings:
Hairs in the test sites
Subjects not matching one of the above-described eligibility criteria or whose participation is deemed inappropriate,
Female volunteers either pregnant or breast feeding,
Subject presenting acute skin irritation, skin diseases (exception a slight acne vulgaris) or dermatological disorders (scars, sunburn, tattoos, moles) which would interfere with the evaluation of the product tested,
Known allergies to cosmetic products and ingredients of cosmetic test products,
Subjects with experience of severe reactions from exposure to sunlight (photo sensitization)
Topical medication in the test site within 1 month prior to test starting.
Skin diseases,
Systemic medication with anti-inflammatory agents and antibiotics; such systemic medication must have been stopped at least 2 weeks prior to starting of the study,
Systemic medication with corticoids and/or antihistamines; such systemic medication must have been stopped at least 4 weeks prior to starting of the study.
Insulin-dependent diabetes,
Currently receiving anti-allergy injections, final injection within the last week, or expecting to begin injections during the course of the study,
Immune deficiency or autoimmune disease, e.g. HIV positives or volunteers with infectious hepatitis etc.,
Subjects with severe disorders within the last 6 months, e.g. cancer, circulatory disorders, serious diabetes (if necessary consultation of the physician),
Subjects with disorders within the last 8 days before starting the study with fever period of more than 24 hours,
Intellectual/mental inability to follow study instructions (if suspected) or incapacitation,
Subject with clinically significant dermographism or generalized skin disorders that could compromise evaluation of skin reactions,
A condition or medication which, in the investigator's judgment, makes the subject ineligible or places the subject at undue risk,
Subjects participating in a study with pharmaceuticals within a period of at least 15 days prior to this,
Current participation in another clinical study of any kind,
Subject who cannot be contacted in case of emergency,
Application of care cosmetic product less than 72h before the beginning of the study,
Application of make-up product in the day of the visit,
Personnel of the investigating laboratory.

Protocol Deviations Protocol deviations were not reported during the study.

### Visit 1—Day 0 (Baseline)

Subjects came to the study site. Having the face cleansed with clear water, without soap and without having applied any cosmetic product since previous 48 hours and without using shampoo on the previous evening and in the morning of the visit.
Subjects were acclimatized 15 minutes in the waiting room.
Explanation about study organization and requirements.
The dermatologist investigator fully informed and explained the study to the subjects. After the information session with the dermatologist investigator, the subjects signed the information sheet and consent form in 2 copies. These documents were also signed and dated by the dermatologist investigator. The subjects received each a signed copy.
Verification of inclusion and non-inclusion criteria by the dermatologist investigator.
Dermatological evaluation (assessment of physical and functional signs).
Clinical scoring of Skin Dryness using the Overall dry skin score (ODS) by the dermatologist investigator.
Clinical grading of density and contrast of pigmentary spots by the dermatologist investigator.
Clinical grading of skin redness by the dermatologist investigator.
Clinical grading of tactile roughness, skin suppleness, skin evenness, clarity of skin tone and skin radiance by the dermatologist investigator.
Assessment of the forehead fine lines and wrinkles; and Crow's feet wrinkles by Dermatologist using Skin ageing atlas by the dermatologist investigator.
Assessment of pores by the dermatologist investigator.
Subject evaluation of itch/pruritus severity using the 11-point NRS itch scale.
Evaluation of Skin barrier repair properties by Trans-epidermal water loss measurements—face and forearm.
Evaluation of Skin hydration over using Corneometer CM 825®/MPA 6—face and forearms.
Evaluation of skin redness using the a*parameter of Chromameter over face & forearm.
Digital photography of face using VISIA-CR/DSLR for illustrative purposes.
Distribution of the daily log to the subjects to note any intolerance.
Distribution of the studied product to the subjects who to use it, two times a day on the whole face and forearm (avoiding eye contours).
Self-Assessment Questionnaire for efficacy, safety, and organoleptic properties of test product
Application of the investigational product under Clinical Trial coordinator supervision.

### Visit 2—Day 14 (D14)

Subjects came to the study site. Having the face cleansed with clear water, without soap, and without having applied any cosmetic product since previous evening.
Subjects were acclimatized for 15 minutes in the waiting room.
Verification of the daily log and the investigational product.
Verification of adverse events, local intolerance, and concomitant medication by the dermatologist.
Dermatological evaluation (assessment of physical and functional signs).
Clinical scoring of Skin Dryness using the Overall dry skin score (ODS) by the dermatologist investigator
Clinical grading of density and contrast of pigmentary spots by the dermatologist investigator
Clinical grading of skin redness by the dermatologist investigator
Clinical grading of tactile roughness, skin suppleness, skin evenness, clarity of skin tone and skin radiance by the dermatologist investigator
Assessment of the forehead fine lines and wrinkles; and Crow's feet wrinkles by Dermatologist using Skin ageing atlas by the dermatologist investigator
Assessment of pores by the dermatologist investigator
Self-Assessment Questionnaire for efficacy, safety, and organoleptic properties of test product
Subject evaluation of itch/pruritus severity using the 11-point NRS itch scale
Evaluation of Skin barrier repair properties by Trans-epidermal water loss measurements—face and forearm
Evaluation of Skin hydration over using Corneometer CM 825®/MPA 6—face and forearms
Evaluation of skin redness using the a*parameter of Chromameter over face & forearm
Digital photography of face using VISIA-CR/DSLR for illustrative purposes.

### Visit 3—Day 28 (D28)

Subjects came to the study site. Having the face cleansed with clear water, without soap, and without having applied any cosmetic product since previous evening.
Subjects were acclimatized for 15 minutes in the waiting room.
Verification of the daily log and the investigational product.
Verification of adverse events, local intolerance, and concomitant medication by the dermatologist.
Dermatological evaluation (assessment of physical and functional signs).
Clinical scoring of Skin Dryness using the Overall dry skin score (ODS) by the dermatologist investigator
Clinical grading of density and contrast of pigmentary spots by the dermatologist investigator
Clinical grading of skin redness by the dermatologist investigator
Clinical grading of tactile roughness, skin suppleness, skin evenness, clarity of skin tone and skin radiance by the dermatologist investigator
Assessment of the forehead fine lines and wrinkles; and Crow's feet wrinkles by Dermatologist using Skin ageing atlas by the dermatologist investigator
Assessment of pores by the dermatologist investigator
Self-Assessment Questionnaire for efficacy, safety, and organoleptic properties of test product
Subject evaluation of itch/pruritus severity using the 11-point NRS itch scale
Evaluation of Skin barrier repair properties by Trans-epidermal water loss measurements—face and forearm
Evaluation of Skin hydration over using Corneometer CM 825®/MPA 6—face and forearms
Evaluation of skin redness using the a*parameter of Chromameter over face & forearm
Digital photography of face using VISIA-CR/DSLR for illustrative purposes.

### Visit 4—Day 56 (D56)

Subjects came to the study site. Having the face cleansed with clear water, without soap, and without having applied any cosmetic product since the previous evening.
Subjects were acclimatized for 15 minutes in the waiting room.
Verification of the daily log and the investigational product.
Verification of adverse events, local intolerance, and concomitant medication by the dermatologist.
Dermatological evaluation (assessment of physical and functional signs).
Clinical scoring of Skin Dryness using the Overall dry skin score (ODS) by the dermatologist investigator
Clinical grading of density and contrast of pigmentary spots by the dermatologist investigator
Clinical grading of skin redness by the dermatologist investigator
Clinical grading of tactile roughness, skin suppleness, skin evenness, clarity of skin tone and skin radiance by the dermatologist investigator
Assessment of the forehead fine lines and wrinkles; and Crow's feet wrinkles by Dermatologist using Skin ageing atlas by the dermatologist investigator
Assessment of pores by the dermatologist investigator
Self-Assessment Questionnaire for efficacy, safety, and organoleptic properties of test product
Subject evaluation of itch/pruritus severity using the 11-point NRS itch scale
Evaluation of Skin barrier repair properties by Trans-epidermal water loss measurements—face and forearm
Evaluation of Skin hydration over using Corneometer CM 825®/MPA 6—face and forearms
Evaluation of skin redness using the a*parameter of Chromameter over face & forearm
Digital photography of face using VISIA-CR/DSLR for illustrative purposes.

### 11. Results

### 11.1. Population Description

66 female and male subjects were included in the study.

The analyzed panel consisted of:
63 Caucasian female and male: 61 female subjects and 2 male subjects
Aged between 37 to 65 years old (mean age: 51±1 years)

| | | | | | | | Normal | |
|---|---|---|---|---|---|---|---|---|
| | | | | Skin | | | study | |
| Subject ID | Age | Gender | Ethnicity | type | Date of visit | End date | completion | Comments |
| CIDP-ROM-0001 | 62 | F | C | VD | 20 Mar. 2023 | 15 May 2023 | Yes | — |
| CIDP-ROM-0002 | 51 | F | C | VD | 20 Mar. 2023 | 15 May 2023 | Yes | — |
| CIDP-ROM-0003 | 52 | F | C | VD | 20 Mar. 2023 | 15 May 2023 | Yes | — |
| CIDP-ROM-0004 | 42 | F | C | VD | 20 Mar. 2023 | 15 May 2023 | Yes | — |
| CIDP-ROM-0005 | 38 | F | C | VD | 20 Mar. 2023 | 15 May 2023 | Yes | — |
| CIDP-ROM-0006 | 54 | F | C | VD | 20 Mar. 2023 | 15 May 2023 | Yes | — |
| CIDP-ROM-0007 | 65 | F | C | VD | 20 Mar. 2023 | 15 May 2023 | Yes | — |
| CIDP-ROM-0008 | 58 | F | C | VD | 21 Mar. 2023 | 15 May 2023 | Yes | — |
| CIDP-ROM-0009 | 60 | F | C | VD | 21 Mar. 2023 | 17 May 2023 | Yes | — |
| CIDP-ROM-0010 | 55 | F | C | VD | 21 Mar. 2023 | 15 May 2023 | Yes | — |
| CIDP-ROM-0011 | 51 | F | C | VD | 21 Mar. 2023 | 15 May 2023 | Yes | — |
| CIDP-ROM-0012 | 49 | F | C | VD | 27 Mar. 2023 | 22 May 2023 | Yes | — |
| CIDP-ROM-0013 | 57 | F | C | VD | 27 Mar. 2023 | 22 May 2023 | Yes | — |
| CIDP-ROM-0014 | 60 | F | C | VD | 27 Mar. 2023 | 22 May 2023 | Yes | — |
| CIDP-ROM-0015 | 54 | F | C | VD | 27 Mar. 2023 | 22 May 2023 | Yes | — |
| CIDP-ROM-0016 | 45 | F | C | VD | 28 Mar. 2023 | 22 May 2023 | Yes | — |
| CIDP-ROM-0017 | 49 | F | C | VD | 28 Mar. 2023 | 22 May 2023 | Yes | — |
| CIDP-ROM-0019 | 58 | F | C | VD | 28 Mar. 2023 | 22 May 2023 | Yes | — |
| CIDP-ROM-0020 | 45 | F | C | VD | 28 Mar. 2023 | 22 May 2023 | Yes | — |
| CIDP-ROM-0021 | 52 | F | C | VD | 3 Apr. 2023 | 29 May 2023 | Yes | — |
| CIDP-ROM-0022 | 55 | F | C | VD | 3 Apr. 2023 | 29 May 2023 | Yes | — |
| CIDP-ROM-0023 | 52 | F | C | VD | 3 Apr. 2023 | 29 May 2023 | Yes | — |
| CIDP-ROM-0024 | 55 | F | C | VD | 3 Apr. 2023 | 29 May 2023 | Yes | — |
| CIDP-ROM-0025 | 64 | F | C | VD | 3 Apr. 2023 | 29 May 2023 | Yes | — |
| CIDP-ROM-0026 | 54 | F | C | VD | 3 Apr. 2023 | 29 May 2023 | Yes | — |
| CIDP-ROM-0027 | 41 | F | C | VD | 3 Apr. 2023 | 29 May 2023 | Yes | — |
| CIDP-ROM-0028 | 45 | F | C | VD | 4 Apr. 2023 | 30 May 2023 | Yes | — |
| CIDP-ROM-0029 | 40 | F | C | VD | 4 Apr. 2023 | 30 May 2023 | Yes | — |
| CIDP-ROM-0030 | 64 | F | C | VD | 4 Apr. 2023 | 30 May 2023 | Yes | — |
| CIDP-ROM-0031 | 41 | F | C | VD | 4 Apr. 2023 | 30 May 2023 | Yes | — |
| CIDP-ROM-0032 | 58 | F | C | VD | 4 Apr. 2023 | 30 May 2023 | Yes | — |
| CIDP-ROM-0033 | 52 | F | C | VD | 4 Apr. 2023 | 30 May 2023 | Yes | — |
| CIDP-ROM-0034 | 55 | F | C | VD | 4 Apr. 2023 | 30 May 2023 | Yes | — |
| CIDP-ROM-0035 | 64 | F | C | VD | 4 Apr. 2023 | 30 May 2023 | Yes | — |
| CIDP-ROM-0036 | 44 | F | C | VD | 10 Apr. 2023 | 6 Jun. 2023 | Yes | — |
| CIDP-ROM-0037 | 59 | F | C | VD | 10 Apr. 2023 | 6 Jun. 2023 | Yes | — |
| CIDP-ROM-0038 | 65 | F | C | VD | 10 Apr. 2023 | 6 Jun. 2023 | Yes | — |
| CIDP-ROM-0039 | 50 | F | C | VD | 10 Apr. 2023 | 6 Jun. 2023 | Yes | — |
| CIDP-ROM-0040 | 43 | F | C | VD | 10 Apr. 2023 | 6 Jun. 2023 | Yes | — |
| CIDP-ROM-0041 | 45 | F | C | VD | 10 Apr. 2023 | 6 Jun. 2023 | Yes | — |
| CIDP-ROM-0042 | 49 | F | C | VD | 10 Apr. 2023 | 6 Jun. 2023 | Yes | — |
| CIDP-ROM-0043 | 46 | F | C | VD | 10 Apr. 2023 | 7 Jun. 2023 | Yes | — |
| CIDP-ROM-0044 | 44 | F | C | VD | 10 Apr. 2023 | 7 Jun. 2023 | Yes | — |
| CIDP-ROM-0046 | 62 | F | C | VD | 11 Apr. 2023 | 6 Jun. 2023 | Yes | — |
| CIDP-ROM-0047 | 63 | F | C | VD | 11 Apr. 2023 | 6 Jun. 2023 | Yes | — |
| CIDP-ROM-0048 | 45 | F | C | VD | 11 Apr. 2023 | 6 Jun. 2023 | Yes | — |
| CIDP-ROM-0049 | 48 | F | C | VD | 18 Apr. 2023 | 13 Jun. 2023 | Yes | — |
| CIDP-ROM-0050 | 65 | F | C | VD | 18 Apr. 2023 | 13 Jun. 2023 | Yes | — |
| CIDP-ROM-0051 | 51 | F | C | VD | 18 Apr. 2023 | 13 Jun. 2023 | Yes | — |
| CIDP-ROM-0052 | 50 | F | C | VD | 24 Apr. 2023 | 20 Jun. 2023 | Yes | — |
| CIDP-ROM-0053 | 56 | F | C | VD | 24 Apr. 2023 | 20 Jun. 2023 | Yes | — |
| CIDP-ROM-0054 | 37 | F | C | VD | 24 Apr. 2023 | 20 Jun. 2023 | Yes | — |
| CIDP-ROM-0055 | 43 | F | C | VD | 25 Apr. 2023 | 20 Jun. 2023 | Yes | — |
| CIDP-ROM-0056 | 46 | M | C | VD | 25 Apr. 2023 | 20 Jun. 2023 | Yes | — |
| CIDP-ROM-0057 | 51 | F | C | VD | 25 Apr. 2023 | 20 Jun. 2023 | Yes | — |
| CIDP-ROM-0058 | 55 | F | C | VD | 25 Apr. 2023 | 20 Jun. 2023 | Yes | — |
| CIDP-ROM-0059 | 39 | M | C | VD | 25 Apr. 2023 | 20 Jun. 2023 | Yes | — |
| CIDP-ROM-0061 | 39 | F | C | VD | 2 May 2023 | 27 Jun. 2023 | Yes | — |
| CIDP-ROM-0062 | 39 | F | C | VD | 2 May 2023 | 28 Jun. 2023 | Yes | — |
| CIDP-ROM-0063 | 43 | F | C | VD | 2 May 2023 | 27 Jun. 2023 | Yes | — |
| CIDP-ROM-0064 | 57 | F | C | VD | 3 May 2023 | 28 Jun. 2023 | Yes | — |
| CIDP-ROM-0065 | 40 | F | C | VD | 3 May 2023 | 27 Jun. 2023 | Yes | — |
| CIDP-ROM-0066 | 48 | F | C | VD | 3 May 2023 | 27 Jun. 2023 | Yes | — |
| (CIDP-ROM-0018)* | (55)* | (F)* | (C)* | (VD)* | (28 Mar. 2023)* | (19 Jun. 2023)* | (No)* | (acute bronchitis)* |
| (CIDP-ROM-0045)* | (47)* | (F)* | (C)* | (VD)* | (11 Apr. 2023)* | (19 Jun. 2023)* | (No)* | (ACUTE BRONCHITIS)* |
| (CIDP-ROM-0060)* | (39)* | (F)* | (C)* | (VD)* | (25 Apr. 2023)* | (20 Jun. 2023)* | (No)* | (Lost-to follow up)* |

### Legend:

(*) values not included in data analysis
Gender:
   F: Female subject
   M: Male subject
Type of skin:
   VD: Very dry
Ethnicity:
   C: Caucasian

### Descriptive Statistics:

### Age

Mean: 51
Median: 51
Minimum: 37
Maximum: 65
SD: 8
SEM: 1
95% CI: 2

### Gender

Female: 61→97%
Male: 2→3%
TOTAL: 63→100.00%

### Ethnicity

Caucasian: 63→100%
Mixed race: 0→0%
Indian: 0→0%
Asian: 0→0%
African: 0→0%
TOTAL: 63→100.00%

### Skin Type

Normal: 0→0%
Combination: 0→0%
Dry: 0→0%
Very dry: 63→100%
Oily: 0→0%
TOTAL: 63→100.00%

### Normal Study Termination

Yes: 63→95%
No: 3→5%
TOTAL: 66→100.00%

### Additional Testing Data

### Study: Consumer Perception Study on SPF Protection and Skin Benefits of Silk Anti-Aging Lotion

### Participants

A total of 30 male and female participants aged 18 or over were recruited for this study.

All participants satisfied the following inclusion and exclusion criteria.

Inclusion criteria
Male or female
Aged 18 years and older
Have concerns about signs of skin hydration and facial appearance, including:
   Skin dryness
   Uneven skin tone
   Unhealthy skin tone
   Dull skin
   Skin aging
   Skin inflammation
   Large pores
   Skin redness
   Skin protection
Willing to use the product as directed and comply with the study protocol
Have been using the same skincare routine for at least one month prior to the study
Willing to stop using current SPF cream for the duration of the study period
Access to a smartphone or computer for virtual participation
Willing to maintain the same skincare routine and products throughout the 30-day trial (e.g., moisturizer and cleanser)
If taking medications, oral supplements, or herbal remedies targeted at skin health and appearance, have been consistently taking these for at least 3 months prior to starting the study, and are willing to maintain this routine for the study duration
Agree to avoid using any other products or new forms of regular medication or supplements that target skin health and appearance
Not planning to undergo facial treatments during the study period, including botox, dermal filler, or chemical peels

### Exclusion Criteria:

Have known allergies to any ingredients in the study product: Zinc oxide (19.3%), Caprylic/capric triglyceride, Water, Mauritia Flexuosa Fruit Oil, Coffee Arabica (Coffee) Seed Oil, Polyglyceryl-2-Dipolyhydroxystearate, Glycerin, Silica, Argania Spinosa Kernel Oil, Tocopheryl Acetate, Polyglyceryl-3-Diisostearate, Oryza Sativa (Rice) Bran Wax, Polyhydroxystearic Acid, Xylityl Sesquicaprylate, Jojoba Esters, Physalis Angulata Extract, Hydrolyzed Fibroin, Tocopherol, Arginine, Hydrogenated Vegetable Glycerides, Xanthan Gum, Magnesium Sulfate, Phytic Acid, Anhydroxylitol, Iron Oxides (CI 77492), Stearoyl Glutamic Acid, Caprylyl Glycol, Bisabolol
Anyone with any known serious allergic reactions that require the use of an Epi-Pen
Anyone with any chronic skin conditions on the face, including eczema or psoriasis
Anyone with a history of skin cancer or precancerous skin lesions on the face
Anyone currently pregnant, trying to conceive, or breastfeeding
Anyone currently testing or planning to in the next 30 days participate in another clinical study
Have any chronic medical conditions like oncological or psychiatric disorders that might interfere with the study or pose a risk to the participant
Anyone with a history of substance abuse
Anyone who is currently, or has been in the past 3 months, a smoker
Anyone who resides in the United States

### Study Design

This virtual consumer perception study required participants to complete questionnaires at home. Before study onboarding, consent forms describing the study process, instructions, evaluation methods, and bill of rights were provided to participants. Following the consent process, participants completed the Baseline questionnaire.

All participants were asked to use the SilkLyfe Daily Defense Sunscreen+SPF 28.

Throughout the study, participants were required to apply the product daily in the morning before going outdoors to assess its impact on skin hydration and appearance. Study-specific questionnaires were completed at Baseline, First Use, Day 15, and Day 30.

### Data Analysis or Data Reporting

In this study, participant responses were collected using a 5-point Likert scale, ranging from ‘strongly agree’ to ‘strongly disagree.’ For the purposes of analysis, responses indicating ‘agree’ and ‘strongly agree’ were combined to create a composite ‘agree’ score. This combined percentage was used to assess the overall agreement among participants, providing a clear measure of positive sentiment toward the variables under investigation.

### Results

A total of 30 participants were recruited for this study. Three participants did not complete the study, so data from 27 participants were included in the final analysis. The participants were primarily white and female. The average age of the participants was 36.5 yrs. Participant demographics are presented in Table 1.

At Baseline, participants were asked questions about the severity of their skin issues. These questions were then asked on Day 15 and Day 30. Reductions in the participants' perception of skin issues were observed in all 10 parameters by Day 30. In particular, participants showed an 84.67% reduction in the combined agree score for the parameter “my skin looks dull” and a 73.54% reduction in the combined agree score for the parameter “my skin feels dry” (FIG. 1, Table 2).

Further questions were asked about improvements after using the test products for the First time on Day 15 and Day 30. After the First use, notable scores were observed in participant perception of the product's ability to instantly moisturize (82.76%), protect (72.41%), nourish (68.97%), and hydrate/moisturize (75.86%) the skin after using the test product.

On Days 15 and 30, the product was further well-received. Twenty of 27 parameters showed a combined agree score of >65% by Day 30. Most notable was 100% of participants who agreed that their skin felt more protected, 96.3% who agreed that their skin felt more hydrated, moisturized, and softer, and 88.89% who agreed that their skin felt more nourished. The product also indicated good safety, with many participants agreeing that the product does not cause breakouts, stinging, or irritation. Furthermore, participants agreed that the product was better than other SPF creams (66.67%), that they wanted to continue using the product (77.78%), and that they would recommend the product to their friends and family (77.78%) (Table 2).

### Gender

Male: 2 participants→7.4%
Female: 25 participants→83.3%

### Race

Hispanic or Latino: 2 participants→7.4%
Black or African American: 0 participants→0%
White: 22 participants→81.5%
Asian: 2 participants→7.4%
American Indian or Alaska Native: 1 participant→3.7%

### Age (Years)

Median Age: 37.9
Mean Age: 38.5
Range: 30-70

| | Base- | First | | | Relative |
|---|---|---|---|---|---|
| Statement | line | Use | Day 15 | Day 30 | % Change |
| My skin feels dry. | 70.00% | 18.52% | 18.52% | 18.52% | −73.54 |
| My skin looks dull. | 90.67% | 14.81% | 14.81% | 14.81% | −84.67 |
| I have an uneven | 68.67% | 29.63% | 37.04% | 37.04% | −44.44 |
| skin tone. | | | | | |
| I have poor skin | 83.33% | 22.22% | 18.52% | 18.52% | −70.78 |
| texture. | | | | | |
| I have noticeable | 78.67% | 55.56% | 29.63% | 29.63% | −81.35 |
| fine lines and | | | | | |
| wrinkles on my | | | | | |
| face. | | | | | |
| My skin often feels | 33.33% | 18.52% | 18.52% | 18.52% | −44.44 |
| inflamed. | | | | | |
| I have noticeably | 70.00% | 40.74% | 44.44% | 44.44% | −38.51 |
| large pores on my | | | | | |
| face. | | | | | |
| My skin often | 83.33% | 29.63% | 22.22% | 22.22% | −84.91 |
| looks red. | | | | | |
| My skin looks | 43.33% | 18.52% | 7.41% | 7.41% | −82.91 |
| unhealthy. | | | | | |
| I have noticeable | 70.00% | 37.04% | 44.44% | 44.44% | −38.51 |
| blemishes on my | | | | | |
| skin. | | | | | |
| The test product | 82.76% | 88.89% | 100.00% | — | |
| instantly | | | | | |
| moisturizes my | | | | | |
| skin. | | | | | |
| My skin feels more | 72.41% | 85.19% | 85.19% | — | |
| protected since | | | | | |
| using the test | | | | | |
| product. | | | | | |
| My skin feels more | 68.97% | 92.59% | 88.89% | — | |
| nourished since | | | | | |
| using the test | | | | | |
| product. | | | | | |
| My skin feels | 58.62% | 66.67% | 70.37% | — | |
| rejuvenated since | | | | | |
| using the test | | | | | |
| product. | | | | | |
| My skin feels more | 75.86% | 85.19% | 96.30% | — | |
| hydrated and | | | | | |
| moisturized since | | | | | |
| using the test | | | | | |
| product. | | | | | |
| My skin feels more | 58.62% | 62.96% | 77.78% | — | |
| radiant since using | | | | | |
| the test product. | | | | | |
| My skin feels satin | 62.07% | 77.78% | 62.96% | — | |
| smooth since using | | | | | |
| the test product. | | | | | |
| My skin feels more | 51.72% | 74.07% | 81.48% | — | |
| supple since using | | | | | |
| the test product. | | | | | |
| My skin feels | 55.17% | 81.48% | 96.30% | — | |
| softer since using | | | | | |
| the test product. | | | | | |
| My fine lines and | 10.34% | 25.93% | 44.44% | — | |
| wrinkles have | | | | | |
| reduced since | | | | | |
| using the test | | | | | |
| product. | | | | | |
| My skin | 20.69% | 29.63% | 48.15% | — | |
| inflammation has | | | | | |
| reduced since | | | | | |
| using the test | | | | | |
| product. | | | | | |
| I have fewer large | 20.69% | 29.63% | 48.15% | — | |
| pores on my face | | | | | |
| since using the test | | | | | |
| product. | | | | | |
| My skin redness | 24.14% | 37.04% | 59.26% | — | |
| has reduced since | | | | | |
| using the test | | | | | |
| product. | | | | | |
| My skin feels | 48.28% | 70.37% | 81.48% | — | |
| healthier since | | | | | |
| using the test | | | | | |
| product. | | | | | |
| My blemishes have | 17.24% | 37.04% | 37.04% | — | |
| been reduced since | | | | | |
| using the test | | | | | |
| product. | | | | | |
| The look and feel | 34.48% | 70.37% | 81.48% | — | |
| of my skin has | | | | | |
| improved since | | | | | |
| using the test | | | | | |
| product. | | | | | |
| My skin has more | 55.17% | 74.07% | 81.48% | — | |
| of a glow since | | | | | |
| using the test | | | | | |
| product. | | | | | |
| My skin feels more | 24.14% | 55.56% | 74.07% | — | |
| elastic and firm | | | | | |
| since using the test | | | | | |
| product. | | | | | |
| My skin has a | 24.14% | 40.74% | 55.56% | — | |
| more even tone | | | | | |
| since using the test | | | | | |
| product. | | | | | |
| My skin looks | 31.03% | 44.44% | 66.67% | — | |
| more youthful | | | | | |
| since using the test | | | | | |
| product. | | | | | |
| The test product | 37.93% | 66.67% | 85.19% | — | |
| improved the look | | | | | |
| and feel of my | | | | | |
| skin. | | | | | |
| The test product | 37.93% | 55.56% | 74.07% | — | |
| gave me visibly | | | | | |
| healthier skin. | | | | | |
| This product does | — | — | 81.48% | — | |
| not cause | | | | | |
| breakouts. | | | | | |
| This product does | — | — | 92.59% | — | |
| not cause stinging | | | | | |
| or irritation. | | | | | |
| This product is | — | — | 66.67% | — | |
| better than other | | | | | |
| SPF creams that I | | | | | |
| have used. | | | | | |
| I want to continue | — | — | 77.78% | — | |
| using this product. | | | | | |
| I would | — | — | 77.78% | — | |
| recommend this | | | | | |
| product to my | | | | | |
| friends and family. | | | | | |

### Additional Testing Data: Assessment of the Skin Primary and Cumulative Irritation Potential and Skin Sensitization Potential of a Product, Under Controlled and Maximized Conditions (HRIPT)

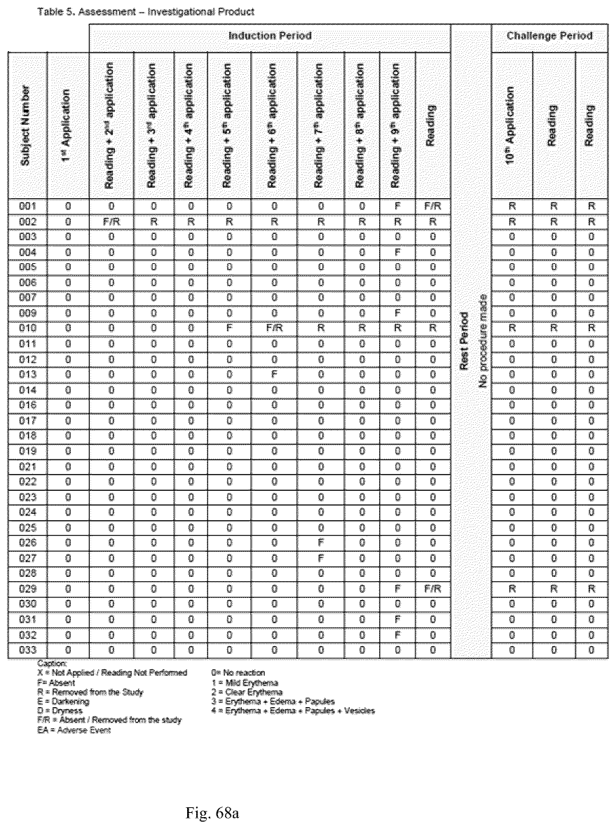

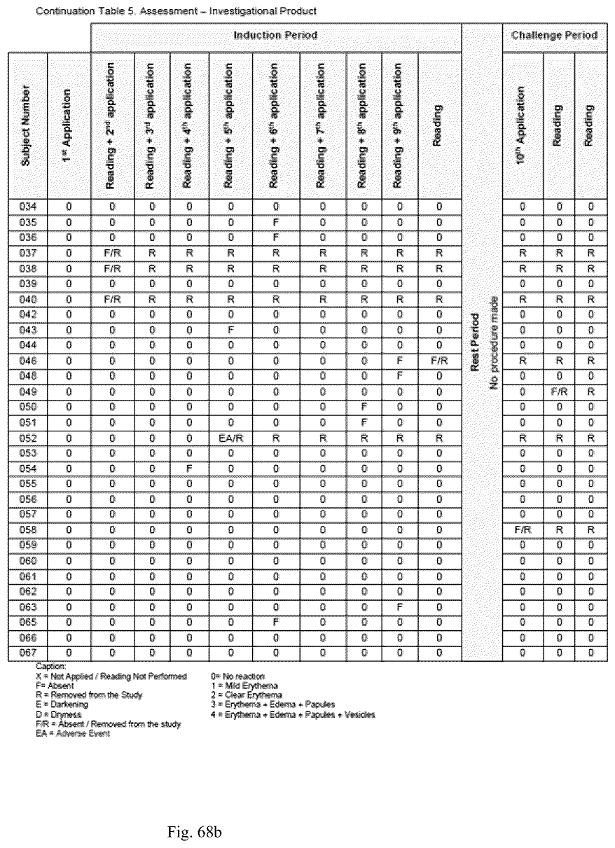

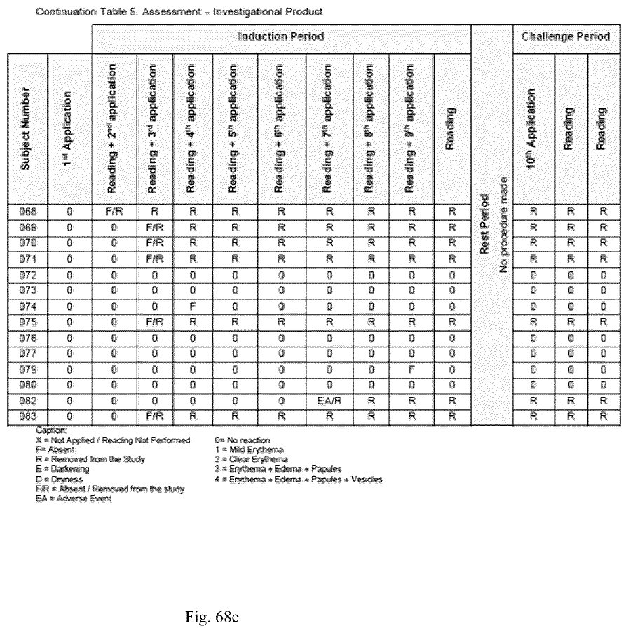

Investigational Product: Silk Soothing Lotion (CAS-009) (as seen in FIGS. 68a-68c)

Methodology: Both the test-product and control were applied to patch test filter paper discs and, then, attached to the right or left back (scapular area) of the study subjects.

Induction Period: The applications were performed during 3 consecutive weeks. Forty-eight hours (48h), or 72h (on weekends) after the application, the product was removed by expert technicians and the site was assessed in order to check the presence of possible clinical signs.

Rest Period: After the induction there was a minimum 10 day-period when no product was applied to the study subjects' dorsum.

Challenge Test: Then, the challenge period started. One single application was made, followed by readings after 48h and 72h of it being attached to the subject's dorsum.

The dermatological clinical assessment was made in the beginning and end of the study and the subjects were supervised by a dermatologist throughout the study.

### Investigational Product

### Legend

X=Not Applied/Reading Not Performed
F=Absent
R=Removed from the Study
E=Darkening
D=Dryness
F/R=Absent/Removed from the study
EA=Adverse Event

### Numeric Scale:

0=No reaction
1=Mild Erythema
2=Clear Erythema
3=Erythema+Edema+Papules
4=Erythema+Edema+Papules+Vesicles

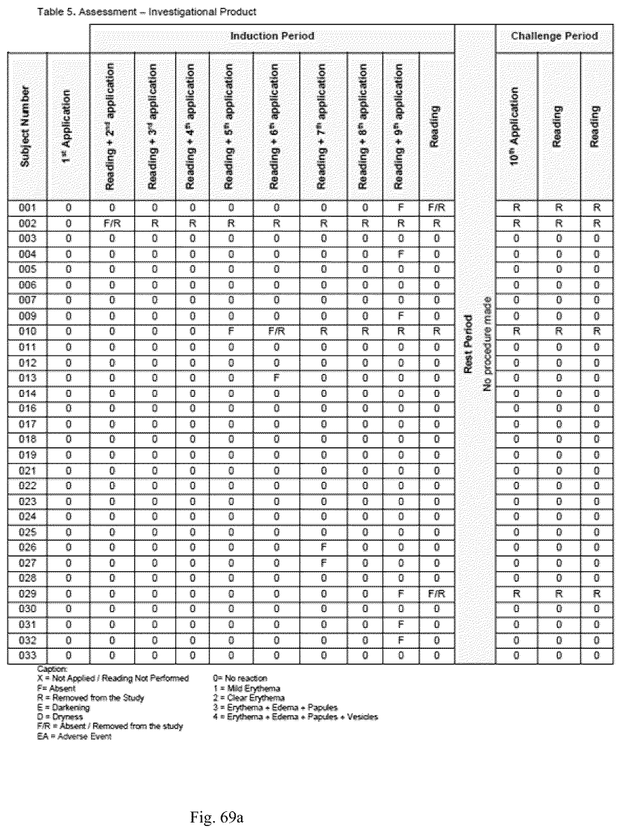

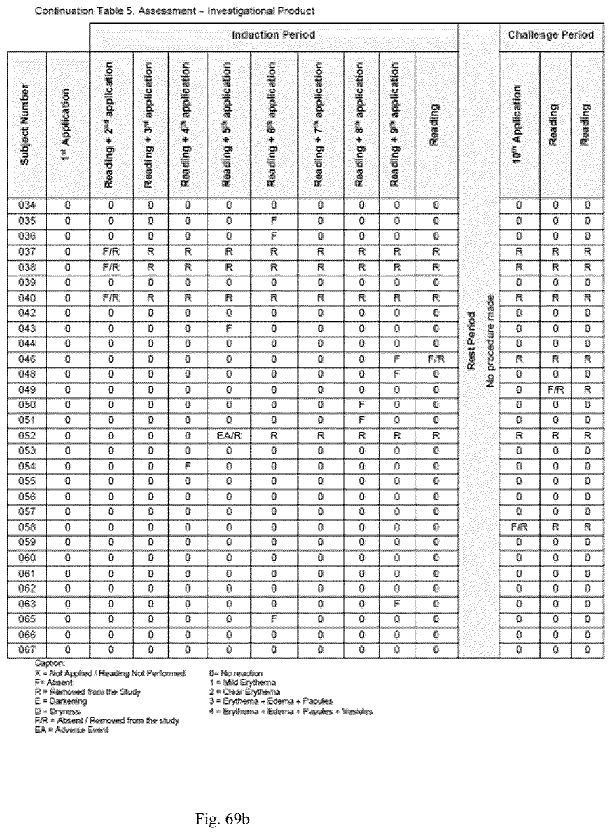

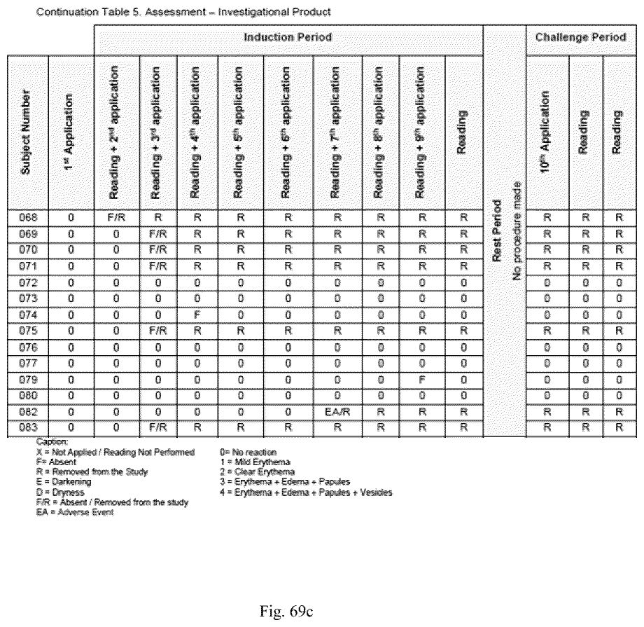

Investigational Product: Silk Face Serum (#CFS-021) (as seen in FIGS. 69a-69c)

Methodology: Both the test-product and control were applied to patch test filter paper discs and, then, attached to the right or left back (scapular area) of the study subjects.

Induction Period: The applications were performed during 3 consecutive weeks. Forty-eight hours (48h), or 72h (on weekends) after the application, the product was removed by expert technicians and the site was assessed in order to check the presence of possible clinical signs.

Rest Period: After the induction there was a minimum 10 day-period when no product was applied to the study subjects' dorsum.

Challenge Test: Then, the challenge period started. One single application was made, followed by readings after 48h and 72h of it being attached to the subject's dorsum.

The dermatological clinical assessment was made in the beginning and end of the study and the subjects were supervised by a dermatologist throughout the study.

### Legend

X=Not Applied/Reading Not Performed
F=Absent
R=Removed from the Study
E=Darkening
D=Dryness
F/R=Absent/Removed from the study
EA=Adverse Event

### Numeric Scale:

0=No reaction
1=Mild Erythema
2=Clear Erythema
3=Erythema+Edema+Papules
4=Erythema+Edema+Papules+Vesicles

### HRIPT Testing for Anti-Ageing/SPF

### 50 Human Subject Repeat Insult Patch Test Skin Irritation/Sensitization Evaluation (Semi-Occlusive Patch)

### 1.0 Objective

Consumer products or raw materials designed for consistent reapplication to areas of the skin may, under proper conditions, prove to be contact sensitizers and/or irritants in certain individuals. It is the intention of a Repeat Insult Patch Test (RIPT) to provide a basis for evaluation of this irritation/sensitization potential if such exists.

### 2.0 Test Material

### 2.1 Test Material Description

On Oct. 11, 2023 one test sample labeled FR #14-31SLK SilkLyfe Anti-Aging Mineral SPF 25 Face Lotion/OTC was received from Dermacare Research Labs, LLC and assigned ADVANCED Lab No.: A-1545.

### 2.2 Handling

Upon arrival at Advanced Science Laboratories, Inc., the test material is assigned a unique laboratory code number and entered into a daily log identifying the lot number, sample description, sponsor, date received and tests requested.

Samples are retained for a period of three months beyond submission of final report unless otherwise specified by the sponsor or, if sample is known to be in support of governmental applications, representative retained samples are kept two years beyond final report submission.

Sample disposal is conducted in compliance with appropriate federal, state and local ordinances.

### 2.3 Test Material Evaluation Prerequisite

Prior to induction of a human test panel, toxicology, microbiology or in-vitro performance spectra may be required to assess the feasibility of commencement as dictated by an Institutional Review Board (IRB) described in Section 3.0.

Sponsor purports that prior to sample submission the following tests were conducted with no adverse results and that the test data are on file on their premises but may not have been made available to Advanced Science personnel:
USP or CTFA Preservative Efficacy Test or equivalent
90 Day Accelerated Stability and Container Compatibility Study 3.0 Institutional Review Board: [IORG 0011153][IRB00013226]

Reference: CFR Title 21 Part 56, Subparts A, B, C, and D. The IRB of Advanced Science Laboratories Inc. consists of five or more individuals, chosen from within the company for technical expertise and from the local community for lay interaction. The list of IRB members is kept on file at Advance Science Laboratories, Inc. and is available for inspection during the hours of operation.

### 4.0 Panel Selection

### 4.1 Standards for Inclusion in a Study

Individuals free of any dermatological or systemic disorder which would interfere with the results, at the discretion of the Study Director.
Individuals free of any acute or chronic disease that might interfere with or increase the risk of study participation.
Individuals who completed a preliminary medical history form mandated by Advanced Science Laboratories, Inc. and are in general good health.
Individuals, who read, understood and signed an informed consent document relating to the specific type of study they are subscribing. Consent forms are kept on file and are available for examination on the premises of Advanced Science Laboratories, Inc. only.
Individuals able to cooperate with the Study Director and research staff, willing to have test materials applied according to the protocol, and complete the full course of the study.

### 4.2 Standards for Exclusion from a Study

Individuals under 18 years of age.
Individuals who are currently taking any medication (topical or systemic) that may mask or interfere with the test results, at the discretion of the Study Director.
Subjects with a history of any acute or chronic disease that might interfere with or increase the risk associated with study participation.
Individuals diagnosed with chronic skin allergies.
Female volunteers who indicate that they are pregnant or lactating.

### 4.3 Recruitment

Panel selection is accomplished by advertisements in local periodicals, community bulletin boards, phone solicitation, electronic media or any combination thereof.

### 4.4 Informed Consent and Medical History Forms

An informed consent was obtained from each volunteer prior to initiating the study describing reasons for the study, possible adverse effects, associated risks and potential benefits of the treatment and their limits of liability. Panelists signed and dated the informed consent document to indicate their authorization to proceed and acknowledge their understanding of the contents. Each subject was assigned a permanent identification number and completed an extensive medical history form. These forms along with the signed consent forms, are available for inspection on the premises of Advance Science Laboratories, Inc. only. Reference 21 CFR Ch. 1 Part 50, Subpart B.

The parties agreed to comply with applicable state and federal privacy laws for the use and disclosure of a subject's personal health information by taking reasonable steps to protect the confidentiality of this information. This obligation shall survive the termination or expiration of this Agreement.

### 5.0 Population Demographics

Number of subjects enrolled . . . 64
Number of subjects completing study . . . 57
Age Range . . . 21-76
Sex:
   Male . . . 14
   Female . . . 43
Race:
   Caucasian . . . 34
Hispanic . . . 9
Asian . . . 2
African American/Black . . . 11
Mixed . . . 1

### 10.0 Observations

No adverse reactions to this product were noted during the course of this study.

### 11.0 Archiving

All original samples, raw data sheets, technician's notebooks, correspondence files, copies of final reports and remaining specimens are maintained on the premises of Advance Science Laboratories, Inc. in limited access marked storage files for a period not to exceed two years. Sponsors are encouraged to keep all original signed, dated and certified reports. Advanced will not be responsible, and it will not be possible to provide duplicate original hard copies of final reports once the documents leave our premises.

### 12.0 Reference

Appraisal of the Safety of Chemicals in Food, Drugs and Cosmetics, published by The Association of Food and Drug Officials of The United States, 1965 (modified).

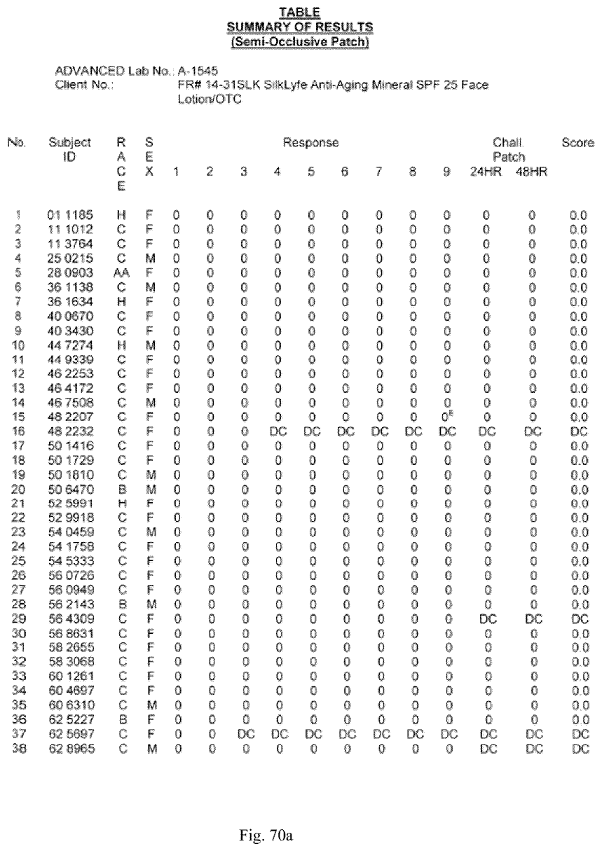

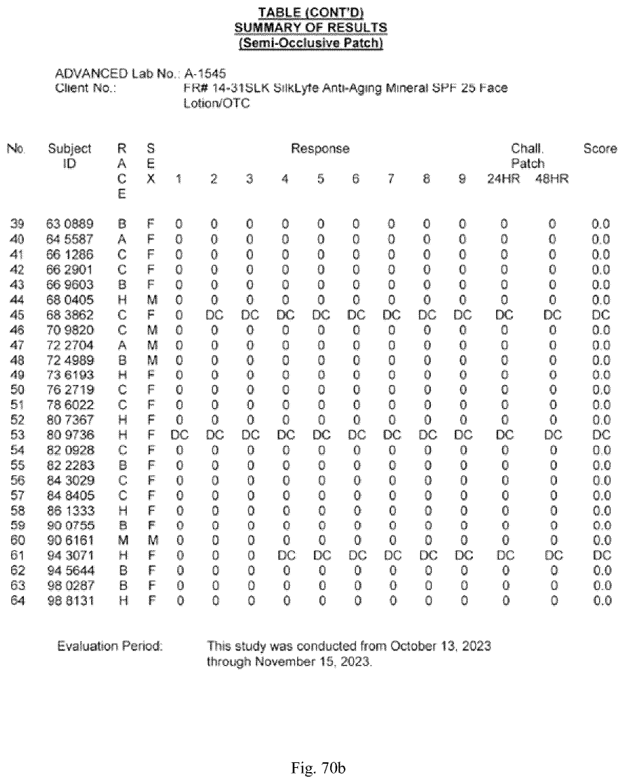

Table—Summary of Results—Semi-Occlusive Patch (as Seen at FIGS. 70a-70b)
Advanced Lab No.: A-1545
Client No. FR #14-31SLK SilkLyfe Anti-Aging Mineral SPF 25 Face Lotion/OTC
Evaluation Period: Oct. 13, 2023-Nov. 15, 2023
Scoring Scale and Definition of Symbols Shown in Table:
0.0—No evidence of any effect
0.5—(Barely perceptible) minimal faint (light pink) uniform or spotty erythema
1.0—(Mild) pink uniform erythema covering most of contact site
2.0—(Moderate) pinkred erythema visibly uniform in entire contact area
3.0—(Marked) bright red erythema with or without petechiae, papules or edema,
4.0—(Severe) deep red erythema with or without vesiculation, weeping or edema
e—accompanying edema
DC—Subject discontinued, unrelated to sample
S—Skin stained from pigment in product, unable to grade reaction
T—Tan, unable to grade reaction
ad—adjacent patch
[ ]^{E}—image of the patch site was electronically transmitted
M—Mixed Heritage
AA—African American
A—Asian
H—Hispanic
B—Black
C—Caucasian

### HRIPT Testing for Silk Body Lotion

### A Modified Draize Repeat Insult Patch Test in a Shared Panel of 106 Healthy Volunteers, to Investigate the Irritation and Sensitisation Potential of 1 Test Article Following Repeated Cutaneous Patch Applications

### 2. Introduction and Objective

The objective of this study was to investigate the irritation and sensitisation potential of 1 cosmetic test article, in a shared panel of 106 healthy volunteers by means of repeated cutaneous occlusive patch applications based on the modified Draize method of Jordan and King (1977)1 to support claims such as “Hypoallergenic”, “Allergy Tested”, “Non-Irritating”, “Clinically Tested”, “Clinically Proven”, “Kind to Skin”, “Mild for Skin”, “Safe for Skin”, “Dermatologically Tested”, “Dermatologist Approved” and “Safe for Sensitive Skin.”

### 3. Study Design

The study was conducted single blind, at a single centre according to Master Protocol: PCRRIP1.

The test article was patched under occlusive conditions using Finn chambers or equivalent occlusive patches. A total of nine inductions patches worn for 47 hours or 71 hours (patching occurred Mondays, Wednesdays, and Fridays) for three weeks (a make-up day was allowed to ensure subjects had all 9 induction patches). Subjects had a rest period of 14 days. Challenge patches were applied for 48 hours, and readings were made 1 hour, and 48 hours post removal.

### 4. Test Materials

### 4.1 Test Articles

The test article was supplied by the Sponsor and labelled as follows:

| | Test Article Name/ | ID Code | Dilution/ |
|---|---|---|---|
| TA# | Description | (Batch/Lot #) | special handling |
| 1 | CFP954 Silklyfe | Lot: LB22-084-24093 | Use as supplied - |
| | Body Lotion | | occlusive patch |

### 5. Study Ethics

### 5.1 Declaration of Helsinki

The study conformed to the requirements of the 1964 Declaration of Helsinki and its subsequent amendments (World Medical Association; 2013)2.

### 5.2 Indemnity Provision

The Sponsor shall be responsible, without regard to legal liability, and shall indemnify PCR Corp, or any of their respective officers or employees in the event of claims for compensation from subjects suffering injury arising out of the administration or use of the test article, or of any procedure required under this protocol as a result of a subject participating in this study, except and insofar as such claims arise as a result of any negligent act or omission on the part of PCR Corp employees or any persons undertaking or involved in the study by arrangement with PCR Corp.

### 5.3 ICH GCP

The study was conducted in accordance with applicable International Council for Harmonization. 2016. Integrated Addendum to ICH E6(R1): Guideline for Good Clinical Practice E6(R2)3 in as much as they apply to cosmetic and consumer product testing/research.

### 9. RESULTS

### 9.1 Location and Dates of the Study

The study was performed at PCR Corp, located in Manchester between w/c 6 May 2024 and w/c 17 Jun. 2024.

### 9.2 Subjects

111 male and female subjects were enrolled into the study. 106 subjects completed the study. The age and gender of these subjects is presented in Appendix 2. 50% of subject panel had self-assessed sensitive skin.

### 9.3 Adverse Events, Adverse Reactions, Subjects not Completing the Study and Deviations

No adverse events or reactions were reported.

5 subjects withdrew for personal reasons.

There were no deviations that occurred during the conduct of the study.

### 9.4 Assessments

Individual reactions to the test article are presented in Appendix 1.

As demonstrated by the individual skin responses to the test article:

Test Article 1—CFP954 Silklyfe Body Lotion Lot: LB22-084-24093 elicited no visible erythematous reactions during the induction phase of the study.

There were no questionable reactions observed during the Challenge Phase (Days 38 and 40) by any of the subjects to the test article. These results support the assessment that under the conditions of the study, the test article have demonstrated a low potential for irritation and sensitization.

### 10. Conclusions

The test article can be considered as safe for use under the conditions of the study, and claims such as, “Hypoallergenic”, “Allergy Tested”, “Non-Irritating”, “Clinically Tested”, “Clinically Proven”, “Kind to Skin”, “Mild for Skin”, “Safe for Skin”, “Dermatologically Tested”, “Dermatologist Approved” and “Safe for Sensitive Skin” are substantiated.

### Test Subject Demographics

| SUBJECT | MALE | | |
|---|---|---|---|
| NUMBER | OR FEMALE | AGE | SKIN TYPE |
| 1 | Female | 38 | Normal |
| 2 | Female | 26 | Normal |
| 3 | Female | 41 | Sensitive |
| 4 | Male | 25 | Sensitive |
| 5 | Female | 28 | Normal |
| 6 | Male | 49 | Sensitive |
| 7 | Female | 52 | Normal |
| 8 | Female | 34 | Normal |
| 9 | Female | 26 | Normal |
| 10 | Female | 31 | Sensitive |
| 11 | Female | 43 | Normal |
| 12 | Female | 37 | Sensitive |
| 13 | Female | 26 | Normal |
| 14 | Male | 29 | Sensitive |
| 15 | Female | 23 | Sensitive |
| 16 | Male | 41 | Normal |
| 17 | Female | 49 | Sensitive |
| 18 | Female | 22 | Sensitive |
| 19 | Male | 27 | Normal |
| 20 | Female | 54 | Sensitive |
| 21 | Female | 28 | Sensitive |
| 22 | Female | 31 | Normal |
| 23 | Female | 29 | Normal |
| 24 | Male | 24 | Sensitive |
| 25 | Male | 27 | Normal |
| 26 | Female | 19 | Sensitive |
| 27 | Female | 20 | Normal |
| 28 | Male | 31 | Sensitive |
| 29 | Female | 31 | Normal |
| 30 | Female | 35 | Sensitive |
| 31 | Male | 49 | Sensitive |
| 32 | Female | 56 | Normal |
| 33 | Female | 38 | Sensitive |
| 34 | Female | 51 | Normal |
| 35 | Female | 29 | Sensitive |
| 36 | Male | 24 | Normal |
| 37 | Male | 27 | Normal |
| 38 | Female | 19 | Sensitive |
| 39 | Female | 23 | Normal |
| 40 | Male | 39 | Sensitive |
| 41 | Female | 48 | Normal |
| 42 | Male | 27 | Sensitive |
| 43 | Female | 48 | Normal |
| 44 | Female | 38 | Sensitive |
| 45 | Female | 55 | Normal |
| 46 | Female | 30 | Normal |
| 47 | Male | 22 | Sensitive |
| 48 | Female | 27 | Normal |
| 49 | Female | 46 | Sensitive |
| 50 | Female | 30 | Sensitive |
| 51 | Female | 28 | Normal |
| 52 | Male | 21 | Sensitive |
| 53 | Male | 42 | Normal |
| 54 | Female | 27 | Sensitive |
| 55 | Male | 33 | Sensitive |
| 56 | Female | 29 | Normal |
| 57 | Female | 20 | Sensitive |
| 58 | Male | 27 | Sensitive |
| 59 | Female | 46 | Normal |
| 60 | Male | 53 | Sensitive |
| 61 | Female | 43 | Sensitive |
| 62 | Female | 22 | Normal |
| 63 | Male | 51 | Normal |
| 64 | Female | 20 | Sensitive |
| 65 | Female | 38 | Normal |
| 66 | Female | 21 | Sensitive |
| 67 | Male | 25 | Normal |
| 68 | Female | 26 | Sensitive |
| 69 | Male | 23 | Normal |
| 70 | Female | 43 | Sensitive |
| 71 | Female | 30 | Normal |
| 72 | Male | 32 | Sensitive |
| 73 | Female | 37 | Normal |
| 74 | Female | 48 | Sensitive |
| 75 | Male | 24 | Normal |
| 76 | Female | 49 | Sensitive |
| 77 | Female | 23 | Normal |
| 78 | Female | 38 | Sensitive |
| 79 | Male | 27 | Normal |
| 80 | Female | 64 | Sensitive |
| 81 | Female | 30 | Normal |
| 82 | Female | 21 | Sensitive |
| 83 | Male | 22 | Normal |
| 84 | Female | 27 | Normal |
| 85 | Female | 40 | Normal |
| 86 | Female | 39 | Sensitive |
| 87 | Male | 26 | Normal |
| 88 | Male | 25 | Sensitive |
| 89 | Female | 36 | Sensitive |
| 90 | Female | 24 | Normal |
| 91 | Male | 29 | Sensitive |
| 92 | Male | 19 | Sensitive |
| 93 | Male | 24 | Sensitive |
| 94 | Female | 29 | Sensitive |
| 95 | Female | 29 | Normal |
| 96 | Female | 34 | Sensitive |
| 97 | Female | 30 | Sensitive |
| 98 | Male | 42 | Normal |
| 99 | Female | 37 | Sensitive |
| 100 | Male | 23 | Normal |
| 101 | Female | 18 | Normal |
| 102 | Female | 56 | Sensitive |
| 103 | Female | 49 | Normal |
| 104 | Male | 33 | Sensitive |
| 105 | Female | 26 | Normal |
| 106 | Male | 28 | Normal |
| 107 | Female | 43 | Sensitive |
| 108 | Female | 39 | Normal |
| 109 | Male | 46 | Sensitive |
| 110 | Female | 33 | Normal |
| 111 | Female | 38 | Sensitive |

### Sun Protection Factor Final Report

### SUMMARY

The sponsor test product sample, Formula #14-31SLK, was tested under FSTI (Florida Suncare Testing, Inc.) SOP #2021-01, Static SPF Testing, and SOP #2021-02 Water Resistant SPF testing as set forth by the FDA, Over the counter monograph M020:Sunscreen Drug Products for Over-the-counter Human use (posted Sep. 24, 2021) as part of the Final Administrative order (OTC000006) effective upon enactment of the Coronavirus Aid, Relief, and Economic Security Act (CARES Act), Public Law 116-136 on Mar. 27, 2020. Part D—Testing Procedure. M020.80 Sun Protection Factor (SPF) test procedure.

The ten (10) subject test panel study yielded a mean Static SPF value of 32.22 (Label SPF 31) and a mean 80 Minute Water Resistant SPF value of 30.14 (Label SPF 28). See Page 9 (Table 1) of this report for a summary of all test results obtained in this study.

The FDA Standard Sunscreen Product tested concurrently with the experimental test sample yielded a mean Static SPF value of 17.80, within the allowable guidelines of 16.3±3.43.

### I. Objective

To measure the Sun Protection Factor (SPF) value “Static” (without water immersion) and also following an 80 Minute water immersion sequence for the sponsor test sample, and the static SPF value for the FDA standard sunscreen under the guidelines of FSTI (Florida Suncare Testing, Inc.) SOPs #2021-01 and 2021-02 using Over the counter monograph M020:Sunscreen Drug Products for Over-the-counter Human use (posted Sep. 24, 2021) as part of the Final Administrative order (OTC000006) effective upon enactment of the Coronavirus Aid, Relief, and Economic Security Act (CARES Act), Public Law 116-136 on Mar. 27, 2020. Part D—Testing Procedure. M020.80 Sun Protection Factor (SPF) test procedure.

### II. Study Type

Ten (10) subject Static and 80 Minute Water Resistant SPF study, with a final report furnished to the sponsor, which includes subject demographics, individual, mean and Label SPF values of the sponsor's test sample.

### III. Sample Description

Silklyfe Anti-Aging Mineral SPF 25 Face Lotion/OTC, Formula #14-31SLK, Lot #5/18/2023, FSTI Sample #23-947

### IV. Test Material Handling

The SBR Labs test sample, labeled Formula #14-31SLK, was assigned Florida Suncare Testing, Inc. sample number 23-947 and entered into the SPF test submission log. The FDA standard 16.3 SPF sunscreen, as described in M020.80 Sun Protection Factor (SPF) test procedure (b) SPF standard, was used concomitantly as the control test product for this study.

### V. Archiving

All original protocols, raw data sheets, and copies of final reports are maintained on the premises of Florida Suncare Testing, Inc., in limited access storage files in accordance with FSTI SOP #2008-10. A duplicate copy of all final reports is kept on a secured, password-protected, Florida Suncare Testing, Inc. computer hard drive and also backed up offsite.

### VI. Panel Design

Number of Subjects enrolled: 10
Number of Subjects completing study: 10
Age Range: 29-71
Male: 4
Female: 6
Skin Types: I (1), II (3), III (6)

### VII. Panel Composition

A. Fair-skinned subjects, male and female, eighteen years of age or older, of skin types I, II, or III as defined in M020.80 Sun Protection Factor (SPF) test procedure.
Type I—Always burns easily; never tans (sensitive)
Type II—Always burns easily; tans minimally (sensitive)
Type III—Burns moderately; tans gradually (normal)

### B. Inclusion Criteria:

1. Individuals eighteen years of age or older.
2. Individuals with fair, uniformly-colored skin on the lower area of the back.
3. Individuals free of any dermatological/systemic disorder that would interfere.
4. Individuals in good health with completed medical history.
5. Individuals who have signed informed consent in compliance with 21 CFR
50.

### Exclusion Criteria:

1. Visible skin disease at the study site.
2. Medications affecting study results.
3. Pregnant/nursing females.
4. History of skin cancer.
5. History of hepatitis/other blood disease.
6. Known sensitivity to cosmetics/skin care/topical drugs.
7. Recent sun exposure on test areas.

### VIII. Informed Consent

Each subject signed informed consent prior to study, describing purpose, risks, benefits, and liability. Medical history completed; subject ID number assigned.

### IX. Light Source

SPF testing followed FDA 2021 guidelines using Solar Light Company Model 16S Single-Port Solar Simulators with 150W xenon arc lamp (290-400 nm spectrum). Max irradiance limit 1500 W/m². Output monitored via Model DCS-2 Dose Control System with Model 2105 UVB detector.

### X. General Testing Procedure

### Day 1—Subject Enrollment

Subjects reported, consent signed, health info provided. Technician examined test area.

### MEDu UV Dose Administration

5 UV doses (J/m²), +25% increments, applied to unprotected skin to determine Minimal Erythemal Dose (MEDu).
Subjects instructed to avoid UV exposure and photosensitizing meds.
Dose Series:
   Dose 1=0.64×
   Dose 2=0.80×
   Dose 3=1.00×
   Dose 4=1.25×
   Dose 5=1.56×

### Day 2—MEDu Determination

Subjects returned 16-24h post-dose.
MED defined as first unambiguous erythema reaction with clear borders.

### Table A—MED Scoring

“−” No perceptible erythema
“?” Barely perceptible erythema
“+” Unambiguous erythema with clear borders
“++” Moderate erythema with sharp borders
“+++” Dark red erythema with sharp borders

### Application of Product for SPF Determination

Three 10 cm×5 cm test areas on back.
Product density: 2 mg/cm².
Applied via finger cot.
Dried 15 minutes before exposure.

### 80 Minute Water Immersion Sequence

1. Jacuzzi at 23-32° C. with clean water.
2. Pool air temp, humidity recorded.
3. Water immersion: 20 min swim+15 min rest, repeated until 80 min achieved.
4. Exposures began post-immersion.

### MEDp UV Dose Administration

5 doses in +15% increments, based on Day 1 MEDu and expected SPF range.
Projected SPF (FDA Standard Static)=16.3
Projected SPF (Test Product Static+80 min Immersion)=25.0

### MEDu Repeat UV Dose Determination

Repeat doses applied on Day 2 in +25% increments.
Multiple of original MEDu (X):
   Dose 1=0.64×
   Dose 2=0.80×
   Dose 3=1.00×
   Dose 4=1.25×
   Dose 5=1.56×

### Day 3—Evaluation of Responses

Subjects returned 16-24h post-Day 2 exposure.
MED scored for all sites.
Double-blinded: evaluators did not know which site/product.

### Conditions:

a. Illumination ≥450 lux fluorescent.
b. Subject seated during evaluation.

### XI. Calculation of SPF Values

### SPF=MEDp/MEDu

Mean SPF and SD calculated.
Label SPF=largest whole number ≤(mean−SD).

### XII. Rejection of Study Data

Reasons: inadequate UV exposure, invalid test data, non-compliance.

### XIII. Results

10 subjects completed study. SPF results in Table 1, Section V.

### XIV. Adverse Experiences

No adverse events reported.

### XV. Conclusions

### SPF Values:

Formula #14-31SLK, FSTI Sample #23-947
Mean Static SPF: 32.22→Label SPF 31
Mean 80 Min Water Resistant SPF: 30.14—Label SPF 28 FDA Standard SPF: 17.80 (within 16.3±3.43 guideline).

What is claimed is:

1. A composition comprising fragments of silk fibroin peptides for use in the treatment of a disorder or condition associated with a skin disorder, wherein the fragments of silk-fibroin peptides have an average molecular weight of less than 90 kDa when measured by exclusion chromatography and less than 20 kDa when measured by dynamic light scattering.

2. The composition for use of claim 1, wherein disorder or condition associated with a skin disorder is selected from the group consisting of hair loss, psoriasis, atopic dermatitis, pain, redness, itchiness, acne, viral infection, wound healing in the cases of intact and/or non-intact skin, anti-aging, and skin whitening.

3. The composition for use of claim 1, where the composition is selected from the group consisting of a hydrogel, powder, suspension, emulsion, foam, film, or solution.

4. The composition for use of claim 1, wherein the composition is a topical composition.

5. The composition for use of claim 1, wherein the fragments of silk fibroin peptides are derived from natural sources, synthetic sources, or a combination thereof.

6. The composition for use of claim 1, wherein the use is in the prophylactic treatment of the disorder or condition associated with a skin disorder.

7. The composition for use of claim 1, wherein the use maintains homeostasis of the skin and/or provides an improvement in the condition of the skin.

8. The composition for use of claim 1, wherein the composition further comprising one or more additional cosmetic and/or pharmaceutical ingredients.

9. The composition for use of claim 8, wherein the one or more additional cosmetic and/or pharmaceutical ingredients are selected from the group consisting of anti-seborrheic agents, anti-acne agents, antioxidants, skin lightening agents, depigmenting agents, anti-wrinkle agents, hair growth agents, sunscreen agents, self-tanning agents, topical analgesics, anti-inflammatory agents, antipruritic agents, deodorants, pigments, water soluble emollients, humectants, stabilizers, fragrances, or the like.

10. The composition for use of claim 1, wherein the viscosity of the composition can be adjusted for multiple use types that include but are not limited to leave on as well as wash off products.

11. The composition for use of claim 10, wherein the viscosity of the composition may have higher or lower shear rates dependent on the desired end use of the composition.

12. The composition for use of claim 1, where the composition can work with multiple skin phototypes including but not limited to, melano-compromised, melano-competent, and melano-protected skin types.

13. The composition for use of claim 1, wherein the composition is applied using a microneedle array.

14. The composition for use of claim 1, wherein the fragments of silk fibroin peptides are encapsulated in nanoparticles to aid in delivery of silk fibroin peptides to the effected site.

15. the composition for use of claim 14, wherein the nanoparticles are selected from the group consisting of solid nanoparticles, semi-solid nanoparticles, and micelle nanoparticles.

16. The composition for use of claim 1, wherein the composition comprises an aqueous phase comprising a therapeutic effective amount of the fragments of silk fibroin peptides.

17. The composition for use of claim 16, wherein the therapeutically effective amount of the fragments of silk fibroin peptides is from about 0.01 pg/kg to about 10 mg/kg based upon the weight of the patient.

18. Use of a composition comprising fragments of silk fibroin peptides to maintain skin health, wherein the fragments of silk-fibroin peptides have an average molecular weight of less than 90 kDa when measured by exclusion chromatography and less than 20 kDa when measured by dynamic light scattering.

19. The use of claim 18 in anti-aging, and/or skin whitening.

20. The use of claim 18, wherein the composition is applied toto skin tissue, such as the face, the including peri-ocular tissues, the head, the scalp, the body, and the extremities.

21. The use of claim 18, wherein the composition is a skin improving or protective silk fibroin peptide composition.

22. A method of treating a disorder or condition associated with a skin disorder in a subject in need thereof, comprising administering to the subject a therapeutically effective amount of a composition comprising fragments of silk-fibroin peptides having an average molecular weight of less than 90 kDa when measured by exclusion chromatography and less than 20 kDa when measured by dynamic light scattering, wherein the composition and the disorder or condition is as defined in claim 1.

23. Use of a composition comprising fragments of silk-fibroin peptides in the manufacture of a medicament for use in the treatment of a disorder or condition associated with a skin disorder, wherein the fragments of silk-fibroin peptides have an average molecular weight of less than 90 kDa when measured by exclusion chromatography and less than 20 kDa when measured by dynamic light scattering, and the composition and the disorder or condition is as defined in claim 1.

24. The composition of claim 1 wherein the silk-fibroin peptides have an average polydispersity of less than 1.4 as determined by dynamic light scattering.
